(19) United States
(12) Patent Application Publication
(10) Pub. No.: US 2014/0357499 A1
(43) Pub. Date: Dec. 4, 2014

(54) METHODS OF LOW ERROR AMPLICON SEQUENCING (LEA-Seq) AND THE USE THEREOF

(71) Applicant: Washington University, St. Louis, MO (US)

(72) Inventors: Jeffrey I. Gordon, St. Louis, MO (US); Jeremiah J. Faith, St. Louis, MO (US)

(21) Appl. No.: 14/292,403

(22) Filed: May 30, 2014

Related U.S. Application Data
(60) Provisional application No. 61/829,206, filed on May 30, 2013.

(51) Int. Cl. C12Q 1/68; C12N 15/10

(52) U.S. Cl. CPC C12Q 1/6869; C12Q 1/689; C12N 15/1065; C12Q 2600/156
USPC 506/2

(57) ABSTRACT

This invention is related to nucleic acid sequencing. In particular, the invention relates to manipulative and analytic steps for analyzing and verifying the products of low frequency events.

### CROSS REFERENCE TO RELATED APPLICATIONS

This application claims the priority of U.S. provisional application No. 61/829,206, filed May 30, 2013, which is hereby incorporated by reference in its entirety.

### GOVERNMENTAL RIGHTS

This invention was made with government support under DK30292, DK078669, DK70977, DK64774 and UL1TR000040 awarded by the NIH. The government has certain rights in the invention.

### FIELD OF THE INVENTION

This invention is related to nucleic acid sequencing. In particular, the invention relates to manipulative and analytic steps for analyzing and verifying the products of low frequency events.

### BACKGROUND OF THE INVENTION

Genetic mutations underlie many aspects of life and death—through evolution and disease, respectively. Accordingly, their measurement is critical to several fields of research. Counting de novo mutations in humans, not present in their parents, have similarly led to new insights into the rate at which our species can evolve. Similarly, counting genetic or epigenetic changes in tumors can inform fundamental issues in cancer biology. Mutations lie at the core of current problems in managing patients with viral diseases such as AIDS and hepatitis by virtue of the drug-resistance they can cause. Detection of such mutations, particularly at a stage prior to their becoming dominant in the population, will likely be essential to optimize therapy. Detection of donor DNA in the blood of organ transplant patients is an important indicator of graft rejection and detection of fetal DNA in maternal plasma can be used for prenatal diagnosis in a non-invasive fashion. In neoplastic diseases, which are all driven by somatic mutations, the applications of rare mutant detection are manifold; they can be used to help identify residual disease at surgical margins or in lymph nodes, to follow the course of therapy when assessed in plasma, and perhaps to identify patients with early, surgically curable disease when evaluated in stool, sputum, plasma, and other bodily fluids. These examples highlight the importance of identifying rare mutations for both basic and clinical research.

Our growing understanding of the human gut microbiota as an indicator of and contributor to human health suggests that it will play important roles in the diagnosis, treatment, and ultimately prevention of human disease. These applications require an understanding of the dynamics and stability of the microbiota over the lifespan of an individual. Amplicon sequencing of the bacterial 16S rRNA gene from fecal microbial communities (microbiota) has revealed that each individual harbors a unique collection of species. Estimates of the number of species present in an individual's microbiota have varied greatly; from ˜100 with culture-based techniques to ˜160 with culture-independent deep shotgun sequencing of fecal community DNA to several fold higher based on 16S rRNA amplicon sequencing.

Massively parallel sequencing represents a particularly powerful form of Digital PCR in that hundreds of millions of template molecules can be analyzed one-by-one. It has the advantage over conventional Digital PCR methods in that multiple bases can be queried sequentially and easily in an automated fashion. However, massively parallel sequencing cannot generally be used to detect rare variants because of the high error rate associated with the sequencing process. For example, with the commonly used Illumina sequencing instruments, this error rate varies from ˜1% to ˜0.05%, depending on factors such as the read length, use of improved base calling algorithms and the type of variants detected. Some of these errors presumably result from mutations introduced during template preparation, during the pre-amplification steps required for library preparation and during further solid-phase amplification on the instrument itself. Other errors are due to base mis-incorporation during sequencing and base-calling errors. Advances in base-calling can enhance confidence, but instrument-based errors are still limiting, particularly in clinical samples wherein the mutation prevalence can be 0.01% or less.

There is a continuing need in the art to improve the sensitivity and accuracy of sequence determinations for investigative, clinical, forensic, and genealogical purposes.

### SUMMARY OF THE INVENTION

In one aspect, the invention encompasses a method of sequencing that improves sequence quality. The method comprises contacting sample comprising nucleic acid with a finite amount of linear primer. The linear primer comprises: (i) an adapter, (ii) a random component, and (iii) a target specific sequence. Linear PCR is then performed to generate a finite number of products. A product of linear PCR comprises the adapter, the random component and the target specific sequence. Next, the linear PCR product is contacted with 3 types of primers: primer type 1 comprises an adapter complementary to the adapter from the linear primer; primer type 2 comprises a target specific sequence that is 3′ of the target specific sequence in the linear primer and an adapter; and primer type 3 comprising an adapter complementary to the adapter in primer type 2 and an index sequence. Primer type 2 is diluted relative to primer type 1 and primer type 3. Then exponential PCR is performed to amplify the linear PCR product. The product of exponential PCR comprises in the 5′ to 3′ direction: the adapter, the random component, the target specific sequences, the downstream adapter, and the index sequence. Notably, both linear PCR and exponential PCR are performed in one reaction vial. Next the exponential PCR product is sequenced to generate redundant reads. The redundant reads are separated by the random component and a consensus sequence is identified such that the entire methodology improves the sequence quality.

In another aspect, the invention encompasses a method of sequencing gut microbial communities. The method comprises contacting sample comprising nucleic acid with a finite amount of linear primer. The linear primer comprises: (i) an adapter, (ii) a random component, and (iii) a 16S sequence. Linear PCR is then performed to generate a finite number of products. A product of linear PCR comprises the adapter, the random component and the 16S sequence. Next, the linear PCR product is contacted with 3 types of primers: primer type 1 comprises an adapter complementary to the adapter from the linear primer; primer type 2 comprises a 16S sequence that is 3′ of the 16S sequence in the linear primer and an adapter; and primer type 3 comprises an adapter complementary to the adapter in primer type 2 and an index sequence. Primer type 2 is diluted relative to primer type 1 and primer type 3. Then exponential PCR is performed to amplify the linear PCR product. The product of exponential PCR comprises in the 5′ to 3′ direction: the adapter, the random component, the 16S sequences, the downstream adapter, and the index sequence. Notably, both linear PCR and exponential PCR are performed in one reaction vial. Next the exponential PCR product is sequenced to generate redundant reads. The redundant reads are separated by the random component and a consensus sequence is identified such that the entire methodology improves the sequence quality enabling sequencing of gut microbial communities.

In yet another aspect, the invention encompasses a method to improve sequencing quality and depth. The method comprises performing linear PCR, wherein the linear PCR reaction comprises sample comprising nucleic acid and a finite amount of linear primer. The linear primer comprises a random component and a target specific sequence. The linear PCR generates less product than the sequencing depth. Next, exponential PCR is performed, wherein the exponential PCR reaction amplifies the linear PCR product. The exponential PCR product is then sequenced such that the methodology improves the sequence quality and depth.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts multiplex bacterial 16S rRNA gene sequencing using LEA-Seq; comparison with previous methods using mock communities composed of sequenced gut bacterial species. (A) Schematic of how the LEA-Seq method is used to redundantly sequence PCR amplicons from a set of linear PCR template extensions of bacterial 16S rDNA. This approach results in amplicon sequences with a higher precision than standard amplicon sequencing at lower abundance thresholds. (B) Performance of 16S rRNA amplicon sequencing methods assayed as the precision obtained for different sequence abundance thresholds. Standard methods for amplicon sequencing using the 454 pyrosequencer and the Illumina MiSeq instrument exhibit increased precision as less abundant reads are filtered out. By redundantly sequencing each amplicon with LEA-Seq, the precision of amplicon sequencing is increased at lower abundance thresholds for both the V1V2 region of the bacterial 16S rRNA gene (compare red and blue lines) and the V4 region (compare magenta and blue lines), thereby enabling detection of lower-abundance bacterial taxa at high precision.

FIG. 2 depicts measuring the stability of an individual's fecal microbiota over time with LEA-Seq. (A) The Jaccard Index (fraction of shared strains) was calculated between all possible pairwise combinations of fecal samples collected from each individual, where bacterial strains were considered shared if the nucleotide sequence was 100% identical across 100% of the length of the V1V2 region of their 16S rRNA genes. Jaccard Indexes were binned into intervals of <3 weeks, 3-6 weeks, 6-9 weeks, 9-12 weeks, 12-32 weeks, 32-52 weeks, 52-104 weeks, 104-156 weeks, 156-208 weeks, 208-260 weeks, and >260 weeks apart (mean±SE for each bin is shown). The decay in the Jaccard Index as a function of time between two samples best fits a power law (blue line). (B) Four individuals losing 10% of their body weight in the study involving consumption of a monotonous low calorie liquid diet (magenta) had significantly less stable microbiota than the mean of the 33 remaining individuals (blue). Mean±SE for the Jaccard Index are plotted. (C) At the phylum level, Bacteroidetes (blue) and Actinobacteria (red) were more stable components of the microbiota than the Proteobacteria and Firmicutes (hypergeometric distribution).

FIG. 3 depicts the relationship between weight stability, time, and fecal microbiota stability. (A) The microbiota sampled from a given individual during periods of weight loss or gain has decreased stability (lower Jaccard Index). (B) The Jaccard Index decreased as the time between samples increased (also see FIG. 2). (C) Across samples from 37 individuals, a linear model of microbiota stability as a function of changes in InBMI and changes in time explained 46% of the variation in the stability of the microbiota (Jaccard Index). Note that changes in InBMI explained more of the variation in microbiota stability than did the passage of time. Color changes correspond to the Jaccard Index values in the color bar on the right. Blue dots show the change in Jaccard Index, time, and InBMI between two samples from a given individual.

FIG. 4 depicts comparison of genome stability in fecal bacterial isolates recovered from individuals over time. The fraction of aligned nucleotides between any two microbial genomes was calculated using the coverage score (see text for definition). (A-C) Histogram of the fraction of aligned genome content between all sequenced bacterial isolates from unrelated individuals (A; blue; only coverage scores ≧0.01 are shown) shows that the alignable genome content never exceeded 96% (dotted line). However, highly conserved strains with coverage scores exceeding this threshold were readily detected in the microbiota of individuals at a single time point (B; red) or between samples from an individual taken up to 15 months apart (C; green). The y-axis “Counts” represent the number of times a sample fell into each coverage score bin. (D-I) Sequencing the genomes of M. smithii strains (D-F) and B. thetaiotaomicron strains (G-I) revealed that no two isolates from unrelated individuals had more than 96% shared (alignable) gene content (D, G; blue), while highly conserved strains above this threshold were found between isolates obtained from a single individual's fecal microbiota at a single time point (E, H; red), as well as from isolates taken from different members of the same family (F, I; brown).

FIG. 5 depicts a schematic overview of LEA-Seq at the nucleotide level. Phasing and indexing are performed according to the phased amplicon sequencing scheme described in FIG. 10. LEA-Seq adds an additional linear PCR step with a finite number of primers containing a 16-18nt random sequence prior to the template specific primer. Every fourth nucleotide in the random primer is H or W, as we empirically found our initial random primer containing only “N”s resulted in a high proportion of barcodes with G or C.

FIG. 6 depicts defining depth limitations of LEA-Seq 16S rRNA amplicon sequencing. All samples for a given 16S rRNA variable region/sequencing run combination were pooled, thus providing 10 times or more reads than our typical target depth of 150,000 reads (V4 run=4,055,875 reads; V1V2 run 1=1,150,528 reads; V1V2 run 2=1,224,195 reads). The extra reads enabled high precision at lower abundance than our target depth (compare with FIG. 1B), but precision dropped precipitously at depths near 1:100,000 reads, suggesting this represents a lower limit to the LEA-Seq method with current Illumina sequencing error rates and data processing pipelines.

FIG. 7 depicts the relative abundance of strains that were shared or not shared across time. (A) Strains that were shared between two samples from a given individual are ˜3-fold more abundant than strains that are not shared. In this box plot, the red central mark is the median. The edges of the box represent the 25^{th} and 75^{th} percentiles. Whiskers represent the most extreme points that were not considered outliers, while each outlier is plotted individually in red. (B) The probability that a strain is shared between fecal samples from a given individual (i.e., P(shared)) is directly correlated with the strains abundance in the fecal microbiota, with more abundant strains being more likely to be shared between any two samples from any individual.

FIG. 8 depicts the distribution of coverage scores for organisms in the same genus or species. The distribution of the coverage scores (fraction of aligned bases) between all pairwise comparisons of genomes from unrelated individuals shows distinct distributions for bacteria belonging to a given species and bacteria belonging to the same genus. Only comparisons between genomes having both a species name and a genus name are included. Coverage scores ≧0.1 are shown. Genus and species names were identified by 16S rRNA amplicon sequencing with the double-barcode strategy described in Methods.

FIG. 9 depicts extrapolating the stability of the microbiota over time. Using the parameters of the power law fit from empirical data generated from 37 females in the present study whose fecal microbiota were sampled over time spans of less than a week to over five years, the decay in the Jaccard Index was extrapolated over a 10-year and a 50-year (inset) period (95% confidence bounds are indicated with dotted lines).

FIG. 10 depicts a schematic overview of phased amplicon sequencing at the nucleotide level for the MiSeq instrument platform. Phases (green bases) are introduced into each primer to increase the complexity at each base and lower the error rate of the image-based Illumina MiSeq sequencing platform. The sample index (blue bases) is added via a third primer during the exponential PCR. (A) To enrich for the full-length amplicon rather than the preferentially amplified shorter amplicon, the inner primer (PE2a) is diluted 1 to 30 relative to the outer (flanking) ones. (B) Shows the full length Final PCR product.

FIG. 11 depicts the effect of k-mer size on assembly quality (N50). (A,B) For the 30 assemblies with the highest coverage (panel A) and all sequenced genomes for the tested fecal microbiota donor (panel B), increases to the k-mer parameter leads to slight increases in N50. This is particularly true for higher coverage assemblies. However, performance begins to decline if k-mer is increased too far (k-mer=63 for high coverage; k-mer=45 for low coverage). On the box plot, the central mark is the median and the edges of the box represent the upper and lower quartiles. The whiskers represent the most extreme points that were not considered outliers, while each outlier is plotted individually.

FIG. 12 depicts the effect of k-mer size on assembly quality (% genes mapping to a reference genome). (A,B) For both the 30 assemblies with the highest coverage (panel A) and all of the genomes for the tested fecal microbiota donor (panel B), increases to the k-mer parameter leads to decreases in the proportion of genes in the assembly that map to a reference genome from the same species. On the box plot, the central mark is the median and the edges of the box represent the upper and lower quartiles. The whiskers represent the most extreme points that were not considered outliers, while each outlier is plotted individually.

### DETAILED DESCRIPTION OF THE INVENTION

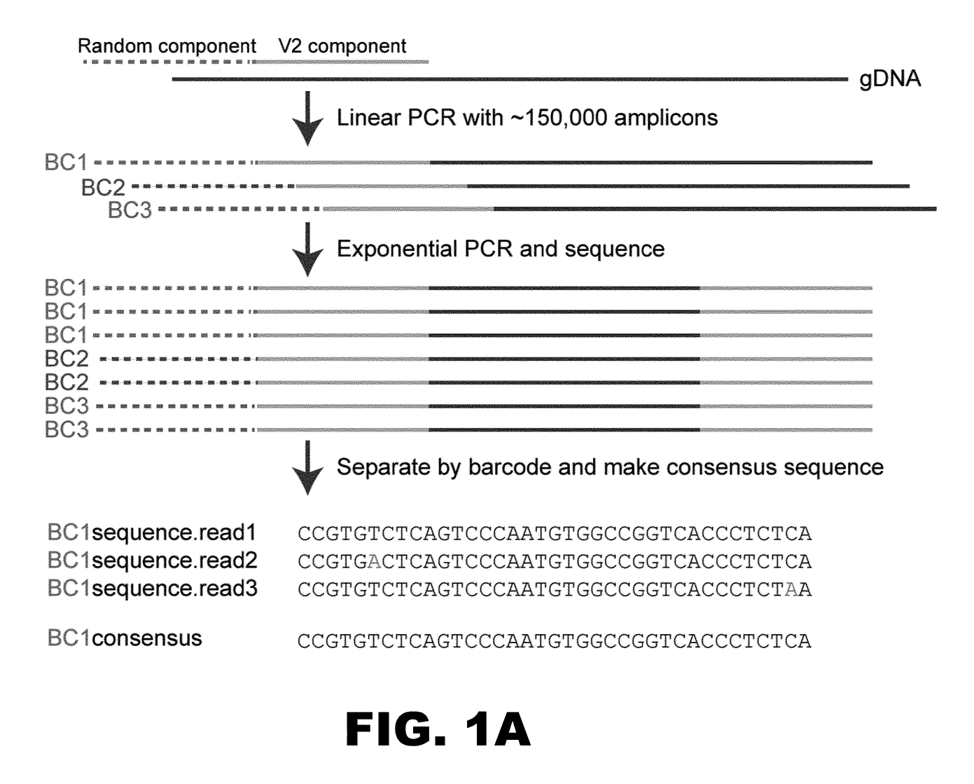

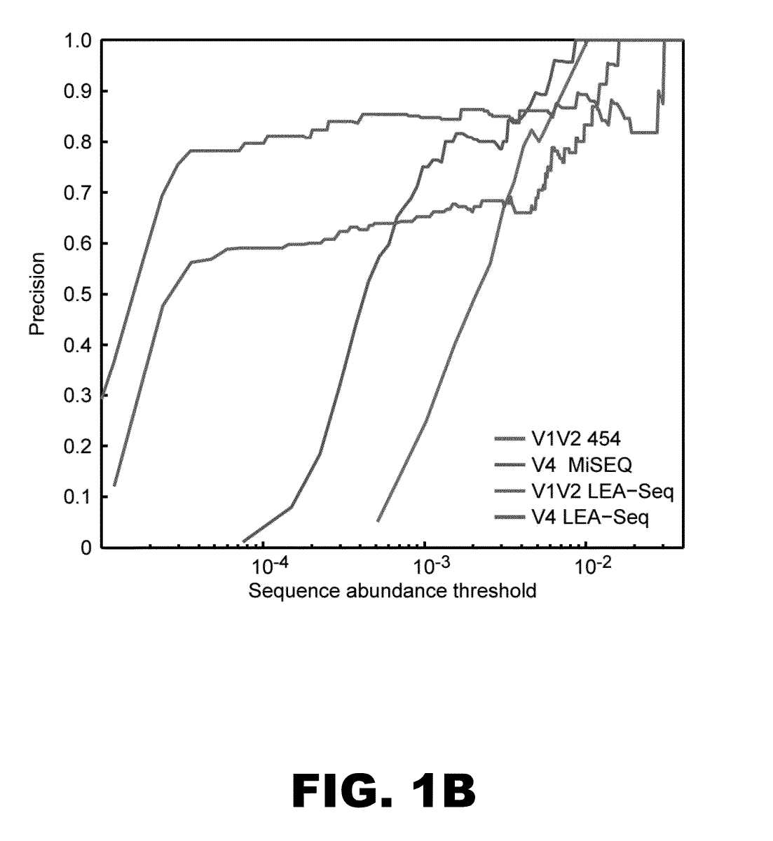

The inventors have developed an approach called LEA-Seq (Low-Error Amplicon Sequencing). In one embodiment, it involves two basic steps (FIG. 1). The first step is linear PCR to simultaneously tag with a random component the nucleic acid to be analyzed and create a finite nucleic acid pool that is less than the sequencing depth. This finite pool is known as a bottleneck. The second step is exponential PCR of each uniquely tagged nucleic acid from the finite pool of linear PCR products, so that a plurality of products with the identical sequence is generated. If a mutation or specific sequence existed in the template nucleic acid used for amplification, that mutation or specific sequence should be present in a certain proportion, or even all, of the products containing the random tag. Having sequencing depth that exceeds the number of linear PCR products ensures that multiple copies of these products can be sequenced, and the random component on each molecule enables the multiple copies of each amplicon to be collected and error-corrected computationally to generate a consensus sequence with higher fidelity than the raw error-rate of the DNA sequencing technology. This approach can be employed for any purpose where a very high level of accuracy and sensitivity is required from sequence data. As shown below, this approach can be used to study the dynamics and stability of a microbiome population.

The LEA-Seq methodology has numerous added benefits over the prior art. First, surprisingly, the entire methodology can be carried out in a single reaction tube. The ability to use a single reaction tube allows the methodology to be easily automated. It was unexpected that adding such a complex mix of starting material, primers and polymerase would result in accurate and precise sequence information. Second, the LEA-Seq methodology eliminates the need to pre-dilute the initial sample to create a finite nucleic acid pool that is smaller than the amount of sequencing available. Instead, LEA-Seq uses the linear PCR reaction to create a bottleneck. This has the added advantage of eliminating the need to determine the actual input for every sample via time consuming and expensive methodologies such as qPCR or flow cytometry. Third, the linear PCR reaction facilitates the application of LEA-Seq to high throughput assays, as the entire process can move from template to final product in an add-only reaction with the linear PCR and exponential PCR reaction occurring in the same tube. Bypassing the need to dilute the amount of starting template reduces labor and costs as there is no need to count cells by flow cytometry or count target molecules by qPCR. Thus the disclosed methodology is cheaper and faster with increased accuracy. The methodology exerts significant benefit wtih extremely complex amples. In these situations, LEA-Seq results in amplicon sequences with a higher precision than standard amplicon sequencing at a lower abundance threshold.

### I. Method of Sequencing

The present invention encompasses a method of sequencing that improves sequence quality. The method comprises contacting sample comprising nucleic acid with a finite amount of linear primer, wherein the linear primer comprises: (i) an adapter, (ii) a random component, and (iii) a target specific sequence. Linear PCR is then performed, wherein performing linear PCR generates a finite number of products and wherein the product of linear PCR comprises the adapter, the random component and the target specific sequence. Next, the linear PCR product is contacted with 3 types of primers: primer type 1 comprises an adapter complementary to the adapter from the linear primer; primer type 2 comprises a target specific sequence that is 3′ of the target specific sequence in the linear primer and an adapter, wherein primer type 2 is diluted relative to primer type 1 and primer type 3; and primer type 3 comprises an adapter complementary to the adapter in primer type 2 and an index sequence. Exponential PCR is then performed, wherein the linear products are amplified and wherein the products of exponential PCR comprise in the 5′ to 3′ direction: the adapter, the random component, the target specific sequences, the downstream adapter, and the index sequence. Importantly, both linear PCR and exponential PCR are performed in one reaction vial. Finally, the exponential PCR products are sequenced, wherein redundant reads are generated during exponential PCR. The redundant reads are then separated by the random component and a consensus sequence is identified such that the redundant reads improve the sequence quality.

### (a) Linear PCR

A method of the invention involves contacting sample comprising nucleic acid with a finite amount of linear primer. The linear primer comprises an adapter, a random component and a target specific sequence.

The linear primer may comprise, in part, an adapter. As used herein, an “adapter” is a sequence that permits universal amplification. A key feature of the adapter is to enable the unique amplification of the linear PCR product only without the need to remove existing template nucleic acid or purify the linear PCR product. This feature enables an “add only” reaction with fewer steps and ease of automation. The adapter is placed on the 5′ end of the linear primer. In an exemplary embodiment, the adapter may be an Illumina adapter for Illumina sequencing.

The linear primer further comprises, in part, a random component. A random component may also be referred to as a barcode. A random component may be composed of random nucleotides to generate a complexity of random components far greater than the number of unique amplicons to be sequenced. This ensures that having the same random component attached to multiple amplicons is an extremely statistically improbable event. The random component design can theoretically generate 9.1×10⁸ to 1.4×10¹⁰ unique random components, which is more than three orders of magnitude more than the number of unique amplicons to be sequenced. This complexity can easily be expanded by increasing the length of the random regions in the linear PCR primer. In addition based on empirical observations, the inventors found that a purely random barcode (IUPAC code N=(A or C or G or T) consisting of any possible nucleotide at every position led to a bias towards barcodes there were high in G/C content. To remedy this bias, the inventors limited the complexity of every fourth base to IUPAC codes of H (A or C or T) or W (A or T). In an embodiment, the random component may be about 5 to about 100 nucleotides. In an embodiment, the random component may be about 10 to about 25 nucleotides. For example, the random component may be about 15 to about 20 nucleotides. In an exemplary embodiment, the random component is about 16 to about 18 nucleotides. Accordingly, the random component may be 10, 11, 12, 13, 14, 15, 16, 17, 18, 19, 20, 21, 22, 23, 24 or 25 or more nucleotides.

The linear primer further comprises, in part, a target specific sequence. The target specific sequence may be at the 3′ end of the linear primer. The target specific sequence is a sequence complementary to a nucleic acid of interest or a target nucleic acid. The target specific sequence may be altered based on the target nucleic acid to be amplified. A target nucleic acid for the target specific sequence may be any nucleic acid amenable to standard PCR. Non-limiting examples of a target nucleic acid may be a nucleic acid used to identify a rare mutation associated with drug-resistance, graft rejection, residual disease, tumors, immune diseases. Alternatively, a target nucleic acid may be a nucleic acid used to identify a bacterial strain. It is known in the art that 16S nucleic acid is a good, widely used nucleic acid to identify a bacterial strain. In a preferred embodiment, the target specific sequence is a sequence complementary to a 16S nucleic acid sequence. In an exemplary embodiment, the target specific sequence is a sequence complementary to the V4 region of the 16S rRNA nucleic acid. In another exemplary embodiment, the target specific sequence is a sequence complementary to the V1V2 region of the 16S rRNA nucleic acid. The target specific sequence may comprise 10 to 100 nucleotides complementary to the target nucleic acid. For example the target specific sequence may comprise 15 to 30 nucleotides complementary to the target nucleic acid. In an embodiment, the target specific sequence may comprise 15 to 25 nucleotides complementary to the target nucleic acid.

In an embodiment, the linear primer may optionally comprise phasing nucleotides to increase sequence complexity. Phasing nucleotides may lower the error rate of the sequencing platform used. For example, phasing nucleotides may lower the error rate of the image-based Illuminia MiSeq sequencing platform. A linear primer may comprise 1, 2, 3, 4, 5, 6, 7, 8, 9, or 10 or more phasing nucleotides. When phasing nucleotides are included in the linear primer, each of the phased linear primers may be evenly mixed. A reaction may comprise 1, 2, 3, 4, 5, 6, 7, 8, 9, or 10 or more differently phased linear primers. In an exemplary embodiment, four phases are used. In another exemplary embodiment, eight phases are used.

A finite amount of linear primer is contacted with sample comprising nucleic acid. Nucleic acid may be, for example, RNA or DNA. Modified forms of RNA or DNA may be used. In an exemplary embodiment, the sample is genomic DNA. The sample comprising nucleic acid may be a sample from a subject, the environment, a laboratory, or any sample in which nucleic acid is present. When the sample is from a subject, the sample may be from stool, sputum, plasma, and other bodily fluids. In general, the LEA-Seq methodology is beneficial for samples comprising highly complex starting material. As used herein, “highly complex” refers to a sample that comprises nucleic acid from multiple sources. For instance, nucleic acid from microbial communities comprising a plurality of species. In an exemplary embodiment, the sample is from at least one microbial community of a subject. Non-limiting examples of microbial communities may be found in the gut of a subject, on the skin of a subject, or in an orifice of a subject. In another exemplary embodiment, a sample comprising nucleic acid is from a gut (e.g. gastrointestinal tract) of a subject. In an embodiment wherein the sample is from a subject, the target specific sequence may be a sequence complementary to the 16S nucleic acid.

The subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

A finite amount of linear primer is contacted with sample comprising nucleic acid. The addition of a finite amount of linear primer creates a finite nucleic acid pool, also known as a bottleneck. To redundantly sequence nucleic acid fragments, it is necessary to create a finite nucleic acid pool that is smaller than the amount of sequencing capacity available. This is so that each nucleic acid in the pool may be sequenced a plurality of times. Previous, less effective methods dilute the initial nucleic acid pool to create a bottleneck. However, this dilution requires the need to empirically determine the input for every sample using, for example, qPCR or flow cytometry. This requires significantly more time, effort and cost. The LEA-Seq methodology bypasses the need to determine the input for every sample by creating a finite nucleic acid pool by contacting a finite amount of linear primer with an undiluted sample comprising nucleic acid. One of skill in the art would be able to empirically determine the amount of linear primer necessary to obtain a proper amount of linear extensions for the sequencing coverage desired. In an exemplary embodiment, a linear primer may be diluted such that approximately 150,000 linear extensions would be sequenced per sample at 20× coverage. As different sequencing methodologies can handle different depths, the linear primer may be diluted accordingly. By way of example, a linear primer may be diluted such that approximately 50,000 to 500,000 linear extensions may be sequenced per sample at 5× to 50× coverage. Alternatively, a linear primer may be diluted such that approximately 100,000 to 300,000 linear extensions would be sequenced per sample at 10× to 30× coverage. A skilled artisan familiar with sequencing methodologies would be able to determine this dilution. For example, a linear primer stock concentration of 200 μM may be diluted 1:400,000,000. For a given application, this dilution can be determined empirically by diluting the linear PCR primer and counting the number of unique labels in the resultant sequences.

For each linear PCR reaction, linear primer is contacted with undiluted sample comprising nucleic acid. In an embodiment, a linear PCR reaction may comprise undiluted sample comprising nucleic acid, linear primer, polymerase, water, buffer, and deoxynucleotide triphosphates (dNTPs) in a single reaction vial. Linear PCR may be performed according to standards methods in the art. By way of non-limiting example, the linear PCR reaction may comprise denaturation, followed by about 5-10 cycles of denaturation, annealing and extension, followed by a final extension. In an exemplary embodiment, the linear PCR reaction comprises denaturation at 98° C. for 30 seconds, followed by 8 cycles of (98° C. for 10 seconds, 50° C. for 30 seconds, 72° C. for 30 seconds), followed by a final extension at 72° C. for 2 minutes.

According to a method of the invention, performing linear PCR generates a finite number of products. The products of linear PCR comprise a linker, a random component and a target specific sequence.

### (b) Exponential PCR

A method of the invention further comprises contacting the linear PCR product with 3 types of primers. Primer type 1 comprises an adapter complementary to the adapter of the linear primer. Primer type 2 comprises a target specific sequence that is 3′ of the target specific sequence utilized in the linear primer and an adapter. Primer type 3 comprises an adapter complementary to the adapter of primer type 2 and an index sequence. Importantly, primer type 2 is diluted relative to primer type 1 and primer type 3.

Primer type 3 comprises, in part, an index sequence. The addition of an index sequence allows pooling of multiple samples into a single sequencing run. This greatly increases experimental scalability, while maintaining extremely low error rates and conserving read length. The index sequence may be about 5 to about 10 nucleotides. Accordingly, the index sequence may be 5, 6, 7, 8, 9 or 10 or more nucleotides. In an exemplary embodiment, the index sequence is about 6 nucleotides.

In an embodiment, primer type 2 may optionally comprise phasing nucleotides to increase sequence complexity. Phasing nucleotides may lower the error rate of the sequencing platform used. For example, phasing nucleotides may lower the error rate of the image-based Illuminia MiSeq sequencing platform. A primer type 2 may comprise 1, 2, 3, 4, 5, 6, 7, 8, 9, or 10 or more phasing nucleotides. When phasing nucleotides are included in primer type 2, each of the phased primer type 2s may be evenly mixed. A reaction may comprise 1, 2, 3, 4, 5, 6, 7, 8, 9, or 10 or more differently phased primer type 2s. In an exemplary embodiment, four phases are used. In another exemplary embodiment, eight phases are used.

Primer type 2 is diluted relative to primer type 1 and primer type 3. Primer type 1 and primer type 3 are the outermost primers whereas primer type 2 is the innermost primer. The purpose of diluting primer type 2 is to ensure the exponential PCR product is enriched for the longest PCR product that will contain the index sequence from primer type 3. In an embodiment, primer type 2 may be diluted from about 1:10 to about 1:60 relative to primer type 1 and primer type 3. For example, primer type 2 may be diluted from about 1:20 to about 1:50 relative to primer type 1 and primer type 3. In an exemplary embodiment, primer type 2 may be diluted 1:30 relative to primer type 1 and primer type 3. For example, the final concentration of primer type 1 and primer type 3 may be 250 nM and the final concentration of primer type 2 may be 8.33 nM.

For each exponential PCR reaction, the linear PCR product is contacted with the 3 types of primers. Importantly, the 3 types of primers may be directly added to the same reaction vial used for linear PCR. In an embodiment, an exponential PCR reaction may comprise linear PCR product, primer type 1, primer type 2, primer type 3, polymerase, water, buffer, and deoxynucleotide triphosphates (dNTPs) in a single reaction vial. Exponential PCR may be performed according to standard methods in the art. By way of non-limiting example, the exponential PCR reaction may comprise denaturation, followed by about 25 cycles of denaturation, annealing and extension, followed by a final extension. In an exemplary embodiment, the exponential PCR reaction comprises denaturation at 98° C. for 30 seconds, followed by 25 cycles of (98° C. for 10 seconds, 50° C. for 30 seconds, 72° C. for 30 seconds), followed by a final extension at 72° C. for 2 minutes.

Upon performing exponential PCR, the linear PCR products are amplified. The exponential PCR products comprise in the 5′ to 3′ direction: an adapter, a random component, target specific sequences, a downstream adapter and an index sequence.

### (c) Sequencing

A method of the invention further comprises sequencing the exponential PCR product. According to the method of the invention, sequencing of the exponential PCR product generates redundant reads. The redundant reads are separated by random component and a consensus sequence is identified such that the redundant reads improve the sequence quality.

Sequencing may be performed according to standard methods in the art. Sequencing is preferably performed on a massively parallel sequencing platform, many of which are commercially available. In an exemplary embodiment, Illumina sequencing is used.

Reads may be separated by the index sequence and trimmed to remove primer sequences and, optionally, phasing nucleotides. Reads may be grouped by the random component. In certain embodiment, groups of reads with less than four reads may be removed. To eliminate ambiguous sequences, the random components may be sorted by abundance and clustered at an identity of 86%. Alternatively, the random components may be sorted by abundance and clustered at an identity of about 65% to about 95%. The random components may be clustered from most abundant to least abundant. Given that most sequencing errors are random and that the correct sequence should occur more often than a variant with sequencing errors, the abundance-weighted clustering provides a means to eliminate spurious random components that are most likely due to sequencing errors while retaining the more abundant (and most likely true positive) random components. Only the sequence reads containing the most abundant random component representative of each identity cluster are retained for further analysis.

Since amplicons with the same random component originated from a linear PCR product of one template molecule that was subsequently amplified by exponential PCR, they should be identical. This redundant sequencing of each linear PCR product allows the error-correction of each amplicon. For example, a consensus sequence is generated for each random component group by scoring and weighing the nucleotide at each base position. Sequences with a consensus sequence that is identical to the most abundant sequence associated with the same random component are kept, this process is called quality filtering. The inventors confirmed that LEA-Seq methodology was as accurate as standard amplicon sequencing. The inventors demonstrated that LEA-Seq with consensus compared to LEA-Seq without consensus resulted in the detection of 3 times more strains due to increased detection depth. Quality filtering of the sequences is critical to accurately estimating the number of target specific sequence or strains.

### II. Methods of Use

A method of the invention may be used to quantitate as well as to determine a sequence. For example, the relative abundance of two or more analyte nucleic acid fragments may be compared. A method of the invention may be used to identify rare mutants in a population of DNA templates, to measure polymerase error rates, or to judge the reliability of oligonucleotide synthesis. Additionally, a method of the invention may be used to diagnose, treat or prevent a disease in a subject. Identification of a rare mutation could facilitate the diagnosis of a disease, enable the proper methodology, such as a therapeutic, to treat the disease, or prevent the onset of disease by administration of prophylactic therapies. Still further, a method of the invention may be used to detect genetic mutations involved in cancer or other diseases, such as immune-mediated diseases. In a preferred embodiment, a method of the invention may be used to identify and quantify a microbial community of a subject. The knowledge gained may be used to assess the health of the subject.

The results described in the examples below describe a method of sequencing gut microbial communities using the LEA-seq methodology described above. The LEA-Seq methodology substantially improves the accuracy and depth of massively parallel sequencing. Thus, the methodology results in an assay to determine the bacterial composition of the gut microbiota of individuals at high depth with high precision. The LEA-Seq approach produces amplicon sequences with higher precision from taxa present at lower abundance thresholds than existing standard approaches (FIG. 1). LEA-Seq may be applied to virtually any sample preparation workflow or sequencing platform. As demonstrated here, the approach can easily be used to identify rare or low abundant bacterial species in a diverse population of bacterial species, such as the environment found in the gut microbiota.

### EXAMPLES

The following examples illustrate various iterations of the invention.

### Introduction to the Examples

Our growing understanding of the human gut microbiota as an indicator of and contributor to human health suggests that it will play important roles in the diagnosis, treatment, and ultimately prevention of human disease. These applications require an understanding of the dynamics and stability of the microbiota over the lifespan of an individual. Amplicon sequencing of the bacterial 16S rRNA gene from fecal microbial communities (microbiota) has revealed that each individual harbors a unique collection of species (1-3). Estimates of the number of species present in an individual's microbiota have varied greatly; from ˜100 with culture-based techniques (4) to ˜160 with culture-independent deep shotgun sequencing of fecal community DNA (5) to several fold higher based on 16S rRNA amplicon sequencing even after in silico attempts to remove chimeric molecules formed during PCR and errors introduced during sequencing. These artifacts complicate tracking of individual bacterial taxa across time by inflating the set of strains in each sample with false positives. Shotgun sequencing of the community's microbiome is another approach for defining diversity (6), but it is difficult to associate gene sequences with their genome of origin. With these limitations in mind, we have developed a method for amplicon sequencing to assay the bacterial composition of the gut microbiota of individuals at high depth with high precision over time. When combined with high throughput methods for culturing and sequencing the genomes of anaerobic bacteria, these results reveal that the majority of the bacterial strains in an individual's microbiota persist for years, and suggest that our gut colonizers have the potential to shape many aspects of our biological features for most if not the entirety of our lives.

### Example 1

### A Method for Low Error Amplicon Sequencing (LEA-Seq) of Bacterial 16S rRNA Genes

A 16S rRNA sequencing method for assaying the stability of an individual's microbiota over time would ideally retain high precision at high sequencing depth

$$( precision = \frac{TruePositives}{TruePositives + FalsePositives} ) .$$

Low precision data complicate comparison of sequences between samples, as it becomes difficult to differentiate species (typically defined as isolates that share 97% sequence identity in their 16S rRNA genes), and strains (isolates of a given species with more minor variations in their 16S rRNA gene sequences) from sequencing errors. Standard amplicon sequencing is limited in its precision by the overall error rate of the sequencing method. Low sequencing depth prevents determining if a strain has dropped out of a given individual's microbiota or has fallen below the limits of detection at the sampling depth employed.

In many applications it would be advantageous to exchange sequence depth for improved sequence quality. Despite several optimizations we developed to increase the precision of standard amplicon sequencing at shallow depths, we found that sequencing a sample beyond 10,000 reads did not substantially increase the lower detection limit possible at high precision (Supplemental Results). Exchanging sequence quantity for sequence quality is inherent to shotgun genome sequencing where redundant sequencing of genomes at 10- to 50-fold coverage enables a far lower error rate than is attainable from single-reads alone. In general, to redundantly sequence DNA fragments it is necessary to create a finite DNA pool that is smaller than the amount of sequencing available (i.e., create a bottleneck) and to have a method of labeling the molecules in the pool (7-9). To adapt these techniques to redundantly sequence PCR amplicons, the initial template DNA could be diluted to create a bottleneck. However, this dilution would likely need to be empirically determined for every input sample (e.g., using qPCR), and one would still need to label each template molecule. As an alternative, we developed a method that we named Low Error Amplicon Sequencing (LEA-Seq).

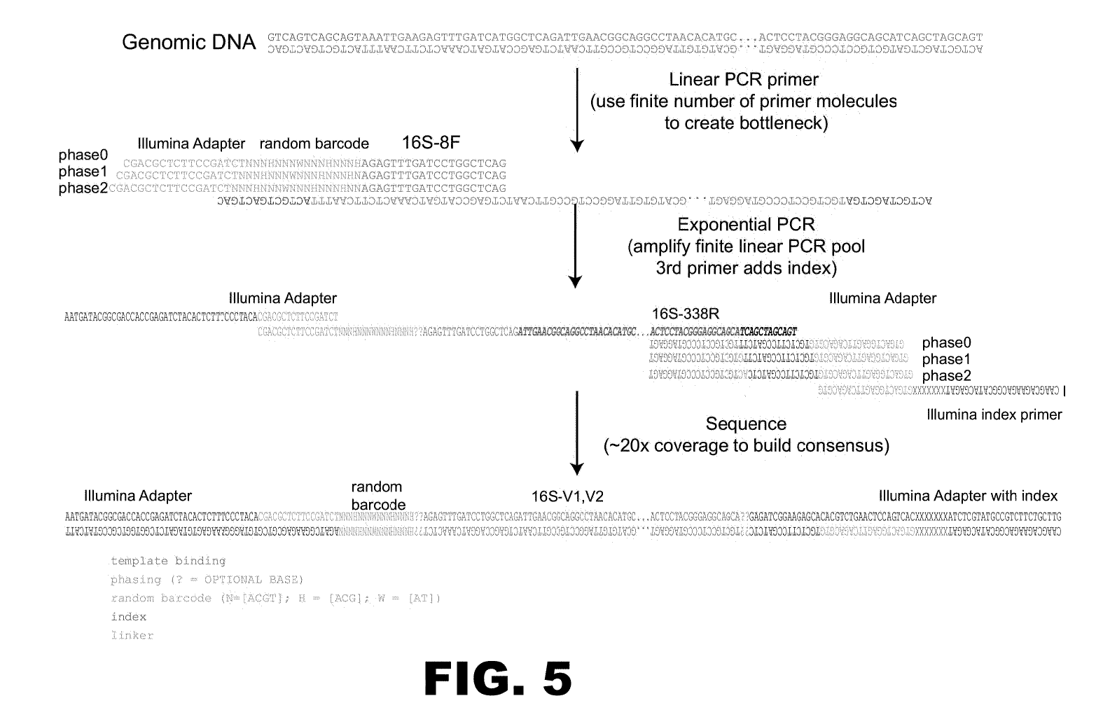

As outlined in FIG. 1A, LEA-Seq is based on redundant sequencing of a set of linear PCR template extensions of 16S rRNA genes to trade sequence quantity for quality. In this method, we create the bottleneck with a linear PCR extension of the template DNA with a dilute, barcoded, oligonucleotide primer solution. Each oligonucleotide is labeled with a random barcode positioned 5′ to the universal 16S rRNA primer sequence (FIG. 1A, FIG. 5). We then amplify the labeled, bottlenecked linear PCR pool with exponential PCR using primers that specifically amplify only the linear PCR molecules. During the exponential PCR, an index primer is added to the amplicons with a third primer to allow pooling of multiple samples in the same sequencing run (FIG. 1A). This exponential PCR pool is then sequenced at sufficient depth to redundantly sequence (˜20× coverage) the bottlenecked linear amplicons. The resulting sequences are separated by sample using the index sequence, and the amplicon sequences within each sample are separated by the unique barcode; the multiple reads for each barcode allow the generation of an error-corrected consensus sequence for the initial template molecule. In LEA-Seq, the linear PCR primers are diluted to a concentration that generates ˜150,000 amplicon reads at 20× coverage per amplicon on an Illumina HiSeq DNA sequencer (FIG. 1A, FIG. 5).

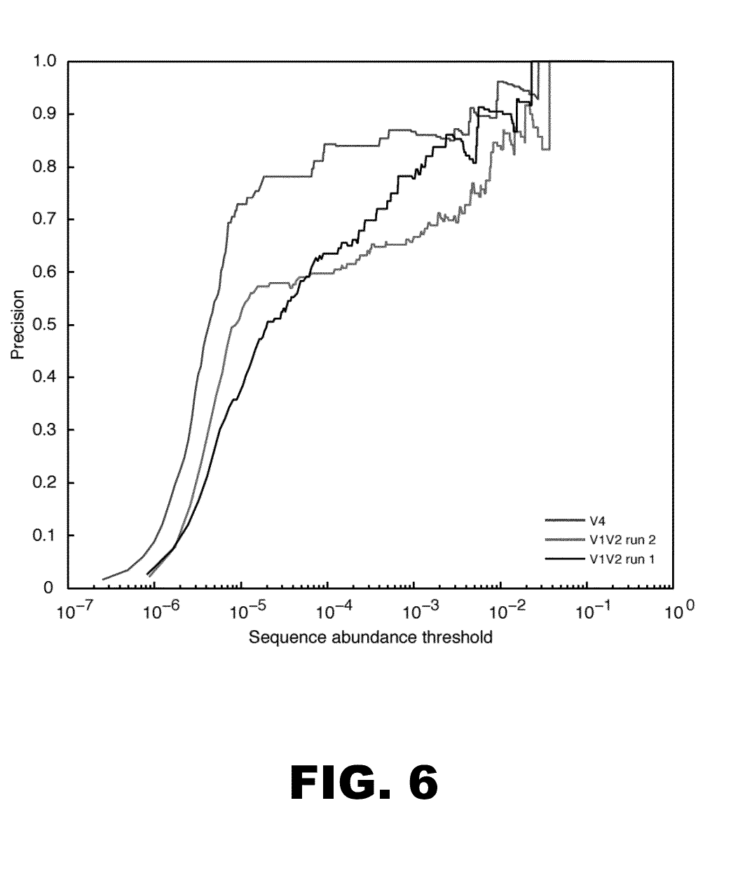

To empirically test LEA-Seq against existing 16S rRNA amplicon sequencing methods, we first generated nine in vitro ‘mock’ communities composed of different proportions of strains from a 48-member collection of phylogenetically diverse, cultured human gut bacteria whose genomes had been characterized (see Methods and Table 2). To calculate precision, we compared amplicons generated using two sequencing platforms (Illumina MiSeq and 454 FLX instruments), targeting different variable (V) regions of the 16S rRNA gene with different PCR primers. We defined a TruePositive sequence as 100% identical across 100% of its length to the 16S rRNA gene sequence(s) in the reference genome. We calculated precision at different abundance thresholds by including only those sequences representing at least a minimal portion of the total sequencing reads (0.5%, 0.1%, 0.05%, 0.01%, or 0.005%). LEA-Seq produced amplicon sequences with higher precision from taxa present at lower abundance thresholds in the mock communities than existing standard approaches (FIG. 1B). For 16S rRNA sequences representing ≧0.01% of the reads, LEA-Seq enabled a precision of 0.83±0.02 (V4) and 0.63±0.03 (V1V2) versus 0.08±0.064 and 0.09±0.005 for the same regions with standard amplicon sequencing (Table 3). These performance improvements are dependent on generating the consensus sequence from the redundant amplicon reads (Table 3; Method=“LEA-Seq without consensus”). LEA-Seq also produced slower saturation in performance (precision of >0.7 for reads representing 0.001% of the total; FIG. 6; Table 3). Similar results were obtained using the several different mock communities (for additional details of the analysis, including V1V2 versus V4 comparisons, see ‘Optimization of bacterial 16S rRNA amplicon sequencing’ below). Based on this assessment of its attributes, we used LEA-Seq to quantify the stability of the gut microbiota within individuals as a function of time and change in body mass index while consuming controlled monotonous and free diets.

### Example 2

### Applying LEA-Seq to Define the Stability of the Fecal Microbiota of 37 Healthy Adults

Stability of a Microbiota Best Fits a Power Law Function—

We used LEA-Seq to characterize the microbiota in 167 fecal samples obtained from 37 healthy adults residing throughout the USA; 33 of these donors were sampled 2-13 times up to 296 weeks apart (1, 10) (Table 4). The remaining four individuals were sampled on average every 16 days for up to 32 weeks while consuming a monotonous liquid diet as part of a controlled in-patient weight-loss study (see Methods) (11-13). None of the individuals took antibiotics for at least two months prior to sampling. All fecal samples were frozen at −20° C. immediately after they were produced and then at −80° C. within 24 h. DNA was isolated from all samples by bead beating in phenol/chloroform.

Employing an Illumina HiSeq2000 instrument to sequence amplicons from the V1V2 region of bacterial 16S rRNA genes, we generated 108,677±60,212 (mean±SD) LEA-Seq reads per fecal DNA sample. Reads were then filtered using a minimum sequence abundance threshold cutoff of eight reads (i.e., to detect strains present in the fecal microbiota at an average relative abundance of 0.007%). Based on our mock community data, the precision at this threshold for the V1V2 region is 0.63. We defined the number of strains in a sample as the number of unique amplicon sequences and the number of species-level OTUs in the sample as the number of clusters with 97% shared sequence identity. To correct for false-positives, the number of strains was multiplied by the precision (i.e., if we detect 100 unique sequences, we expect 63 of them to be true). For individuals sampled over multiple time points, we calculated the number of species and strains for each sample individually and averaged them. The results indicated that individuals in this cohort harbored 195±48 bacterial strains in their fecal gut microbiota, representing 101±27 species.

To study each individual's microbiota over time, we took all possible pairs of samples from the time series of each individual (Table 4) and calculated the time in weeks between the sample dates as well as the fraction of shared strains between them, as measured by the binary Jaccard Index (an unweighted metric of community overlap).

$$JaccardIndex ( sampleA , sampleB ) = \frac{sampleA ⋂ sampleB}{sampleA ⋃ sampleB}$$

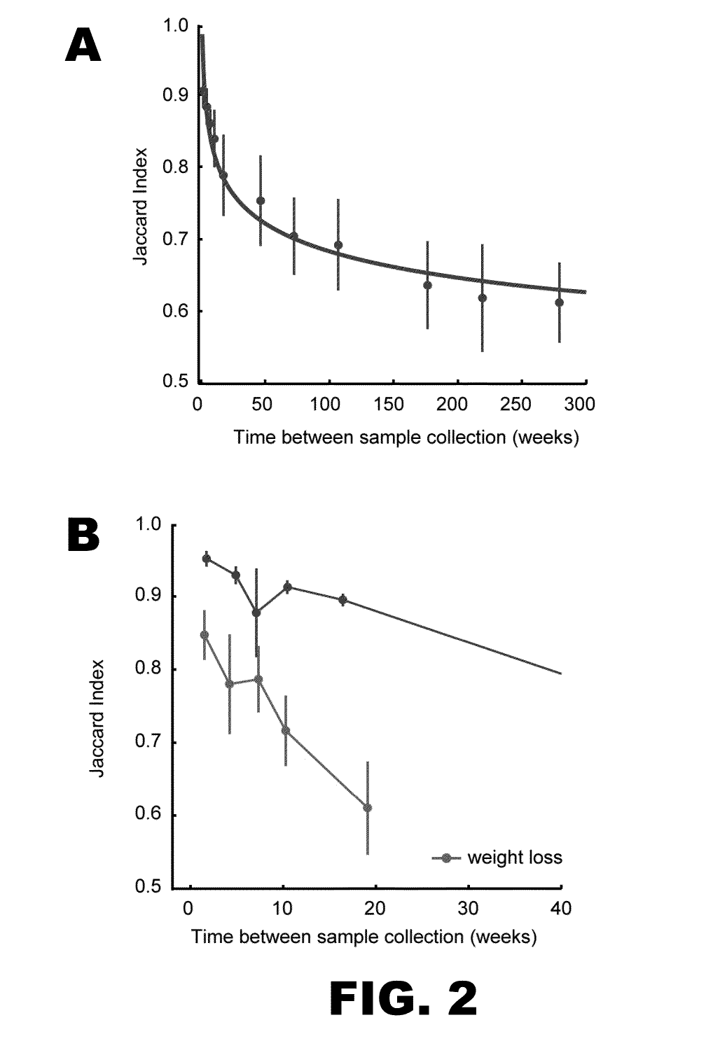

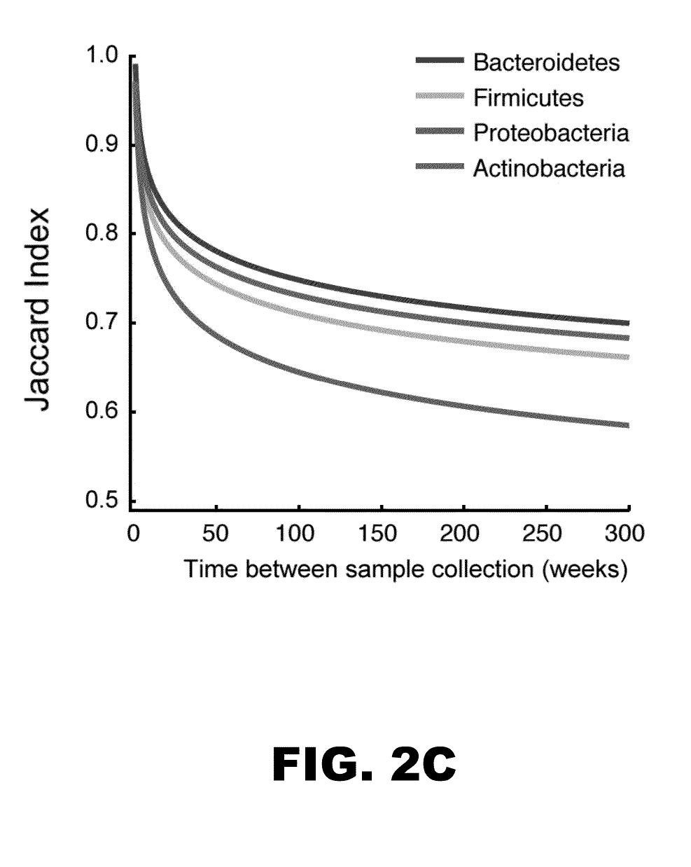

Control experiments using mock communities (Table 2), established that LEA-Seq of V1V2 16S rRNA amplicons produced highly accurate estimates of the Jaccard Index (correlation between known and measured Jaccard Index=0.996). To characterize the stability of an individual's microbiota, fecal samples were binned into intervals (<3 weeks, 3-6 weeks, 6-9 weeks, 9-12 weeks, 12-32 weeks, 32-52 weeks, 52-104 weeks, 104-156 weeks, 156-208 weeks, 208-260 weeks, and >260 weeks) and Jaccard Index values were averaged for each bin. The results disclosed that the bacterial composition of each individual's fecal microbiota changed over time, with more strains shared between closer time intervals compared with long intervals (FIG. 2A). Nonetheless, overall the set of microbial strains was remarkably stable, with over 70% of the same strains remaining after one year and few additional changes occurring over the following four years. The stability of a microbiota best fits a power law function (R²=0.96; FIG. 2A blue line; Table 5) where large differences in community composition occur on shorter time scales, while a stable core set of strains persists at longer time scales.

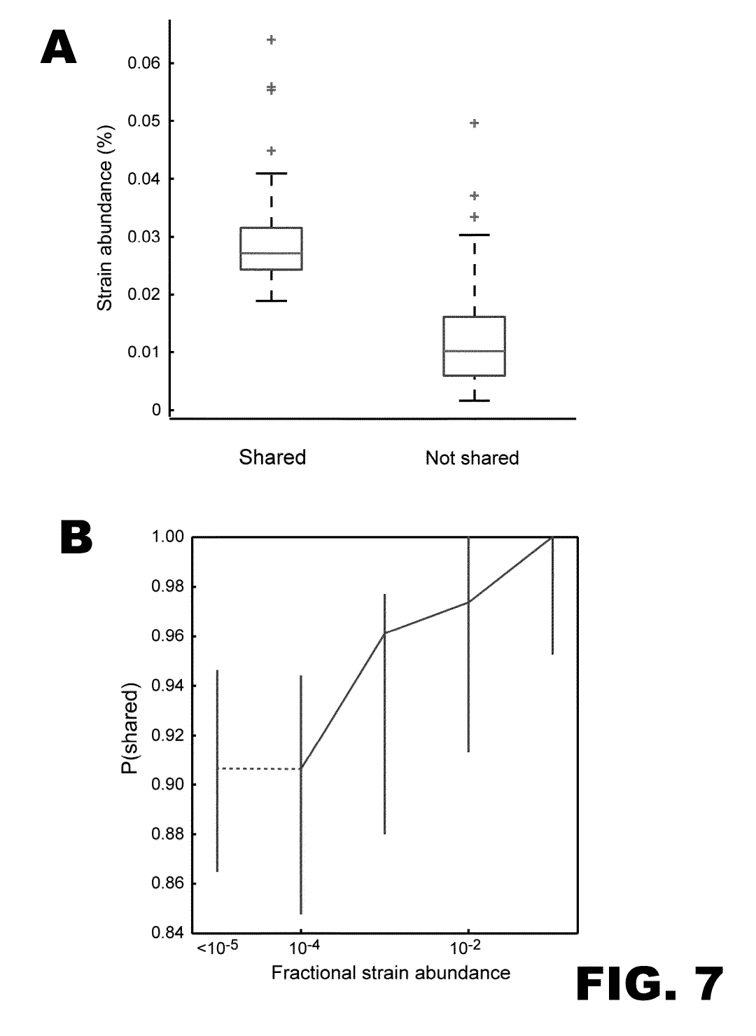

To define the stability of a given strain as a function of its relative abundance in the microbiota, we used all pairwise combinations of fecal samples obtained from each individual to calculate (i) the mean abundance of the strains shared by two or more samples, and (ii) the mean abundance of strains that were not shared between any two samples. Strains that were shared across two time points were roughly three-fold more abundant than those that were not shared [0.030±0.013 fraction of the community versus 0.011±0.011 (mean±SD); p-val=2.2×10⁻⁹ (t-test) FIG. 7A]. We also binned the strain abundances for each donor using five fractional abundance thresholds of 0.1, 0.01, 0.001, 0.0001, and <0.0001 (e.g., bin 0.01 contains all strains ≦0.1 and >0.01) and calculated the probability that strains in a given bin were shared between samples. We found the higher the fractional abundance of a strain, the more likely the strain was shared between samples (r=0.96, p<0.0087; FIG. 7B). Together, these results suggest that the more stable components of the microbiota are also the most abundant members.

Effects of a Monotonous Low Calorie Diet and Associated Weight Loss on Diversity—

To explore the role of weight loss on the microbiota, we applied LEA-Seq to the fecal microbiota of four individuals sampled over the course of a 8- to 32-week period in a three phase study that used different caloric intakes of a defined monotonous liquid diet to first stabilize initial weight, then to decrease weight by 10%, and finally maintain weight at the 10% reduced level (FIG. 2B; Table 4). Daily caloric intake was 2988±290, 800, and 2313±333 kcal for the three phases of the study, respectively (13,14). While on this diet, these four individuals experienced significantly reduced stability of their microbiota, as measured by the Jaccard Index (FIG. 2B). For each individual, we found no significant correlation between time and diversity/richness (i.e., number of strains in a sample; minimum p-value=0.17). Additionally, we found no significant correlation between the change in composition of the microbiota (Jaccard Index between two samples) and the change in diversity/richness (absolute difference in the number of species/strains between two samples) (p-values=0.09 and 0.44 for strains and species, respectively). Considering family-level taxonomic bins, there were several groups whose abundance was strongly correlated with time during the weight loss period including Clostridiaceae [average correlation (r) across donors during weight loss=0.60], Coriobacteriaceae (r=0.53), Bifidobacteriaeceae (r=0.55), and Enterobacteriaceae (r=0.58), Lachnospiraceae (r=−0.65), Oscillospiraceae (r=−0.53), and Oxalobateraceae (r=−0.74).

Modeling the Relationship Between Time, Body Composition, and Microbiota Stability—

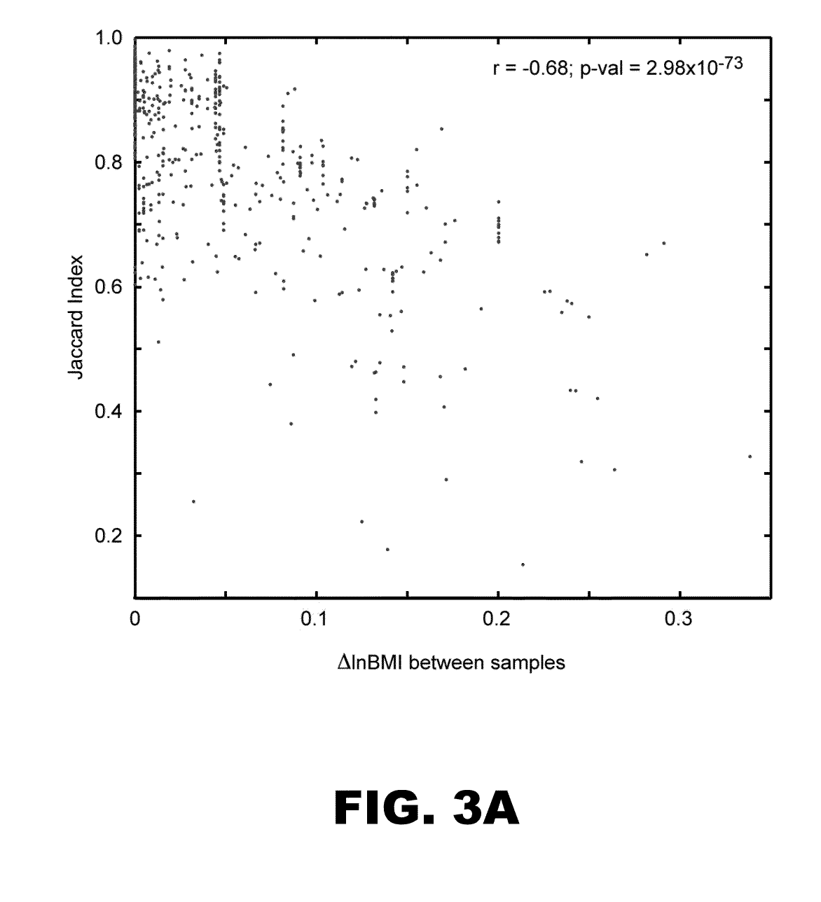

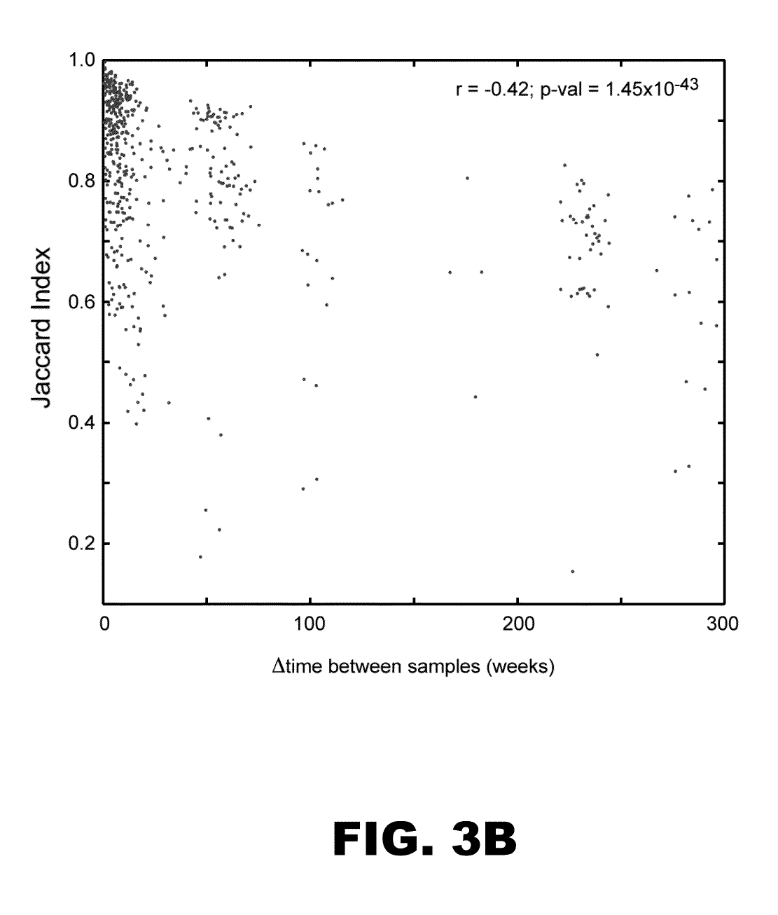

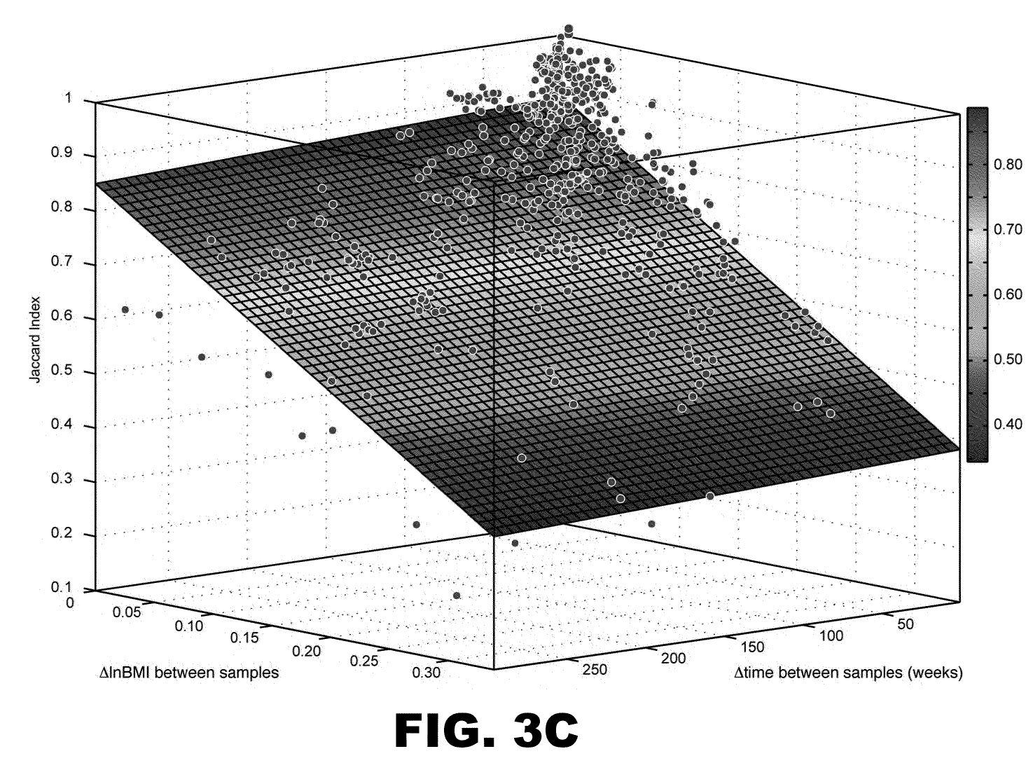

Given the correlation between weight loss and changes in the microbiota of individuals consuming a monotonous 800 kcal/day diet, we took a broader view across all 37 individuals in our study to determine if this correlation was due to the monotonous diet that the four individuals had consumed, or if there is a generalizable and quantifiable relationship between weight stability and microbiota stability. To explore this question, we not only calculated the time (Δtime) and Jaccard Index between all pairs of fecal samples collected from an individual (FIG. 2), but also the absolute value of the change in log(BMI) (abbreviated ΔInBMI) between all pairs. We found a significant negative correlation between ΔInBMI and Jaccard Index (FIG. 3A; r=−0.68; p-val=2.98×10⁻⁷³) that was even greater than Δtime and Jaccard Index (FIG. 3B; r=−0.42; p-val=1.45×10⁻⁴³). These relationships held when we removed the data generated from the four individuals on the monotonous diet (ΔInBMI: r=−0.69; p-val=3.27×10⁻⁵⁴; Δtime: r=−0.65; p-val=9.05×10⁻⁴⁶).

To quantify the relationship between Δtime, ΔInBMI, and the Jaccard Index between samples (FIG. 3C), we fit the following model:

microbiota_stability=β₀+β_{InBMI}X_{InBMI}+βₜᵢₘₑXₜᵢₘₑ

where microbiota_stability is the Jaccard Index between samples, X_{InBMI} is the change in InBMI between any two samples collected from the individual (ΔInBMI), Xₜᵢₘₑ is the time between the two samples being compared (Δtime), β₀ is the estimated parameter for the intercept; and β_{InBMI} and βₜᵢₘₑ are the linear regression estimated parameters for ΔInBMI and Δtime, respectively. Remarkably, this model explained 46% of the variance in the stability of the microbiota (Jaccard Index) within the individuals over time (R²=0.46; p-val=1.94×10⁻⁷² and R²=0.51; p-val=1.40×10⁻⁵⁸ when the monotonous dieters were excluded). Once again the weight stability of an individual (ΔInBMI; ANOVA p-val=1.18×10⁻⁵¹) was a better predictor of fecal microbiota_stability than the time between samples (Δtime; ANOVA p-val=0.09), with Δtime only being a significant predictor of stability when the monotonous dieters were excluded (ANOVA p-val=2.82×10⁻⁷). Together, these relationships between time, BMI, and the stability of an individual's microbiota highlight the role that longitudinal surveys of a microbiota could play in health diagnostics.

### Example 3

### Sequenced Collections of Fecal Bacteria Obtained from Individuals Over Time

As in previous studies (1, 15-18), we found that each individual's microbiota at a given time point was most similar to their own at other time points (Jaccard Index 0.82±0.022), followed by their family members (Jaccard Index 0.38±0.020), and then unrelated individuals (Jaccard Index 0.30±0.005). The accuracy of the Jaccard Index estimates with LEA-Seq suggests that on average any two unrelated individuals share ˜30% of strains in their microbiota. However, it is possible that unrelated individuals on average share no strains in their microbiota and this 30% represents the lower resolving limit of 16S rRNA amplicon sequencing of the targeted variable region (V1V2) and currently available maximum read lengths on the Illumina HiSeq 2000 instrument (paired-end 101 bp).

Whole genome alignments between bacteria isolated and sequenced from different samples provide many orders of magnitude of additional resolving power to determine which strains (now defined at the level of whole genome sequence identity rather 16S rRNA identity) remain in an individual's microbiota over time, or reside in two unrelated individuals. Isolation and sequencing of extensive collections of organisms from the human gut microbiota (19) provides a practical method to look at the plasticity and evolution of the gene content of microbial strains harbored in individuals' intestines over time. Therefore, adapting a high-throughput method we had developed for generating clonally arrayed collections of anaerobic bacteria in multi-well format from frozen fecal samples (19), we produced draft genome sequences for 444 bacterial isolates recovered from the frozen fecal microbiota of five donors who had been sampled across periods from 7-69 weeks apart (n=1-4 time points/donor; 11 total samples; mean coverage/microbial genome=118x; see Tables 6, 7 and Methods). These genomes span a broad phylogenetic range within the four dominant bacterial phyla that comprise the human gut microbiota (Bacteroidetes, Firmicutes, Proteobacteria, and Actinobacteria; Table 7).

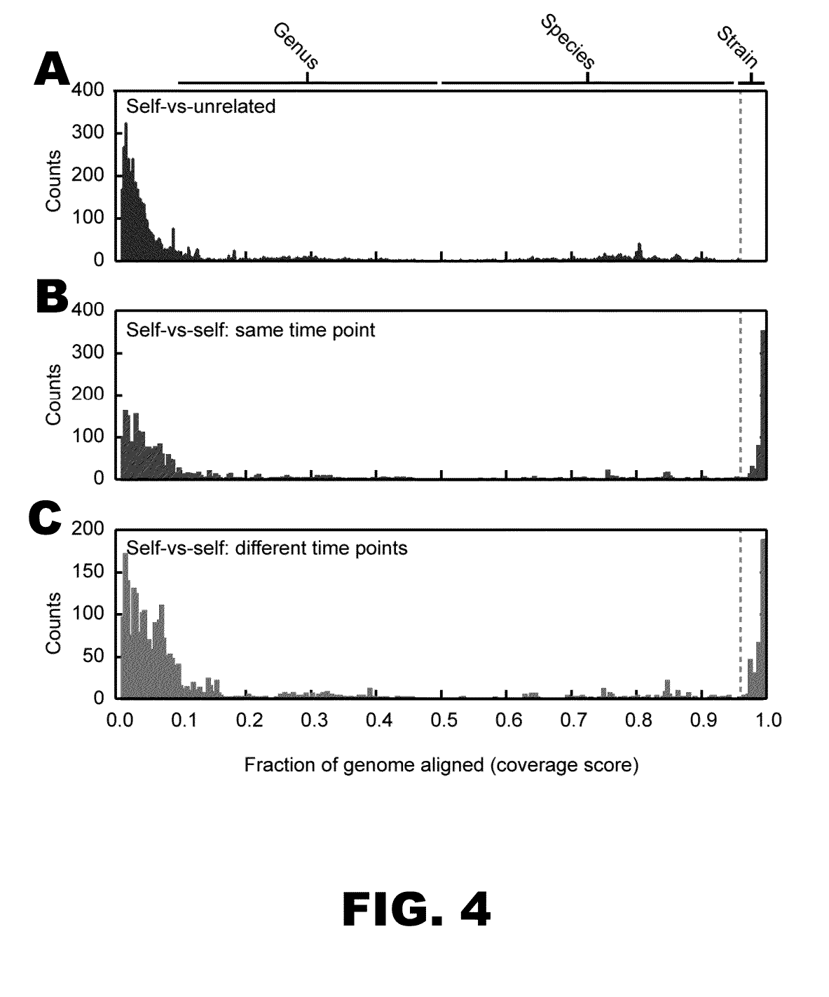

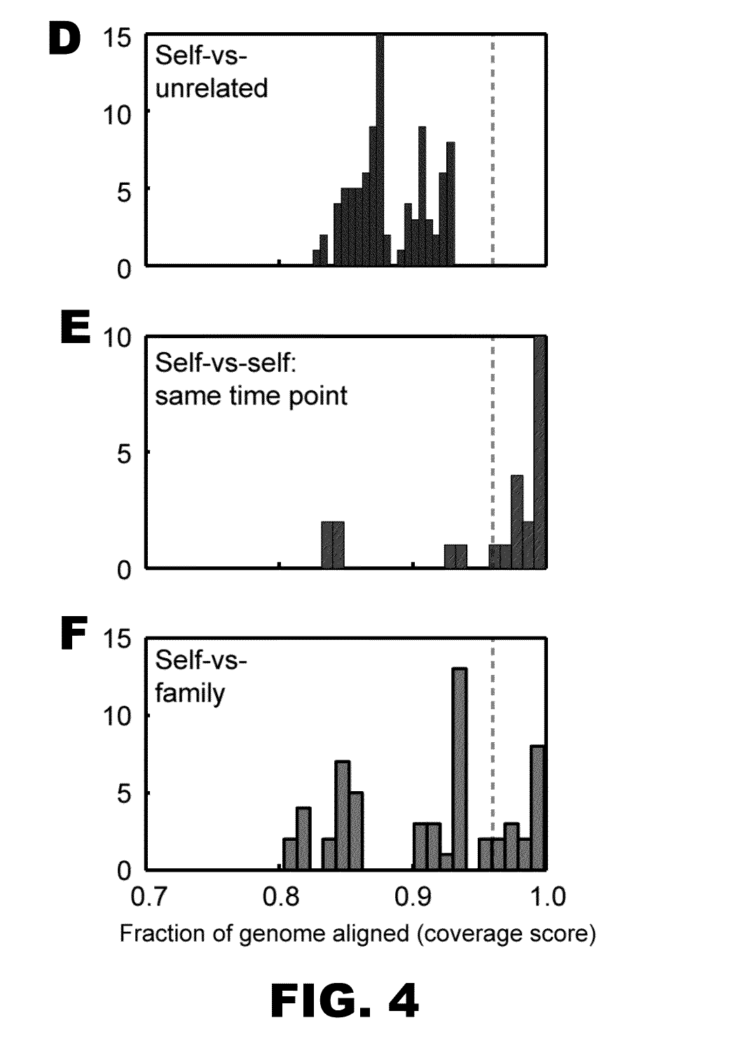

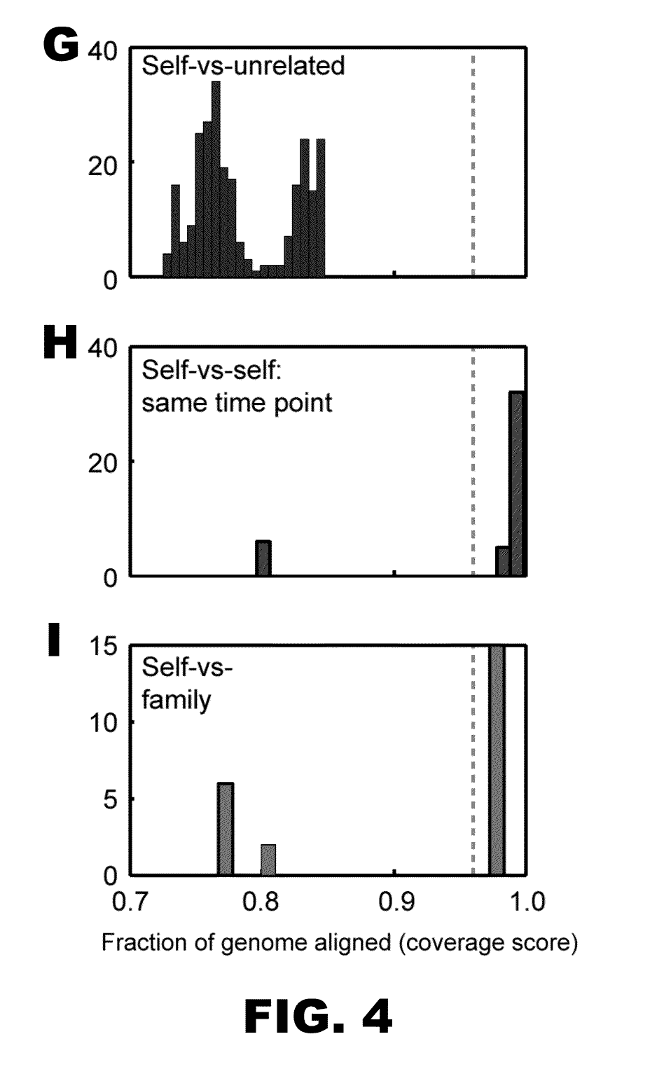

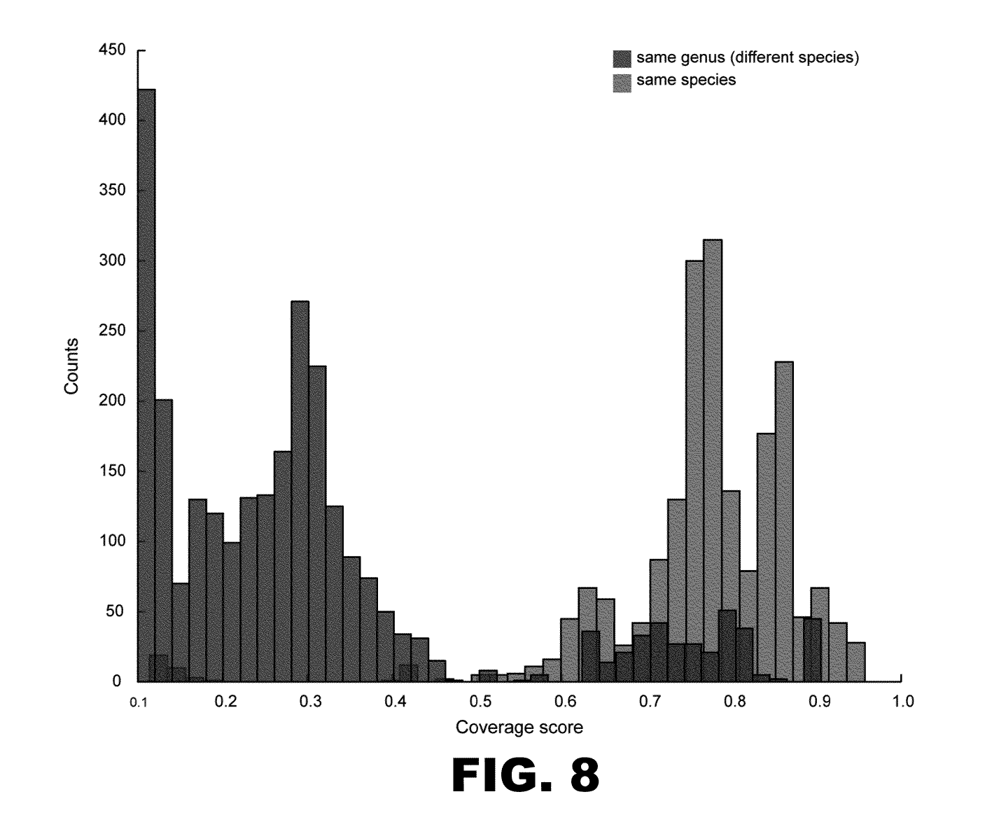

To look for changes in bacterial genome content across time in each individual, we performed whole genome alignment with nucmer (20) and calculated the fraction of DNA sequence aligned between each pair of genomes (coverage score=Xₐₗₙ+Yₐₗₙ/X+Y; where X and Y are the lengths of genome X and Y, respectively, and Xₐₗₙ and Yₐₗₙ are the number of aligned bases of genome X and Y respectively) (21) (see Supplemental Results). We found the shared genome content between isolates from unrelated individuals was broadly distributed for taxa from the same genus (coverage score=0.30±0.20) or species (0.77±0.12), with a maximum of 0.956 (FIG. 4A, blue; FIG. 8). We then compared the shared genome content between isolates within each fecal sample (i.e., self-versus-self at a single time point) and found isolates that shared a very high proportion of their content (0.965-0.999) (FIG. 4A, red). Remarkably, we found the same high proportion of shared genome content between isolates from a given donor between different time points (i.e., self-versus-self over time; FIG. 4A green), suggesting that the same strains of bacteria persisted in these individuals over the course of the sampling period.

Defining replicate bacterial strains as those with a coverage score >0.96 and species as those with a coverage score >0.5 (FIG. 8), we subsequently clustered the genome isolates by sample and by individual (Table 6); this effort yielded a total of 165 strains and 69 species across the five donors (Table 1). Across the four donors with multiple time points, on average 36% of an individual's bacterial strains were isolated from multiple time points. This fraction of shared bacterial strains across time at the level of the genome is lower than that measured by LEA-Seq; however, this likely reflects the increased sampling depth and culture independence of LEA-Seq [detecting isolates at depths of 1:10,000-1:100,000 (0.01-0.001%) compared with 0.14-0.06% for high-throughput culturing]. For the most deeply sampled individual (F3T1 in Table 4), where isolates were sequenced from four samples taken over the course of ˜16 months, over 60% of the strains were isolated from multiple samples.

### Example 4

### Stability Viewed from the Perspective of Phylum-Level Membership

When we assigned phylum-level taxonomy to all LEA-Seq 16S rRNA amplicons from each of the 37 individuals in our study (22), we found that members of the Bacteroidetes and Actinobacteria were significantly more stable components of the microbiota than the population average (hypergeometric distribution comparing the total number of shared/not shared strains within a given phylum for all samples versus the total number of shared/not shared strains across all phyla, except the phylum of interest; p-value=7.54×10⁻²⁸ and 0.0068, respectively), while the Firmicutes and Proteobacteria were significantly less stable (FIG. 2C; p-values=1.83×10⁻¹¹ and 0.0015). The cultured bacterial strains manifested similar trends for the Bacteroidetes and Firmicutes, where 52% and 21%, respectively, of the strains were isolated and sequenced across multiple time points (Table 8), thus demonstrating at a whole genome level the strain stability initially identified when just the 16S rRNA gene was targeted for analysis.

### Example 5

### Strains Shared Between Members of Human Families

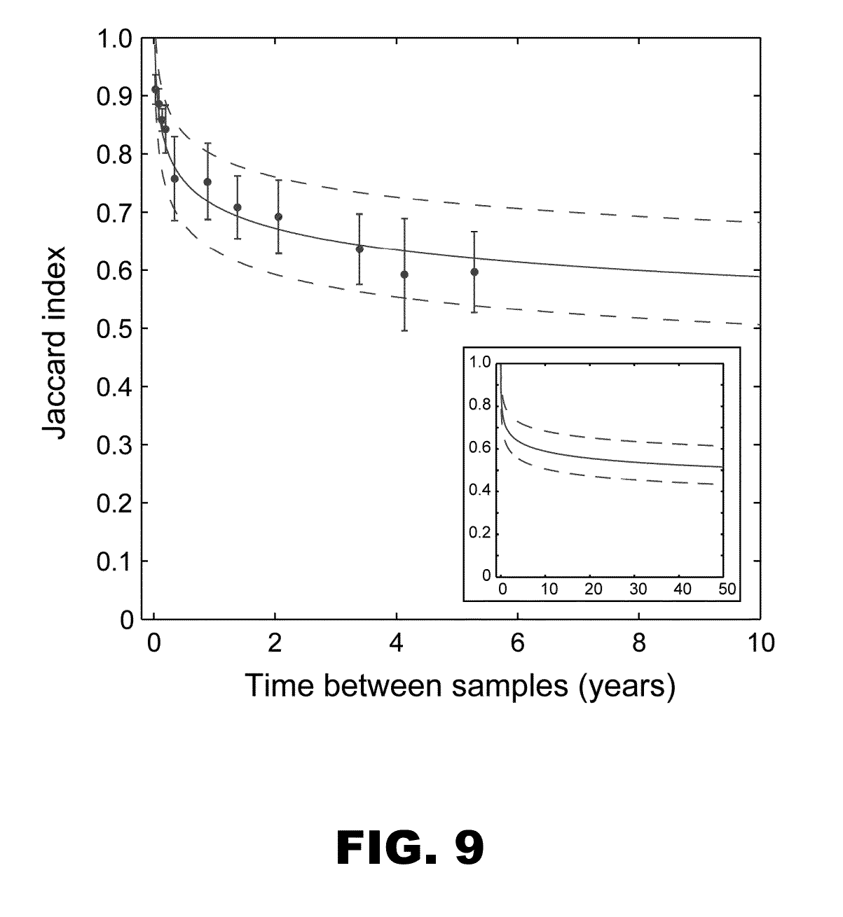

The power law response of the Jaccard Index as a function of the time between sample collection makes it possible to extrapolate beyond the sampling time frame of the current study and suggests that the majority of strains in the microbiota represent a stable core that persists in an individual's intestine for their entire adult life, and could represent strains acquired during childhood from parents or siblings (FIG. 9). Therefore, we used LEA-Seq to measure the fraction of shared strains between family members (sister-sister or mother-daughter). As in previous studies (1), we found the microbiota of related individuals was more similar than unrelated ones with a significantly larger proportion of shared V1V2 16S rRNA sequences [Jaccard Index=0.38±0.020 (related) and 0.30±0.005 (unrelated); p-val=0.00053].

To determine if this increased similarity between family members manifested itself at the level of their gut microbial genome sequences, we used a targeted approach to look at genome content differences in (i) two families using previously sequenced Methanobrevibacter smithii isolates (23) from two sets of twin pairs and their mothers (six total donors; 19 genomes; Table 4), and (ii) five families where 26 Bacteroides thetaiotaomicron strains were isolated with a species-specific monoclonal antibody (Supplemental Methods) (24) from nine donors including sister-sister and mother-daughter pairs (all isolates were from a single sample from each donor; Table 4). M. smithii, a methanogen, is the dominant archaeon in the human gut microbiota and facilitates fermentation of polysaccharides by saccharolytic bacteria such as B. thetaiotaomicron by virtue of its ability to remove hydrogen (23). As with our untargeted large-scale genome sequencing of personal bacterial culture collections described above, we found that unrelated individuals had no pair of isolates of either species that shared >96% of their genome content. However, within an individual we once again found replicate isolates of the same strain (FIG. 4B,C; blue and red). Strikingly, we also found replicate strains of M. smithii or B. thetaiotaomicron shared across family members (FIG. 4B,C; brown and Table 4).

In contrast with the results obtained using this taxon-targeted whole genome sequencing approach, our untargeted sequencing of the clonally arrayed personal bacterial culture collections had only involved two related individuals (female dizygotic co-twins 1 and 2 from family 60; F60T1 and F60T2; Table 4) and had revealed no strains with >96% of their genomes aligned. Therefore, we isolated and sequenced an additional 89 genomes from two timepoints of the dizygotic twin sister (F61T2) of subject F61 T1 (yielding a total of 188 strains and 75 species across the six donors). As with the previous donors, we were able to isolate numerous strains shared across the two time points (8 out of 25=32%). In addition, we were able to isolate two strains (B. thetaiotaomicron and Escherichia coli) in both of the sisters, showing that even non-targeted genome isolation and sequencing is capable of retrieving the same strain across family members. We did not explicitly sample members of our cohort of females during significant physiological transitions such as menarche and menopause. However, the presence of the same bacterial strain in mothers and their adult daughters who had progressed through one or both of these life cycle milestones suggests that components of the microbiota are retained during these events.

### Example 6

### Optimization of Bacterial 16S rRNA Amplicon Sequencing

Assaying Amplicon Sequencing Performance—

The even mock community, composed of equal amounts of DNA from the in vitro cultures of 48 phylogenetically diverse human gut bacteria, was used to assay the performance of various 16S rRNA amplicon sequencing methods. Performance was visualized by plotting precision versus depth, where precision is defined as the fraction of the resulting DNA sequences that are 100% identical to the 16S rRNA region in the complete genomes of the 48 species in the pool, while depth is defined as the minimal fractional abundance a given sequencing read must represent in order to include it in a given analysis (e.g., a threshold of 0.01 includes sequences representing 1% or more of the final sequencing reads). Assuming true sequences will be more frequent than false ones, increasing this threshold should increase the proportion of true-positive sequences. The best 16S rRNA amplicon sequencing methods would produce the highest precision at the lowest threshold. We quantified the precision of each method at depth thresholds (proportional representation) of 1:500, 1:1000, 1:5000, 1:10,000, and 1:50,000.

Most of the reference strains had only draft genome assemblies, raising the possibility that their 16S rRNA genes might not be fully assembled and annotated. Therefore, we generated a gold-standard set of all “true-positive” 16S rRNA sequences using BLAST or bowtie (32) so that we could map the sequencing reads for a given amplicon sequencing method to the reference genomes (bowtie was employed for paired-end reads that do not overlap and thus can not be assembled into a continuous amplicon). All sequences with 100% sequence identity across 100% of the sequence length to a reference bacterial genome were included in the final gold-standard “perfect” set for each pool (mock community).

Masking, Sensitivity, and Resolution—

Analysis of 16S rRNA amplicon sequencing data often involves clustering the reads into “species”-level operational taxonomic units (OTUs) containing sequences that share 97% identity. However, this clustering into OTUs could obfuscate significant associations between bacteria and their host that do not operate on the higher taxonomic levels; e.g., a specific strain of Bacteroides thetaiotaomicron might generate a given phenotypic response in the host, rather than all members that occupy the same 97% identity species-level OTU (33). To track individual species or strains at the highest possible resolution, the strain's genome sequence provides the maximally informative identifier. Nonetheless, the 16S rRNA gene is a good widely used single-gene identifier (34). The current read lengths of next-generation DNA sequences are too short to sequence the entire 16S rRNA gene. Therefore shorter, variable regions of the gene are typically amplified and sequenced (35-38). The suitability of any given region of the 16S rRNA gene to serve as a unique strain-level identifier within an individual's microbiota depends on the generality of the primers designed for the region, combined with the information content/diversity of the region. The most sensitive 16S rRNA region for amplicon sequencing in terms of capturing the largest fraction of diversity in the microbial population would have an available pair of conserved primers that could quantitatively amplify that region from all possible DNA templates in a microbial community of interest (35). The most informative region would be sufficiently diverse at the nucleotide level to uniquely identify all strains present in the DNA pool. Diversity in the conserved regions used to design primers should decrease the method's sensitivity and quantitative accuracy. A lack of diversity in the intervening amplified ‘variable’ region increases the chance of masking, where multiple strains present in the DNA pool have identical amplicon sequences and are thus quantified as a sum of their individual abundances.

To examine the sensitivity and masking associated with different variable regions of the 16S rRNA gene present in various human gut bacterial species, we performed a paired-end alignment to map primers (Table 10) for PCR of the V1V2 region and the V4 region against a diverse reference set of 128 sequenced genomes from human gut bacterial symbionts (Table 11). The most sensitive primer pairs will map to the largest number of reference genomes, while the region with the least masking will uniquely identify the largest proportion of genomes. We used bowtie (32) and allowed no more than three nucleotide mismatches for each primer in a paired-end alignment. Across the 128 human gut microbial genomes, we found that V4 primers were the most sensitive, capturing the 16S rRNA V4 region from 122 genomes (95%) compared to 100 genomes captured by the V1V2 primers (78%). Similar results have been observed in previous studies across a wide-range of ecosystems (35). However, we found the V1V2 amplicon sequence provides higher resolution strain identification; 92 of the 100 genomes captured by the V1V2 primers could be uniquely identified by their amplicon sequence compared to 86 of the 122 genomes (70%) captured by the V4 primers. Even when the V4 amplicons are limited to the subset of 100 genomes that could be captured by the V1V2 primers, only 78 of the genomes (78%) could be uniquely identified. Thus, the decision to amplify the V1V2 or V4 regions of bacterial 16S rRNA genes for a given analysis requires a choice between higher sensitivity (V4) and higher resolution (V1V2). The higher sensitivity of the V4 primers and higher resolution of the V1V2 region was also observed empirically during our quantitative analysis of different 16S rRNA amplicon sequencing methods (see below).

V1V2 16S rRNA Amplicon Sequencing Using the Roche 454 FLX Pyrosequencer—

As an initial benchmark, we measured the performance of a standard method of amplicon sequencing of the V1V2 region with the Roche 454 pyrosequencer using Titanium chemistry. The V1V2 primers (Table 10) were designed to sequence from the 338R primer towards the 8F primer. The 338R primer was trimmed from the resulting amplicon sequences. The 454 pyrosequencer generates variable-length amplicons, so for performance evaluations all 454 amplicon sequences were trimmed to 315 bp (sequences shorter than this were removed). Based on previous studies showing that 2000 reads provide a good balance between cost and coverage (37), we generated 1955 amplicon sequences, using the even mock community, and obtained a precision of 0.48 and 0.24 at abundance thresholds of 1 in 500 (0.2% of the mock community) and 1 in 1000 (0.1%), respectively (FIG. 1 green line, and Table 3). Although this sequencing platform, primer set, and sequencing depth has been quality-controlled with numerous phylogenetic and clustering metrics (26, 36, 37), it has an unsuitably low precision if the goal is to track individual strains in longitudinal studies of the human microbiota at high depths.

V4 16S rRNA Amplicon Sequencing Using the Illumina MiSeq Instrument—

A second widely targeted region of the bacterial 16S rRNA gene is V4. Although this region has a slightly higher masking rate in human gut bacteria than the V1V2 region, the primers are more sensitive (see above). Another advantage of the V4 region is that its slightly shorter length enables coverage with an Illumina MiSeq instrument (38) using a paired-end 150nt kit for reduced cost and labor per sample. To generate a full length V4 16S rRNA amplicon sequence with a paired-end Illumina MiSeq sequencing run, the paired-end reads were joined into a single sequence (using the overlap between the two reads) with the flash algorithm (version 1.0.2) (39).

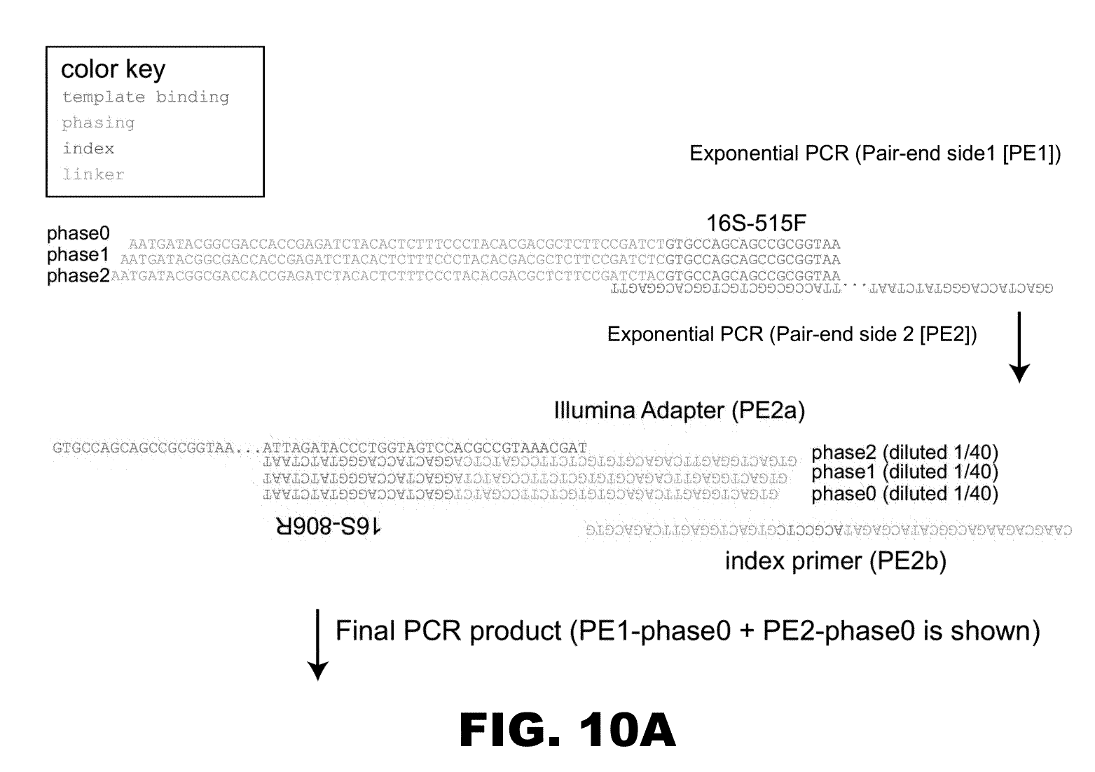

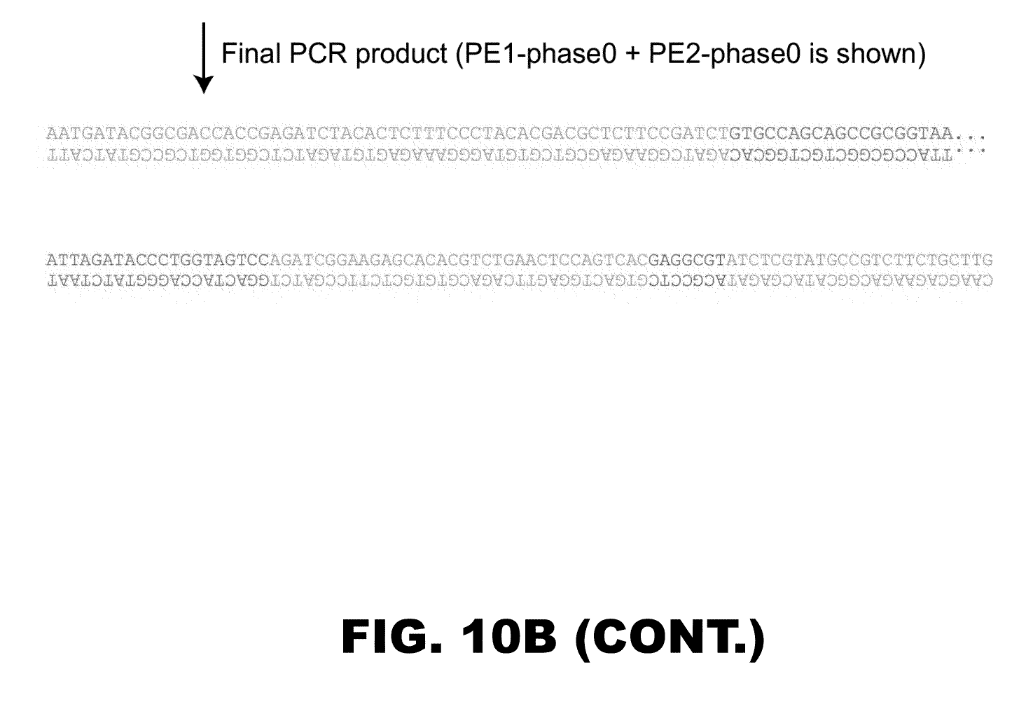

A current limitation of the image-based hardware and algorithms associated with the Illumina next-generation sequencing platforms is the need for an even distribution of the four nucleotide bases at each sequencing position. This presents a significant hurdle for sequencing the evolutionarily conserved 16S rRNA gene. The base distribution complexity can be increased by adding genomic DNA to the sequencing run (e.g., from phi X174 bacteriophage), but at a cost of reduced yield for the amplicon sequences of interest. To decrease the amount of phi X174 DNA necessary for each run, we generated primer pools with different amounts of phasing (FIG. 10), with the phase nucleotides hand-picked to maximize the evenness of each base during the first 13 bases of each paired-end sequencing read (these initial bases are used by the Illumina software to estimate the phasing and pre-phasing values that are critical for accurate base calling; Table 12). Moreover, to further increase nucleotide diversity at each base, we amplified the V4 16S rRNA region from both directions separately and sequenced them simultaneously [i.e., read1 and read2 both contained sequences that began with the primer binding at base 515 of the 16S rRNA gene and sequences that began with the primer binding at position 806; FIG. 10]. We found that increasing the amount of phasing and sequencing the amplicon in both directions allowed us to generate sequencing runs with a lower error rate and less phi X174 spike-in DNA, as measured by the percentage of phi X174 bases that matched perfectly to the phi X174 reference genome by Illumina quality control software (Table 12). An index was added to each sample with a third PCR primer (FIG. 10) to allow pooling of multiple samples in a single MiSeq run. Phase nucleotides and primers were trimmed from the sequences prior to analysis and the amplicons were reverse complemented as necessary to put them in the same orientation.

Overall, V4 16S rRNA sequencing on the Illumina MiSeq platform obtained substantially higher precision at a given threshold than V1V2 sequencing on the Roche 454 FLX platform (precision at a threshold of 1:1000 was 0.76±0.097 compared to 0.24 for the Roche 454; Table 3). This increase in performance was partially attributed to the increased depth of sequencing provided by the MiSeq instrument, as sequencing replicate samples on the 454 FLX platform to a depth of >40,000 reads increased performance (0.57±0.021; Table 3), while subsampling the MiSeq data to the same depth as the 454 data (2000 reads/sample) produced a similar though less substantial decrease in performance, dropping the precision at a threshold of 1:1000 down to 0.45 compared with 0.24 with the 454 FLX platform (Table 13A). This result suggests that increased sequencing depth enables a more accurate estimate of which sequences are more/less abundant than a given abundance threshold. Further support for the idea that increased sequence depth allows more accurate filtering and increased precision at a given threshold came when we found that as we subsampled the reads from an amplicon dataset performance converged to its maximum with larger numbers of reads (Table 13B). For the MiSeq instrument, we found that sequencing to a depth of ≧10,000 reads per sample provides a reasonable balance between precision and throughput per run (384 samples can easily be pooled in a single run and sequenced in one day). At this depth of sequencing, taxa present at an abundance ≧1:1000 (0.1%) can be detected with a precision of 0.78±0.051. We found no large changes in performance when testing different DNA polymerases for the PCR reaction, different primers, or the uneven pools of genomic DNA (Table 13C; each sample was subsampled to 10,000 reads for comparison).

Quantitative Performance of V1V2 and V4 Targeted Amplicon Sequencing—

The eight uneven DNA pools (mock communities) generated from 48 diverse gut microbial species provided an opportunity to measure the quantitative performance of 16S rRNA amplicon sequencing. We tested two DNA polymerases and two primer sets (one consensus primer with degenerate nucleotide positions to better represent diversity of the variable region, and one with the most abundant sequence for the variable region in the gut bacterial genomes being tested; Table 10). We calculated the quantitative performance of a 16S rRNA amplicon sequencing method as the correlation between the natural log of the known fractional abundance of each strain in each pool and the natural log of the measured fractional abundance of each strain. The correlation (r) between the known and the observed fractions across the pools was ˜0.8, regardless of the primers or the DNA polymerase (Table 14), which is comparable to the quantitative performance measured in a large “spike-in control” study using Affymetrix GeneChips (40).

Since each species was present at four or more concentrations across the eight pools, we could measure the species-level quantitative performance of different 16S rRNA amplicon sequencing methods. In addition to the correlation between known and expected abundances of each strain described above, we could also determine the slope of a line fit by linear regression of the log of the known fractions versus the log of the observed fractions of each species. Deviations away from 1.0 provided information about which strain abundances might be under- or overestimated with a given 16S rRNA amplicon sequencing protocol. While there were a few outliers with particularly low or high correlations and estimated slopes, we found that overall at the level of individual species the average correlation and slope was very high (>0.98; Table 15).

### Example 7

### Additional Details about Low Error Amplicon Sequencing (LEA-Seq)

Data Processing and Performance—

16S rRNA reads are separated by the indexing read and trimmed to remove primer sequences and extra phasing nucleotides. For each sample, sequence reads are grouped by the random barcode, and groups with less than four reads are removed. Although theoretically the length and redundancy of the synthesized random nucleotides on each linear PCR primer should generate an enormous potential complexity (from 9.1×10⁸ to 1.4×10¹⁰ potential barcodes), sequencing errors and bias during DNA synthesis or PCR could make it difficult to distinguish true barcodes from false positives. To eliminate ambiguous sequences, the random barcode sequences are sorted by abundance and clustered at an identity of 86% using the uclust algorithm (41). Running the uclust algorithm with the—usersort option on the abundance-sorted barcode set forces the algorithm to preferentially cluster the barcodes from most abundant to least abundant. Given that most sequencing errors are random and that the correct sequence should occur more often than a variant with sequencing errors, the abundance-weighted clustering algorithm provides a means to eliminate spurious barcodes that are most likely due to sequencing errors while retaining the more abundant (and most likely true positive) barcode sequences. Only the sequence reads containing the most abundant barcode representative of each uclust 0.86 identity cluster are retained for further analysis.

Since amplicons with the same random barcode sequence originated from a linear PCR extension of one template molecule that was subsequently amplified by exponential PCR, they should be identical. This redundant sequencing of each linear PCR molecule allows us to error-correct each amplicon. In the present study, as an initial filter the sequences associated with each random barcode were clustered with uclust at an identity of 0.98. Amplicon groups where the most abundant sequence cluster was less than 2.5 times the second most abundant sequence cluster were eliminated. We then generated a consensus sequence from each group using all of the sequences present in the most abundant sequence cluster. The score for each nucleotide at each base position was weighted by the square root of the abundance of the amplicon sequence (e.g., if sequence AAAA is present in the cluster four times and TAAA is present in the cluster one time, nucleotides in the first sequence would get a weight of 2 and those in the second sequence would get a weight of 1). The quality of each position was measured as the score for the most abundant nucleotide at that position divided by the sum of the scores for all nucleotides at that position. Consensus sequences where one or more bases received a score below ⅔ were excluded. We kept only those sequences whose consensus sequence was identical to the most abundant sequence associated with the same random barcode.

Because the performance saturation of LEA-Seq was beyond the depth of sequencing employed for this study (FIG. 6), we found that a simple counts-based threshold (i.e., to be retained a sequence must occur at least N times in the set of sequencing reads) was an efficient way to filter reads as it allowed increased sensitivity for samples that were sequenced more deeply.

Quantitative Performance and Masking with LEA-Seq—

Given the extra linear PCR step and computational processing involved in the LEA-Seq method, we wanted to verify that the resulting quantitation of each strain in a community was as accurate as standard amplicon sequencing. As above, we compared the log of the known fraction of each of the 48 strains with the log of their fraction measured using LEA-Seq and targeting either the V1V2 region or the V4 region (using both the abundant and consensus primers; Table 10). The correlation between the known and measured fraction of each strain was once again ˜0.8 (Table 14).

The uneven pools (mock communities) also provide an empirical dataset to compare with our computational analysis of masking and resolution above. As noted earlier, LEA-Seq requires approximately 20-fold coverage of each linear PCR reaction. Therefore, we used the Illumina HiSeq 2000 instrument to sequence pools of up to 24 samples per lane at significantly less cost per base than what is incurred with the Roche 454 FLX or Illumina MiSeq instruments. The maximum current read length of the Illumina HiSeq 2000 platform is paired-end 101 nt, which is too short to assemble into a continuous amplicon sequence for the V1V2 or V4 region. After removing the random barcode and two primer sequences, we ended up with a 63 bp×79 bp fragment for the V1V2 region and a 64 bp×77 bp fragment for the V4 region. We found these shorter regions were difficult to assign taxonomy below the family level. However, for use as a strain identifier, the shorter regions have only slightly reduced performance compared to the full amplicon sequence of the V1V2 and V4 regions. With the 48-member mock community, the V4 full-length amplicon uniquely identified 82% of the strains while the shorter V4 LEA-Seq amplicons uniquely identified 78% (Table 14). Similar to the computational analysis of masking on V1V2 versus V4 above, we found empirically that the V1V2 region had a lower masking rate than the V4 region; it uniquely identified 87% of the strains in the community. Finally, the primer sensitivity on this empirical dataset from the 48-member consortium also mirrors our computational analysis above; the V1V2 region amplified 87% of the strains in the pool compared to 96% for the V4 region.

### Example 8

### Shared Community Membership in Longitudinal Studies of Twins

By retaining high precision at high depths, LEA-Seq provides an opportunity to track strains of bacteria within an individual over time. As an initial benchmark, we ran LEA-Seq on four mock communities containing 3, 6, 32, and 48 different bacterial strains (species) respectively (Table 2) with differing number of overlapping strains between the four communities. Using the set of known 16S rRNA sequences extracted from the genomes of each of the strains, we calculated the Jaccard Index between all six possible pairwise comparisons between the four mock community datasets. The proportion of shared strains between the four mock communities ranged from 0.111 to 1.000 (Table 16A).

To empirically test our ability to assay the shared microbiota between two samples, we performed LEA-Seq of the V1V2 region of the 16S rRNA gene for each of the pools (n=25 samples; 202,227±164,646 reads/sample; all samples had >50,000 reads except the three-member mock community [4,165 reads] and the six-member community [6,506 reads] where sequencing depth was less important). As above, we chose eight sequencing reads as the minimum threshold to include the sequence in the analysis. However, to calculate the Jaccard Index we only required the sequence to have at least the minimum number of reads in one of the two samples; to consider the strain present in the second sample, it needed to have a read at any abundance that was 100% identical across 100% of its length.

We calculated the Jaccard Index between all 300 pairwise comparisons of the 25 samples and calculated our ability to correctly estimate the proportion of shared strains between any two samples. Overall, the correlation between the known and the measured values of the Jaccard Index was high (r=0.9349) with the mean absolute difference between the known and measured values (i.e., mean(abs(known−measured))) being 0.11±0.13 (Table 16B). However, the correlation and the mean absolute difference was clearly different between samples on the same HiSeq2000 run compared to those run separately, with the Jaccard Index measured from samples on the same run having lower deviation from the known value (mean absolution difference of samples on the same HiSeq2000 run=0.027±0.024, r=0.9963 versus 0.18±0.13, r=0.9894 for samples on different runs). Therefore, for comparisons with human samples we placed all samples from the same donor on the same sequencing run. Our ability to estimate the proportion of shared strains between two samples with such fidelity is somewhat surprising given that we measured a precision of 0.60 with a minimum threshold of eight reads for the V1V2 regions represented in the 48-member mock community, suggesting many of the false positive sequences in each sample are consistently generated on the same Illumina HiSeq2000 run.

### Example 9

### Comparing LEA-Seq with Standard Amplicon Sequencing on Longitudinal Samples from Two Human Donors

The cost of reagents and the experimental time required by standard amplicon sequencing and LEA-Seq are virtually identical. LEA-Seq is significantly more expensive than standard amplicon sequencing due to the need to redundantly sequence each amplicon (10-20× depending on the desired depth). This cost difference will become negligible as next-generation sequencing costs drop. For the present, it is interesting to compare the differences in results obtained by LEA-Seq and standard amplicon sequencing on the same human samples. To do so, we processed LEA-Seq data for nine samples from two donors without generating a consensus sequence (donors F22T1 and F3T2; samples F22T1.1, T22T1.1, F22T1.3, F22T1.4, F22T1.5, F3T2.1, F3T2.2, F3T2.3, and F3T2.13). As noted in the main text, without generating the consensus sequence, LEA-Seq data are experimentally and computationally equivalent to standard amplicon sequencing (with only an extra linear PCR step) and yield similar performance (see Table 3; Method=“LEA-Seq without consensus”). To correspond to the optimum sequencing depths we identified for standard amplicon sequencing, we randomly selected 10,000 LEA-Seq reads from each sample and filtered the reads at a threshold of 0.1%. After correcting for the precision of LEA-Seq (0.63 at a threshold of 8 reads) and LEA-Seq without consensus (0.56 at a threshold of 0.1%), we identified, on average, three-fold more strains in samples analyzed by ‘LEA-Seq with consensus” compared those analyzed using LEA-Seq without a consensus (269 versus 89 strains, respectively). This ‘increase” in the number of strains is due to the increased detection depth. We found a high correlation (r=0.94) in the Jaccard index between samples processed by LEA-Seq and LEA-Seq without consensus, suggesting that the stability of the microbiota is similar enough between low-abundance and high-abundance taxa to enable stability to be accurately measured using only high-abundance taxa. The results also indicate that high abundance and low abundance strains tend to remain at similar abundances across time, otherwise frequent drops below the detection depth for high-abundance microbes would have led to decreases in the calculated Jaccard index. Finally, quality filtering of the sequences is critical to accurately estimating both the number of strains in a microbiota and its stability, as unfiltered LEA-Seq data without a consensus yield an average of >4000 strains across the two donors and an average Jaccard index of 0.075 versus an average of 0.78 and 0.77 for filtered LEA-Seq and filtered LEA-Seq without a consensus, respectively—vastly overinflating richness and underestimating stability by more than 10-fold: in other words, without filtering the microbiota appears much more diverse and much less stable.

### Prospectus of the Examples

The objects we touch and consume during the course of our lives are covered with diverse microbial life. Despite this, we find with LEA-Seq that on average 60% of the approximately 200 microbial strains harbored in each adult's intestine is retained in their host over the course of a five-year sampling period. Our results are supported by a microarray-based profiling of fecal microbiota collected from three males and two females over ˜8 years (18), but differ from a similar analysis using standard 16S rRNA amplicon sequencing that found high variability in microbiota composition in two individuals sampled for up to 15 months (25). This difference likely reflects the fact that the sequencing depth and precision limitations of standard 16S rRNA amplicon sequencing are overcome to some extent with microarrays where amplicons are mapped/hybridized to a finite pool of target sequences (i.e., sacrificing resolution for precision). The differences could also be due to true differences in the stabilities in microbiota of the individuals, as both studies surveyed only a small number of individuals. Our findings are also supported by a recent report that mapped deep shotgun sequencing datasets of the fecal microbiome to a set of reference bacterial genomes (6) and found that the gut communities of these individuals were more similar to each other at the microbiome level than to unrelated individuals (average maximum time between samples=32 weeks with two individuals sampled over a period >1 year). Applying LEA-Seq to longitudinal surveys of the fecal microbiota of 37 twins sampled for up to five years allowed us to identify that the stability of an individual's microbiota follows a power-law function. Using this function, we could extrapolate the stability of the microbiota over decades. The resolution and accuracy of these predictions should improve as advances in sequencing chemistry enable longer regions of 16S rRNA genes to be characterized. LEA-Seq itself can be generalized to any application that requires deep amplicon sequencing with high precision (e.g., the VDJ regions of immunoglobulin and T-cell receptor genes, or targeted searches for variants in candidate or known disease-producing genes).

Our study also illustrates how a highly personalized analysis of the gut community, at strain-level microbial genome resolution can be conducted using collections of cultured bacteria (or archaea) generated from frozen fecal samples collected over time from a given subject. We demonstrate that this strain-level analysis can be part of a broad phylogenetic survey, or it can target a particular species.

The stability of the microbiota that we document in healthy individuals has important implications for future use of the microbiota (and microbiome) as a diagnostic tool as well as a therapeutic target for individuals of various ages. Our findings suggest that obtaining a routine fecal sample as part of a yearly physical examination designed to promote disease prevention would be sufficient to monitor changes in the composition and stability of an individual's fecal microbiota. For example, in the case of inflammatory bowel diseases, the concordance for Crohn's disease and ulcerative colitis among monozygotic twin pairs is only 38% and 15% respectively (26). Our results suggest that these twins likely share identifiable unique subsets of their microbiota that represent long term environmental exposures for their immune systems that should be considered when trying to predict disease risk, or infer which species/strains may have a causal role in disease initiation, progression, relapse and treatment responses. Moreover, the effects of travel, changes in diet, weight gain and loss, diarrheal disease, antibiotics, immunosuppressive therapy, or clinical trials designed to deliberately manipulate the microbiota (e.g., through administration of existing or new prebiotics, probiotics, synbiotics, antibiotics or transplantation of microbiota from healthy individuals to those with various diseases attributed to a dysfunctional microbiota) can be more accurately quantified by applying the methods we describe. Finally, the stability we document highlights the impact of early colonization events on our microbiota in later life; earlier colonizers, such as those acquired from our parents and siblings, have the potential to provide their metabolic products and exert their immunologic effects for our entire lives.

### Methods for the Examples

### Diet Studies

Four obese (BMI >30 kg/m²) female subjects with a mean (±SD) age of 26±3 years were admitted to the General Clinical Research Center at Columbia University Medical Center and remained as inpatients throughout the study. The protocol for recruitment, and the weight loss study was approved by the Institutional Review Board of the New York Presbyterian Medical Center and is consistent with guiding principles for research involving humans. Written informed consent was obtained from all subjects. The diet protocol has been described in detail previously (11, 12). Briefly, subjects were fed a liquid-formula diet with 40% of energy as fat (corn oil), 45% as carbohydrate (glucose polymer), and 15% as protein (casein hydrolysate). Diet composition but not quantity was constant throughout the study. The diet had a caloric density of 1.25 digestible kcal of energy/g and was supplemented with vitamins and minerals in quantities sufficient to maintain a stable weight, defined as an average daily weight variation of <10 g/d for ≧2 weeks. This weight plateau is designated as Wtᵢₙᵢₜᵢₐₗ. The four individuals in this study consumed 2600-3300 Kcal/day of the diet to maintain Wtᵢₙᵢₜᵢₐₗ. After a brief period at Wtᵢₙᵢₜᵢₐₗ, subjects were provided 800 kcal energy/d of the same liquid-formula diet until they had lost ˜10% of Wtᵢₙᵢₜᵢₐₗ. The duration of the weight-loss phase ranged from 36 to 62 days (Table 4). Once 10% weight loss had been achieved, intake was adjusted upward until subjects were again weight stable. Weight maintenance calories were disproportionately reduced (˜22%) below those required to maintain initial weight and ranged from 2050-2800 Kcal/day for the four individuals. Subject F72 also received 25 μg/day triiodothyronine during this second weight stable period (Table 4). Fecal samples were obtained throughout the study (Table 4) and frozen at −80° C. until processed for DNA extraction (1).

### Twin Participants

Twins were selected from a general population cohort of female like-sex twin pairs, born in Missouri to Missouri-resident parents between Jul. 1, 1975 and Jun. 30, 1985, and first assessed at median age 15 with multiple waves of follow-up (27, 28). Selected twins were drawn from (i) a study, which included biological mothers where available, contrasting stably concordant can twin pairs (both twins had BMIs in the range 18.5-24.9 by self-report at all completed assessments) and concordant obese twin pairs (both twins had BMI's ≧30, but with pairs prioritized where at least one twin had BMI>35, to maximize separation from the concordant can pairs) (1); (ii) a small-scale study of concordant can MZ pairs contrasting free diet with free diet supplemented by twice daily consumption of a fermented milk product (10); and (iii) an ongoing study of twin pairs selected for BMI discordance (either discordant lean/obese, or quantitatively discordant).

### Creating Mock Communities

A set of 64 sequenced human gut bacterial isolates (Table 2) were grown at 37° C. in TYGS medium (20, 30) in a deep 96-well polypropylene plate (Nunc) under anaerobic conditions (defined as an atmosphere of 5% H2, 20% CO₂, and 75% N₂) in an anaerobic chamber (Coy Laboratories, Grass Lake, Mich.). After a 72 h incubation, the contents of each well were aliquoted into shallow 96-well polystyrene plates (TPP) and stored at −80° C. in 15% glycerol under an aluminum foil seal. Although many gut bacteria require a strict anaerobic environment for growth, we found that cultures frozen on dry ice in the anaerobic Coy chamber prior to storage in a −80° C. freezer could be recovered at a future date by thawing the plates in the chamber and then immediately transferring an aliquot (5-20 μL) from each well into anaerobic plates containing reduced TYGS medium [transfer done in the anaerobic chamber using a Precision XS robot (BioTek); for details see below].

The availability of complete genome sequences for each of the strains, combined with the diversity of the strain consortium, provided a resource to test and validate different methods of 16S rRNA amplicon sequencing. Therefore, we grew the clonally arrayed collection of different bacteria in two replicate deep 96-well plates in TYGₛ medium under anaerobic conditions. We then extracted DNA from each well in both plates by transferring the contents into individual 2 mL screw cap tubes and performing bead-beating in the presence of phenol/chloroform (5 min at 25° C.), followed by a clean up step that used a Qiagen 96-well PCR purification column.

The quantity of DNA extracted from each of the 64 organisms was assayed using Quant-IT broad range dye (Life Technologies). An equal amount of DNA from each of 48 of the 64 species was combined into a single tube (final concentration 2 ng/L). We also generated two sets of four pools where in a given pool each strain was present at one of eight different dilutions (1:12, 1:24, 1:48, 1:96, 1:191, 1:383, 1:765, 1:1530) with six total strains at each dilution (Table 9). To ensure that each species was observed at multiple concentrations across the pools, we used a greedy algorithm to randomly assign the concentration of each species in each pool such that within each of the two sets of four pools, a given strain was assigned to four different concentrations. Across the two pools of four uneven dilutions, each of the 48 strains was present at a mean of 5.9 different concentrations (minimum=4; maximum=8). Finally, we generated three additional mock communities containing even concentrations of 3, 6 or 32 bacterial strains that were partially overlapping in composition to the 48-member panel (note that 64 unique bacterial strains were used across the four community subsets; Table 2).

Phased Bi-Directional 16S rRNA Gene Amplicon Sequencing on the Illumina MiSeq

For each PCR reaction, 4 ng of purified template DNA was amplified in a reaction volume of 20 μL. Three primers were used in each reaction (FIG. 10) with the two outermost primers (PE1 and PE2b) at a final concentration of 250 nM and the innermost primer (PE2a) at a final concentration of 8.33 nM ( 1/30^{th} the concentration of the outer primers to ensure the final product is enriched for the longest PCR amplicon that will contain the index from primer PE2b). Each of the primers that bind the 16S rRNA gene of the template DNA (PE1 and PE2a) contains a pool of evenly mixed oligos, each at a different phase (FIG. 10). There are four phases for the primers that bind at position 515 of the bacterial 16S rRNA gene and eight phases for the primers binding at position 806 (Table 10). For each sample, two PCR reactions were run: one with the PE1 and PE2a primers binding at positions 515 and 806 respectively, and the other with the PE1 and PE2a primers binding at positions 806 and 515, respectively. Each reaction was denatured at 98° C. for 30 sec followed by 26 cycles of (98° C.×10 sec, 50° C.×30 sec, 72° C.×30 sec), followed by a final extension at 72° C. for 2 min. After amplification, the two PCR reactions were combined and sequenced together so that for each end of the paired-end read there were twelve different phases and starting position combinations (four for 515 and eight for 806) being sequenced simultaneously to increase the complexity at each position. DNA was quantified for each sample (Qubit HS) and combined in equal proportions. Pools were purified with 60 μL AmpureXP beads added to 100 μL of sample (i.e., a beads to sample ratio of 0.6) and sequenced on an Illumina MiSeq instrument at a loading concentration of 10 pM.

### LEA-Seq Amplicon Sequencing on the Illumina HiSeq2000 Instrument

A linear PCR primer was diluted such that approximately 150,000 linear extensions would be sequenced per sample at 20× coverage. As with the phased amplicon protocol for the Illumina MiSeq instrument, we added phased nucleotides to the LEA-Seq primers to increase sequence complexity (FIG. 5). The Illumina HiSeq2000 instrument was less sensitive to low sequence complexity in the input sample (i.e., having a large proportion of the sequence clusters with the same base), as one lane of the eight-lane flow cell was devoted to training the base-calling algorithms. As a consequence, we were able to use only three phases to retain as many nucleotides as possible for sequencing the 16S rRNA gene. Each of the three phased linear PCR primers (200 μM stock concentration) (FIG. 5) were evenly mixed and the pool was diluted 1:400,000,000 to create a linear PCR oligonucleotide stock. For each linear PCR reaction, 4 ng of purified template DNA was amplified in a reaction volume of 21.5 μL containing 12.5 μL of Phusion HF PCR master mix, 5 μL of H2O, 2 μL of the linear PCR oligo stock, and 2 μL of template DNA (from a 2 ng/μL stock). The linear PCR reaction was denatured for 30 sec at 98° C. followed by 8 cycles of (98° C.×10 sec, 50° C.×30 sec, 72° C.×30 sec), followed by a final extension at 72° C. for 2 minutes. The exponential PCR primers were then added to each tube using the same three primer setup and oligonucleotide concentrations as the phased MiSeq amplicon sequencing PCR protocol described above (outer primer concentrations=250 nM; inner primer concentration=8.3 nM) in a final volume of 25 μL. The exponential PCR reaction was incubated for 30 sec at 98° C. followed by 25 cycles of (98° C.×10 sec, 50° C.×30 sec, 72° C.×30 sec), followed by a final extension at 72° C. for 2 minutes. Pools of LEA-Seq reactions were purified twice with AmpureXP beads at a beads to sample ratio of 1.2 and 0.6 for the first and second purifications, respectively.

Robotically Arrayed Personal Bacterial Culture Collections Generated from Human Fecal Samples

Building upon our previously described methods for creating clonally arrayed personal culture collections from frozen fecal samples (20), we created a set of interfaces for a Precision XS robot (BioTek) so that picking, arraying, and archiving of fecal bacterial culture collections could be done with speed and economy under anaerobic conditions in a Coy chamber. Taxonomies were assigned to each strain in an arrayed collection by 454 Titanium V1V2 16S rRNA pyrosequencing or V4 16S rRNA sequencing on the Illumina MiSeq platform using a double-barcode strategy (20).

For a given culture collection, most strains (i.e., isolates with V1V2 or V4 16S rRNA amplicon sequences that are 100% identical across 100% of their length) were found in more than one well across the arrayed library. Therefore, several replicate wells of each strain were picked robotically from a 384-well plate and incubated for 3 d at 37° C. under anaerobic conditions (Coy chamber) on an 8-well TYGₛ-agar plate (Nunc). A single colony from each agar well was picked, grown in TYGS and archived as a TYGₛ/15% glycerol stock at −80° C.

Isolating Bacteroides thetaiotaomicron with a Species-Specific Monoclonal Antibody

A 10 μL aliquot of a frozen fecal sample obtained from each donor was recovered with a hot wire loop, serially diluted in sterile PBS (pH 7.6), and streaked onto Brain Heart Infusion (BHI) blood agar supplemented with 200 μg/mL gentamicin. Plates were incubated at 37° C. under anaerobic conditions (5% H2, 20% CO2, 75% N2) in a Coy chamber. Bacteria were subsequently transferred to sterile nitrocellulose membranes (Whatman, Protran BA85, 0.45 μm pore diameter) that had been placed over the agar surface. After 5 min, membranes were lifted off the agar, washed under running water for 1 min, followed by three washes in PBS/0.01% Tween 20 (5 min/wash) to remove any unbound colony fragments. Membranes were then incubated for 30 min in PBS/1% BSA to prevent non-specific binding. Filters were exposed for 2 h to a monoclonal antibody specific for B. thetaiotaomicron (mAb 260.8) followed by a 1 h incubation with HRP-labeled goat anti-mouse IgA (Southern Biotech, #1040-05). The monoclonal antibody represents a naturally primed antibody response to a bacterial surface epitope that was generated in gnotobiotic mice after mono-colonization with the type strain, B. thetaiotaomicron VPI-5482 and subsequently immortalized by fusing intestinal lamina propria lymphocytes from the mouse to a myeloma fusion partner (25). Bound antibody-antigen complexes were detected using the Bio-Rad Opti-4CN substrate kit (catalog #170-8235). Membranes were then washed in PBS/0.01% Tween 20 and dried. All steps were carried out at room temperature.

Four to eight colonies were recovered from each individual donor and tested by ELISA for 260.8 reactivity. Colonies verified to be positive were grown overnight at 37° C. in 200 μL of TYG medium in a 96-well plate (TRP, Switzerland) and DNA was prepared for microbial genome sequencing.

### Microbial Genome Sequencing

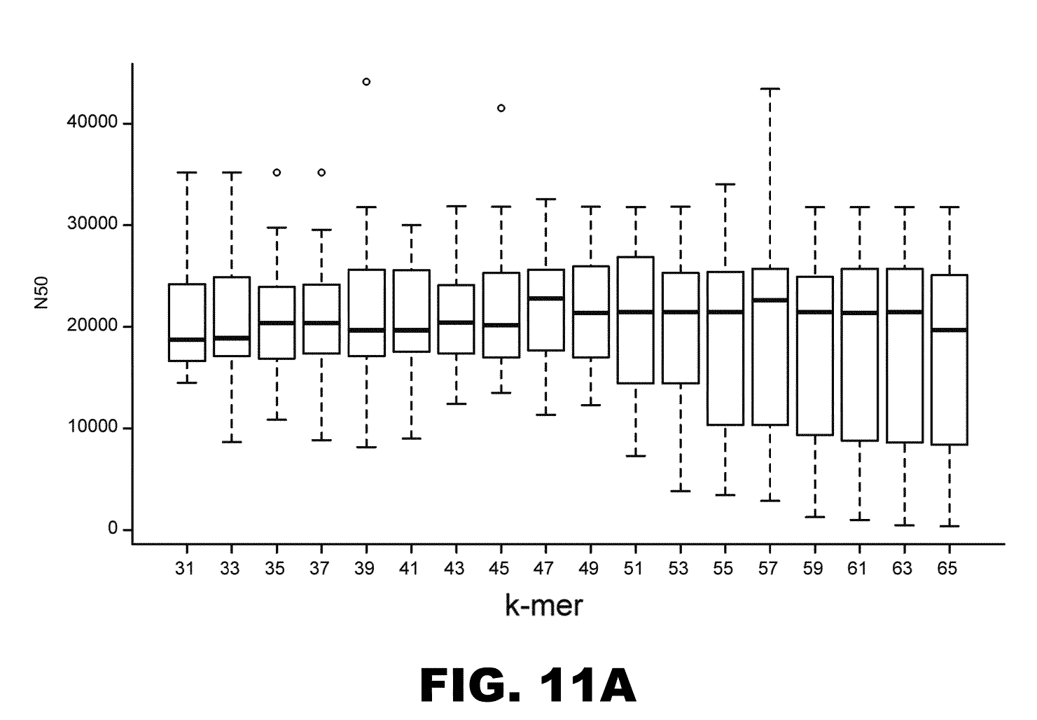

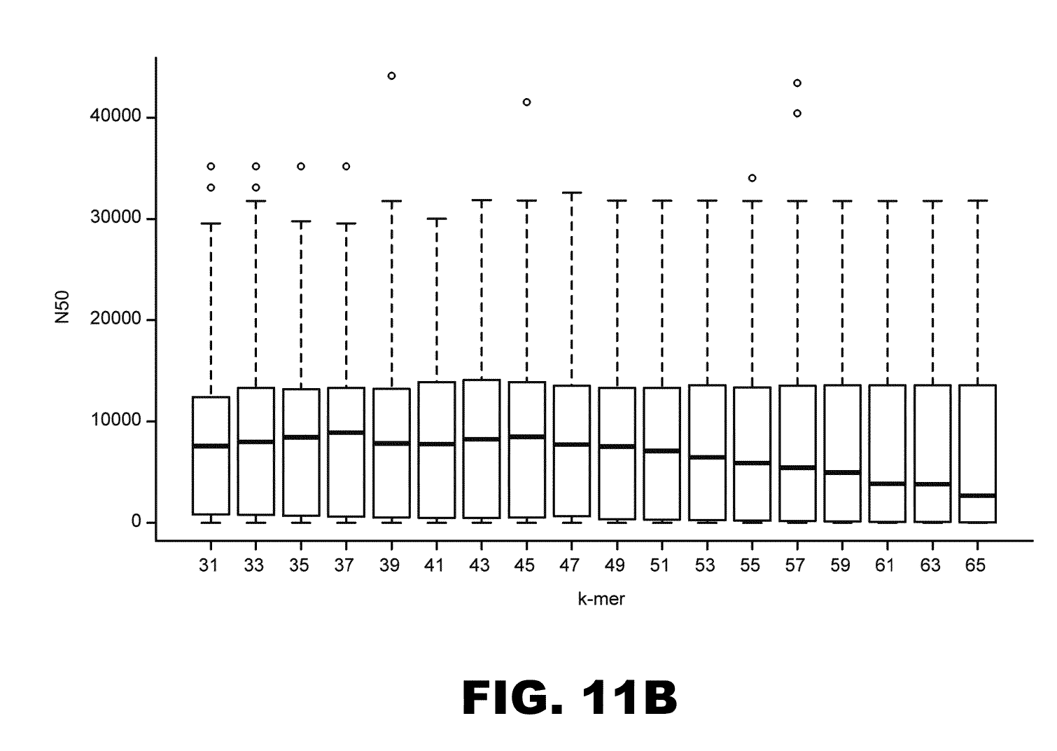

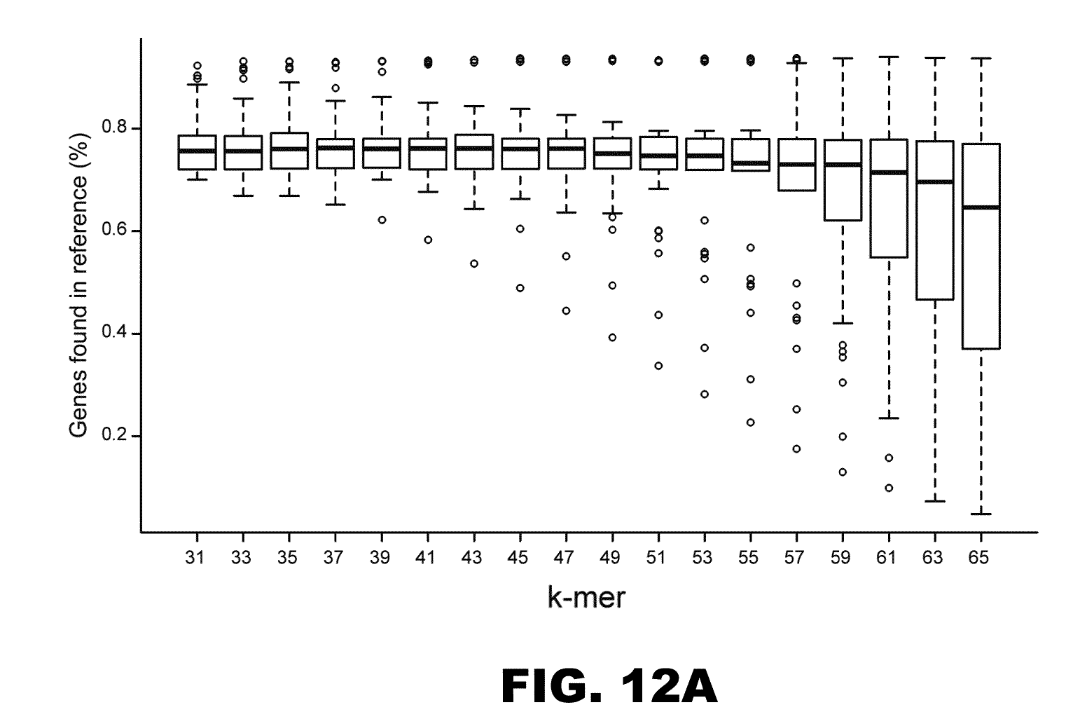

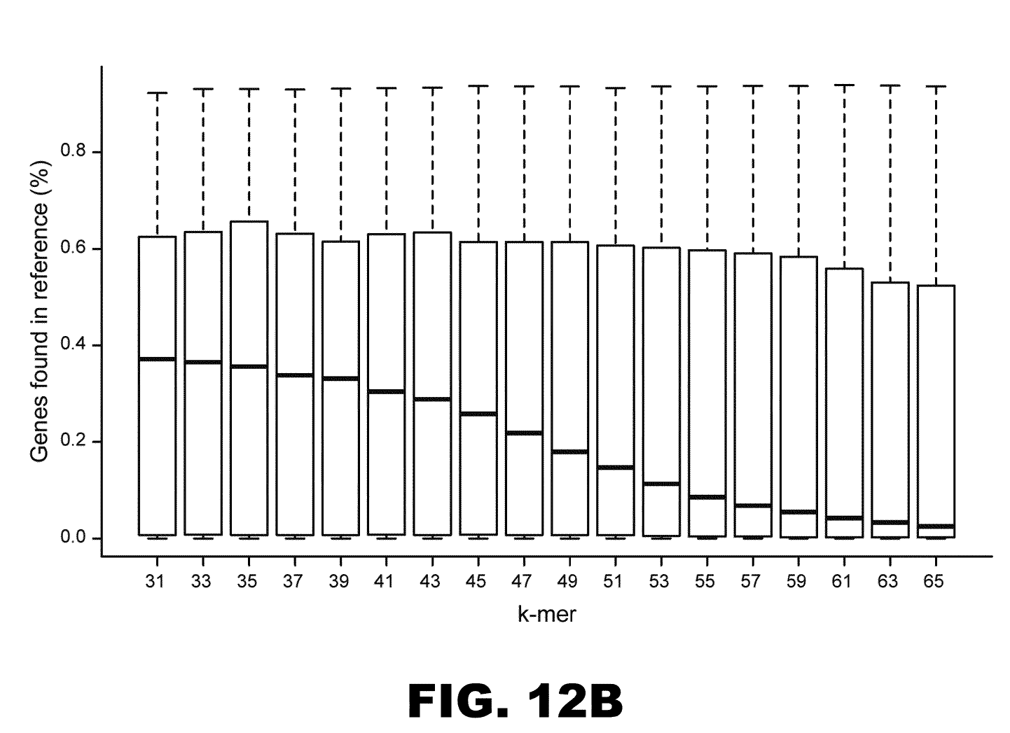

A small aliquot of each bacterial culture collection stock was taken for DNA extraction and subjected to multiplex genome sequencing with an Illumina HiSeq 2000 instrument (paired-end 101 nt reads; Tables 6, 7). Using the sequence reads from all isolates of one donor (F61T2; Table 4), we performed a series of tests to optimize the assembly parameters using Velvet 1.2.07 and VelvetOptimiser 2.2.5 (31). Given the wide-range of coverage when pooling up to 192 samples into a single line of Illumina HiSeq2000 flow cell, we performed our analyses both with all of the genomes from this donor and with the 30 genomes with the largest number of reads in order to explore both the overall and the high coverage assembly parameters. We tested a range of k-mer values (k=31 to k=65) to determine the optimal value for assembly. Assembly quality was judged by both the N50 metric (length N for which 50% of all bases in a set of contigs are in a sequence of length L<N) and by quantifying the fraction of genes present in each set of contigs (the latter by BLASTing against a reference genome of the same species). A gene was tagged as found in an assembly whenever the best BLAST hit in the reference genome had an e-value lower then 10⁻⁵ and the alignment spanned the full length of the reference gene. For the higher coverage genomes, we found no noticeable benefits when we normalized them (i.e., by subsampling to have only 50× coverage). In general, the N50 increased slightly with higher k-mer up to a certain value, after which the N50 decreased (FIG. 11), particularly when lower coverage genomes were included (FIG. 11B). Interestingly, the best assemblies, as determined by highest N50 values, were not the ones for which we were able to find a larger percentage of genes mapping to a reference genome of the same species (FIG. 12); k=31 recovered, in most cases, the largest number of genes. Given the only slight N50 performance benefit of increasing the k-mer beyond 31, the potential detrimental effect it could have on lower coverage genomes combined with the larger number of genes recovered when k=31, we chose this k-mer value for the genome assemblies for all donors.

### REFERENCES FOR THE EXAMPLES

1. P. J. Turnbaugh et aL, A core gut microbiome in obese and lean twins. Nature 457, 480-484 (2009).
2. P. J. Turnbaugh et al., Organismal, genetic, and transcriptional variation in the deeply sequenced gut microbiomes of identical twins. Proc. Natl. Acad. Sci. U.S.A. 107, 7503-7508 (2010).
3. P. B. Eckburg et al., Diversity of the human intestinal microbial flora. Science 308, 1635-1638 (2005).
4. T. Mitsuoka, Intestinal flora and aging. Nutr Rev 50, 438-446 (1992).
5. J. Qin et al., A human gut microbial gene catalogue established by metagenomic sequencing. Nature 464, 59-65 (2010).
6. S. Schloissnig, M. Arumugam, Genomic variation landscape of the human gut microbiome. Nature, 493, 45-50 (2013).
7. J. B. Hiatt, R. P. Patwardhan, E. H. Turner, C. Lee, J. Shendure, Parallel, tag-directed assembly of locally derived short sequence reads. Nat. Methods 7, 119-122 (2010).
8. C. B. Jabara, C. D. Jones, J. Roach, J. A. Anderson, R. Swanstrom, Accurate sampling and deep sequencing of the HIV-1 protease gene using a Primer ID. Proc. Natl. Acad. Sci. U.S.A. 108, 20166-20171 (2011).
9. T. Kivioja et al., Counting absolute numbers of molecules using unique molecular identifiers. Nat. Methods 9, 72-74 (2012).
10. N. P. McNulty et al., The impact of a consortium of fermented milk strains on the gut microbiome of gnotobiotic mice and monozygotic twins. Science Translational Med. 3, 106ra106 (2011).
11. H. R. Kissileff et al., Leptin reverses declines in satiation in weight-reduced obese humans. Am. J. Clin. Nutr. 95, 309-317 (2012).
12. M. Rosenbaum et al., A comparative study of different means of assessing long-term energy expenditure in humans. Am. J. Physiol. 270, R496-504 (1996).
13. M. Rosenbaum, M. Nicolson, J. Hirsch, E. Murphy, F. Chu, and R. L. Leibel. Effects of weight change on plasma leptin concentrations and energy expenditure. J. Clin. Endocrinol. Metab., 82, 3647-3654, (1997)
14. R. L., Leibel, M. Rosenbaum, and J. Hirsch. Changes in energy expenditure resulting from altered body weight. N. Eng. J. Med., 332, 621-628, 1995.
15. E. G. Zoetendal, A. D. Akkermans, W. M. De Vos, Temperature gradient gel electrophoresis analysis of 16S rRNA from human fecal samples reveals stable and host-specific communities of active bacteria. Appl. Environ. Microbiol. 64, 3854-3859 (1998).
16. E. K. Costello et al., Bacterial community variation in human body habitats across space and time. Science 326, 1694-1697 (2009).
17. C. Huttenhower et al Human Microbiome Project Consortium, Stucture, function and diversity of the healthy human microbiome. Nature 486, 207-214 (2012).
18. M. Rajilic-Stojanovic, H. G. H. J. Heilig, T. Tims, E. G. Zoetendal, W. M. de Vos, Long-term monitoring of the human intestinal microbiota composition. Environ. Microbiol. 10.1111/1462-2920.12023 (2012).
19. A. L. Goodman et al., Extensive personal human gut microbiota culture collections characterized and manipulated in gnotobiotic mice. Proc. Natl. Acad. Sci. U.S.A. 108, 6252-6257 (2011).
20. S. Kurtz et al., Versatile and open software for comparing large genomes. Genome Biol. 5, R12 (2004).
21. S. R. Henz, D. H. Huson, A. F. Auch, K. Nieselt-Struwe, S. C. Schuster, Whole-genome prokaryotic phylogeny. Bioinformatics 21, 2329-2335 (2005).
22. Q. Wang, G. M. Garrity, J. M. Tiedje, J. R. Cole, Naive Bayesian classifier for rapid assignment of rRNA sequences into the new bacterial taxonomy. Appl. Environ. Microbiol. 73, 5261-5267 (2007).
23. E. E. Hansen et al., Pan-genome of the dominant human gut-associated archaeon, Methanobrevibacter smithii, studied in twins. Proc. Natl. Acad. Sci. U.S.A. 108, 4599-4606 (2011).
24. D. A. Peterson, N. P. McNulty, J. L. Guruge, J. I. Gordon, IgA response to symbiotic bacteria as a mediator of gut homeostasis. Cell Host & Microbe 2, 328-329 (2007).
25. J. G. Caporaso et al., Moving pictures of the human microbiome. Genome Biol 12, R50 (2011).
26. J. Halfvarson, Genetics in twins with Crohn's disease: less pronounced than previously believed? Inflammatory Bowel Dis. 17, 6-12 (2011).
27. W. S., Slutske, E. E. Hunt-Carter, R. E. Nabors-Oberg, K. J. Sher, K. K. Bucholz, P. A. Madden, A. Anokhin, A. C. Heath. Do college students drink more than their non-college-attending peers? Evidence from a population-based longitudinal female twin study. J Abnorm. Psychol. 113, 530-540 (2004).
28. M. Waldron, K. K. Bucholz, M. T. Lynskey, P. A. Madden, A. C. Heath Alcoholism and timing of separation in parents: findings in a midwestern birth cohort. J. Stud. Alcohol Drugs 74, 337-348 (2013)
29. A. L. Goodman et al., Identifying genetic determinants needed to establish a human gut symbiont in its habitat. Cell Host & Microbe 6, 279-289 (2009).
30. D. R. Zerbino, G. K. McEwen, E. H. Margulies, E. Birney, Pebble and rock band: heuristic resolution of repeats and scaffolding in the velvet short-read de novo assembler. PloS One 4, e8407 (2009).
31. B. Langmead, C. Trapnell, M. Pop, S. L. Salzberg, Ultrafast and memory-efficient alignment of short DNA sequences to the human genome. Genome Biol. 10, R25 (2009).
32. C. Lozupone et al., Identifying genomic and metabolic features that can underlie early successional and opportunistic lifestyles of human gut symbionts. Genome Res. 22, 1974-1984 (2012).
33. C. R. Woese, G. E. Fox, Phylogenetic structure of the prokaryotic domain: the primary kingdoms. Proc. Natl. Acad. Sci. U.S.A. 74, 5088-5090 (1977).
34. W. A. Walters et al., PrimerProspector: de novo design and taxonomic analysis of barcoded PCR primers. Bioinformatics 27, 1159-1161 (2011).
35. B. D. Muegge et al., Diet drives convergence in gut microbiome functions across mammalian phylogeny and within humans. Science 332, 970-974 (2011).
36. Z. Liu, C. Lozupone, M. Hamady, F. D. Bushman, R. Knight, Short pyrosequencing reads suffice for accurate microbial community analysis. Nucleic Acids Res. 35, e120 (2007).
37. J. G. Caporaso et al., Ultra-high-throughput microbial community analysis on the Illumina HiSeq and MiSeq platforms. ISME J. 6, 1621-1624 (2012).
38. T. Magoc, S. L. Salzberg, FLASH: fast length adjustment of short reads to improve genome assemblies. Bioinformatics 27, 2957-2963 (2011).
39. S. E. Choe, M. Boutros, A. M. Michelson, G. M. Church, M. S. Halfon, Preferred analysis methods for Affymetrix GeneChips revealed by a wholly defined control dataset. Genome Biol 6, R16 (2005).
40. R. C. Edgar, Search and clustering orders of magnitude faster than BLAST. Bioinformatics 26, 2460-2461 (2010).

TABLE 1
Species composition of the sequenced arrayed culture collections from six donors.

| species | | | donor | | | | | |
|---|---|---|---|---|---|---|---|---|
| ID | species | alternative name | F3T1 | F58T1 | F60T1 | F60T2 | F61T1 | F61T2 |
| 1 | Alistipes indistinctus | | + | | | | + | |
| 2 | Anaerococcus vaginalis | Anaerococcus | | + | + | + | | + |
| 3 | Anaerofustis stercorihominis | | | + | | | | + |
| 4 | Anaerofustis stercorihominis | | | | | | + | |
| 5 | Bacteroides | | | | | | + | |
| 6 | Bacteroides caccae | | | + | | + | | + |
| 7 | Bacteroides finegoldii | | | | | + | + | |
| 8 | Bacteroides fragilis | | | | | | + | + |
| 9 | Bacteroides intestinalis | Bacteroides cellulosilyticus | + | + | | + | | + |
| 10 | Bacteroides massiliensis | | + | | | + | | |
| 11 | Bacteroides ovatus | | + | | + | + | + | |
| 12 | Bacteroides salyersiae | | + | | | | | |
| 13 | Bacteroides thetaiotaomicron | Bacteroides faecis | + | | | + | + | + |
| 14 | Bacteroides uniformis | Bacteroides acidifaciens | | + | | + | + | + |
| 15 | Bacteroides vulgatus | Bacteroides dorei | + | + | + | + | + | + |
| 16 | Barnesiella intestinihominis | | | + | | | | |
| 17 | Bifidobacterium adolescentis | | | | | + | + | |
| 18 | Bifidobacterium bifidum | | | | | | + | |
| 19 | Bifidobacterium longum | | + | | | + | | + |
| 20 | Bifidobacterium [symbol] atum | | | | | + | + | |
| 21 | Blautia | | | + | | | | |
| 22 | Blautia schinkii | | | | | + | + | |
| 23 | Butyricimonas virosa | | + | + | | + | + | |
| 24 | Clostridiales | | | | | | + | |
| 25 | Clostridiales | | | | | | + | |
| 26 | Clostridiales | | | | | | + | |
| 27 | Clostridiales | | | | | | + | |
| 28 | Clostridiales | | | + | | | | |
| 29 | Clostridiales | | | | | | | + |
| 30 | Clostridiales | | | | | | + | |
| 31 | Clostridium | | + | + | | | + | |
| 32 | Clostridium | | | | | | + | |
| 33 | Clostridium bolteae | | | | | | | + |
| 34 | Clostridium hylemonae | | | | | | | + |
| 35 | Clostridium leptum | | | | | + | + | |
| 36 | Clostridium scindens | | | | | + | | + |
| 37 | Clostridium scindens | | | | + | | | |
| 38 | Collinsella | | | + | | | | |
| 39 | Collinsella aerofaciens | | + | | | + | + | |
| 40 | Coprococcus comes | | + | + | | + | | |
| 41 | Dorea formicigenerans | | + | + | | + | + | |
| 42 | Dorea longicatena | | + | + | | + | | + |
| 43 | Dorea longicatena | | | + | | | | |
| 44 | Eggerthella lenta | Subdoligranulum variabile | | | | | | + |
| 45 | Escherichia coli | | + | + | | + | + | + |
| 46 | Eubacterium biforme | | | + | | | | |
| 47 | Eubacterium callanderi | | | | | + | | |
| 48 | Eubacterium contortum | | | | | | + | |
| 49 | Eubacterium eligens | | | + | | | | |
| 50 | Finegoldia magna | Dialister invisus | | | | | | + |
| 51 | Lactobacillus | | | + | | | | |
| 52 | Lactobacillus casei | | | + | | | | |
| 53 | Megasphaera elsdenii | | | | + | | | |
| 54 | Odoribacter splanchnicus | | | + | | + | + | |
| 55 | Parabacteroides distasonis | | + | + | + | + | + | |
| 56 | Parabacteroides goldsteinii | | | + | | | + | |
| 57 | Parabacteroides merdae | | | + | | | + | |
| 58 | Peptoniphilus harei | | | + | | + | | |
| 59 | Roseburia intestinalis | | | | | | + | |
| 60 | Ruminococcaceae | | | | | + | | |
| 61 | Ruminococcus | Lachnospiraceae | | + | | + | | |
| 62 | Ruminococcus albus | | | | | + | | |
| 63 | Ruminococcus bromii | | | + | | + | | |
| 64 | Ruminococcus gauvreauii | | + | | | | | |
| 65 | Ruminococcus gnavus | | + | | + | | | + |
| 66 | Ruminococcus obeum | | + | | | | | |
| 67 | Ruminococcus sp CCUG 37327 A | | + | + | | | | |
| 68 | Ruminococcus sp DJF VR70k1 | | | | | | + | |
| 69 | Ruminococcus torques | | + | | | | + | |
| 70 | Streptococcus | | | | + | | | |
| 71 | Streptococcus gordonii | | | | | | + | |
| 72 | Streptococcus parasanguinis | | | | | | + | |
| 73 | Streptococcus thermophilus | | | | | | + | |
| 74 | Subdoligranulum variabile | | + | + | + | + | | + |
| 75 | Veillonella parvula | | | | | | | + |

[symbol]

indicates data missing or illegible when filed

TABLE 2
A human gut microbe reference community of 64 bacterial strains.

| Phylogenetic and identifier data | | | | | | | | | | |
|---|---|---|---|---|---|---|---|---|---|---|
| | | | | Genome | | | Membership | | | |
| | | | | Project | Taxono- | | Member community | | | |
| Phylum | Genus | Species | strain | ID | my ID | Accession | 48 | 32 | 6 | 3 |
| Actinobacteria | Bifidobacterium | angulatum | DSM20098 | 55113 | 518635 | NZ_ABYS00000000 | | + | | |
| Actinobacteria | Bifidobacterium | bifidum | DSM20456 | 28655 | 500634 | | + | + | | |
| Actinobacteria | Bifidobacterium | dentium | ATCC27678 | 54901 | 473819 | NZ_ABIX00000000 | | + | | |
| Actinobacteria | Bifidobacterium | pseudocatenulatum | DSM20438 | 55303 | 547043 | NZ_ABXX00000000 | + | | | |
| Actinobacteria | Collinsella | aerofaciens | ATCC25986 | 54525 | 411903 | NZ_AAVN00000000 | | + | + | |
| Actinobacteria | Collinsella | intestinalis | DSM13280 | 55125 | 521003 | NZ_ABXH00000000 | + | | | |
| Bacteroidetes | Alistipes | indistinctus | DSM 22520 | 75115 | 742725 | NZ_ADLD00000000 | + | + | | |
| Bacteroidetes | Bacteroides | caccae | ATCC43185 | 54521 | 411901 | NZ_AAVM00000000 | | + | | |
| Bacteroidetes | Bacteroides | cellulosilyticus | DSM14838 | 55279 | 537012 | NZ_ACCH00000000 | + | | | |
| Bacteroidetes | Bacteroides | dorei | DSM17855 | 54993 | 483217 | NZ_ABWZ00000000 | + | | | |
| Bacteroidetes | Bacteroides | eggerthii | DSM20697 | 54989 | 483216 | NZ_ABVO00000000 | + | | | |
| Bacteroidetes | Bacteroides | finegoldii | DSM17565 | 54985 | 483215 | NZ_ABXI00000000 | + | | | |
| Bacteroidetes | Bacteroides | intestinalis | DSM17393 | 54881 | 471870 | NZ_ABJL00000000 | + | | | |
| Bacteroidetes | Bacteroides | ovatus | ATCC8483 | 54543 | 411476 | NZ_AAXF00000000 | + | + | + | |
| Bacteroidetes | Bacteroides | thetaiotaomicron | 3737 | | | NC_Bthetaiotaomicron3731 | + | | | |
| Bacteroidetes | Bacteroides | thetaiotaomicron | 7330 | | | NC_Bthetaiotaomicron7330 | + | | | |
| Bacteroidetes | Bacteroides | thetaiotaomicron | VPI-5482 | 62913 | 226186 | NC_004663 | + | + | + | + |
| Bacteroidetes | Bacteroides | uniformis | ATCC8492 | 54547 | 411479 | NZ_AAYH00000000 | + | | | |
| Bacteroidetes | Bacteroides | vulgatus | ATCC8482 | 58253 | 435590 | NC_009614 | + | | | |
| Bacteroidetes | Bacteroides | xylanisolvens | DSM18836 | 39177 | 657309 | FP929033 | + | | | |
| Bacteroidetes | Parabacteroides | johnsonii | DSM18315 | 55269 | 537006 | NZ_ABYH00000000 | + | | | |
| Firmicute | Anaerococcus | hydrogenalis | DSM7454 | 55367 | 561177 | NZ_ABXA00000000 | + | + | | |
| Firmicute | Anaerotruncus | colihominis | DSM17241 | 54807 | 445972 | NZ_ABGD00000000 | + | | | |
| Firmicute | Blautia | hansenii | DSM20583 | 55275 | 537007 | NZ_ABYU00000000 | + | | | |
| Firmicute | Blautia | luti | DSM14534 | 38333 | 649762 | | + | | | |
| Firmicute | Clostridium | asparagiforme | DSM15981 | 55115 | 518636 | NZ_ACCJ00000000 | + | | | |
| Firmicute | Clostridium | hathewayi | DSM13479 | 55373 | 566550 | NZ_ACIO00000000 | + | | | |
| Firmicute | Clostridium | leptum | DSM753 | 54605 | 428125 | NZ_ABCB00000000 | + | | | |
| Firmicute | Clostridium | nexile | DSM1787 | 55077 | 500632 | NZ_ABWO00000000 | + | | | |
| Firmicute | Clostridium | nexile-related | A2-232 | 18209 | 411488 | | + | | | |
| Firmicute | Clostridium | spiroforme | DSM1552 | 54607 | 428126 | NZ_ABIK00000000 | | + | | |
| Firmicute | Clostridium | sporogenes | ATCC15579 | 54895 | 471871 | NZ_ABKW00000000 | + | + | | |
| Firmicute | Clostridium | symbiosum | ATCC14940 | 18183 | 411472 | NC_Csymbiosum | | + | + | |
| Firmicute | Clostridium | | M62/1 | 54557 | 411486 | NZ_ACFX00000000 | + | + | | |
| Firmicute | Coprococcus | comes | ATCC27758 | 54883 | 470146 | NZ_ABVR00000000 | + | + | | |
| Firmicute | Coprococcus | eutactus | ATCC27759 | 54541 | 411474 | NZ_ABEY00000000 | | + | | |
| Firmicute | Dorea | formicigenerans | ATCC27755 | 54513 | 411461 | NZ_AAXA00000000 | + | + | | |
| Firmicute | Dorea | longicatena | DSM13814 | 54515 | 411462 | NZ_AAXB00000000 | + | + | | |
| Firmicute | Eubacterium | biforme | DSM3989 | 55117 | 518637 | NZ_ABYT00000000 | + | | | |
| Firmicute | Eubacterium | eligens | ATCC27750 | 59171 | 515620 | NC_012778 | + | + | | |
| Firmicute | Eubacterium | rectale | ATCC33656 | 59169 | 515619 | NC_012781 | | + | + | + |
| Firmicute | Eubacterium | ventriosum | ATCC27560 | 54517 | 411463 | NZ_AAVL00000000 | + | + | | |
| Firmicute | Faecalibacterium | prausnitzii | M21/2 | 54555 | 411485 | NZ_ABED00000000 | + | | | |
| Firmicute | Lactobacillus | reuteri | DSM20016 | 58471 | 557436 | NC_009513 | | + | | |
| Firmicute | Lactobacillus | ruminis | ATCC25644 | 71361 | 525362 | NZ_ACGS00000000 | | + | | |
| Firmicute | Roseburia | intestinalis | L1-82 | 55267 | 536231 | NZ_ABYJ00000000 | + | + | | |
| Firmicute | Ruminococcus | gnavus | ATCC29149 | 54537 | 411470 | NZ_AAYG00000000 | + | | | |
| Firmicute | Ruminococcus | hydrogenotrophicus | DSM10507 | 54939 | 476272 | NZ_ACBZ00000000 | | + | | |
| Firmicute | Ruminococcus | lactaris | ATCC29176 | 54903 | 471875 | NZ_ABOU00000000 | + | | | |
| Firmicute | Ruminococcus | obeum | ATCC29174 | 54509 | 411459 | NZ_AAVO00000000 | | + | | |
| Firmicute | Ruminococcus | torques | ATCC27756 | 54511 | 411460 | NZ_AAVP00000000 | + | | | |
| Firmicute | Streptococcus | infantarius | ATCC BAA- | 54885 | 471872 | NZ_ABJK00000000 | + | | | |
| | | | 102 | | | | | | | |
| Firmicute | Subdoligranulum | variabile | DSM15176 | 54539 | 411471 | NZ_ACBY00000000 | + | | | |
| Lentisphaerae | Victivallis | vadensis | ATCC BAA- | 54305 | 340101 | NZ_ABDE00000000 | | + | | |
| | | | 548 | | | | | | | |
| Proteobacteria | Edwardsiella | tarda | ATCC23685 | 47355 | 500638 | NZ_ADGK00000000 | + | + | | |
| Proteobacteria | Enterobacter | cancerogenus | ATCC35316 | 55079 | 500639 | NC_Ecancerogenus | + | | | |
| Proteobacteria | Escherichia | coli | K12 | 57779 | 511145 | NC_000913 | + | + | + | + |
| Proteobacteria | Escherichia | fergusonii | ATCC35469 | 59375 | 585054 | NC_011740 | + | | | |
| Proteobacteria | Proteus | penneri | ATCC35198 | 54897 | 471881 | NZ_ABVP00000000 | + | | | |
| Proteobacteria | Providencia | alcalifaciens | DSM30120 | 55119 | 520999 | NZ_ABXW00000000 | + | + | | |
| Proteobacteria | Providencia | rettgeri | DSM1131 | 55121 | 521000 | NZ_ACCI00000000 | | + | | |
| Proteobacteria | Providencia | rustigianii | DSM4541 | 55071 | 500637 | NZ_ABXV00000000 | | + | | |
| Proteobacteria | Providencia | stuartii | ATCC25827 | 54899 | 471874 | NZ_ABJD00000000 | | + | | |
| Verrucomicrobia | Akkermansia | muciniphila | ATCC BAA- | 58985 | 349741 | NC_010655 | + | | | |
| | | | 835 | | | | | | | |

TABLE 3
Performance of standard 16S rRNA amplicon sequencing methods versus LEA-Seq defined using mock communities.

| | | | | | Precision at various minimum abundance thresholds | | | | | |
|---|---|---|---|---|---|---|---|---|---|---|
| | | | | | Total | | | | | |
| | Mock | | | | Number of | | | | | |
| | community | | | Repli- | Reads | | | | | |
| Region | type | Method | Platform | cates | Generated | 1:500 | 1:1000 | 1:5000 | 1:10000 | 1:50000 |
| V1V2 | 48 member | standard | 454 | 1 | 1955 | 0.48 | 0.24 | | | |
| | | | Titanium | | | | | | | |
| V4 | 48 member | standard with | MiSeq | 11 | 74231 ± | 0.79 ± | 0.76 ± | 0.25 ± | 0.08 ± | 0.01 ± |
| | | phasing | | | 123305 | 0.033 | 0.097 | 0.064 | 0.064 | 0.007 |
| V1V2 | 48 member | standard with | 454 | 2 | 45278 ± | 0.73 ± | 0.57 ± | 0.22 ± | 0.09 ± | |
| | | deeper | Titanium | | 2312 | 0.031 | 0.021 | 0.001 | 0.005 | |
| | | sequencing | | | | | | | | |
| V1V2 | 48 member | LEA-Seq | HiSeq 2000 | 16 | 148420 ± | 0.76 ± | 0.74 ± | 0.66 ± | 0.63 ± | 0.45 ± |
| | | | | | 51669 | 0.059 | 0.064 | 0.041 | 0.034 | 0.059 |
| V1V2 | 48 member | LEA-Seq | HiSeq 2000 | 7 | 3670857 ± | 0.68 ± | 0.56 ± | 0.14± | 0.08 ± | 0.02 ± |
| | | without | | | 885032 | 0.062 | 0.121 | 0.023 | 0.012 | 0.003 |
| | | consensus | | | | | | | | |
| V1V2 | 32 member | LEA-Seq | HiSeq 2000 | 7 | 146100 ± | 0.79 ± | 0.77 ± | 0.65 ± | 0.57 ± | 0.26 ± |
| | | | | | 67381 | 0.037 | 0.036 | 0.014 | 0.030 | 0.124 |
| V1V2 | 6 member | LEA-Seq | HiSeq 2000 | 1 | 6506 | 0.78 | 0.78 | 0.22 | | |
| V1V2 | 3 member | LEA-Seq | HiSeq 2000 | 1 | 4165 | 0.86 | 0.86 | | | |
| V4 | 48 member | LEA-Seq | HiSeq 2000 | 19 | 213467 ± | 0.89 ± | 0.88 ± | 0.84 ± | 0.83 ± | 0.68 ± |
| | | | | | 89391 | 0.059 | 0.064 | 0.041 | 0.024 | 0.059 |
| V1V2 | 48 member | LEA-Seq | HiSeq 2000 | pooled | 1224195 | 0.84 | 0.78 | 0.66 | 0.63 | 0.50 |
| | | | | run 1 | | | | | | |
| V1V2 | 48 member | LEA-Seq | HiSeq 2000 | pooled | 1150528 | 0.71 | 0.67 | 0.62 | 0.60 | 0.57 |
| | | | | run 2 | | | | | | |
| V4 | 48 member | LEA-Seq | HiSeq 2000 | pooled | 4055875 | 0.86 | 0.87 | 0.84 | 0.84 | 0.78 |
| | | | | run 1 | | | | | | |

Performance at each threshold was estimated by linear interpolation of the precision vs threshold curve.

TABLE 4
Human subject sampling information, sample usage, and diversity (richness).

| | | | | | | | | | Analytic methods applied to sample | | | | | | |
|---|---|---|---|---|---|---|---|---|---|---|---|---|---|---|---|
| | | | | | | time of | Number | Number | | | M. smithii | B. thetaiotaomicron | | Weight loss study | |
| | | | | | | sample | of 97% | of 100% | | | pan genome | pan genome | Previous | parameters | |
| Sub- | Sam- | | | | family | collection | ID OTUs | ID OTUs | | Arrayed | (strains shared | (strains shared | Publica- | Weight | Triiodo- |
| ject | ple | Alter- | | BMI | relation- | (days after | “species” | “strains” | LEA- | culture | with family | with family | tion | loss (800 | thyronine |
| ID | ID | native ID | BMI | category | ship | first sample) | (LEA-Seq) | (LEA-Seq) | Seq | collection | member) | member) | (PMID) | kcal/day) | (25 ug/day) |
| F70 | F70.1 | LR1335.1 | 31.1 | obese I | singleton | 0 | 224 | 113 | Y | | | | | | |
| F70 | F70.2 | LR1335.2 | 31.0 | obese I | singleton | 7 | 176 | 92 | Y | | | | | | |
| F70 | F70.3 | LR1335.4 | 31.3 | obese I | singleton | 25 | 161 | 89 | Y | | | | | | |
| F70 | F70.4 | LR1335.5 | 31.4 | obese I | singleton | 30 | 200 | 99 | Y | | | | | | |
| F70 | F70.5 | LR1335.7 | 31.8 | obese I | singleton | 47 | 132 | 70 | Y | | | | | | |
| F70 | F70.6 | LR1335.8 | 31.3 | obese I | singleton | 53 | 255 | 121 | Y | | | | | | |
| F71 | F71.1 | LR4535.0 | 42.7 | obese III | singleton | 0 | 134 | 70 | Y | | | | | | |
| F71 | F71.2 | LR4535.1 | 42.5 | obese III | singleton | 13 | 108 | 56 | Y | | | | | | |
| F71 | F71.3 | LR4535.1B | 42.1 | obese III | singleton | 19 | 64 | 43 | Y | | | | | | |
| F71 | F71.4 | LR4535.1C | 38.8 | obese II | singleton | 63 | 97 | 53 | Y | | | | | + | |
| F71 | F71.5 | LR4535.2 | 37.6 | obese II | singleton | 70 | 112 | 67 | Y | | | | | + | |
| F71 | F71.6 | LR4535.3 | 37.1 | obese II | singleton | 77 | 124 | 68 | Y | | | | | + | |
| F71 | F71.7 | LR4535.4 | 33.6 | obese I | singleton | 117 | 69 | 43 | Y | | | | | + | |
| F71 | F71.8 | LR4535.5 | 33.1 | obese I | singleton | 138 | 96 | 55 | Y | | | | | + | |
| F71 | F71.9 | LR4535.7 | 33.5 | obese I | singleton | 222 | 115 | 70 | Y | | | | | | |
| F72 | F72.1 | LR6510.1 | 46.0 | obese III | singleton | 0 | 207 | 95 | Y | | | | | | |
| F72 | F72.2 | LR6510.1B | 45.2 | obese III | singleton | 7 | 140 | 81 | Y | | | | | | |
| F72 | F72.3 | LR6510.2 | 45.4 | obese III | singleton | 21 | 137 | 79 | Y | | | | | | |
| F72 | F72.4 | LR6510.3B | 42.7 | obese III | singleton | 70 | 190 | 108 | Y | | | | | + | |
| F72 | F72.5 | LR6510.4 | 42.0 | obese III | singleton | 80 | 180 | 106 | Y | | | | | + | |
| F72 | F72.6 | LR6510.7 | 38.1 | obese II | singleton | 132 | 190 | 109 | Y | | | | | + | |
| F72 | F72.7 | LR6510.8 | 38.4 | obese II | singleton | 137 | 217 | 124 | Y | | | | | | |
| F72 | F72.8 | LR6510.9 | 38.5 | obese II | singleton | 159 | 197 | 113 | Y | | | | | | |
| F72 | F72.9 | LR6510.10 | 38.7 | obese II | singleton | 161 | 122 | 79 | Y | | | | | | + |
| F72 | F72.10 | LR6510.11 | 38.2 | obese II | singleton | 170 | 202 | 126 | Y | | | | | | + |
| F72 | F72.11 | LR6510.13 | 38.1 | obese II | singleton | 183 | 157 | 104 | Y | | | | | | + |
| F72 | F72.12 | LR6510.15 | 37.9 | obese II | singleton | 211 | 196 | 118 | Y | | | | | | |
| F73 | F73.1 | LR7145.1 | 45.2 | obese III | singleton | 0 | 118 | 59 | Y | | | | | | |
| F73 | F73.2 | LR7145.2 | 45.8 | obese III | singleton | 8 | 175 | 89 | Y | | | | | | |
| F73 | F73.3 | LR7145.3 | 45.2 | obese III | singleton | 15 | 94 | 52 | Y | | | | | | |
| F73 | F73.4 | LR7145.4 | 45.5 | obese III | singleton | 22 | 129 | 69 | Y | | | | | | |
| F73 | F73.5 | LR7145.5 | 45.1 | obese III | singleton | 29 | 91 | 42 | Y | | | | | | |
| F73 | F73.6 | LR7145.6 | 45.8 | obese III | singleton | 38 | 115 | 55 | Y | | | | | | |
| F73 | F73.7 | LR7145.7 | 45.1 | obese III | singleton | 49 | 186 | 102 | Y | | | | | | |
| F73 | F73.8 | LR7145.8 | 45.1 | obese III | singleton | 58 | 193 | 91 | Y | | | | | + | |
| F73 | F73.9 | LR7145.9 | 44.6 | obese III | singleton | 64 | 180 | 91 | Y | | | | | + | |
| F73 | F73.10 | LR7145.10 | 43.1 | obese III | singleton | 85 | 148 | 73 | Y | | | | | + | |
| F73 | F73.11 | LR7145.11 | 42.2 | obese III | singleton | 94 | 122 | 71 | Y | | | | | + | |
| F73 | F73.12 | LR7145.12 | 39.5 | obese II | singleton | 141 | 101 | 51 | Y | | | | | + | |
| F2T1 | F2T1.1 | TS4.1 | 21.0 | lean | twin (MZ) | 0 | 97 | 59 | Y | | | | 19043404 | | |
| F2T1 | F2T1.2 | TS4.2 | 21.0 | lean | twin (MZ) | 46 | 174 | 96 | Y | | | | 19043404 | | |
| F2T1 | F2T1.3 | TS4.3 | 21.0 | lean | twin (MZ) | 352 | 140 | 77 | Y | | | | 19043404 | | |
| F2T1 | F2T1.4 | TSDA9.1 | 21.0 | lean | twin (MZ) | 400 | 192 | 98 | Y | | | | 22030749 | | |
| F2T1 | F2T1.5 | TSDA9.2 | 21.0 | lean | twin (MZ) | 415 | 180 | 92 | Y | | | | 22030749 | | |
| F2T1 | F2T1.6 | TSDA9.3 | 21.4 | lean | twin (MZ) | 422 | 178 | 93 | Y | | | | 22030749 | | |
| F2T1 | F2T1.7 | TSDA9.4 | 22.0 | lean | twin (MZ) | 435 | 160 | 85 | Y | | | | 22030749 | | |
| F2T1 | F2T1.8 | TSDA9.5 | 22.0 | lean | twin (MZ) | 442 | 166 | 84 | Y | | | | 22030749 | | |
| F2T1 | F2T1.9 | TSDA9.6 | 22.0 | lean | twin (MZ) | 457 | 163 | 82 | Y | | | | 22030749 | | |
| F2T1 | F2T1.10 | TSDA9.8 | 22.0 | lean | twin (MZ) | 499 | 151 | 81 | Y | | | | 22030749 | | |
| F2T1 | F2T1.11 | TSDA9.9 | 22.0 | lean | twin (MZ) | 513 | 124 | 67 | Y | | | | 22030749 | | |
| F2T1 | F2T1.12 | TS4.5 | 24.4 | lean | twin (MZ) | 2059 | 143 | 70 | Y | | | | | | |
| F2T2 | F2T2.1 | TS5.1 | 20.9 | lean | twin (MZ) | 0 | 130 | 65 | Y | | | | 19043404 | | |
| F2T2 | F2T2.2 | TS5.2 | 20.0 | lean | twin (MZ) | 52 | 125 | 63 | Y | | | | 19043404 | | |
| F2T2 | F2T2.3 | TS5.3 | 21.0 | lean | twin (MZ) | 366 | 169 | 79 | Y | | | | 19043404 | | |
| F2T2 | F2T2.4 | TSDA10.1 | 21.0 | lean | twin (MZ) | 413 | 195 | 93 | Y | | | | 22030749 | | |
| F2T2 | F2T2.5 | TSDA10.2 | 21.0 | lean | twin (MZ) | 429 | 193 | 95 | Y | | | | 22030749 | | |
| F2T2 | F2T2.6 | TSDA10.3 | 21.0 | lean | twin (MZ) | 436 | 191 | 91 | Y | | | | 22030749 | | |
| F2T2 | F2T2.7 | TSDA10.4 | 21.0 | lean | twin (MZ) | 449 | 164 | 81 | Y | | | | 22030749 | | |
| F2T2 | F2T2.8 | TSDA10.5 | 21.0 | lean | twin (MZ) | 462 | 216 | 99 | Y | | | | 22030749 | | |
| F2T2 | F2T2.9 | TSDA10.6 | 21.0 | lean | twin (MZ) | 470 | 175 | 87 | Y | | | | 22030749 | | |
| F2T2 | F2T2.10 | TSDA10.7 | 21.0 | lean | twin (MZ) | 491 | 188 | 95 | Y | | | | 22030749 | | |
| F2T2 | F2T2.11 | TSDA10.9 | 21.0 | lean | twin (MZ) | 527 | 185 | 91 | Y | | | | 22030749 | | |
| F2T2 | F2T2.12 | TS5.5 | 24.2 | lean | twin (MZ) | 2073 | 186 | 93 | Y | | | | | | |
| F2M | F2M.1 | TS6.1 | 40.4 | obese III | mom | 0 | 211 | 110 | Y | | | | 19043404 | | |
| F2M | F2M.2 | TS6.2 | 37.0 | obese II | mom | 56 | 202 | 98 | Y | | | | 19043404 | | |
| F2M | F2M.3 | TS6.3 | 36.9 | obese II | mom | 365 | 219 | 108 | Y | | | | 19043404 | | |
| F3T1 | F3T1.1 | TS7.1 | 21.0 | lean | twin (MZ) | 0 | 145 | 81 | Y | Y | | | 19043404 | | |
| F3T1 | F3T1.2 | TS7.2 | 21.2 | lean | twin (MZ) | 203 | 161 | 79 | Y | | | | 19043404 | | |
| F3T1 | F3T1.3 | TS7.3 | 23.0 | lean | twin (MZ) | 364 | 174 | 89 | Y | Y | | | 19043404 | | |
| F3T1 | F3T1.4 | TSDA1.1 | 23.0 | lean | twin (MZ) | 391 | 135 | 75 | Y | | | | 22030749 | | |
| F3T1 | F3T1.5 | TSDA1.2 | 23.0 | lean | twin (MZ) | 399 | 166 | 91 | Y | Y | | | 22030749 | | |
| F3T1 | F3T1.6 | TSDA1.3 | 23.0 | lean | twin (MZ) | 405 | 186 | 99 | Y | | | | 22030749 | | |
| F3T1 | F3T1.7 | TSDA1.4 | 23.0 | lean | twin (MZ) | 419 | 184 | 96 | Y | | | | | | |
| F3T1 | F3T1.8 | TSDA1.5 | 23.0 | lean | twin (MZ) | 427 | 169 | 95 | Y | | | | | | |
| F3T1 | F3T1.9 | TSDA1.6 | 23.0 | lean | twin (MZ) | 441 | 171 | 91 | Y | | | | | | |
| F3T1 | F3T1.10 | TSDA1.7 | 23.0 | lean | twin (MZ) | 463 | 131 | 73 | Y | | | | | | |
| F3T1 | F3T1.11 | TSDA1.8 | 23.0 | lean | twin (MZ) | 484 | 183 | 96 | Y | Y | | | 22030749 | | |
| F3T1 | F3T1.12 | TSDA1.9 | 23.0 | lean | twin (MZ) | 497 | 149 | 77 | Y | | | | | | |
| F3T1 | F3T1.13 | TS7.5 | 28.1 | overweight | twin (MZ) | 2074 | 215 | 117 | Y | | | | | | |
| F3T2 | F3T2.1 | TS8.1 | 22.0 | lean | twin (MZ) | 0 | 164 | 91 | Y | | | | 19043404 | | |
| F3T2 | F3T2.2 | TS8.2 | 22.1 | lean | twin (MZ) | 57 | 198 | 109 | Y | | | | 19043404 | | |
| F3T2 | F3T2.3 | TS8.3 | 23.0 | lean | twin (MZ) | 353 | 231 | 124 | Y | | | | 19043404 | | |
| F3T2 | F3T2.4 | TSDA2.1 | 23.0 | lean | twin (MZ) | 373 | 120 | 67 | Y | | | | 22030749 | | |
| F3T2 | F3T2.5 | TSDA2.2 | 23.0 | lean | twin (MZ) | 387 | 197 | 99 | Y | | | | 22030749 | | |
| F3T2 | F3T2.6 | TSDA2.3 | 22.7 | lean | twin (MZ) | 395 | 130 | 76 | Y | | | | 22030749 | | |
| F3T2 | F3T2.7 | TSDA2.4 | 22.0 | lean | twin (MZ) | 410 | 170 | 89 | Y | | | | | | |
| F3T2 | F3T2.8 | TSDA2.5 | 22.0 | lean | twin (MZ) | 416 | 193 | 97 | Y | | | | | | |
| F3T2 | F3T2.9 | TSDA2.6 | 22.0 | lean | twin (MZ) | 429 | 198 | 105 | Y | | | | | | |
| F3T2 | F3T2.10 | TSDA2.7 | 22.0 | lean | twin (MZ) | 451 | 148 | 86 | Y | | | | | | |
| F3T2 | F3T2.11 | TSDA2.8 | 22.0 | lean | twin (MZ) | 470 | 198 | 112 | Y | | | | | | |
| F3T2 | F3T2.12 | TSDA2.9 | 22.0 | lean | twin (MZ) | 498 | 188 | 103 | Y | | | | | | |
| F3T2 | F3T2.13 | TS8.5 | 25.1 | overweight | twin (MZ) | 2049 | 210 | 109 | Y | | | | | | |
| F3M | F3M.1 | TS9.1 | 29.4 | overweight | mom | 0 | 205 | 98 | Y | | | | 19043404 | | |
| F3M | F3M.2 | TS9.2 | 30.5 | obese I | mom | 41 | 220 | 103 | Y | | | | 19043404 | | |
| F3M | F3M.3 | TS9.3 | 29.0 | overweight | mom | 356 | 226 | 103 | Y | | | | 19043404 | | |
| F7M | F7M.1 | TS21.1 | 38.0 | obese II | mom | 0 | | | | | | Y | 19043404 | | |
| F9T1 | F9T1.1 | TS25.1 | 21.3 | lean | twin (MZ) | 0 | 345 | 203 | Y | | | | 19043404 | | |
| F9T1 | F9T1.2 | TS25.2 | 21.8 | lean | twin (MZ) | 75 | 175 | 112 | Y | | | | 19043404 | | |
| F9T1 | F9T1.4 | TS25.4 | 19.9 | lean | twin (MZ) | 804 | 219 | 128 | Y | | | | | | |
| F9T2 | F9T2.1 | TS26.4 | 22.0 | lean | twin (MZ) | 0 | 175 | 98 | Y | | | | | | |
| F9M | F9M.1 | TS27.1 | 33.0 | obese I | mom | 0 | 174 | 106 | Y | | | | 19043404 | | |
| F9M | F9M.2 | TS27.2 | 32.9 | obese I | mom | 63 | 253 | 152 | Y | | | | 19043404 | | |
| F9M | F9M.3 | TS27.4 | 32.0 | obese I | mom | 788 | 278 | 181 | Y | | | | | | |
| F11T2 | F11T2.1 | TS32.1 | 20.4 | lean | twin (MZ) | 0 | | | | | | Y | 19043404 | | |
| F13T1 | F13T1.1 | TS37.1 | 36.2 | obese II | twin (MZ) | 0 | 200 | 99 | Y | | | | 19043404 | | |
| F13T1 | F13T1.2 | TS37.2 | 36.7 | obese II | twin (MZ) | 63 | 168 | 88 | Y | | | | 19043404 | | |
| F13T1 | F13T1.3 | TS37.3 | 31.0 | obese I | twin (MZ) | 364 | 164 | 88 | Y | | | | 19043404 | | |
| F13T1 | F13T1.4 | TS37.4 | 32.0 | obese I | twin (MZ) | 755 | 222 | 101 | Y | | | | | | |
| F13T1 | F13T1.5 | TS37.5 | 30.6 | obese I | twin (MZ) | 2034 | 271 | 125 | Y | | | | | | |
| F13T2 | F13T2.1 | TS38.1 | 25.6 | overweight | twin (MZ) | 0 | 249 | 118 | Y | | | | 19043404 | | |
| F13T2 | F13T2.2 | TS38.2 | 26.1 | overweight | twin (MZ) | 84 | 208 | 112 | Y | | | | 19043404 | | |
| F13T2 | F13T2.3 | TS38.3 | 27.0 | overweight | twin (MZ) | 364 | 277 | 126 | Y | | | | 19043404 | | |
| F13T2 | F13T2.4 | TS38.4 | 25.5 | overweight | twin (MZ) | 775 | 177 | 90 | Y | | | | | | |
| F13M | F13M.1 | TS39.1 | 39.3 | obese II | mom | 0 | 246 | 123 | Y | | | | 19043404 | | |
| F13M | F13M.2 | TS39.2 | 40.5 | obese III | mom | 77 | 233 | 113 | Y | | | | 19043404 | | |
| F13M | F13M.3 | TS39.3 | 40.0 | obese III | mom | 371 | 177 | 99 | Y | | | | 19043404 | | |
| F13M | F13M.4 | TS39.4 | 41.0 | obese III | mom | 775 | 198 | 100 | Y | | | | | | |
| F17T1 | F17T1.1 | TS61.1 | 42.7 | obese III | twin (DZ) | 0 | | | | | | Y | 19043404 | | |
| | | | | | | | | | | | | (none) | | | |
| F17T2 | F17T2.1 | TS62.1 | 40.4 | obese III | twin (DZ) | 0 | | | | | | Y | 19043404 | | |
| | | | | | | | | | | | | (none) | | | |
| F22T1 | F22T1.1 | TS76.1 | >55 | obese III | twin (MZ) | 0 | 285 | 125 | Y | | | | 19043404 | | |
| F22T1 | F22T1.2 | TS76.2 | >55 | obese III | twin (MZ) | 48 | 260 | 112 | Y | | | | 19043404 | | |
| F22T1 | F22T1.3 | TS76.3 | >55 | obese III | twin (MZ) | 363 | 256 | 112 | Y | | | | 19043404 | | |
| F22T1 | F22T1.4 | TS76.4 | >55 | obese III | twin (MZ) | 809 | 245 | 111 | Y | | | | | | |
| F22T1 | F22T1.5 | TS76.5 | 54.6 | obese III | twin (MZ) | 1981 | 286 | 124 | Y | | | | | | |
| F22T2 | F22T2.1 | TS77.1 | 27.8 | overweight | twin (MZ) | 0 | 101 | 48 | Y | | | | 19043404 | | |
| F22T2 | F22T2.2 | TS77.2 | 30.5 | obese I | twin (MZ) | 46 | 87 | 39 | Y | | | | 19043404 | | |
| F22T2 | F22T2.3 | TS77.3 | 31.5 | obese I | twin (MZ) | 393 | 51 | 28 | Y | | | | 19043404 | | |
| F22T2 | F22T2.4 | TS77.4 | 36.2 | obese II | twin (MZ) | 722 | 157 | 92 | Y | | | | | | |
| F22T2 | F22T2.5 | TS77.5 | 39.0 | obese II | twin (MZ) | 1980 | 195 | 82 | Y | | | | | | |
| F22M | F22M.1 | TS78.1 | 41.3 | obese III | mom | 0 | 145 | 71 | Y | | | | 19043404 | | |
| F22M | F22M.2 | TS78.2 | 44.0 | obese III | mom | 49 | 153 | 67 | Y | | | | 19043404 | | |
| F22M | F22M.3 | TS78.4 | 43.0 | obese III | mom | 722 | 181 | 80 | Y | | | | | | |
| F23T1 | F23T1.1 | TS82.1 | >55 | obese III | twin (MZ) | 0 | 183 | 76 | Y | | | | 19043404 | | |
| F23T1 | F23T1.2 | TS82.2 | >55 | obese III | twin (MZ) | 47 | 183 | 93 | Y | | | | | | |
| F23T1 | F23T1.3 | TS82.3 | >55 | obese III | twin (MZ) | 368 | 166 | 81 | Y | | | | 19043404 | | |
| F23T1 | F23T1.4 | TS82.4 | >55 | obese III | twin (MZ) | 748 | 200 | 89 | Y | | | | | | |
| F23T1 | F23T1.5 | TS82.5 | >55 | obese III | twin (MZ) | 1979 | 168 | 77 | Y | | | | | | |
| F23T2 | F23T2.1 | TS83.1 | 55.0 | obese III | twin (MZ) | 0 | 246 | 100 | Y | | | | 19043404 | | |
| F23T2 | F23T2.2 | TS83.2 | 54.9 | obese III | twin (MZ) | 92 | 226 | 97 | Y | | | | 19043404 | | |
| F23T2 | F23T2.3 | TS83.3 | 52.1 | obese III | twin (MZ) | 414 | 125 | 57 | Y | | | | 19043404 | | |
| F23M | F23M.1 | TS84.1 | 42.0 | obese III | mom | 0 | 168 | 88 | Y | | | | 19043404 | | |
| F23M | F23M.2 | TS84.2 | 41.0 | obese III | mom | 46 | 292 | 146 | Y | | | | 19043404 | | |
| F23M | F23M.3 | TS84.3 | 40.5 | obese III | mom | 361 | 316 | 164 | Y | | | | 19043404 | | |
| F23M | F23M.4 | TS84.4 | 41.0 | obese III | mom | 725 | 234 | 120 | Y | | | | | | |
| F27T1 | F27T1.1 | TS94.1 | 39.0 | obese III | twin (MZ) | 0 | 273 | 151 | Y | | Y | | 19043404 | | |
| | | | | | | | | | | | (none) | | | | |
| F27T1 | F27T1.2 | TS94.2 | 39.0 | obese II | twin (MZ) | 41 | 275 | 146 | Y | | | | 19043404 | | |
| F27T1 | F27T1.3 | TS94.3 | 40.4 | obese III | twin (MZ) | 391 | 360 | 198 | Y | | | | 19043404 | | |
| F27T1 | F27T1.4 | TS94.4 | 39.0 | obese II | twin (MZ) | 719 | 265 | 140 | Y | | | | | | |
| F27T2 | F27T2.1 | TS95.1 | 40.5 | obese III | twin (MZ) | 0 | 255 | 152 | Y | | Y | | 19043404 | | |
| | | | | | | | | | | | (F27M) | | | | |
| F27T2 | F27T2.2 | TS95.2 | 40.0 | obese II | twin (MZ) | 41 | 241 | 140 | Y | | | | 19043404 | | |
| F27T2 | F27T2.3 | TS95.3 | 41.5 | obese III | twin (MZ) | 390 | 238 | 120 | Y | | | | 19043404 | | |
| F27T2 | F27T2.4 | TS95.4 | 35.5 | obese II | twin (MZ) | 720 | 137 | 67 | Y | | | | | | |
| F27M | F27M.1 | TS96.1 | >55 | obese III | mom | 0 | 260 | 142 | Y | | Y | | 19043404 | | |
| | | | | | | | | | | | (F27T2) | | | | |
| F27M | F27M.2 | TS96.2 | 51.2 | obese III | mom | 42 | 200 | 117 | Y | | | | 19043404 | | |
| F27M | F27M.3 | TS96.3 | >55 | obese III | mom | 398 | 212 | 108 | Y | | | | 19043404 | | |
| F34T1 | F34T1.1 | TS118.1 | 41.6 | obese III | twin (DZ) | 0 | | | | | | Y | 19043404 | | |
| | | | | | | | | | | | | (F34T2) | | | |
| F34T2 | F34T2.1 | TS119.1 | 37.9 | obese II | twin (DZ) | 0 | | | | | | Y | 19043404 | | |
| | | | | | | | | | | | | (F34T1) | | | |
| F34M | F34M.1 | TS120.1 | >55 | obese III | mom | 0 | | | | | | Y | 19043404 | | |
| | | | | | | | | | | | | (none) | | | |
| F37T2 | F37T2.1 | TS131.1 | 46.0 | obese III | twin (DZ) | 0 | | | | | | Y | 19043404 | | |
| | | | | | | | | | | | | (F37M) | | | |
| F37M | F37M.1 | TS132.1 | 43.0 | obese III | mom | 0 | | | | | | Y | 19043404 | | |
| | | | | | | | | | | | | (F37T2) | | | |
| F42T1 | F42T1.1 | TS145.1 | 47.9 | obese III | twin (DZ) | 0 | | | | | Y | | 19043404 | | |
| | | | | | | | | | | | (F42T2) | | | | |
| F42T2 | F42T2.1 | TS146.1 | 37.3 | obese II | twin (DZ) | 0 | | | | | Y | | 19043404 | | |
| | | | | | | | | | | | (F42T1, | | | | |
| | | | | | | | | | | | F42M) | | | | |
| F42M | F42M.1 | TS147.1 | 31.8 | overweight | mom | 0 | | | | | Y | | 19043404 | | |
| | | | | | | | | | | | (F42T2) | | | | |
| F55T1 | F55T1.1 | TSDC1.1 | 30.5 | obese I | twin (MZ) | 0 | 277 | 147 | Y | | | | | | |
| F55T1 | F55T1.2 | TSDC1.2 | 30.5 | obese I | twin (MZ) | 35 | 215 | 121 | Y | | | | | | |
| F55T2 | F55T2.1 | TSDC2.1 | 27.0 | overweight | twin (MZ) | 0 | 246 | 124 | Y | | | | | | |
| F55T2 | F55T2.2 | TSDC2.2 | 27.0 | overweight | twin (MZ) | 1 | 271 | 131 | Y | | | | | | |
| F57T1 | F57T1.1 | TSDC7.1 | 32.0 | obese I | twin (MZ) | 0 | 207 | 118 | Y | | | | | | |
| F57T1 | F57T1.2 | TSDC7.2 | 33.0 | obese I | twin (MZ) | 43 | 215 | 112 | Y | | | | | | |
| F57T2 | F57T2.1 | TSDC8.1 | 24.0 | lean | twin (MZ) | 0 | 203 | 112 | Y | | | | | | |
| F57T2 | F57T2.2 | TSDC8.2 | 24.0 | lean | twin (MZ) | 35 | 282 | 153 | Y | | | | | | |
| F58T1 | F58T1.1 | TSDC10.1 | 25.0 | lean | twin (MZ) | 0 | | | | Y | | | | | |
| F58T1 | F58T1.2 | TSDC10.2 | 25.5 | overweight | twin (MZ) | 42 | | | | Y | | | | | |
| F59T1 | F59T1.1 | TSDC13.1 | 24.0 | lean | twin (MZ) | 0 | 144 | 92 | Y | | | | | | |
| F59T1 | F59T1.2 | TSDC13.2 | 24.0 | lean | twin (MZ) | 49 | 210 | 122 | Y | | | | | | |
| F59T2 | F59T2.1 | TSDC14.1 | 28.0 | overweight | twin (MZ) | 0 | 175 | 90 | Y | | | | | | |
| F59T2 | F59T2.2 | TSDC14.2 | 28.0 | overweight | twin (MZ) | 48 | 183 | 94 | Y | | | | | | |
| F60T1 | F60T1.1 | TSDC16.1 | 33.0 | obese I | twin (DZ) | 0 | 93 | 43 | Y | Y | | | | | |
| F60T1 | F60T1.2 | TSDC16.2 | 32.0 | obese I | twin (DZ) | 28 | 62 | 30 | Y | | | | | | |
| F60T2 | F60T2.1 | TSDC17.1 | 23.0 | lean | twin (DZ) | 0 | 178 | 93 | Y | Y | | | | | |
| F60T2 | F60T2.2 | TSDC17.2 | 23.0 | lean | twin (DZ) | 49 | 208 | 110 | Y | Y | | | | | |
| F61T1 | F61T1.1 | TSDC19.1 | 25.5 | overweight | twin (DZ) | 0 | | | | Y | | | | | |
| F61T1 | F61T1.2 | TSDC19.2 | 25.5 | overweight | twin (DZ) | 47 | | | | Y | | | | | |
| F61T2 | F61T2.1 | TSDC20.1 | 29.0 | overweight | twin (DZ) | 0 | | | | Y | | | | | |
| F61T2 | F61T2.2 | TSDC20.2 | 31.0 | obese I | twin (DZ) | 57 | | | | Y | | | | | |
| F62T1 | F62T1.1 | TSDC22.1 | 20.0 | lean | twin (DZ) | 0 | 208 | 103 | Y | | | | | | |
| F62T1 | F62T1.2 | TSDC22.2 | 21.0 | lean | twin (DZ) | 29 | 245 | 119 | Y | | | | | | |
| F62T2 | F62T2.1 | TSDC23.1 | 30.5 | obese I | twin (DZ) | 0 | 156 | 77 | Y | | | | | | |
| F62T2 | F62T2.2 | TSDC23.2 | 30.5 | obese I | twin (DZ) | 34 | 143 | 74 | Y | | | | | | |
| F64T1 | F64T1.1 | TSDC28.1 | 32.0 | obese I | twin (DZ) | 0 | 136 | 76 | Y | | | | | | |
| F64T1 | F64T1.2 | TSDC28.2 | 33.0 | obese I | twin (DZ) | 51 | 70 | 37 | Y | | | | | | |
| F64T2 | F64T2.1 | TSDC29.1 | 24.0 | lean | twin (DZ) | 0 | 200 | 111 | Y | | | | | | |
| F64T2 | F64T2.2 | TSDC29.2 | 24.0 | lean | twin (DZ) | 49 | 221 | 118 | Y | | | | | | |

Subject IDs are of the form: family ID, relationship (twin = T, mom = M), timepoint. Naming conventions where adapted from Turnbaugh et.al, Nature 2009 and common samples share the same family id, relationship, and timepoint designation.

TABLE 5
Performance of different models of the stability of the individual gut
microbiota as a function of time between samples.

| model | R² | Akaike information criterion |
|---|---|---|
| linear | 0.84 | −65.7 |
| exponential | 0.87 | −68.0 |
| power-law | 0.96 | −81.2 |

TABLE 6
Number of bacterial isolates sequenced from each
donor culture collection.
A. Summary statistics by sample

| | | | number of |
|---|---|---|---|
| | sample collection | number of | unique strains in |
| donor | date | sequenced isolates | collection |
| F3T1 | Mar. 26, 2007 | 23 | 13 |
| F3T1 | Mar. 24, 2008 | 32 | 19 |
| F3T1 | Apr. 28, 2008 | 19 | 11 |
| F3T1 | Jul. 22, 2008 | 47 | 19 |
| F58T1 | Sep. 30, 2008 | 34 | 23 |
| F58T1 | Nov. 11, 2008 | 50 | 25 |
| F60T1 | Sep. 22, 2008 | 36 | 14 |
| F60T2 | Sep. 22, 2008 | 68 | 27 |
| F60T2 | Nov. 10, 2008 | 53 | 28 |
| F61T1 | Oct. 15, 2008 | 29 | 18 |
| F61T1 | Dec. 1, 2008 | 53 | 32 |
| F61T2 | Sep. 16, 2008 | 40 | 15 |
| F61T2 | Nov. 12, 2008 | 49 | 21 |

B. Summary statistics by donor
(for donors with culture collection from >1 time point)

| | number of | total unique strains from all | unique strains |
|---|---|---|---|
| donor | collections | collections | from >1 sample |
| F3T1 | 4 | 31 | 20 |
| F58T1 | 2 | 37 | 10 |
| F60T2 | 2 | 41 | 14 |
| F61T1 | 2 | 42 | 10 |
| F61T2 | 2 | 25 | 8 |

TABLE 7
Assembly statistics for the 533 genomes isolated and sequenced from 6 donors.

| | | | | | | | | | N50 |
|---|---|---|---|---|---|---|---|---|---|
| | Sample | | | 16S rRNA assigned | Strain | Species | Genome | | contig |
| Donor | Date | Strain name | Species name | name (RDP) | ID | ID | length | Coverage | size |
| F3T1 | Mar. 26, 2007 | Bacteroides | Bacteroides | Bacteroides massiliensis | 29 | 10 | 4287413 | 5.0 | 967 |
| | | massiliensis | massiliensis | TS7.1-1.3 | | | | | |
| | | TS7.1-1.1 | | | | | | | |
| F3T1 | Mar. 26, 2007 | Bacteroides | Bacteroides | Bacteroides massiliensis | 21 | 10 | 4436093 | 53.6 | 74301 |
| | | massiliensis | massiliensis | TS7.1-1.4 | | | | | |
| | | TS7.1-1.2 | | | | | | | |
| F3T1 | Mar. 26, 2007 | Bacteroides | Bacteroides | Bacteroides massiliensis | 21 | 10 | 4618562 | 11.8 | 16853 |
| | | massiliensis | massiliensis | TS7.1-1.5 | | | | | |
| | | TS7.1-1.3 | | | | | | | |
| F3T1 | Mar. 26, 2007 | Bacteroides | Bacteroides | Bacteroides ovatus | 27 | 11 | 6981685 | 16.2 | 19037 |
| | | ovatus TS7.1-1.1 | ovatus | TS7.1-1.3 | | | | | |
| F3T1 | Mar. 26, 2007 | Bacteroides | Bacteroides | Bacteroides ovatus | 27 | 11 | 7306480 | 11.4 | 8916 |
| | | ovatus TS7.1-1.2 | ovatus | TS7.1-1.4 | | | | | |
| F3T1 | Mar. 26, 2007 | Bacteroides | Bacteroides | Bacteroides | 18 | 13 | 6564986 | 16.4 | 22765 |
| | | thetaiotaomicron | thetaiotaomicron | thetaiotaomicron TS7.1- | | | | | |
| | | TS7.1-1.1 | | 1.2 | | | | | |
| F3T1 | Mar. 26, 2007 | Bacteroides | Bacteroides | Bacteroides massiliensis | 11 | 15 | 4793791 | 5.9 | 1749 |
| | | vulgatus | vulgatus | TS7.1-1.1 | | | | | |
| | | TS7.1-1.1 | | | | | | | |
| F3T1 | Mar. 26, 2007 | Bacteroides | Bacteroides | Bacteroides vulgatus | 30 | 15 | 5530622 | 5.4 | 3017 |
| | | vulgatus | vulgatus | TS7.1-1.13 | | | | | |
| | | TS7.1-2.1 | | | | | | | |
| F3T1 | Mar. 26, 2007 | Butyricimonas | Butyricimonas | Butyricimonas virosa | 8 | 23 | 4729566 | 57.5 | 120965 |
| | | virosa | virosa | TS7.1-1.1 | | | | | |
| | | TS7.1-1.1 | | | | | | | |
| F3T1 | Mar. 26, 2007 | Butyricimonas | Butyricimonas | Butyricimonas virosa | 8 | 23 | 4994763 | 6.3 | 2996 |
| | | virosa | virosa | TS7.1-1.6 | | | | | |
| | | TS7.1-1.2 | | | | | | | |
| F3T1 | Mar. 26, 2007 | Butyricimonas | Butyricimonas | Butyricimonas virosa | 15 | 23 | 4870989 | 38.3 | 80370 |
| | | virosa TS7.1-2.1 | virosa | TS7.1-2.8 | | | | | |
| F3T1 | Mar. 26, 2007 | Coprococcus | Coprococcus | Coprococcus comes | 3 | 40 | 3745752 | 106.8 | 92364 |
| | | comes TS7.1-1.1 | comes | TS7.1-2.9 | | | | | |
| F3T1 | Mar. 26, 2007 | Coprococcus | Coprococcus | Coprococcus comes | 3 | 40 | 3763142 | 108.7 | 46279 |
| | | comes TS7.1-1.2 | comes | TS7.1-3.16 | | | | | |
| F3T1 | Mar. 26, 2007 | Coprococcus | Coprococcus | Coprococcus comes | 3 | 40 | 3748194 | 194.7 | 23828 |
| | | comes TS7.1-1.3 | comes | TS7.1-3.20 | | | | | |
| F3T1 | Mar. 26, 2007 | Parabacteroides | Parabacteroides | Parabacteroides | 9 | 55 | 5280062 | 40.4 | 61426 |
| | | distasonis TS7.1- | distasonis | distasonis TS7.1-3.2 | | | | | |
| | | 1.1 | | | | | | | |
| F3T1 | Mar. 26, 2007 | Parabacteroides | Parabacteroides | Parabacteroides | 9 | 55 | 5257711 | 9.6 | 10494 |
| | | distasonis TS7.1- | distasonis | distasonis TS7.1-5.7 | | | | | |
| | | 1.2 | | | | | | | |
| F3T1 | Mar. 26, 2007 | Parabacteroides | Parabacteroides | Parabacteroides | 9 | 55 | 5240157 | 29.6 | 61505 |
| | | distasonis TS7.1- | distasonis | distasonis TS7.1-5.8 | | | | | |
| | | 1.3 | | | | | | | |
| F3T1 | Mar. 26, 2007 | Parabacteroides | Parabacteroides | Parabacteroides | 9 | 55 | 5242174 | 123.9 | 117950 |
| | | distasonis TS7.1- | distasonis | distasonis TS7.1-5.9 | | | | | |
| | | 1.4 | | | | | | | |
| F3T1 | Mar. 26, 2007 | Ruminococcus | Ruminococcus | Ruminococcus obeum | 33 | 66 | 4256951 | 43.3 | 20184 |
| | | obeum TS7.1-1.1 | obeum | TS7.1-4.3 | | | | | |
| F3T1 | Mar. 26, 2007 | Ruminococcus | Ruminococcus | Ruminococcus TS7.1- | 26 | 69 | 2986984 | 59.4 | 16452 |
| | | torques TS7.1-1.1 | torques | 2.3 | | | | | |
| F3T1 | Mar. 26, 2007 | Ruminococcus | Ruminococcus | Ruminococcus TS7.1- | 26 | 69 | 2980331 | 27.3 | 8273 |
| | | torques TS7.1-1.2 | torques | 2.4 | | | | | |
| F3T1 | Mar. 26, 2007 | Ruminococcus | Ruminococcus | Ruminococcus TS7.1- | 26 | 69 | 3006394 | 39.1 | 58494 |
| | | torques TS7.1-1.3 | torques | 3.2 | | | | | |
| F3T1 | Mar. 26, 2007 | Subdoligranulum | Subdoligranulum | Clostridiaceae TS7.1-1.1 | 22 | 74 | 3501702 | 270.4 | 37488 |
| | | variabile TS7.1- | variabile | | | | | | |
| | | 1.1 | | | | | | | |
| F3T1 | Mar. 24, 2008 | Alistipes | Alistipes | Alistipes indistinctus | 7 | 1 | 2891162 | 16.2 | 261834 |
| | | indistinctus TS7.3- | indistinctus | TS7.3-1.1 | | | | | |
| | | 1.1 | | | | | | | |
| F3T1 | Mar. 24, 2008 | Bacteroides | Bacteroides | Bacteroides ovatus | 27 | 11 | 6866053 | 39.8 | 162477 |
| | | ovatus TS7.3-1.1 | ovatus | TS7.3-1.2 | | | | | |
| F3T1 | Mar. 24, 2008 | Bacteroides | Bacteroides | Bacteroides salyersiae | 14 | 12 | 5393044 | 15.4 | 84312 |
| | | salyersiae TS7.3- | salyersiae | TS7.3-1.2 | | | | | |
| | | 1.1 | | | | | | | |
| F3T1 | Mar. 24, 2008 | Bacteroides | Bacteroides | Bacteroides | 18 | 13 | 6575415 | 60.4 | 116654 |
| | | thetaiotaomicron | thetaiotaomicron | thetaiotaomicron TS7.3- | | | | | |
| | | TS7.3-1.1 | | 1.1 | | | | | |
| F3T1 | Mar. 24, 2008 | Bacteroides | Bacteroides | Bacteroides faecis | 12 | 13 | 6238892 | 76.5 | 100490 |
| | | thetaiotaomicron | thetaiotaomicron | TS7.3-1.2 | | | | | |
| | | TS7.3-1.2 | | | | | | | |
| F3T1 | Mar. 24, 2008 | Bacteroides | Bacteroides | Bacteroides faecis | 12 | 13 | 6234521 | 96.5 | 109726 |
| | | thetaiotaomicron | thetaiotaomicron | TS7.3-1.4 | | | | | |
| | | TS7.3-1.3 | | | | | | | |
| F3T1 | Mar. 24, 2008 | Bacteroides | Bacteroides | Bacteroides faecis | 12 | 13 | 6233685 | 36.7 | 85958 |
| | | thetaiotaomicron | thetaiotaomicron | TS7.3-1.6 | | | | | |
| | | TS7.3-1.4 | | | | | | | |
| F3T1 | Mar. 24, 2008 | Bacteroides | Bacteroides | Bacteroides faecis | 12 | 13 | 6237250 | 19.2 | 47309 |
| | | thetaiotaomicron | thetaiotaomicron | TS7.3-1.1 | | | | | |
| | | TS7.3-1.5 | | | | | | | |
| F3T1 | Mar. 24, 2008 | Bifidobacterium | Bifidobacterium | Bifidobacterium longum | 4 | 19 | 2398405 | 616.7 | 80461 |
| | | longum TS7.3-1.1 | longum | TS7.3-2.1 | | | | | |
| F3T1 | Mar. 24, 2008 | Bifidobacterium | Bifidobacterium | Bifidobacterium longum | 4 | 19 | 2403275 | 133.3 | 80228 |
| | | longum TS7.3-1.2 | longum | TS7.3-2.2 | | | | | |
| F3T1 | Mar. 24, 2008 | Bifidobacterium | Bifidobacterium | Bifidobacterium longum | 4 | 19 | 2397301 | 45.9 | 70140 |
| | | longum TS7.3-1.3 | longum | TS7.3-2.3 | | | | | |
| F3T1 | Mar. 24, 2008 | Bifidobacterium | Bifidobacterium | Bifidobacterium longum | 4 | 19 | 2348671 | 121.6 | 80465 |
| | | longum TS7.3-1.4 | longum | TS7.3-2.4 | | | | | |
| F3T1 | Mar. 24, 2008 | Bifidobacterium | Bifidobacterium | Bifidobacterium longum | 4 | 19 | 2397117 | 647.4 | 60972 |
| | | longum TS7.3-1.5 | longum | TS7.3-2.6 | | | | | |
| F3T1 | Mar. 24, 2008 | Clostridium | Clostridium | Clostridium TS7.3-1.1 | 2 | 31 | 3737021 | 27.3 | 141280 |
| | | TS7.3-1.1 | | | | | | | |
| F3T1 | Mar. 24, 2008 | Clostridium | Clostridium | Clostridium TS7.3-1.3 | 2 | 31 | 3792005 | 66.1 | 171168 |
| | | TS7.3-1.2 | | | | | | | |
| F3T1 | Mar. 24, 2008 | Coprococcus | Coprococcus | Coprococcus comes | 3 | 40 | 3704369 | 157.9 | 81152 |
| | | comes TS7.3-1.1 | comes | TS7.3-1.2 | | | | | |
| F3T1 | Mar. 24, 2008 | Coprococcus | Coprococcus | Coprococcus comes | 3 | 40 | 3830393 | 100.8 | 84662 |
| | | comes TS7.3-1.10 | comes | TS7.3-1.23 | | | | | |
| F3T1 | Mar. 24, 2008 | Coprococcus | Coprococcus | Coprococcus comes | 3 | 40 | 3827481 | 242.0 | 81097 |
| | | comes TS7.3-1.11 | comes | TS7.3-1.24 | | | | | |
| F3T1 | Mar. 24, 2008 | Coprococcus | Coprococcus | Coprococcus comes | 3 | 40 | 3680731 | 119.0 | 93836 |
| | | comes TS7.3-1.2 | comes | TS7.3-2.4 | | | | | |
| F3T1 | Mar. 24, 2008 | Coprococcus | Coprococcus | Coprococcus comes | 3 | 40 | 3678945 | 64.4 | 76400 |
| | | comes TS7.3-1.3 | comes | TS7.3-4.5 | | | | | |
| F3T1 | Mar. 24, 2008 | Coprococcus | Coprococcus | Coprococcus comes | 3 | 40 | 3683935 | 249.5 | 100072 |
| | | comes TS7.3-1.4 | comes | TS7.3-4.8 | | | | | |
| F3T1 | Mar. 24, 2008 | Coprococcus | Coprococcus | Coprococcus comes | 3 | 40 | 3675279 | 266.5 | 79606 |
| | | comes TS7.3-1.5 | comes | TS7.3-2.11 | | | | | |
| F3T1 | Mar. 24, 2008 | Coprococcus | Coprococcus | Coprococcus comes | 3 | 40 | 3680230 | 139.0 | 74423 |
| | | comes TS7.3-1.6 | comes | TS7.3-2.12 | | | | | |
| F3T1 | Mar. 24, 2008 | Coprococcus | Coprococcus | Coprococcus comes | 3 | 40 | 3765967 | 259.1 | 81289 |
| | | comes TS7.3-1.7 | comes | TS7.3-4.13 | | | | | |
| F3T1 | Mar. 24, 2008 | Coprococcus | Coprococcus | Coprococcus comes | 3 | 40 | 3681541 | 183.1 | 93265 |
| | | comes TS7.3-1.8 | comes | TS7.3-4.14 | | | | | |
| F3T1 | Mar. 24, 2008 | Coprococcus | Coprococcus | Coprococcus comes | 3 | 40 | 3763616 | 174.5 | 103019 |
| | | comes TS7.3-1.9 | comes | TS7.3-1.19 | | | | | |
| F3T1 | Mar. 24, 2008 | Ruminococcus | Ruminococcus | Ruminococcus | 39 | 64 | 3729731 | 68.0 | 115153 |
| | | gauvreauii TS7.3- | gauvreauii | gauvreauii TS7.3-1.1 | | | | | |
| | | 1.1 | | | | | | | |
| F3T1 | Mar. 24, 2008 | Ruminococcus | Ruminococcus | Ruminococcus gnavus | 37 | 65 | 3166345 | 251.9 | 112393 |
| | | gnavus TS7.3-1.1 | gnavus | TS7.3-1.2 | | | | | |
| F3T1 | Mar. 24, 2008 | Ruminococcus | Ruminococcus | Ruminococcus obeum | 24 | 66 | 4098091 | 127.0 | 56575 |
| | | obeum TS7.3-1.1 | obeum | TS7.3-2.2 | | | | | |
| F3T1 | Mar. 24, 2008 | Ruminococcus sp | Ruminococcus sp | Ruminococcus TS7.3- | 38 | 67 | 2936686 | 65.4 | 127801 |
| | | CCUG 37327 A | CCUG 37327 A | 1.1 | | | | | |
| | | TS7.3-1.1 | | | | | | | |
| F3T1 | Mar. 24, 2008 | Subdoligranulum | Subdoligranulum | Clostridiaceae TS7.3-1.4 | 22 | 74 | 3506503 | 58.7 | 67848 |
| | | variabile TS7.3- | variabile | | | | | | |
| | | 1.1 | | | | | | | |
| F3T1 | Mar. 24, 2008 | Subdoligranulum | Subdoligranulum | Clostridiaceae TS7.3-1.6 | 22 | 74 | 3500309 | 37.9 | 53240 |
| | | variabile TS7.3- | variabile | | | | | | |
| | | 1.2 | | | | | | | |
| F3T1 | Apr. 28, 2008 | Alistipes | Alistipes | Alistipes indistinctus | 7 | 1 | 2962961 | 12.1 | 127190 |
| | | indistinctus | indistinctus | TSDA1.2-1.1 | | | | | |
| | | TSDA1.2-1.1 | | | | | | | |
| F3T1 | Apr. 28, 2008 | Alistipes | Alistipes | Alistipes indistinctus | 7 | 1 | 2943630 | 6.7 | 11587 |
| | | indistinctus | indistinctus | TSDA1.2-1.2 | | | | | |
| | | TSDA1.2-1.2 | | | | | | | |
| F3T1 | Apr. 28, 2008 | Bifidobacterium | Bifidobacterium | Bifidobacterium longum | 1 | 19 | 2238981 | 329.5 | 75129 |
| | | longum TSDA1.2- | longum | TSDA1.2-1.5 | | | | | |
| | | 1.1 | | | | | | | |
| F3T1 | Apr. 28, 2008 | Bifidobacterium | Bifidobacterium | Bifidobacterium longum | 1 | 19 | 2240635 | 799.0 | 88804 |
| | | longum TSDA1.2- | longum | TSDA1.2-1.6 | | | | | |
| | | 1.2 | | | | | | | |
| F3T1 | Apr. 28, 2008 | Butyricimonas | Butyricimonas | Butyricimonas virosa | 8 | 23 | 4729711 | 65.7 | 217032 |
| | | virosa TSDA1.2- | virosa | TSDA1.2-1.8 | | | | | |
| | | 1.1 | | | | | | | |
| F3T1 | Apr. 28, 2008 | Clostridium | Clostridium | Clostridium TSDA1.2-1.4 | 2 | 31 | 3794591 | 99.6 | 174763 |
| | | TSDA1.2-1.1 | | | | | | | |
| F3T1 | Apr. 28, 2008 | Collinsella | Collinsella | Collinsella aerofaciens | 16 | 39 | 2224752 | 40.0 | 41285 |
| | | aerofaciens | aerofaciens | TSDA1.2-1.1 | | | | | |
| | | TSDA1.2-1.1 | | | | | | | |
| F3T1 | Apr. 28, 2008 | Collinsella | Collinsella | Collinsella aerofaciens | 16 | 39 | 2225809 | 228.5 | 41438 |
| | | aerofaciens | aerofaciens | TSDA1.2-2.3 | | | | | |
| | | TSDA1.2-1.2 | | | | | | | |
| F3T1 | Apr. 28, 2008 | Collinsella | Collinsella | Collinsella aerofaciens | 16 | 39 | 2223764 | 183.0 | 49881 |
| | | aerofaciens | aerofaciens | TSDA1.2-2.4 | | | | | |
| | | TSDA1.2-1.3 | | | | | | | |
| F3T1 | Apr. 28, 2008 | Dorea | Dorea | Dorea formicigenerans | 10 | 41 | 3184825 | 91.8 | 126475 |
| | | formicigenerans | formicigenerans | TSDA1.2-1.3 | | | | | |
| | | TSDA1.2-1.1 | | | | | | | |
| F3T1 | Apr. 28, 2008 | Dorea | Dorea | Dorea formicigenerans | 10 | 41 | 3294637 | 107.9 | 204909 |
| | | formicigenerans | formicigenerans | TSDA1.2-2.6 | | | | | |
| | | TSDA1.2-1.2 | | | | | | | |
| F3T1 | Apr. 28, 2008 | Dorea longicatena | Dorea longicatena | Dorea longicatena | 25 | 42 | 3395516 | 132.1 | 45026 |
| | | TSDA1.2-1.1 | | TSDA1.2-1.2 | | | | | |
| F3T1 | Apr. 28, 2008 | Escherichia coli | Escherichia coli | Escherichia coli | 5 | 45 | 4923103 | 67.4 | 242488 |
| | | TSDA1.2-1.1 | | TSDA1.2-1.3 | | | | | |
| F3T1 | Apr. 28, 2008 | Escherichia coli | Escherichia coli | Escherichia coli | 5 | 45 | 4919246 | 32.4 | 229742 |
| | | TSDA1.2-1.2 | | TSDA1.2-1.8 | | | | | |
| F3T1 | Apr. 28, 2008 | Escherichia coli | Escherichia coli | Escherichia coli | 5 | 45 | 4920896 | 78.3 | 246809 |
| | | TSDA1.2-1.3 | | TSDA1.2-2.7 | | | | | |
| F3T1 | Apr. 28, 2008 | Escherichia coli | Escherichia coli | Escherichia coli | 5 | 45 | 4967042 | 10.7 | 54875 |
| | | TSDA1.2-1.4 | | TSDA1.2-2.9 | | | | | |
| F3T1 | Apr. 28, 2008 | Subdoligranulum | Subdoligranulum | Clostridium TSDA1.2-1.2 | 32 | 74 | 3651524 | 121.9 | 48017 |
| | | variabile | variabile | | | | | | |
| | | TSDA1.2-1.1 | | | | | | | |
| F3T1 | Apr. 28, 2008 | Subdoligranulum | Subdoligranulum | Clostridiaceae TSDA1.2- | 22 | 74 | 3521263 | 7.6 | 7405 |
| | | variabile | variabile | 2.1 | | | | | |
| | | TSDA1.2-2.1 | | | | | | | |
| F3T1 | Apr. 28, 2008 | Subdoligranulum | Subdoligranulum | Subdoligranulum | 36 | 74 | 6417665 | 93.2 | 740 |
| | | variabile | variabile | variabile TSDA1.2-2.4 | | | | | |
| | | TSDA1.2-3.1 | | | | | | | |
| F3T1 | Jul. 22, 2008 | Alistipes | Alistipes | Alistipes indistinctus | 7 | 1 | 2885734 | 34.3 | 172362 |
| | | indistinctus | indistinctus | TSDA1.8-1.4 | | | | | |
| | | TSDA1.8-1.1 | | | | | | | |
| F3T1 | Jul. 22, 2008 | Bacteroides | Bacteroides | Bacteroides intestinalis | 28 | 9 | 6275116 | 5.2 | 2245 |
| | | intestinalis | intestinalis | TSDA1.8-1.1 | | | | | |
| | | TSDA1.8-1.1 | | | | | | | |
| F3T1 | Jul. 22, 2008 | Bacteroides | Bacteroides | Bacteroides intestinalis | 28 | 9 | 6387123 | 80.8 | 182479 |
| | | intestinalis | intestinalis | TSDA1.8-1.2 | | | | | |
| | | TSDA1.8-1.2 | | | | | | | |
| F3T1 | Jul. 22, 2008 | Bacteroides | Bacteroides | Bacteroides massiliensis | 21 | 10 | 4582279 | 29.0 | 63143 |
| | | massiliensis | massiliensis | TSDA1.8-1.1 | | | | | |
| | | TSDA1.8-1.1 | | | | | | | |
| F3T1 | Jul. 22, 2008 | Bacteroides | Bacteroides | Bacteroides massiliensis | 21 | 10 | 4570863 | 16.1 | 41860 |
| | | massiliensis | massiliensis | TSDA1.8-1.2 | | | | | |
| | | TSDA1.8-1.2 | | | | | | | |
| F3T1 | Jul. 22, 2008 | Bacteroides | Bacteroides | Bacteroides massiliensis | 21 | 10 | 4590387 | 262.2 | 91596 |
| | | massiliensis | massiliensis | TSDA1.8-1.3 | | | | | |
| | | TSDA1.8-1.3 | | | | | | | |
| F3T1 | Jul. 22, 2008 | Bacteroides | Bacteroides | Bacteroides massiliensis | 21 | 10 | 4587203 | 49.5 | 84967 |
| | | massiliensis | massiliensis | TSDA1.8-1.4 | | | | | |
| | | TSDA1.8-1.4 | | | | | | | |
| F3T1 | Jul. 22, 2008 | Bacteroides | Bacteroides | Bacteroides salyersiae | 14 | 12 | 5376317 | 14.3 | 47319 |
| | | salyersiae | salyersiae | TSDA1.8-1.1 | | | | | |
| | | TSDA1.8-1.1 | | | | | | | |
| F3T1 | Jul. 22, 2008 | Bacteroides | Bacteroides | Bacteroides salyersiae | 14 | 12 | 5370493 | 34.6 | 120299 |
| | | salyersiae | salyersiae | TSDA1.8-1.2 | | | | | |
| | | TSDA1.8-1.2 | | | | | | | |
| F3T1 | Jul. 22, 2008 | Bacteroides | Bacteroides | Bacteroides salyersiae | 14 | 12 | 5369996 | 9.2 | 28443 |
| | | salyersiae | salyersiae | TSDA1.8-1.3 | | | | | |
| | | TSDA1.8-1.3 | | | | | | | |
| F3T1 | Jul. 22, 2008 | Bacteroides | Bacteroides | Bacteroides faecis | 12 | 13 | 6231854 | 35.7 | 78126 |
| | | thetaiotaomicron | thetaiotaomicron | TSDA1.8-1.1 | | | | | |
| | | TSDA1.8-1.1 | | | | | | | |
| F3T1 | Jul. 22, 2008 | Bacteroides | Bacteroides | Bacteroides faecis | 12 | 13 | 6227896 | 56.6 | 77526 |
| | | thetaiotaomicron | thetaiotaomicron | TSDA1.8-1.2 | | | | | |
| | | TSDA1.8-1.2 | | | | | | | |
| F3T1 | Jul. 22, 2008 | Bacteroides | Bacteroides | Bacteroides | 18 | 13 | 6570364 | 26.6 | 71866 |
| | | thetaiotaomicron | thetaiotaomicron | thetaiotaomicron | | | | | |
| | | TSDA1.8-2.1 | | TSDA1.8-1.1 | | | | | |
| F3T1 | Jul. 22, 2008 | Bacteroides | Bacteroides | Bacteroides vulgatus | 11 | 15 | 4897510 | 11.7 | 28149 |
| | | vulgatus | vulgatus | TSDA1.8-1.5 | | | | | |
| | | TSDA1.8-1.1 | | | | | | | |
| F3T1 | Jul. 22, 2008 | Bacteroides | Bacteroides | Bacteroides vulgatus | 11 | 15 | 4872592 | 29.3 | 121559 |
| | | vulgatus | vulgatus | TSDA1.8-2.1 | | | | | |
| | | TSDA1.8-1.2 | | | | | | | |
| F3T1 | Jul. 22, 2008 | Bacteroides | Bacteroides | Bacteroides vulgatus | 11 | 15 | 4878788 | 12.5 | 39417 |
| | | vulgatus | vulgatus | TSDA1.8-2.2 | | | | | |
| | | TSDA1.8-1.3 | | | | | | | |
| F3T1 | Jul. 22, 2008 | Bacteroides | Bacteroides | Bacteroides vulgatus | 11 | 15 | 4888192 | 35.5 | 102355 |
| | | vulgatus | vulgatus | TSDA1.8-2.3 | | | | | |
| | | TSDA1.8-1.4 | | | | | | | |
| F3T1 | Jul. 22, 2008 | Bacteroides | Bacteroides | Bacteroides vulgatus | 35 | 15 | 5444668 | 35.3 | 67450 |
| | | vulgatus | vulgatus | TSDA1.8-2.4 | | | | | |
| | | TSDA1.8-2.1 | | | | | | | |
| F3T1 | Jul. 22, 2008 | Bifidobacterium | Bifidobacterium | Bifidobacterium longum | 1 | 19 | 2297949 | 74.5 | 86312 |
| | | longum TSDA1.8- | longum | TSDA1.8-1.7 | | | | | |
| | | 1.1 | | | | | | | |
| F3T1 | Jul. 22, 2008 | Bifidobacterium | Bifidobacterium | Bifidobacterium longum | 1 | 19 | 2296412 | 168.9 | 71135 |
| | | longum TSDA1.8- | longum | TSDA1.8-1.8 | | | | | |
| | | 1.2 | | | | | | | |
| F3T1 | Jul. 22, 2008 | Bifidobacterium | Bifidobacterium | Bifidobacterium longum | 4 | 19 | 2395891 | 226.3 | 48169 |
| | | longum TSDA1.8- | longum | TSDA1.8-2.3 | | | | | |
| | | 2.1 | | | | | | | |
| F3T1 | Jul. 22, 2008 | Butyricimonas | Butyricimonas | Butyricimonas virosa | 8 | 23 | 4727442 | 46.1 | 166699 |
| | | virosa TSDA1.8- | virosa | TSDA1.8-1.3 | | | | | |
| | | 1.1 | | | | | | | |
| F3T1 | Jul. 22, 2008 | Butyricimonas | Butyricimonas | Butyricimonas virosa | 8 | 23 | 4731645 | 39.8 | 178127 |
| | | virosa TSDA1.8- | virosa | TSDA1.8-1.6 | | | | | |
| | | 1.2 | | | | | | | |
| F3T1 | Jul. 22, 2008 | Butyricimonas | Butyricimonas | Butyricimonas virosa | 15 | 23 | 4863826 | 37.6 | 91029 |
| | | virosa TSDA1.8- | virosa | TSDA1.8-3.4 | | | | | |
| | | 2.1 | | | | | | | |
| F3T1 | Jul. 22, 2008 | Clostridium | Clostridium | Clostridium TSDA1.8-1.1 | 2 | 31 | 3789466 | 54.0 | 127760 |
| | | TSDA1.8-1.1 | | | | | | | |
| F3T1 | Jul. 22, 2008 | Coprococcus | Coprococcus | Coprococcus comes | 3 | 40 | 3677934 | 308.4 | 79517 |
| | | comes TSDA1.8- | comes | TSDA1.8-2.1 | | | | | |
| | | 1.1 | | | | | | | |
| F3T1 | Jul. 22, 2008 | Coprococcus | Coprococcus | Coprococcus comes | 3 | 40 | 3763018 | 255.3 | 38679 |
| | | comes TSDA1.8- | comes | TSDA1.8-6.13 | | | | | |
| | | 1.10 | | | | | | | |
| F3T1 | Jul. 22, 2008 | Coprococcus | Coprococcus | Coprococcus comes | 3 | 40 | 3762868 | 122.5 | 89892 |
| | | comes TSDA1.8- | comes | TSDA1.8-2.5 | | | | | |
| | | 1.2 | | | | | | | |
| F3T1 | Jul. 22, 2008 | Coprococcus | Coprococcus | Coprococcus comes | 3 | 40 | 3673213 | 200.1 | 81102 |
| | | comes TSDA1.8- | comes | TSDA1.8-2.7 | | | | | |
| | | 1.3 | | | | | | | |
| F3T1 | Jul. 22, 2008 | Coprococcus | Coprococcus | Coprococcus comes | 3 | 40 | 3680893 | 154.1 | 100074 |
| | | comes TSDA1.8- | comes | TSDA1.8-3.2 | | | | | |
| | | 1.4 | | | | | | | |
| F3T1 | Jul. 22, 2008 | Coprococcus | Coprococcus | Coprococcus comes | 3 | 40 | 3679753 | 268.4 | 100069 |
| | | comes TSDA1.8- | comes | TSDA1.8-4.6 | | | | | |
| | | 1.5 | | | | | | | |
| F3T1 | Jul. 22, 2008 | Coprococcus | Coprococcus | Coprococcus comes | 3 | 40 | 3677585 | 201.5 | 54340 |
| | | comes TSDA1.8- | comes | TSDA1.8-4.8 | | | | | |
| | | 1.6 | | | | | | | |
| F3T1 | Jul. 22, 2008 | Coprococcus | Coprococcus | Coprococcus comes | 3 | 40 | 3678219 | 171.5 | 81249 |
| | | comes TSDA1.8- | comes | TSDA1.8-4.10 | | | | | |
| | | 1.7 | | | | | | | |
| F3T1 | Jul. 22, 2008 | Coprococcus | Coprococcus | Coprococcus comes | 3 | 40 | 3678479 | 209.2 | 76822 |
| | | comes TSDA1.8- | comes | TSDA1.8-4.11 | | | | | |
| | | 1.8 | | | | | | | |
| F3T1 | Jul. 22, 2008 | Coprococcus | Coprococcus | Coprococcus comes | 3 | 40 | 3762995 | 170.3 | 86758 |
| | | comes TSDA1.8- | comes | TSDA1.8-5.3 | | | | | |
| | | 1.9 | | | | | | | |
| F3T1 | Jul. 22, 2008 | Dorea | Dorea | Dorea formicigenerans | 10 | 41 | 3288343 | 29.4 | 81388 |
| | | formicigenerans | formicigenerans | TSDA1.8-1.1 | | | | | |
| | | TSDA1.8-1.1 | | | | | | | |
| F3T1 | Jul. 22, 2008 | Dorea | Dorea | Dorea formicigenerans | 10 | 41 | 3288923 | 42.5 | 98410 |
| | | formicigenerans | formicigenerans | TSDA1.8-2.2 | | | | | |
| | | TSDA1.8-2.2 | | | | | | | |
| F3T1 | Jul. 22, 2008 | Dorea longicatena | Dorea longicatena | Dorea longicatena | 25 | 42 | 3452874 | 123.1 | 37212 |
| | | TSDA1.8-1.1 | | TSDA1.8-1.1 | | | | | |
| F3T1 | Jul. 22, 2008 | Dorea longicatena | Dorea longicatena | Dorea longicatena | 25 | 42 | 3411767 | 149.8 | 31345 |
| | | TSDA1.8-1.2 | | TSDA1.8-1.4 | | | | | |
| F3T1 | Jul. 22, 2008 | Escherichia coli | Escherichia coli | Escherichia coli | 5 | 45 | 4915044 | 178.7 | 200685 |
| | | TSDA1.8-1.1 | | TSDA1.8-1.1 | | | | | |
| F3T1 | Jul. 22, 2008 | Escherichia coli | Escherichia coli | Escherichia coli | 5 | 45 | 4911941 | 19.4 | 149878 |
| | | TSDA1.8-1.2 | | TSDA1.8-1.5 | | | | | |
| F3T1 | Jul. 22, 2008 | Escherichia coli | Escherichia coli | Escherichia coli | 5 | 45 | 4913634 | 47.0 | 190831 |
| | | TSDA1.8-1.3 | | TSDA1.8-2.2 | | | | | |
| F3T1 | Jul. 22, 2008 | Parabacteroides | Parabacteroides | Parabacteroides | 9 | 55 | 5271570 | 175.4 | 218163 |
| | | distasonis | distasonis | distasonis TSDA1.8-1.2 | | | | | |
| | | TSDA1.8-1.1 | | | | | | | |
| F3T1 | Jul. 22, 2008 | Parabacteroides | Parabacteroides | Parabacteroides | 9 | 55 | 5269350 | 161.1 | 117966 |
| | | distasonis | distasonis | distasonis TSDA1.8-1.3 | | | | | |
| | | TSDA1.8-1.2 | | | | | | | |
| F3T1 | Jul. 22, 2008 | Parabacteroides | Parabacteroides | Parabacteroides | 9 | 55 | 5272494 | 87.1 | 128797 |
| | | distasonis | distasonis | distasonis TSDA1.8-1.4 | | | | | |
| | | TSDA1.8-1.3 | | | | | | | |
| F3T1 | Jul. 22, 2008 | Parabacteroides | Parabacteroides | Parabacteroides | 9 | 55 | 5269229 | 69.5 | 146667 |
| | | distasonis | distasonis | distasonis TSDA1.8-2.5 | | | | | |
| | | TSDA1.8-1.4 | | | | | | | |
| F3T1 | Jul. 22, 2008 | Ruminococcus | Ruminococcus | Ruminococcus obeum | 24 | 66 | 4095340 | 57.0 | 53475 |
| | | obeum TSDA1.8- | obeum | TSDA1.8-3.2 | | | | | |
| | | 1.1 | | | | | | | |
| F58T1 | Sep. 30, 2008 | Anaerococcus | Anaerococcus | Anaerococcus vaginalis | 32 | 2 | 2118035 | 68.1 | 47814 |
| | | vaginalis | vaginalis | TSDC10.1-1.1 | | | | | |
| | | TSDC10.1-1.1 | | | | | | | |
| F58T1 | Sep. 30, 2008 | Anaerofustis | Anaerofustis | Anaerofustis | 40 | 3 | 1982044 | 13.4 | 6402 |
| | | stercorihominis | stercorihominis | stercorihominis | | | | | |
| | | TSDC10.1-1.1 | | TSDC10.1-1.1 | | | | | |
| F58T1 | Sep. 30, 2008 | Bacteroides | Bacteroides caccae | Bacteroides caccae | 7 | 6 | 5051748 | 82.2 | 111574 |
| | | caccae | | TSDC10.1-1.3 | | | | | |
| | | TSDC10.1-1.1 | | | | | | | |
| F58T1 | Sep. 30, 2008 | Bacteroides | Bacteroides | Bacteroides | 12 | 9 | 6900698 | 15.4 | 99130 |
| | | intestinalis | intestinalis | cellulosilyticus | | | | | |
| | | TSDC10.1-1.1 | | TSDC10.1-1.3 | | | | | |
| F58T1 | Sep. 30, 2008 | Bacteroides | Bacteroides | Bacteroides vulgatus | 13 | 15 | 5038595 | 14.8 | 68837 |
| | | vulgatus | vulgatus | TSDC10.1-1.1 | | | | | |
| | | TSDC10.1-1.1 | | | | | | | |
| F58T1 | Sep. 30, 2008 | Bacteroides | Bacteroides | Bacteroides vulgatus | 13 | 15 | 5022815 | 20.1 | 73571 |
| | | vulgatus | vulgatus | TSDC10.1-1.2 | | | | | |
| | | TSDC10.1-1.2 | | | | | | | |
| F58T1 | Sep. 30, 2008 | Bacteroides | Bacteroides | Bacteroides dorei | 20 | 15 | 5535217 | 66.6 | 166293 |
| | | vulgatus | vulgatus | TSDC10.1-1.2 | | | | | |
| | | TSDC10.1-2.1 | | | | | | | |
| F58T1 | Sep. 30, 2008 | Butyricimonas | Butyricimonas | Butyricimonas virosa | 33 | 23 | 4949056 | 10.5 | 27575 |
| | | virosa TSDC10.1- | virosa | TSDC10.1-1.1 | | | | | |
| | | 1.1 | | | | | | | |
| F58T1 | Sep. 30, 2008 | Collinsella | Collinsella | Collinsella TSDC10.1- | 3 | 38 | 1833865 | 233.2 | 103469 |
| | | TSDC10.1-1.1 | | 1.1 | | | | | |
| F58T1 | Sep. 30, 2008 | Collinsella | Collinsella | Collinsella TSDC10.1- | 3 | 38 | 1834506 | 350.9 | 102339 |
| | | TSDC10.1-2.2 | | 2.2 | | | | | |
| F58T1 | Sep. 30, 2008 | Coprococcus | Coprococcus | Coprococcus comes | 22 | 40 | 3413726 | 332.1 | 70574 |
| | | comes TSDC10.1- | comes | TSDC10.1-1.1 | | | | | |
| | | 1.1 | | | | | | | |
| F58T1 | Sep. 30, 2008 | Dorea longicatena | Dorea longicatena | Dorea longicatena | 19 | 42 | 2903016 | 107.8 | 66389 |
| | | TSDC10.1-1.1 | | TSDC10.1-1.1 | | | | | |
| F58T1 | Sep. 30, 2008 | Dorea longicatena | Dorea longicatena | Dorea longicatena | 19 | 42 | 2880575 | 68.9 | 64642 |
| | | TSDC10.1-1.2 | | TSDC10.1-1.4 | | | | | |
| F58T1 | Sep. 30, 2008 | Escherichia coli | Escherichia coli | Escherichia coli | 14 | 45 | 5240364 | 24.1 | 175825 |
| | | TSDC10.1-1.1 | | TSDC10.1-1.1 | | | | | |
| F58T1 | Sep. 30, 2008 | Escherichia coli | Escherichia coli | Escherichia coli | 14 | 45 | 5243657 | 84.0 | 175698 |
| | | TSDC10.1-1.2 | | TSDC10.1-1.2 | | | | | |
| F58T1 | Sep. 30, 2008 | Escherichia coli | Escherichia coli | Escherichia coli | 14 | 45 | 5243985 | 102.0 | 175732 |
| | | TSDC10.1-1.3 | | TSDC10.1-1.3 | | | | | |
| F58T1 | Sep. 30, 2008 | Eubacterium | Eubacterium | Eubacterium biforme | 23 | 46 | 2791900 | 68.1 | 26370 |
| | | biforme | biforme | TSDC10.1-1.2 | | | | | |
| | | TSDC10.1-1.1 | | | | | | | |
| F58T1 | Sep. 30, 2008 | Eubacterium | Eubacterium | Eubacterium biforme | 23 | 46 | 2719719 | 80.6 | 27685 |
| | | biforme | biforme | TSDC10.1-1.4 | | | | | |
| | | TSDC10.1-1.2 | | | | | | | |
| F58T1 | Sep. 30, 2008 | Eubacterium | Eubacterium | Eubacterium biforme | 25 | 46 | 2274800 | 141.8 | 17217 |
| | | biforme | biforme | TSDC10.1-2.1 | | | | | |
| | | TSDC10.1-2.1 | | | | | | | |
| F58T1 | Sep. 30, 2008 | Eubacterium | Eubacterium | Eubacterium biforme | 24 | 46 | 2303590 | 162.0 | 17352 |
| | | biforme | biforme | TSDC10.1-2.3 | | | | | |
| | | TSDC10.1-3.1 | | | | | | | |
| F58T1 | Sep. 30, 2008 | Lactobacillus | Lactobacillus casei | Lactobacillus casei | 21 | 52 | 2844244 | 356.2 | 25671 |
| | | casei TSDC10.1- | | TSDC10.1-1.1 | | | | | |
| | | 1.1 | | | | | | | |
| F58T1 | Sep. 30, 2008 | Lactobacillus | Lactobacillus casei | Lactobacillus casei | 21 | 52 | 2961453 | 251.6 | 22068 |
| | | casei TSDC10.1- | | TSDC10.1-1.2 | | | | | |
| | | 1.2 | | | | | | | |
| F58T1 | Sep. 30, 2008 | Lactobacillus | Lactobacillus casei | Lactobacillus casei | 21 | 52 | 2970861 | 258.0 | 16971 |
| | | casei TSDC10.1- | | TSDC10.1-1.3 | | | | | |
| | | 1.3 | | | | | | | |
| F58T1 | Sep. 30, 2008 | Lactobacillus | Lactobacillus | Lactobacillus TSDC10.1- | 29 | 51 | 3058302 | 93.2 | 59622 |
| | | TSDC10.1-1.1 | | 1.1 | | | | | |
| F58T1 | Sep. 30, 2008 | Parabacteroides | Parabacteroides | Bacteroides sp 20 3 | 5 | 55 | 5043373 | 56.7 | 210409 |
| | | distasonis | distasonis | TSDC10.1-1.1 | | | | | |
| | | TSDC10.1-1.1 | | | | | | | |
| F58T1 | Sep. 30, 2008 | Parabacteroides | Parabacteroides | Bacteroides sp 3 1 19 | 5 | 55 | 5028749 | 72.5 | 177275 |
| | | distasonis | distasonis | TSDC10.1-1.2 | | | | | |
| | | TSDC10.1-1.2 | | | | | | | |
| F58T1 | Sep. 30, 2008 | Parabacteroides | Parabacteroides | Bacteroides TSDC10.1- | 5 | 55 | 5046842 | 87.5 | 216052 |
| | | distasonis | distasonis | 1.1 | | | | | |
| | | TSDC10.1-1.3 | | | | | | | |
| F58T1 | Sep. 30, 2008 | Parabacteroides | Parabacteroides | Parabacteroides sp D13 | 5 | 55 | 5046355 | 110.1 | 169725 |
| | | distasonis | distasonis | TSDC10.1-1.3 | | | | | |
| | | TSDC10.1-1.4 | | | | | | | |
| F58T1 | Sep. 30, 2008 | Parabacteroides | Parabacteroides | Parabacteroides | 17 | 56 | 7318715 | 31.9 | 94590 |
| | | goldsteinii | goldsteinii | goldsteinii TSDC10.1- | | | | | |
| | | TSDC10.1-1.1 | | 1.1 | | | | | |
| F58T1 | Sep. 30, 2008 | Parabacteroides | Parabacteroides | Parabacteroides merdae | 10 | 57 | 4678654 | 23.7 | 83217 |
| | | merdae | merdae | TSDC10.1-1.3 | | | | | |
| | | TSDC10.1-1.1 | | | | | | | |
| F58T1 | Sep. 30, 2008 | Peptoniphilus | Peptoniphilus harei | Peptoniphilus harei | 28 | 58 | 1814904 | 12.3 | 10145 |
| | | harei TSDC10.1- | | TSDC10.1-1.4 | | | | | |
| | | 1.1 | | | | | | | |
| F58T1 | Sep. 30, 2008 | Peptoniphilus | Peptoniphilus harei | Peptoniphilus harei | 27 | 58 | 1885045 | 20.1 | 16767 |
| | | harei TSDC10.1- | | TSDC10.1-2.5 | | | | | |
| | | 2.1 | | | | | | | |
| F58T1 | Sep. 30, 2008 | Ruminococcus | Ruminococcus | Ruminococcus bromii | 11 | 63 | 2330482 | 75.0 | 70167 |
| | | bromii TSDC10.1- | bromii | TSDC10.1-1.4 | | | | | |
| | | 1.1 | | | | | | | |
| F58T1 | Sep. 30, 2008 | Subdoligranulum | Subdoligranulum | Subdoligranulum | 16 | 74 | 4061511 | 37.1 | 30008 |
| | | variabile | variabile | variabile TSDC10.1-1.2 | | | | | |
| | | TSDC10.1-1.1 | | | | | | | |
| F58T1 | Nov. 11, 2008 | Bacteroides | Bacteroides caccae | Bacteroides caccae | 7 | 6 | 5045833 | 30.2 | 84946 |
| | | caccae | | TSDC10.2-1.1 | | | | | |
| | | TSDC10.2-1.1 | | | | | | | |
| F58T1 | Nov. 11, 2008 | Bacteroides | Bacteroides caccae | Bacteroides caccae | 7 | 6 | 5056025 | 30.2 | 110547 |
| | | caccae | | TSDC10.2-1.2 | | | | | |
| | | TSDC10.2-1.2 | | | | | | | |
| F58T1 | Nov. 11, 2008 | Bacteroides | Bacteroides caccae | Bacteroides caccae | 7 | 6 | 5052459 | 47.7 | 94800 |
| | | caccae | | TSDC10.2-1.4 | | | | | |
| | | TSDC10.2-1.3 | | | | | | | |
| F58T1 | Nov. 11, 2008 | Bacteroides | Bacteroides caccae | Bacteroides caccae | 7 | 6 | 5052151 | 35.7 | 106349 |
| | | caccae | | TSDC10.2-1.5 | | | | | |
| | | TSDC10.2-1.4 | | | | | | | |
| F58T1 | Nov. 11, 2008 | Bacteroides | Bacteroides | Bacteroides | 12 | 9 | 6902803 | 76.3 | 148896 |
| | | intestinalis | intestinalis | cellulosilyticus | | | | | |
| | | TSDC10.2-1.1 | | TSDC10.2-1.1 | | | | | |
| F58T1 | Nov. 11, 2008 | Bacteroides | Bacteroides | Bacteroides | 12 | 9 | 6993199 | 20.7 | 73698 |
| | | intestinalis | intestinalis | cellulosilyticus | | | | | |
| | | TSDC10.2-1.2 | | TSDC10.2-1.4 | | | | | |
| F58T1 | Nov. 11, 2008 | Bacteroides | Bacteroides | Bacteroides uniformis | 4 | 14 | 4787555 | 75.7 | 178289 |
| | | uniformis | uniformis | TSDC10.2-1.2 | | | | | |
| | | TSDC10.2-1.1 | | | | | | | |
| F58T1 | Nov. 11, 2008 | Bacteroides | Bacteroides | Bacteroides uniformis | 4 | 14 | 4691812 | 80.8 | 193345 |
| | | uniformis | uniformis | TSDC10.2-1.4 | | | | | |
| | | TSDC10.2-1.2 | | | | | | | |
| F58T1 | Nov. 11, 2008 | Bacteroides | Bacteroides | Bacteroides uniformis | 4 | 14 | 4794347 | 136.3 | 216281 |
| | | uniformis | uniformis | TSDC10.2-1.5 | | | | | |
| | | TSDC10.2-1.3 | | | | | | | |
| F58T1 | Nov. 11, 2008 | Bacteroides | Bacteroides | Bacteroides uniformis | 4 | 14 | 4797236 | 73.7 | 194616 |
| | | uniformis | uniformis | TSDC10.2-1.7 | | | | | |
| | | TSDC10.2-1.4 | | | | | | | |
| F58T1 | Nov. 11, 2008 | Bacteroides | Bacteroides | Bacteroides uniformis | 4 | 14 | 4797160 | 110.8 | 244969 |
| | | uniformis | uniformis | TSDC10.2-1.11 | | | | | |
| | | TSDC10.2-1.5 | | | | | | | |
| F58T1 | Nov. 11, 2008 | Bacteroides | Bacteroides | Bacteroides uniformis | 2 | 14 | 4850070 | 81.9 | 208536 |
| | | uniformis | uniformis | TSDC10.2-2.1 | | | | | |
| | | TSDC10.2-2.1 | | | | | | | |
| F58T1 | Nov. 11, 2008 | Bacteroides | Bacteroides | Bacteroides uniformis | 2 | 14 | 4850208 | 110.1 | 183792 |
| | | uniformis | uniformis | TSDC10.2-2.6 | | | | | |
| | | TSDC10.2-2.2 | | | | | | | |
| F58T1 | Nov. 11, 2008 | Bacteroides | Bacteroides | Bacteroides uniformis | 2 | 14 | 4850690 | 144.2 | 208517 |
| | | uniformis | uniformis | TSDC10.2-2.8 | | | | | |
| | | TSDC10.2-2.3 | | | | | | | |
| F58T1 | Nov. 11, 2008 | Bacteroides | Bacteroides | Bacteroides uniformis | 2 | 14 | 4844282 | 58.2 | 195845 |
| | | uniformis | uniformis | TSDC10.2-2.10 | | | | | |
| | | TSDC10.2-2.4 | | | | | | | |
| F58T1 | Nov. 11, 2008 | Bacteroides | Bacteroides | Bacteroides uniformis | 2 | 14 | 4848009 | 68.9 | 158273 |
| | | uniformis | uniformis | TSDC10.2-2.12 | | | | | |
| | | TSDC10.2-2.5 | | | | | | | |
| F58T1 | Nov. 11, 2008 | Bacteroides | Bacteroides | Bacteroides vulgatus | 13 | 15 | 5025344 | 61.1 | 110415 |
| | | vulgatus | vulgatus | TSDC10.2-1.4 | | | | | |
| | | TSDC10.2-1.1 | | | | | | | |
| F58T1 | Nov. 11, 2008 | Bacteroides | Bacteroides | Bacteroides dorei | 20 | 15 | 5545706 | 11.7 | 32865 |
| | | vulgatus | vulgatus | TSDC10.2-1.4 | | | | | |
| | | TSDC10.2-2.1 | | | | | | | |
| F58T1 | Nov. 11, 2008 | Bacteroides | Bacteroides | Bacteroides dorei | 20 | 15 | 5451263 | 67.6 | 147645 |
| | | vulgatus | vulgatus | TSDC10.2-1.3 | | | | | |
| | | TSDC10.2-2.2 | | | | | | | |
| F58T1 | Nov. 11, 2008 | Bacteroides | Bacteroides | Bacteroides vulgatus | 26 | 15 | 5027107 | 67.9 | 82737 |
| | | vulgatus | vulgatus | TSDC10.2-1.3 | | | | | |
| | | TSDC10.2-3.1 | | | | | | | |
| F58T1 | Nov. 11, 2008 | Barnesiella | Barnesiella | Barnesiella | 38 | 16 | 3502125 | 24.3 | 100953 |
| | | intestinihominis | intestinihominis | intestinihominis | | | | | |
| | | TSDC10.2-1.1 | | TSDC10.2-1.5 | | | | | |
| F58T1 | Nov. 11, 2008 | Blautia | Blautia | Blautia TSDC10.2-1.1 | 34 | 21 | 2738394 | 183.7 | 85920 |
| | | TSDC10.2-1.1 | | | | | | | |
| F58T1 | Nov. 11, 2008 | Clostridiales | Clostridiales | Clostridiales TSDC10.2- | 36 | 28 | 3164952 | 217.8 | 427089 |
| | | TSDC10.2-2.1 | | 1.2 | | | | | |
| F58T1 | Nov. 11, 2008 | Clostridium | Clostridium | Clostridiales TSDC10.2- | 9 | 31 | 3852009 | 134.7 | 67869 |
| | | TSDC10.2-1.1 | | 1.4 | | | | | |
| F58T1 | Nov. 11, 2008 | Clostridium | Clostridium | Clostridium TSDC10.2- | 9 | 31 | 3851428 | 131.5 | 59627 |
| | | TSDC10.2-1.2 | | 1.1 | | | | | |
| F58T1 | Nov. 11, 2008 | Clostridium | Clostridium | Clostridium TSDC10.2- | 9 | 31 | 3838739 | 87.9 | 75932 |
| | | TSDC10.2-1.3 | | 1.3 | | | | | |
| F58T1 | Nov. 11, 2008 | Clostridium | Clostridium | Clostridium TSDC10.2- | 9 | 31 | 3833493 | 206.8 | 71469 |
| | | TSDC10.2-1.4 | | 1.4 | | | | | |
| F58T1 | Nov. 11, 2008 | Clostridium | Clostridium | Clostridium TSDC10.2- | 9 | 31 | 3852992 | 87.2 | 66937 |
| | | TSDC10.2-1.5 | | 1.5 | | | | | |
| F58T1 | Nov. 11, 2008 | Clostridium | Clostridium | Clostridium TSDC10.2- | 9 | 31 | 3876090 | 252.8 | 56522 |
| | | TSDC10.2-1.6 | | 1.6 | | | | | |
| F58T1 | Nov. 11, 2008 | Coprococcus | Coprococcus | Coprococcus comes | 22 | 40 | 3366740 | 216.1 | 94970 |
| | | comes TSDC10.2- | comes | TSDC10.2-1.3 | | | | | |
| | | 1.1 | | | | | | | |
| F58T1 | Nov. 11, 2008 | Dorea | Dorea | Dorea formicigenerans | 31 | 41 | 3276033 | 247.0 | 99794 |
| | | formicigenerans | formicigenerans | TSDC10.2-1.1 | | | | | |
| | | TSDC10.2-1.1 | | | | | | | |
| F58T1 | Nov. 11, 2008 | Dorea longicatena | Dorea longicatena | Dorea longicatena | 19 | 42 | 2822603 | 82.5 | 61059 |
| | | TSDC10.2-1.1 | | TSDC10.2-1.3 | | | | | |
| F58T1 | Nov. 11, 2008 | Dorea longicatena | Dorea longicatena | Dorea longicatena | 19 | 42 | 2870795 | 122.6 | 65018 |
| | | TSDC10.2-1.2 | | TSDC10.2-1.5 | | | | | |
| F58T1 | Nov. 11, 2008 | Dorea longicatena | Dorea longicatena | Dorea longicatena | 19 | 42 | 2868740 | 68.7 | 71046 |
| | | TSDC10.2-1.3 | | TSDC10.2-1.6 | | | | | |
| F58T1 | Nov. 11, 2008 | Dorea longicatena | Dorea longicatena | Dorea longicatena | 30 | 43 | 2799331 | 104.8 | 101237 |
| | | TSDC10.2-2.1 | | TSDC10.2-2.2 | | | | | |
| F58T1 | Nov. 11, 2008 | Eubacterium | Eubacterium | Eubacterium eligens | 39 | 49 | 3024507 | 37.8 | 216845 |
| | | eligens | eligens | TSDC10.2-1.1 | | | | | |
| | | TSDC10.2-1.1 | | | | | | | |
| F58T1 | Nov. 11, 2008 | Odoribacter | Odoribacter | Odoribacter | 35 | 54 | 4590827 | 30.2 | 114283 |
| | | splanchnicus | splanchnicus | splanchnicus TSDC10.2- | | | | | |
| | | TSDC10.2-1.1 | | 1.2 | | | | | |
| F58T1 | Nov. 11, 2008 | Parabacteroides | Parabacteroides | Bacteroides sp 3 1 19 | 5 | 55 | 5049191 | 50.7 | 218052 |
| | | distasonis | distasonis | TSDC10.2-1.1 | | | | | |
| | | TSDC10.2-1.1 | | | | | | | |
| F58T1 | Nov. 11, 2008 | Parabacteroides | Parabacteroides | Parabacteroides sp D13 | 5 | 55 | 5018099 | 151.4 | 192031 |
| | | distasonis | distasonis | TSDC10.2-1.1 | | | | | |
| | | TSDC10.2-1.2 | | | | | | | |
| F58T1 | Nov. 11, 2008 | Parabacteroides | Parabacteroides | Parabacteroides sp D13 | 5 | 55 | 5047097 | 55.6 | 243019 |
| | | distasonis | distasonis | TSDC10.2-2.2 | | | | | |
| | | TSDC10.2-1.3 | | | | | | | |
| F58T1 | Nov. 11, 2008 | Parabacteroides | Parabacteroides | Parabacteroides merdae | 5 | 55 | 5016499 | 304.7 | 195986 |
| | | distasonis | distasonis | TSDC10.2-1.2 | | | | | |
| | | TSDC10.2-1.4 | | | | | | | |
| F58T1 | Nov. 11, 2008 | Parabacteroides | Parabacteroides | Parabacteroides | 17 | 56 | 7348839 | 17.8 | 64938 |
| | | goldsteinii | goldsteinii | goldsteinii TSDC10.2- | | | | | |
| | | TSDC10.2-1.1 | | 1.2 | | | | | |
| F58T1 | Nov. 11, 2008 | Parabacteroides | Parabacteroides | Parabacteroides merdae | 10 | 57 | 4680901 | 243.2 | 122096 |
| | | merdae | merdae | TSDC10.2-1.4 | | | | | |
| | | TSDC10.2-1.1 | | | | | | | |
| F58T1 | Nov. 11, 2008 | Parabacteroides | Parabacteroides | Parabacteroides merdae | 10 | 57 | 4679130 | 103.1 | 121791 |
| | | merdae | merdae | TSDC10.2-1.5 | | | | | |
| | | TSDC10.2-1.2 | | | | | | | |
| F58T1 | Nov. 11, 2008 | Ruminococcus | Ruminococcus | Ruminococcus bromii | 11 | 63 | 2334323 | 166.1 | 89084 |
| | | bromii TSDC10.2- | bromii | TSDC10.2-1.2 | | | | | |
| | | 1.1 | | | | | | | |
| F58T1 | Nov. 11, 2008 | Ruminococcus sp | Ruminococcus sp | Ruminococcus sp | 1 | 67 | 2821086 | 162.2 | 100645 |
| | | CCUG 37327 A | CCUG 37327 A | CCUG 37327 A | | | | | |
| | | TSDC10.2-1.1 | | TSDC10.2-1.2 | | | | | |
| F58T1 | Nov. 11, 2008 | Ruminococcus sp | Ruminococcus sp | Ruminococcus sp | 1 | 67 | 2820349 | 299.9 | 146733 |
| | | CCUG 37327 A | CCUG 37327 A | CCUG 37327 A | | | | | |
| | | TSDC10.2-1.2 | | TSDC10.2-1.4 | | | | | |
| F58T1 | Nov. 11, 2008 | Ruminococcus sp | Ruminococcus sp | Ruminococcus sp | 1 | 67 | 2820902 | 150.1 | 146890 |
| | | CCUG 37327 A | CCUG 37327 A | CCUG 37327 A | | | | | |
| | | TSDC10.2-1.3 | | TSDC10.2-1.5 | | | | | |
| F58T1 | Nov. 11, 2008 | Ruminococcus | Ruminococcus | Ruminococcus | 37 | 61 | 3483604 | 85.9 | 68552 |
| | | TSDC10.2-1.1 | | TSDC10.2-1.1 | | | | | |
| F58T1 | Nov. 11, 2008 | Subdoligranulum | Subdoligranulum | Subdoligranulum | 16 | 74 | 4066098 | 54.4 | 37921 |
| | | variabile | variabile | variabile TSDC10.2-1.5 | | | | | |
| | | TSDC10.2-1.1 | | | | | | | |
| F60T1 | Sep. 22, 2008 | Anaerococcus | Anaerococcus | Anaerococcus | 14 | 2 | 2044031 | 205.5 | 31820 |
| | | hydrogenalis | vaginalis | hydrogenalis TSDC16.1- | | | | | |
| | | TSDC16.1-1.1 | | 1.3 | | | | | |
| F60T1 | Sep. 22, 2008 | Bacteroides | Bacteroides | Bacteroides TSDC16.1- | 1 | 11 | 6274074 | 48.1 | 49740 |
| | | ovatus TSDC16.1- | ovatus | 1.1 | | | | | |
| | | 1.1 | | | | | | | |
| F60T1 | Sep. 22, 2008 | Bacteroides | Bacteroides | Bacteroides TSDC16.1- | 1 | 11 | 6304326 | 136.8 | 69785 |
| | | ovatus TSDC16.1- | ovatus | 1.2 | | | | | |
| | | 1.2 | | | | | | | |
| F60T1 | Sep. 22, 2008 | Bacteroides | Bacteroides | Bacteroides TSDC16.1- | 1 | 11 | 6303816 | 443.0 | 60990 |
| | | ovatus TSDC16.1- | ovatus | 1.3 | | | | | |
| | | 1.3 | | | | | | | |
| F60T1 | Sep. 22, 2008 | Bacteroides | Bacteroides | Bacteroides TSDC16.1- | 1 | 11 | 6301937 | 165.0 | 46908 |
| | | ovatus TSDC16.1- | ovatus | 1.8 | | | | | |
| | | 1.4 | | | | | | | |
| F60T1 | Sep. 22, 2008 | Bacteroides | Bacteroides | Bacteroides TSDC16.1- | 1 | 11 | 6304988 | 117.2 | 35653 |
| | | ovatus TSDC16.1- | ovatus | 2.17 | | | | | |
| | | 1.5 | | | | | | | |
| F60T1 | Sep. 22, 2008 | Bacteroides | Bacteroides | Bacteroides TSDC16.1- | 1 | 11 | 6310301 | 236.0 | 29897 |
| | | ovatus TSDC16.1- | ovatus | 1.13 | | | | | |
| | | 1.6 | | | | | | | |
| F60T1 | Sep. 22, 2008 | Bacteroides | Bacteroides | Bacteroides ovatus | 9 | 11 | 6185829 | 68.4 | 3245 |
| | | ovatus TSDC16.1- | ovatus | TSDC16.1-1.3 | | | | | |
| | | 2.1 | | | | | | | |
| F60T1 | Sep. 22, 2008 | Bacteroides | Bacteroides | Bacteroides vulgatus | 2 | 15 | 4738141 | 73.7 | 92510 |
| | | vulgatus | vulgatus | TSDC16.1-1.4 | | | | | |
| | | TSDC16.1-1.1 | | | | | | | |
| F60T1 | Sep. 22, 2008 | Bacteroides | Bacteroides | Bacteroides vulgatus | 2 | 15 | 4744694 | 66.6 | 18805 |
| | | vulgatus | vulgatus | TSDC16.1-1.8 | | | | | |
| | | TSDC16.1-1.2 | | | | | | | |
| F60T1 | Sep. 22, 2008 | Bacteroides | Bacteroides | Bacteroides vulgatus | 2 | 15 | 4737872 | 214.7 | 87417 |
| | | vulgatus | vulgatus | TSDC16.1-1.9 | | | | | |
| | | TSDC16.1-1.3 | | | | | | | |
| F60T1 | Sep. 22, 2008 | Bacteroides | Bacteroides | Bacteroides vulgatus | 2 | 15 | 4761391 | 110.7 | 16396 |
| | | vulgatus | vulgatus | TSDC16.1-1.12 | | | | | |
| | | TSDC16.1-1.4 | | | | | | | |
| F60T1 | Sep. 22, 2008 | Bacteroides | Bacteroides | Bacteroides vulgatus | 10 | 15 | 4533253 | 51.0 | 1741 |
| | | vulgatus | vulgatus | TSDC16.1-1.13 | | | | | |
| | | TSDC16.1-2.1 | | | | | | | |
| F60T1 | Sep. 22, 2008 | Clostridium | Clostridium | Clostridium scindens | 4 | 37 | 4191388 | 116.4 | 86189 |
| | | scindens | scindens | TSDC16.1-1.1 | | | | | |
| | | TSDC16.1-1.1 | | | | | | | |
| F60T1 | Sep. 22, 2008 | Clostridium | Clostridium | Clostridium scindens | 4 | 37 | 4194589 | 508.6 | 98153 |
| | | scindens | scindens | TSDC16.1-1.3 | | | | | |
| | | TSDC16.1-1.2 | | | | | | | |
| F60T1 | Sep. 22, 2008 | Megasphaera | Megasphaera | Megasphaera elsdenii | 8 | 53 | 2642411 | 91.5 | 5680 |
| | | elsdenii | elsdenii | TSDC16.1-1.7 | | | | | |
| | | TSDC16.1-1.1 | | | | | | | |
| F60T1 | Sep. 22, 2008 | Megasphaera | Megasphaera | Megasphaera elsdenii | 5 | 53 | 2682790 | 106.4 | 104064 |
| | | elsdenii | elsdenii | TSDC16.1-2.19 | | | | | |
| | | TSDC16.1-2.1 | | | | | | | |
| F60T1 | Sep. 22, 2008 | Megasphaera | Megasphaera | Megasphaera elsdenii | 5 | 53 | 2678466 | 302.6 | 16372 |
| | | elsdenii | elsdenii | TSDC16.1-2.2 | | | | | |
| | | TSDC16.1-2.2 | | | | | | | |
| F60T1 | Sep. 22, 2008 | Megasphaera | Megasphaera | Megasphaera elsdenii | 5 | 53 | 2797405 | 176.4 | 19761 |
| | | elsdenii | elsdenii | TSDC16.1-2.9 | | | | | |
| | | TSDC16.1-2.3 | | | | | | | |
| F60T1 | Sep. 22, 2008 | Megasphaera | Megasphaera | Megasphaera elsdenii | 5 | 53 | 2682549 | 642.7 | 73065 |
| | | elsdenii | elsdenii | TSDC16.1-3.32 | | | | | |
| | | TSDC16.1-2.4 | | | | | | | |
| F60T1 | Sep. 22, 2008 | Parabacteroides | Parabacteroides | Parabacteroides | 6 | 55 | 5067627 | 131.4 | 169820 |
| | | distasonis | distasonis | distasonis TSDC16.1- | | | | | |
| | | TSDC16.1-1.1 | | 2.8 | | | | | |
| F60T1 | Sep. 22, 2008 | Parabacteroides | Parabacteroides | Parabacteroides | 6 | 55 | 5074014 | 52.9 | 61184 |
| | | distasonis | distasonis | distasonis TSDC16.1- | | | | | |
| | | TSDC16.1-1.2 | | 2.9 | | | | | |
| F60T1 | Sep. 22, 2008 | Parabacteroides | Parabacteroides | Parabacteroides | 6 | 55 | 5070433 | 145.8 | 144026 |
| | | distasonis | distasonis | distasonis TSDC16.1- | | | | | |
| | | TSDC16.1-1.3 | | 3.4 | | | | | |
| F60T1 | Sep. 22, 2008 | Ruminococcus | Ruminococcus | Ruminococcus gnavus | 13 | 65 | 3255486 | 70.9 | 50538 |
| | | gnavus | gnavus | TSDC16.1-2.2 | | | | | |
| | | TSDC16.1-1.1 | | | | | | | |
| F60T1 | Sep. 22, 2008 | Ruminococcus | Ruminococcus | Ruminococcus gnavus | 12 | 65 | 2745770 | 198.4 | 100528 |
| | | gnavus | gnavus | TSDC16.1-3.3 | | | | | |
| | | TSDC16.1-2.1 | | | | | | | |
| F60T1 | Sep. 22, 2008 | Ruminococcus | Ruminococcus | Ruminococcus gnavus | 11 | 65 | 3390079 | 131.5 | 70084 |
| | | gnavus | gnavus | TSDC16.1-4.1 | | | | | |
| | | TSDC16.1-3.1 | | | | | | | |
| F60T1 | Sep. 22, 2008 | Streptococcus | Streptococcus | Streptococcus | 3 | 70 | 1848118 | 138.4 | 54867 |
| | | TSDC16.1-1.1 | | TSDC16.1-1.2 | | | | | |
| F60T1 | Sep. 22, 2008 | Streptococcus | Streptococcus | Streptococcus | 3 | 70 | 1846812 | 96.3 | 47427 |
| | | TSDC16.1-1.2 | | TSDC16.1-1.3 | | | | | |
| F60T1 | Sep. 22, 2008 | Streptococcus | Streptococcus | Streptococcus | 3 | 70 | 1912233 | 230.0 | 43181 |
| | | TSDC16.1-1.3 | | TSDC16.1-1.8 | | | | | |
| F60T1 | Sep. 22, 2008 | Streptococcus | Streptococcus | Streptococcus | 3 | 70 | 1951978 | 105.7 | 45674 |
| | | TSDC16.1-1.4 | | TSDC16.1-1.16 | | | | | |
| F60T1 | Sep. 22, 2008 | Subdoligranulum | Subdoligranulum | Clostridiaceae | 7 | 74 | 3626954 | 150.1 | 66239 |
| | | variabile | variabile | TSDC16.1-1.1 | | | | | |
| | | TSDC16.1-1.1 | | | | | | | |
| F60T1 | Sep. 22, 2008 | Subdoligranulum | Subdoligranulum | Clostridiaceae | 7 | 74 | 3627900 | 75.4 | 26168 |
| | | variabile | variabile | TSDC16.1-1.2 | | | | | |
| | | TSDC16.1-1.2 | | | | | | | |
| F60T1 | Sep. 22, 2008 | Subdoligranulum | Subdoligranulum | Clostridiaceae | 7 | 74 | 3615243 | 196.2 | 19995 |
| | | variabile | variabile | TSDC16.1-1.4 | | | | | |
| | | TSDC16.1-1.3 | | | | | | | |
| F60T1 | Sep. 22, 2008 | Subdoligranulum | Subdoligranulum | Clostridiaceae | 7 | 74 | 3626552 | 356.7 | 45296 |
| | | variabile | variabile | TSDC16.1-1.9 | | | | | |
| | | TSDC16.1-1.4 | | | | | | | |
| F60T1 | Sep. 22, 2008 | Subdoligranulum | Subdoligranulum | Clostridiaceae | 7 | 74 | 3620735 | 206.5 | 76475 |
| | | variabile | variabile | TSDC16.1-1.11 | | | | | |
| | | TSDC16.1-1.5 | | | | | | | |
| F60T1 | Sep. 22, 2008 | Subdoligranulum | Subdoligranulum | Clostridiaceae | 7 | 74 | 4268026 | 43.9 | 18271 |
| | | variabile | variabile | TSDC16.1-1.14 | | | | | |
| | | TSDC16.1-1.6 | | | | | | | |
| F60T2 | Sep. 22, 2008 | Bacteroides | Bacteroides caccae | Bacteroides caccae | 29 | 6 | 5724607 | 25.0 | 4974 |
| | | caccae | | TSDC17.1-1.2 | | | | | |
| | | TSDC17.1-1.1 | | | | | | | |
| F60T2 | Sep. 22, 2008 | Bacteroides | Bacteroides | Bacteroides finegoldii | 18 | 7 | 4517428 | 78.7 | 91315 |
| | | finegoldii | finegoldii | TSDC17.1-1.1 | | | | | |
| | | TSDC17.1-1.1 | | | | | | | |
| F60T2 | Sep. 22, 2008 | Bacteroides | Bacteroides | Bacteroides finegoldii | 18 | 7 | 4468437 | 231.3 | 68335 |
| | | finegoldii | finegoldii | TSDC17.1-1.4 | | | | | |
| | | TSDC17.1-1.2 | | | | | | | |
| F60T2 | Sep. 22, 2008 | Bacteroides | Bacteroides | Bacteroides intestinalis | 7 | 9 | 7352665 | 109.3 | 210856 |
| | | intestinalis | intestinalis | TSDC17.1-1.1 | | | | | |
| | | TSDC17.1-1.1 | | | | | | | |
| F60T2 | Sep. 22, 2008 | Bacteroides | Bacteroides | Bacteroides massiliensis | 22 | 10 | 4561652 | 394.2 | 77118 |
| | | massiliensis | massiliensis | TSDC17.1-1.1 | | | | | |
| | | TSDC17.1-1.1 | | | | | | | |
| F60T2 | Sep. 22, 2008 | Bacteroides | Bacteroides | Bacteroides ovatus | 21 | 11 | 7109951 | 164.9 | 146479 |
| | | ovatus TSDC17.1- | ovatus | TSDC17.1-1.4 | | | | | |
| | | 1.1 | | | | | | | |
| F60T2 | Sep. 22, 2008 | Bacteroides | Bacteroides | Bacteroides ovatus | 21 | 11 | 7154053 | 65.7 | 122292 |
| | | ovatus TSDC17.1- | ovatus | TSDC17.1-1.6 | | | | | |
| | | 1.2 | | | | | | | |
| F60T2 | Sep. 22, 2008 | Bacteroides | Bacteroides | Bacteroides ovatus | 21 | 11 | 7121114 | 64.3 | 46366 |
| | | ovatus TSDC17.1- | ovatus | TSDC17.1-1.8 | | | | | |
| | | 1.3 | | | | | | | |
| F60T2 | Sep. 22, 2008 | Bacteroides | Bacteroides | Bacteroides ovatus | 1 | 11 | 6841024 | 93.1 | 140944 |
| | | ovatus TSDC17.1- | ovatus | TSDC17.1-1.5 | | | | | |
| | | 2.1 | | | | | | | |
| F60T2 | Sep. 22, 2008 | Bacteroides | Bacteroides | Bacteroides ovatus | 1 | 11 | 6839214 | 162.2 | 99606 |
| | | ovatus TSDC17.1- | ovatus | TSDC17.1-2.10 | | | | | |
| | | 2.2 | | | | | | | |
| F60T2 | Sep. 22, 2008 | Bacteroides | Bacteroides | Bacteroides | 25 | 13 | 6345966 | 122.4 | 80044 |
| | | thetaiotaomicron | thetaiotaomicron | thetaiotaomicron | | | | | |
| | | TSDC17.1-1.1 | | TSDC17.1-1.1 | | | | | |
| F60T2 | Sep. 22, 2008 | Bacteroides | Bacteroides | Bacteroides uniformis | 5 | 14 | 5018648 | 248.6 | 106904 |
| | | uniformis | uniformis | TSDC17.1-1.1 | | | | | |
| | | TSDC17.1-1.1 | | | | | | | |
| F60T2 | Sep. 22, 2008 | Bacteroides | Bacteroides | Bacteroides uniformis | 5 | 14 | 5032983 | 98.2 | 85123 |
| | | uniformis | uniformis | TSDC17.1-1.3 | | | | | |
| | | TSDC17.1-1.2 | | | | | | | |
| F60T2 | Sep. 22, 2008 | Bacteroides | Bacteroides | Bacteroides uniformis | 5 | 14 | 5022608 | 266.1 | 122758 |
| | | uniformis | uniformis | TSDC17.1-1.4 | | | | | |
| | | TSDC17.1-1.3 | | | | | | | |
| F60T2 | Sep. 22, 2008 | Bacteroides | Bacteroides | Bacteroides uniformis | 5 | 14 | 5025432 | 130.8 | 123392 |
| | | uniformis | uniformis | TSDC17.1-1.7 | | | | | |
| | | TSDC17.1-1.4 | | | | | | | |
| F60T2 | Sep. 22, 2008 | Bacteroides | Bacteroides | Bacteroides vulgatus | 8 | 15 | 5221044 | 102.0 | 73873 |
| | | vulgatus | vulgatus | TSDC17.1-1.1 | | | | | |
| | | TSDC17.1-1.1 | | | | | | | |
| F60T2 | Sep. 22, 2008 | Bacteroides | Bacteroides | Bacteroides vulgatus | 8 | 15 | 5227532 | 266.3 | 87924 |
| | | vulgatus | vulgatus | TSDC17.1-1.2 | | | | | |
| | | TSDC17.1-1.2 | | | | | | | |
| F60T2 | Sep. 22, 2008 | Bacteroides | Bacteroides | Bacteroides vulgatus | 8 | 15 | 5228085 | 147.7 | 73261 |
| | | vulgatus | vulgatus | TSDC17.1-1.5 | | | | | |
| | | TSDC17.1-1.3 | | | | | | | |
| F60T2 | Sep. 22, 2008 | Bacteroides | Bacteroides | Bacteroides vulgatus | 8 | 15 | 5301995 | 304.1 | 98326 |
| | | vulgatus | vulgatus | TSDC17.1-1.8 | | | | | |
| | | TSDC17.1-1.4 | | | | | | | |
| F60T2 | Sep. 22, 2008 | Bacteroides | Bacteroides | Bacteroides vulgatus | 30 | 15 | 5102801 | 18.6 | 1760 |
| | | vulgatus | vulgatus | TSDC17.1-1.3 | | | | | |
| | | TSDC17.1-2.1 | | | | | | | |
| F60T2 | Sep. 22, 2008 | Bifidobacterium | Bifidobacterium | Bifidobacterium | 2 | 17 | 2620091 | 159.6 | 236265 |
| | | adolescentis | adolescentis | adolescentis TSDC17.1- | | | | | |
| | | TSDC17.1-1.1 | | 1.5 | | | | | |
| F60T2 | Sep. 22, 2008 | Bifidobacterium | Bifidobacterium | Bifidobacterium | 2 | 17 | 2618354 | 117.2 | 73370 |
| | | adolescentis | adolescentis | TSDC17.1-1.1 | | | | | |
| | | TSDC17.1-1.10 | | | | | | | |
| F60T2 | Sep. 22, 2008 | Bifidobacterium | Bifidobacterium | Bifidobacterium | 2 | 17 | 2619815 | 127.9 | 127379 |
| | | adolescentis | adolescentis | TSDC17.1-1.4 | | | | | |
| | | TSDC17.1-1.11 | | | | | | | |
| F60T2 | Sep. 22, 2008 | Bifidobacterium | Bifidobacterium | Bifidobacterium | 2 | 17 | 2629582 | 188.3 | 142362 |
| | | adolescentis | adolescentis | TSDC17.1-1.6 | | | | | |
| | | TSDC17.1-1.12 | | | | | | | |
| F60T2 | Sep. 22, 2008 | Bifidobacterium | Bifidobacterium | Bifidobacterium | 2 | 17 | 2617185 | 159.9 | 85649 |
| | | adolescentis | adolescentis | TSDC17.1-1.8 | | | | | |
| | | TSDC17.1-1.13 | | | | | | | |
| F60T2 | Sep. 22, 2008 | Bifidobacterium | Bifidobacterium | Bifidobacterium | 2 | 17 | 2617375 | 188.4 | 127218 |
| | | adolescentis | adolescentis | adolescentis TSDC17.1- | | | | | |
| | | TSDC17.1-1.2 | | 1.8 | | | | | |
| F60T2 | Sep. 22, 2008 | Bifidobacterium | Bifidobacterium | Bifidobacterium | 2 | 17 | 2619242 | 68.6 | 180886 |
| | | adolescentis | adolescentis | adolescentis TSDC17.1- | | | | | |
| | | TSDC17.1-1.3 | | 1.11 | | | | | |
| F60T2 | Sep. 22, 2008 | Bifidobacterium | Bifidobacterium | Bifidobacterium | 2 | 17 | 2619708 | 154.9 | 87587 |
| | | adolescentis | adolescentis | adolescentis TSDC17.1- | | | | | |
| | | TSDC17.1-1.4 | | 1.13 | | | | | |
| F60T2 | Sep. 22, 2008 | Bifidobacterium | Bifidobacterium | Bifidobacterium | 2 | 17 | 2618943 | 72.5 | 180768 |
| | | adolescentis | adolescentis | adolescentis TSDC17.1- | | | | | |
| | | TSDC17.1-1.5 | | 2.1 | | | | | |
| F60T2 | Sep. 22, 2008 | Bifidobacterium | Bifidobacterium | Bifidobacterium | 2 | 17 | 2617631 | 133.4 | 133818 |
| | | adolescentis | adolescentis | adolescentis TSDC17.1- | | | | | |
| | | TSDC17.1-1.6 | | 2.2 | | | | | |
| F60T2 | Sep. 22, 2008 | Bifidobacterium | Bifidobacterium | Bifidobacterium | 2 | 17 | 2620949 | 244.8 | 180802 |
| | | adolescentis | adolescentis | adolescentis TSDC17.1- | | | | | |
| | | TSDC17.1-1.7 | | 2.4 | | | | | |
| F60T2 | Sep. 22, 2008 | Bifidobacterium | Bifidobacterium | Bifidobacterium | 2 | 17 | 2616442 | 156.1 | 134076 |
| | | adolescentis | adolescentis | adolescentis TSDC17.1- | | | | | |
| | | TSDC17.1-1.8 | | 2.6 | | | | | |
| F60T2 | Sep. 22, 2008 | Bifidobacterium | Bifidobacterium | Bifidobacterium | 2 | 17 | 2634745 | 18.8 | 15167 |
| | | adolescentis | adolescentis | adolescentis TSDC17.1- | | | | | |
| | | TSDC17.1-1.9 | | 2.15 | | | | | |
| F60T2 | Sep. 22, 2008 | Bifidobacterium | Bifidobacterium | Bifidobacterium longum | 19 | 19 | 2425493 | 121.2 | 71097 |
| | | longum | longum | TSDC17.1-1.4 | | | | | |
| | | TSDC17.1-1.1 | | | | | | | |
| F60T2 | Sep. 22, 2008 | Bifidobacterium | Bifidobacterium | Bifidobacterium longum | 19 | 19 | 2413685 | 106.2 | 124423 |
| | | longum | longum | TSDC17.1-1.7 | | | | | |
| | | TSDC17.1-1.2 | | | | | | | |
| F60T2 | Sep. 22, 2008 | Blautia schinkii | Blautia schinkii | Clostridiales TSDC17.1- | 40 | 22 | 3567921 | 252.7 | 104102 |
| | | TSDC17.1-1.1 | | 1.1 | | | | | |
| F60T2 | Sep. 22, 2008 | Butyricimonas | Butyricimonas | Butyricimonas virosa | 43 | 23 | 5636395 | 128.8 | 193917 |
| | | virosa TSDC17.1- | virosa | TSDC17.1-1.1 | | | | | |
| | | 1.1 | | | | | | | |
| F60T2 | Sep. 22, 2008 | Collinsella | Collinsella | Collinsella aerofaciens | 9 | 39 | 2252712 | 255.4 | 75503 |
| | | aerofaciens | aerofaciens | TSDC17.1-1.14 | | | | | |
| | | TSDC17.1-1.1 | | | | | | | |
| F60T2 | Sep. 22, 2008 | Collinsella | Collinsella | Collinsella aerofaciens | 9 | 39 | 2241617 | 102.1 | 17260 |
| | | aerofaciens | aerofaciens | TSDC17.1-1.18 | | | | | |
| | | TSDC17.1-1.2 | | | | | | | |
| F60T2 | Sep. 22, 2008 | Collinsella | Collinsella | Collinsella aerofaciens | 9 | 39 | 2248131 | 175.0 | 62859 |
| | | aerofaciens | aerofaciens | TSDC17.1-1.4 | | | | | |
| | | TSDC17.1-1.3 | | | | | | | |
| F60T2 | Sep. 22, 2008 | Collinsella | Collinsella | Collinsella aerofaciens | 9 | 39 | 2245742 | 99.3 | 50614 |
| | | aerofaciens | aerofaciens | TSDC17.1-1.8 | | | | | |
| | | TSDC17.1-1.4 | | | | | | | |
| F60T2 | Sep. 22, 2008 | Collinsella | Collinsella | Collinsella aerofaciens | 9 | 39 | 2246413 | 115.7 | 58320 |
| | | aerofaciens | aerofaciens | TSDC17.1-1.9 | | | | | |
| | | TSDC17.1-1.5 | | | | | | | |
| F60T2 | Sep. 22, 2008 | Collinsella | Collinsella | Collinsella aerofaciens | 9 | 39 | 2246518 | 126.7 | 41109 |
| | | aerofaciens | aerofaciens | TSDC17.1-3.1 | | | | | |
| | | TSDC17.1-1.6 | | | | | | | |
| F60T2 | Sep. 22, 2008 | Collinsella | Collinsella | Collinsella aerofaciens | 6 | 39 | 2226964 | 136.5 | 53122 |
| | | aerofaciens | aerofaciens | TSDC17.1-2.3 | | | | | |
| | | TSDC17.1-2.1 | | | | | | | |
| F60T2 | Sep. 22, 2008 | Collinsella | Collinsella | Collinsella aerofaciens | 6 | 39 | 2210884 | 152.1 | 64406 |
| | | aerofaciens | aerofaciens | TSDC17.1-2.5 | | | | | |
| | | TSDC17.1-2.2 | | | | | | | |
| F60T2 | Sep. 22, 2008 | Collinsella | Collinsella | Collinsella aerofaciens | 6 | 39 | 2230008 | 416.7 | 45012 |
| | | aerofaciens | aerofaciens | TSDC17.1-2.13 | | | | | |
| | | TSDC17.1-2.3 | | | | | | | |
| F60T2 | Sep. 22, 2008 | Collinsella | Collinsella | Collinsella aerofaciens | 6 | 39 | 2227499 | 163.3 | 64459 |
| | | aerofaciens | aerofaciens | TSDC17.1-2.15 | | | | | |
| | | TSDC17.1-2.4 | | | | | | | |
| F60T2 | Sep. 22, 2008 | Collinsella | Collinsella | Collinsella aerofaciens | 6 | 39 | 2227282 | 88.7 | 30029 |
| | | aerofaciens | aerofaciens | TSDC17.1-3.19 | | | | | |
| | | TSDC17.1-2.5 | | | | | | | |
| F60T2 | Sep. 22, 2008 | Coprococcus | Coprococcus | Coprococcus comes | 20 | 40 | 3363028 | 55.8 | 15569 |
| | | comes TSDC17.1- | comes | TSDC17.1-1.1 | | | | | |
| | | 1.1 | | | | | | | |
| F60T2 | Sep. 22, 2008 | Coprococcus | Coprococcus | Coprococcus comes | 20 | 40 | 3348715 | 671.7 | 90376 |
| | | comes TSDC17.1- | comes | TSDC17.1-1.2 | | | | | |
| | | 1.2 | | | | | | | |
| F60T2 | Sep. 22, 2008 | Coprococcus | Coprococcus | Coprococcus comes | 20 | 40 | 3437491 | 423.5 | 97617 |
| | | comes TSDC17.1- | comes | TSDC17.1-1.3 | | | | | |
| | | 1.3 | | | | | | | |
| F60T2 | Sep. 22, 2008 | Coprococcus | Coprococcus | Coprococcus comes | 20 | 40 | 3373516 | 390.9 | 91196 |
| | | comes TSDC17.1- | comes | TSDC17.1-1.5 | | | | | |
| | | 1.4 | | | | | | | |
| F60T2 | Sep. 22, 2008 | Dorea | Dorea | Dorea formicigenerans | 23 | 41 | 3374824 | 94.5 | 68605 |
| | | formicigenerans | formicigenerans | TSDC17.1-2.1 | | | | | |
| | | TSDC17.1-1.1 | | | | | | | |
| F60T2 | Sep. 22, 2008 | Dorea | Dorea | Dorea formicigenerans | 23 | 41 | 3396555 | 55.6 | 45025 |
| | | formicigenerans | formicigenerans | TSDC17.1-2.2 | | | | | |
| | | TSDC17.1-1.2 | | | | | | | |
| F60T2 | Sep. 22, 2008 | Dorea | Dorea | Dorea formicigenerans | 23 | 41 | 3390151 | 77.9 | 104011 |
| | | formicigenerans | formicigenerans | TSDC17.1-2.7 | | | | | |
| | | TSDC17.1-1.3 | | | | | | | |
| F60T2 | Sep. 22, 2008 | Dorea | Dorea | Dorea formicigenerans | 28 | 41 | 3390671 | 32.9 | 10285 |
| | | formicigenerans | formicigenerans | TSDC17.1-2.4 | | | | | |
| | | TSDC17.1-2.1 | | | | | | | |
| F60T2 | Sep. 22, 2008 | Dorea longicatena | Dorea longicatena | Dorea longicatena | 26 | 42 | 3120714 | 193.5 | 60964 |
| | | TSDC17.1-1.1 | | TSDC17.1-2.2 | | | | | |
| F60T2 | Sep. 22, 2008 | Dorea longicatena | Dorea longicatena | Dorea longicatena | 26 | 42 | 3125479 | 92.7 | 75391 |
| | | TSDC17.1-1.2 | | TSDC17.1-2.3 | | | | | |
| F60T2 | Sep. 22, 2008 | Dorea longicatena | Dorea longicatena | Dorea longicatena | 26 | 42 | 3076066 | 135.3 | 119946 |
| | | TSDC17.1-1.3 | | TSDC17.1-2.4 | | | | | |
| F60T2 | Sep. 22, 2008 | Dorea longicatena | Dorea longicatena | Dorea longicatena | 26 | 42 | 3096817 | 258.0 | 119360 |
| | | TSDC17.1-1.4 | | TSDC17.1-2.5 | | | | | |
| F60T2 | Sep. 22, 2008 | Escherichia coli | Escherichia coli | Escherichia coli | 14 | 45 | 5097609 | 54.4 | 124139 |
| | | TSDC17.1-1.1 | | TSDC17.1-1.1 | | | | | |
| F60T2 | Sep. 22, 2008 | Escherichia coli | Escherichia coli | Escherichia coli | 14 | 45 | 5104601 | 96.7 | 158946 |
| | | TSDC17.1-1.2 | | TSDC17.1-1.2 | | | | | |
| F60T2 | Sep. 22, 2008 | Eubacterium | Eubacterium | Eubacterium callanderi | 45 | 47 | 4566125 | 81.7 | 84097 |
| | | callanderi | callanderi | TSDC17.1-1.1 | | | | | |
| | | TSDC17.1-1.1 | | | | | | | |
| F60T2 | Sep. 22, 2008 | Odoribacter | Odoribacter | Odoribacter | 11 | 54 | 4527752 | 98.5 | 79785 |
| | | splanchnicus | splanchnicus | splanchnicus TSDC17.1- | | | | | |
| | | TSDC17.1-1.1 | | 1.1 | | | | | |
| F60T2 | Sep. 22, 2008 | Peptoniphilus | Peptoniphilus harei | Peptoniphilus | 37 | 58 | 2064672 | 47.7 | 45464 |
| | | harei TSDC17.1- | | TSDC17.1-1.1 | | | | | |
| | | 1.1 | | | | | | | |
| F60T2 | Sep. 22, 2008 | Peptoniphilus | Peptoniphilus harei | Peptoniphilus | 15 | 58 | 1973446 | 76.5 | 79361 |
| | | harei TSDC17.1- | | TSDC17.1-1.2 | | | | | |
| | | 2.1 | | | | | | | |
| F60T2 | Sep. 22, 2008 | Ruminococcus | Ruminococcus | Lachnospiraceae | 39 | 61 | 3605018 | 154.7 | 102050 |
| | | TSDC17.1-1.1 | | TSDC17.1-1.1 | | | | | |
| F60T2 | Sep. 22, 2008 | Subdoligranulum | Subdoligranulum | Clostridiaceae | 36 | 74 | 3765418 | 107.2 | 39126 |
| | | variabile | variabile | TSDC17.1-1.1 | | | | | |
| | | TSDC17.1-1.1 | | | | | | | |
| F60T2 | Nov. 10, 2008 | Anaerococcus | Anaerococcus | Anaerococcus | 41 | 2 | 2022280 | 111.9 | 50061 |
| | | TSDC17.2-1.1 | vaginalis | TSDC17.2-1.1 | | | | | |
| F60T2 | Nov. 10, 2008 | Bacteroides | Bacteroides caccae | Bacteroides caccae | 16 | 6 | 5618258 | 89.8 | 136635 |
| | | caccae | | TSDC17.2-1.1 | | | | | |
| | | TSDC17.2-1.1 | | | | | | | |
| F60T2 | Nov. 10, 2008 | Bacteroides | Bacteroides caccae | Bacteroides caccae | 16 | 6 | 5621958 | 177.9 | 150372 |
| | | caccae | | TSDC17.2-1.3 | | | | | |
| | | TSDC17.2-1.2 | | | | | | | |
| F60T2 | Nov. 10, 2008 | Bacteroides | Bacteroides caccae | Bacteroides caccae | 16 | 6 | 5630714 | 36.5 | 53526 |
| | | caccae | | TSDC17.2-1.6 | | | | | |
| | | TSDC17.2-1.3 | | | | | | | |
| F60T2 | Nov. 10, 2008 | Bacteroides | Bacteroides caccae | Bacteroides caccae | 16 | 6 | 5638793 | 55.3 | 100002 |
| | | caccae | | TSDC17.2-1.7 | | | | | |
| | | TSDC17.2-1.4 | | | | | | | |
| F60T2 | Nov. 10, 2008 | Bacteroides | Bacteroides | Bacteroides finegoldii | 18 | 7 | 4500782 | 167.0 | 95202 |
| | | finegoldii | finegoldii | TSDC17.2-1.2 | | | | | |
| | | TSDC17.2-1.1 | | | | | | | |
| F60T2 | Nov. 10, 2008 | Bacteroides | Bacteroides | Bacteroides finegoldii | 18 | 7 | 4529914 | 44.3 | 40463 |
| | | finegoldii | finegoldii | TSDC17.2-1.4 | | | | | |
| | | TSDC17.2-1.2 | | | | | | | |
| F60T2 | Nov. 10, 2008 | Bacteroides | Bacteroides | Bacteroides intestinalis | 7 | 9 | 7361472 | 205.9 | 222845 |
| | | intestinalis | intestinalis | TSDC17.2-1.5 | | | | | |
| | | TSDC17.2-1.1 | | | | | | | |
| F60T2 | Nov. 10, 2008 | Bacteroides | Bacteroides | Bacteroides intestinalis | 7 | 9 | 7388481 | 36.4 | 41734 |
| | | intestinalis | intestinalis | TSDC17.2-1.7 | | | | | |
| | | TSDC17.2-1.2 | | | | | | | |
| F60T2 | Nov. 10, 2008 | Bacteroides | Bacteroides | Bacteroides intestinalis | 7 | 9 | 7360958 | 134.1 | 171812 |
| | | intestinalis | intestinalis | TSDC17.2-1.9 | | | | | |
| | | TSDC17.2-1.3 | | | | | | | |
| F60T2 | Nov. 10, 2008 | Bacteroides | Bacteroides | Bacteroides massiliensis | 22 | 10 | 4558331 | 81.7 | 68684 |
| | | massiliensis | massiliensis | TSDC17.2-1.2 | | | | | |
| | | TSDC17.2-1.1 | | | | | | | |
| F60T2 | Nov. 10, 2008 | Bacteroides | Bacteroides | Bacteroides massiliensis | 22 | 10 | 4564604 | 124.5 | 68634 |
| | | massiliensis | massiliensis | TSDC17.2-1.3 | | | | | |
| | | TSDC17.2-1.2 | | | | | | | |
| F60T2 | Nov. 10, 2008 | Bacteroides | Bacteroides | Bacteroides ovatus | 1 | 11 | 6851204 | 263.4 | 151301 |
| | | ovatus TSDC17.2- | ovatus | TSDC17.2-2.2 | | | | | |
| | | 1.1 | | | | | | | |
| F60T2 | Nov. 10, 2008 | Bacteroides | Bacteroides | Bacteroides ovatus | 1 | 11 | 6841173 | 186.5 | 164497 |
| | | ovatus TSDC17.2- | ovatus | TSDC17.2-3.1 | | | | | |
| | | 1.2 | | | | | | | |
| F60T2 | Nov. 10, 2008 | Bacteroides | Bacteroides | Bacteroides | 25 | 13 | 6382599 | 81.3 | 80865 |
| | | thetaiotaomicron | thetaiotaomicron | thetaiotaomicron | | | | | |
| | | TSDC17.2-1.1 | | TSDC17.2-1.3 | | | | | |
| F60T2 | Nov. 10, 2008 | Bacteroides | Bacteroides | Bacteroides | 25 | 13 | 6448600 | 205.8 | 150422 |
| | | thetaiotaomicron | thetaiotaomicron | thetaiotaomicron | | | | | |
| | | TSDC17.2-1.2 | | TSDC17.2-3.1 | | | | | |
| F60T2 | Nov. 10, 2008 | Bacteroides | Bacteroides | Bacteroides | 4 | 13 | 7054537 | 260.8 | 166660 |
| | | thetaiotaomicron | thetaiotaomicron | thetaiotaomicron | | | | | |
| | | TSDC17.2-2.1 | | TSDC17.2-2.4 | | | | | |
| F60T2 | Nov. 10, 2008 | Bacteroides | Bacteroides | Bacteroides | 4 | 13 | 7059190 | 175.6 | 132204 |
| | | thetaiotaomicron | thetaiotaomicron | thetaiotaomicron | | | | | |
| | | TSDC17.2-2.2 | | TSDC17.2-2.5 | | | | | |
| F60T2 | Nov. 10, 2008 | Bacteroides | Bacteroides | Bacteroides acidifaciens | 5 | 14 | 5028122 | 289.0 | 105031 |
| | | uniformis | uniformis | TSDC17.2-1.3 | | | | | |
| | | TSDC17.2-1.1 | | | | | | | |
| F60T2 | Nov. 10, 2008 | Bacteroides | Bacteroides | Bacteroides acidifaciens | 5 | 14 | 5021119 | 86.9 | 110234 |
| | | uniformis | uniformis | TSDC17.2-1.8 | | | | | |
| | | TSDC17.2-1.2 | | | | | | | |
| F60T2 | Nov. 10, 2008 | Bacteroides | Bacteroides | Bacteroides vulgatus | 31 | 15 | 5258550 | 75.4 | 93954 |
| | | vulgatus | vulgatus | TSDC17.2-1.11 | | | | | |
| | | TSDC17.2-1.1 | | | | | | | |
| F60T2 | Nov. 10, 2008 | Bacteroides | Bacteroides | Bacteroides vulgatus | 8 | 15 | 5224985 | 127.0 | 93644 |
| | | vulgatus | vulgatus | TSDC17.2-1.5 | | | | | |
| | | TSDC17.2-2.1 | | | | | | | |
| F60T2 | Nov. 10, 2008 | Bacteroides | Bacteroides | Bacteroides vulgatus | 34 | 15 | 5247453 | 112.6 | 68507 |
| | | vulgatus | vulgatus | TSDC17.2-2.12 | | | | | |
| | | TSDC17.2-3.1 | | | | | | | |
| F60T2 | Nov. 10, 2008 | Bifidobacterium | Bifidobacterium | Bifidobacterium | 38 | 20 | 2384331 | 74.8 | 68758 |
| | | pseudocatenulatum | pseudocatenulatum | pseudocatenulatum | | | | | |
| | | TSDC17.2-1.1 | | TSDC17.2-1.5 | | | | | |
| F60T2 | Nov. 10, 2008 | Clostridium | Clostridium leptum | Ruminococcaceae | 33 | 35 | 3376043 | 53.5 | 95726 |
| | | leptum | | TSDC17.2-3.1 | | | | | |
| | | TSDC17.2-1.1 | | | | | | | |
| F60T2 | Nov. 10, 2008 | Clostridium | Clostridium leptum | Ruminococcaceae | 32 | 35 | 3513280 | 43.5 | 93384 |
| | | leptum | | TSDC17.2-3.2 | | | | | |
| | | TSDC17.2-2.1 | | | | | | | |
| F60T2 | Nov. 10, 2008 | Clostridium | Clostridium | Clostridium scindens | 42 | 36 | 3632357 | 67.4 | 95081 |
| | | scindens | scindens | TSDC17.2-1.1 | | | | | |
| | | TSDC17.2-1.1 | | | | | | | |
| F60T2 | Nov. 10, 2008 | Collinsella | Collinsella | Collinsella aerofaciens | 6 | 39 | 2209479 | 138.4 | 61462 |
| | | aerofaciens | aerofaciens | TSDC17.2-1.9 | | | | | |
| | | TSDC17.2-1.1 | | | | | | | |
| F60T2 | Nov. 10, 2008 | Collinsella | Collinsella | Collinsella aerofaciens | 6 | 39 | 2207148 | 134.9 | 62105 |
| | | aerofaciens | aerofaciens | TSDC17.2-1.10 | | | | | |
| | | TSDC17.2-1.2 | | | | | | | |
| F60T2 | Nov. 10, 2008 | Collinsella | Collinsella | Collinsella aerofaciens | 6 | 39 | 2211497 | 109.4 | 58421 |
| | | aerofaciens | aerofaciens | TSDC17.2-3.20 | | | | | |
| | | TSDC17.2-1.3 | | | | | | | |
| F60T2 | Nov. 10, 2008 | Collinsella | Collinsella | Collinsella aerofaciens | 6 | 39 | 2230977 | 206.5 | 64405 |
| | | aerofaciens | aerofaciens | TSDC17.2-3.23 | | | | | |
| | | TSDC17.2-1.4 | | | | | | | |
| F60T2 | Nov. 10, 2008 | Collinsella | Collinsella | Collinsella aerofaciens | 6 | 39 | 2209280 | 142.5 | 63400 |
| | | aerofaciens | aerofaciens | TSDC17.2-4.22 | | | | | |
| | | TSDC17.2-1.5 | | | | | | | |
| F60T2 | Nov. 10, 2008 | Collinsella | Collinsella | Collinsella aerofaciens | 9 | 39 | 2246595 | 163.0 | 58238 |
| | | aerofaciens | aerofaciens | TSDC17.2-2.24 | | | | | |
| | | TSDC17.2-2.1 | | | | | | | |
| F60T2 | Nov. 10, 2008 | Coprococcus | Coprococcus | Coprococcus comes | 20 | 40 | 3483630 | 47.8 | 51005 |
| | | comes TSDC17.2- | comes | TSDC17.2-1.1 | | | | | |
| | | 1.1 | | | | | | | |
| F60T2 | Nov. 10, 2008 | Coprococcus | Coprococcus | Coprococcus comes | 20 | 40 | 3435355 | 185.1 | 97811 |
| | | comes TSDC17.2- | comes | TSDC17.2-1.2 | | | | | |
| | | 1.2 | | | | | | | |
| F60T2 | Nov. 10, 2008 | Dorea longicatena | Dorea longicatena | Dorea TSDC17.2-1.1 | 26 | 42 | 3112423 | 174.2 | 103776 |
| | | TSDC17.2-1.1 | | | | | | | |
| F60T2 | Nov. 10, 2008 | Dorea longicatena | Dorea longicatena | Clostridiaceae | 26 | 42 | 3105898 | 126.3 | 112604 |
| | | TSDC17.2-1.2 | | TSDC17.2-3.1 | | | | | |
| F60T2 | Nov. 10, 2008 | Escherichia coli | Escherichia coli | Escherichia coli | 14 | 45 | 5161398 | 52.9 | 29397 |
| | | TSDC17.2-1.1 | | TSDC17.2-1.1 | | | | | |
| F60T2 | Nov. 10, 2008 | Escherichia coli | Escherichia coli | Escherichia coli | 14 | 45 | 5151719 | 135.0 | 124205 |
| | | TSDC17.2-1.2 | | TSDC17.2-1.2 | | | | | |
| F60T2 | Nov. 10, 2008 | Odoribacter | Odoribacter | Odoribacter | 11 | 54 | 4524727 | 93.4 | 87399 |
| | | splanchnicus | splanchnicus | splanchnicus TSDC17.2- | | | | | |
| | | TSDC17.2-1.1 | | 1.1 | | | | | |
| F60T2 | Nov. 10, 2008 | Odoribacter | Odoribacter | Odoribacter | 11 | 54 | 4528238 | 155.5 | 93712 |
| | | splanchnicus | splanchnicus | splanchnicus TSDC17.2- | | | | | |
| | | TSDC17.2-1.2 | | 1.2 | | | | | |
| F60T2 | Nov. 10, 2008 | Parabacteroides | Parabacteroides | Parabacteroides | 44 | 55 | 7040869 | 56.9 | 4032 |
| | | distasonis | distasonis | distasonis TSDC17.2- | | | | | |
| | | TSDC17.2-1.1 | | 1.2 | | | | | |
| F60T2 | Nov. 10, 2008 | Peptoniphilus | Peptoniphilus harei | Peptoniphilus | 15 | 58 | 1956141 | 212.0 | 73569 |
| | | harei TSDC17.2- | | TSDC17.2-1.1 | | | | | |
| | | 1.1 | | | | | | | |
| F60T2 | Nov. 10, 2008 | Ruminococcaceae | Ruminococcaceae | Ruminococcaceae | 24 | 60 | 2794122 | 164.4 | 31657 |
| | | TSDC17.2-1.1 | | TSDC17.2-2.1 | | | | | |
| F60T2 | Nov. 10, 2008 | Ruminococcaceae | Ruminococcaceae | Clostridiaceae | 24 | 60 | 2798559 | 48.4 | 30269 |
| | | TSDC17.2-1.2 | | TSDC17.2-2.4 | | | | | |
| F60T2 | Nov. 10, 2008 | Ruminococcus | Ruminococcus | Ruminococcus albus | 17 | 62 | 2931186 | 70.1 | 42581 |
| | | albus TSDC17.2- | albus | TSDC17.2-1.6 | | | | | |
| | | 1.1 | | | | | | | |
| F60T2 | Nov. 10, 2008 | Ruminococcus | Ruminococcus | Ruminococcus albus | 17 | 62 | 2932691 | 159.7 | 37013 |
| | | albus TSDC17.2- | albus | TSDC17.2-1.7 | | | | | |
| | | 1.2 | | | | | | | |
| F60T2 | Nov. 10, 2008 | Ruminococcus | Ruminococcus | Ruminococcus albus | 17 | 62 | 2941538 | 34.5 | 30665 |
| | | albus TSDC17.2- | albus | TSDC17.2-1.16 | | | | | |
| | | 1.3 | | | | | | | |
| F60T2 | Nov. 10, 2008 | Ruminococcus | Ruminococcus | Ruminococcus albus | 17 | 62 | 2950451 | 38.8 | 41990 |
| | | albus TSDC17.2- | albus | TSDC17.2-2.8 | | | | | |
| | | 1.4 | | | | | | | |
| F60T2 | Nov. 10, 2008 | Ruminococcus | Ruminococcus | Ruminococcus bromii | 12 | 63 | 2350848 | 37.1 | 95461 |
| | | bromii TSDC17.2- | bromii | TSDC17.2-1.7 | | | | | |
| | | 1.1 | | | | | | | |
| F60T2 | Nov. 10, 2008 | Ruminococcus | Ruminococcus | Ruminococcus bromii | 12 | 63 | 2350733 | 69.7 | 130729 |
| | | bromii TSDC17.2- | bromii | TSDC17.2-2.2 | | | | | |
| | | 1.2 | | | | | | | |
| F60T2 | Nov. 10, 2008 | Ruminococcus | Ruminococcus | Ruminococcus bromii | 12 | 63 | 2349863 | 54.6 | 84494 |
| | | bromii TSDC17.2- | bromii | TSDC17.2-2.5 | | | | | |
| | | 1.3 | | | | | | | |
| F60T2 | Nov. 10, 2008 | Subdoligranulum | Subdoligranulum | Ruminococcaceae | 35 | 74 | 3857892 | 110.9 | 44401 |
| | | variabile | variabile | TSDC17.2-1.1 | | | | | |
| | | TSDC17.2-1.1 | | | | | | | |
| F61T1 | Oct. 15, 2008 | Alistipes | Alistipes | Alistipes indistinctus | 31 | 1 | 3241282 | 16.1 | 130636 |
| | | indistinctus | indistinctus | TSDC19.1-1.1 | | | | | |
| | | TSDC19.1-1.1 | | | | | | | |
| F61T1 | Oct. 15, 2008 | Anaerofustis | Anaerofustis | Anaerofustis | 43 | 4 | 2354462 | 64.3 | 32954 |
| | | stercorihominis | stercorihominis | stercorihominis | | | | | |
| | | TSDC19.1-1.1 | | TSDC19.1-1.1 | | | | | |
| F61T1 | Oct. 15, 2008 | Bacteroides | Bacteroides | Bacteroides finegoldii | 18 | 7 | 5198005 | 7.7 | 5180 |
| | | finegoldii | finegoldii | TSDC19.1-1.5 | | | | | |
| | | TSDC19.1-1.1 | | | | | | | |
| F61T1 | Oct. 15, 2008 | Bacteroides | Bacteroides fragilis | Bacteroides fragilis | 11 | 8 | 5386949 | 65.6 | 103741 |
| | | fragilis TSDC19.1- | | TSDC19.1-1.3 | | | | | |
| | | 1.1 | | | | | | | |
| F61T1 | Oct. 15, 2008 | Bacteroides | Bacteroides fragilis | Bacteroides fragilis | 11 | 8 | 5389013 | 70.4 | 105979 |
| | | fragilis TSDC19.1- | | TSDC19.1-1.4 | | | | | |
| | | 1.2 | | | | | | | |
| F61T1 | Oct. 15, 2008 | Bacteroides | Bacteroides | Bacteroides ovatus | 17 | 11 | 6781579 | 14.8 | 29818 |
| | | ovatus TSDC19.1- | ovatus | TSDC19.1-1.8 | | | | | |
| | | 1.1 | | | | | | | |
| F61T1 | Oct. 15, 2008 | Bacteroides | Bacteroides | Bacteroides | 19 | 13 | 6558732 | 13.3 | 31839 |
| | | thetaiotaomicron | thetaiotaomicron | thetaiotaomicron | | | | | |
| | | TSDC19.1-1.1 | | TSDC19.1-2.6 | | | | | |
| F61T1 | Oct. 15, 2008 | Bacteroides | Bacteroides | Bacteroides uniformis | 23 | 14 | 5227025 | 140.2 | 228839 |
| | | uniformis | uniformis | TSDC19.1-1.2 | | | | | |
| | | TSDC19.1-1.1 | | | | | | | |
| F61T1 | Oct. 15, 2008 | Bacteroides | Bacteroides | Bacteroides vulgatus | 14 | 15 | 5216830 | 29.5 | 70400 |
| | | vulgatus | vulgatus | TSDC19.1-1.1 | | | | | |
| | | TSDC19.1-1.1 | | | | | | | |
| F61T1 | Oct. 15, 2008 | Bacteroides | Bacteroides | Bacteroides vulgatus | 14 | 15 | 5176903 | 42.4 | 76715 |
| | | vulgatus | vulgatus | TSDC19.1-1.3 | | | | | |
| | | TSDC19.1-1.2 | | | | | | | |
| F61T1 | Oct. 15, 2008 | Bifidobacterium | Bifidobacterium | Bifidobacterium bifidum | 15 | 17 | 2082905 | 431.2 | 167193 |
| | | adolescentis | adolescentis | TSDC19.1-1.3 | | | | | |
| | | TSDC19.1-1.1 | | | | | | | |
| F61T1 | Oct. 15, 2008 | Bifidobacterium | Bifidobacterium | Bifidobacterium | 15 | 17 | 2079535 | 246.9 | 475281 |
| | | adolescentis | adolescentis | TSDC19.1-1.6 | | | | | |
| | | TSDC19.1-1.2 | | | | | | | |
| F61T1 | Oct. 15, 2008 | Bifidobacterium | Bifidobacterium | Bifidobacterium bifidum | 25 | 18 | 2231191 | 130.4 | 135528 |
| | | bifidum | bifidum | TSDC19.1-2.4 | | | | | |
| | | TSDC19.1-1.1 | | | | | | | |
| F61T1 | Oct. 15, 2008 | Bifidobacterium | Bifidobacterium | Bifidobacterium | 8 | 20 | 2046655 | 148.7 | 344537 |
| | | pseudocatenulatum | pseudocatenulatum | TSDC19.1-2.3 | | | | | |
| | | TSDC19.1-1.1 | | | | | | | |
| F61T1 | Oct. 15, 2008 | Bifidobacterium | Bifidobacterium | Bifidobacterium | 8 | 20 | 1948405 | 210.4 | 331747 |
| | | pseudocatenulatum | pseudocatenulatum | TSDC19.1-2.8 | | | | | |
| | | TSDC19.1-1.2 | | | | | | | |
| F61T1 | Oct. 15, 2008 | Bifidobacterium | Bifidobacterium | Bifidobacterium | 8 | 20 | 2033515 | 186.6 | 344672 |
| | | pseudocatenulatum | pseudocatenulatum | TSDC19.1-4.7 | | | | | |
| | | TSDC19.1-1.3 | | | | | | | |
| F61T1 | Oct. 15, 2008 | Clostridium | Clostridium | Clostridium TSDC19.1- | 2 | 31 | 3813144 | 229.1 | 175451 |
| | | TSDC19.1-1.1 | | 1.4 | | | | | |
| F61T1 | Oct. 15, 2008 | Collinsella | Collinsella | Collinsella aerofaciens | 20 | 39 | 2245674 | 96.9 | 73213 |
| | | aerofaciens | aerofaciens | TSDC19.1-1.2 | | | | | |
| | | TSDC19.1-1.1 | | | | | | | |
| F61T1 | Oct. 15, 2008 | Escherichia coli | Escherichia coli | Escherichia coli | 4 | 45 | 5202190 | 68.4 | 155228 |
| | | TSDC19.1-1.1 | | TSDC19.1-1.1 | | | | | |
| F61T1 | Oct. 15, 2008 | Escherichia coli | Escherichia coli | Escherichia coli | 4 | 45 | 5200622 | 93.5 | 131476 |
| | | TSDC19.1-1.2 | | TSDC19.1-1.2 | | | | | |
| F61T1 | Oct. 15, 2008 | Odoribacter | Odoribacter | Odoribacter | 9 | 54 | 4734235 | 27.1 | 65595 |
| | | splanchnicus | splanchnicus | splanchnicus TSDC19.1- | | | | | |
| | | TSDC19.1-1.1 | | 1.1 | | | | | |
| F61T1 | Oct. 15, 2008 | Odoribacter | Odoribacter | Odoribacter | 9 | 54 | 4726829 | 35.6 | 74084 |
| | | splanchnicus | splanchnicus | splanchnicus TSDC19.1- | | | | | |
| | | TSDC19.1-1.2 | | 1.2 | | | | | |
| F61T1 | Oct. 15, 2008 | Odoribacter | Odoribacter | Odoribacter | 9 | 54 | 4730127 | 25.1 | 63335 |
| | | splanchnicus | splanchnicus | splanchnicus TSDC19.1- | | | | | |
| | | TSDC19.1-1.3 | | 1.3 | | | | | |
| F61T1 | Oct. 15, 2008 | Parabacteroides | Parabacteroides | Parabacteroides | 1 | 55 | 5163461 | 158.7 | 268367 |
| | | distasonis | distasonis | distasonis TSDC19.1- | | | | | |
| | | TSDC19.1-1.1 | | 1.5 | | | | | |
| F61T1 | Oct. 15, 2008 | Parabacteroides | Parabacteroides | Bacteroidales | 1 | 55 | 5153338 | 123.5 | 219288 |
| | | distasonis | distasonis | TSDC19.1-1.2 | | | | | |
| | | TSDC19.1-1.2 | | | | | | | |
| F61T1 | Oct. 15, 2008 | Parabacteroides | Parabacteroides | Parabacteroides | 1 | 55 | 5163434 | 92.1 | 236104 |
| | | distasonis | distasonis | distasonis TSDC19.1- | | | | | |
| | | TSDC19.1-1.3 | | 1.2 | | | | | |
| F61T1 | Oct. 15, 2008 | Parabacteroides | Parabacteroides | Parabacteroides | 21 | 56 | 6409331 | 5.4 | 774 |
| | | goldsteinii | goldsteinii | goldsteinii TSDC19.1- | | | | | |
| | | TSDC19.1-1.1 | | 1.3 | | | | | |
| F61T1 | Oct. 15, 2008 | Parabacteroides | Parabacteroides | Parabacteroides merdae | 5 | 57 | 4563362 | 49.9 | 124413 |
| | | merdae | merdae | TSDC19.1-1.2 | | | | | |
| | | TSDC19.1-1.1 | | | | | | | |
| F61T1 | Oct. 15, 2008 | Parabacteroides | Parabacteroides | Parabacteroides merdae | 5 | 57 | 4567248 | 52.9 | 134750 |
| | | merdae | merdae | TSDC19.1-1.3 | | | | | |
| | | TSDC19.1-1.2 | | | | | | | |
| F61T1 | Dec. 1, 2008 | Bacteroides | Bacteroides | Bacteroides finegoldii | 18 | 7 | 5124995 | 54.6 | 100213 |
| | | finegoldii | finegoldii | TSDC19.2-1.3 | | | | | |
| | | TSDC19.2-1.1 | | | | | | | |
| F61T1 | Dec. 1, 2008 | Bacteroides | Bacteroides | Bacteroides finegoldii | 18 | 7 | 5076996 | 21.4 | 45825 |
| | | finegoldii | finegoldii | TSDC19.2-2.2 | | | | | |
| | | TSDC19.2-1.2 | | | | | | | |
| F61T1 | Dec. 1, 2008 | Bacteroides | Bacteroides fragilis | Bacteroides fragilis | 11 | 8 | 5392747 | 51.5 | 114151 |
| | | fragilis TSDC19.2- | | TSDC19.2-1.2 | | | | | |
| | | 1.1 | | | | | | | |
| F61T1 | Dec. 1, 2008 | Bacteroides | Bacteroides | Bacteroides ovatus | 7 | 11 | 7365260 | 29.2 | 92341 |
| | | ovatus TSDC19.2- | ovatus | TSDC19.2-2.1 | | | | | |
| | | 1.1 | | | | | | | |
| F61T1 | Dec. 1, 2008 | Bacteroides | Bacteroides | Bacteroides ovatus | 7 | 11 | 7362078 | 50.7 | 100015 |
| | | ovatus TSDC19.2- | ovatus | TSDC19.2-2.6 | | | | | |
| | | 1.2 | | | | | | | |
| F61T1 | Dec. 1, 2008 | Bacteroides | Bacteroides | Bacteroides ovatus | 17 | 11 | 6813732 | 62.7 | 94697 |
| | | ovatus TSDC19.2- | ovatus | TSDC19.2-4.5 | | | | | |
| | | 2.1 | | | | | | | |
| F61T1 | Dec. 1, 2008 | Bacteroides | Bacteroides | Bacteroides ovatus | 24 | 11 | 6409186 | 94.4 | 111979 |
| | | ovatus TSDC19.2- | ovatus | TSDC19.2-1.3 | | | | | |
| | | 3.1 | | | | | | | |
| F61T1 | Dec. 1, 2008 | Bacteroides | Bacteroides | Bacteroides | 12 | 13 | 7266149 | 41.1 | 131797 |
| | | thetaiotaomicron | thetaiotaomicron | thetaiotaomicron | | | | | |
| | | TSDC19.2-1.1 | | TSDC19.2-1.2 | | | | | |
| F61T1 | Dec. 1, 2008 | Bacteroides | Bacteroides | Bacteroides | 12 | 13 | 7316296 | 33.6 | 139837 |
| | | thetaiotaomicron | thetaiotaomicron | thetaiotaomicron | | | | | |
| | | TSDC19.2-1.2 | | TSDC19.2-2.4 | | | | | |
| F61T1 | Dec. 1, 2008 | Bacteroides | Bacteroides | Bacteroides | 19 | 13 | 6443939 | 128.0 | 144714 |
| | | thetaiotaomicron | thetaiotaomicron | thetaiotaomicron | | | | | |
| | | TSDC19.2-2.1 | | TSDC19.2-2.1 | | | | | |
| F61T1 | Dec. 1, 2008 | Bacteroides | Bacteroides | Bacteroides TSDC19.2- | 13 | 5 | 5822234 | 11.4 | 13771 |
| | | TSDC19.2-1.1 | | 1.11 | | | | | |
| F61T1 | Dec. 1, 2008 | Bacteroides | Bacteroides | Bacteroides TSDC19.2- | 13 | 5 | 5769924 | 148.9 | 117805 |
| | | TSDC19.2-1.2 | | 3.12 | | | | | |
| F61T1 | Dec. 1, 2008 | Bacteroides | Bacteroides | Bacteroides TSDC19.2- | 13 | 5 | 5785752 | 26.2 | 53542 |
| | | TSDC19.2-1.3 | | 3.3 | | | | | |
| F61T1 | Dec. 1, 2008 | Bacteroides | Bacteroides | Bacteroides TSDC19.2- | 13 | 5 | 5838255 | 15.9 | 39380 |
| | | TSDC19.2-1.4 | | 9.4 | | | | | |
| F61T1 | Dec. 1, 2008 | Bacteroides | Bacteroides | Bacteroides uniformis | 22 | 14 | 5144190 | 10.4 | 13598 |
| | | uniformis | uniformis | TSDC19.2-1.1 | | | | | |
| | | TSDC19.2-1.1 | | | | | | | |
| F61T1 | Dec. 1, 2008 | Bacteroides | Bacteroides | Bacteroides vulgatus | 14 | 15 | 5196766 | 55.9 | 84376 |
| | | vulgatus | vulgatus | TSDC19.2-1.2 | | | | | |
| | | TSDC19.2-1.1 | | | | | | | |
| F61T1 | Dec. 1, 2008 | Blautia schinkii | Blautia schinkii | Blautia schinkii | 33 | 22 | 3191770 | 157.0 | 124291 |
| | | TSDC19.2-1.1 | | TSDC19.2-1.1 | | | | | |
| F61T1 | Dec. 1, 2008 | Butyricimonas | Butyricimonas | Butyricimonas virosa | 34 | 23 | 4459643 | 242.7 | 198609 |
| | | virosa TSDC19.2- | virosa | TSDC19.2-1.1 | | | | | |
| | | 1.1 | | | | | | | |
| F61T1 | Dec. 1, 2008 | Clostridiales | Clostridiales | Clostridiales TSDC19.2- | 35 | 24 | 3582709 | 38.0 | 72182 |
| | | TSDC19.2-1.1 | | 2.7 | | | | | |
| F61T1 | Dec. 1, 2008 | Clostridiales | Clostridiales | Clostridiales TSDC19.2- | 37 | 25 | 4094554 | 34.2 | 58870 |
| | | TSDC19.2-2.1 | | 4.9 | | | | | |
| F61T1 | Dec. 1, 2008 | Clostridiales | Clostridiales | Clostridiales TSDC19.2- | 42 | 26 | 3232579 | 40.3 | 127003 |
| | | TSDC19.2-3.1 | | 5.1 | | | | | |
| F61T1 | Dec. 1, 2008 | Clostridiales | Clostridiales | Clostridiales TSDC19.2- | 27 | 27 | 2924363 | 177.6 | 175911 |
| | | TSDC19.2-4.1 | | 6.5 | | | | | |
| F61T1 | Dec. 1, 2008 | Clostridiales | Clostridiales | Clostridiales TSDC19.2- | 36 | 30 | 3993798 | 64.4 | 83848 |
| | | TSDC19.2-5.1 | | 7.8 | | | | | |
| F61T1 | Dec. 1, 2008 | Clostridium | Clostridium leptum | Clostridium leptum | 40 | 35 | 3329804 | 44.6 | 107791 |
| | | leptum | | TSDC19.2-1.1 | | | | | |
| | | TSDC19.2-1.1 | | | | | | | |
| F61T1 | Dec. 1, 2008 | Clostridium | Clostridium | Clostridium TSDC19.2- | 2 | 31 | 3819630 | 60.5 | 181180 |
| | | TSDC19.2-1.1 | | 1.1 | | | | | |
| F61T1 | Dec. 1, 2008 | Clostridium | Clostridium | Clostridium TSDC19.2- | 2 | 31 | 3810704 | 83.2 | 213738 |
| | | TSDC19.2-1.2 | | 1.3 | | | | | |
| F61T1 | Dec. 1, 2008 | Clostridium | Clostridium | Clostridium TSDC19.2- | 38 | 32 | 2569796 | 44.3 | 120126 |
| | | TSDC19.2-2.1 | | 2.2 | | | | | |
| F61T1 | Dec. 1, 2008 | Collinsella | Collinsella | Collinsella aerofaciens | 20 | 39 | 2271087 | 189.6 | 50324 |
| | | aerofaciens | aerofaciens | TSDC19.2-1.1 | | | | | |
| | | TSDC19.2-1.1 | | | | | | | |
| F61T1 | Dec. 1, 2008 | Dorea | Dorea | Dorea formicigenerans | 26 | 41 | 3371716 | 148.0 | 137778 |
| | | formicigenerans | formicigenerans | TSDC19.2-1.1 | | | | | |
| | | TSDC19.2-1.1 | | | | | | | |
| F61T1 | Dec. 1, 2008 | Eubacterium | Eubacterium | Eubacterium contortum | 41 | 48 | 5210527 | 67.2 | 83253 |
| | | contortum | contortum | TSDC19.2-1.1 | | | | | |
| | | TSDC19.2-1.1 | | | | | | | |
| F61T1 | Dec. 1, 2008 | Parabacteroides | Parabacteroides | Bacteroidales | 1 | 55 | 5242154 | 8.2 | 10428 |
| | | distasonis | distasonis | TSDC19.2-1.1 | | | | | |
| | | TSDC19.2-1.1 | | | | | | | |
| F61T1 | Dec. 1, 2008 | Parabacteroides | Parabacteroides | Parabacteroides | 1 | 55 | 5168089 | 144.0 | 273111 |
| | | distasonis | distasonis | distasonis TSDC19.2- | | | | | |
| | | TSDC19.2-1.10 | | 5.7 | | | | | |
| F61T1 | Dec. 1, 2008 | Parabacteroides | Parabacteroides | Bacteroides TSDC19.2- | 1 | 55 | 5156964 | 76.6 | 283925 |
| | | distasonis | distasonis | 6.7 | | | | | |
| | | TSDC19.2-1.11 | | | | | | | |
| F61T1 | Dec. 1, 2008 | Parabacteroides | Parabacteroides | Bacteroides TSDC19.2- | 1 | 55 | 5154790 | 109.8 | 222353 |
| | | distasonis | distasonis | 7.1 | | | | | |
| | | TSDC19.2-1.12 | | | | | | | |
| F61T1 | Dec. 1, 2008 | Parabacteroides | Parabacteroides | Bacteroides TSDC19.2- | 1 | 55 | 5169503 | 158.7 | 275974 |
| | | distasonis | distasonis | 8.10 | | | | | |
| | | TSDC19.2-1.13 | | | | | | | |
| F61T1 | Dec. 1, 2008 | Parabacteroides | Parabacteroides | Parabacteroides | 1 | 55 | 5159296 | 31.0 | 186724 |
| | | distasonis | distasonis | distasonis TSDC19.2- | | | | | |
| | | TSDC19.2-1.2 | | 1.1 | | | | | |
| F61T1 | Dec. 1, 2008 | Parabacteroides | Parabacteroides | Bacteroides caccae | 1 | 55 | 5157341 | 113.2 | 214280 |
| | | distasonis | distasonis | TSDC19.2-1.3 | | | | | |
| | | TSDC19.2-1.3 | | | | | | | |
| F61T1 | Dec. 1, 2008 | Parabacteroides | Parabacteroides | Parabacteroides | 1 | 55 | 5160908 | 36.7 | 198164 |
| | | distasonis | distasonis | distasonis TSDC19.2- | | | | | |
| | | TSDC19.2-1.4 | | 2.2 | | | | | |
| F61T1 | Dec. 1, 2008 | Parabacteroides | Parabacteroides | Bacteroides TSDC19.2- | 1 | 55 | 5163060 | 263.7 | 272699 |
| | | distasonis | distasonis | 2.9 | | | | | |
| | | TSDC19.2-1.5 | | | | | | | |
| F61T1 | Dec. 1, 2008 | Parabacteroides | Parabacteroides | Parabacteroides | 1 | 55 | 5173455 | 80.1 | 223646 |
| | | distasonis | distasonis | distasonis TSDC19.2- | | | | | |
| | | TSDC19.2-1.6 | | 3.6 | | | | | |
| F61T1 | Dec. 1, 2008 | Parabacteroides | Parabacteroides | Parabacteroides | 1 | 55 | 5165221 | 86.6 | 217653 |
| | | distasonis | distasonis | distasonis TSDC19.2- | | | | | |
| | | TSDC19.2-1.7 | | 4.3 | | | | | |
| F61T1 | Dec. 1, 2008 | Parabacteroides | Parabacteroides | Bacteroides TSDC19.2- | 1 | 55 | 5152617 | 56.0 | 214297 |
| | | distasonis | distasonis | 4.8 | | | | | |
| | | TSDC19.2-1.8 | | | | | | | |
| F61T1 | Dec. 1, 2008 | Parabacteroides | Parabacteroides | Bacteroides TSDC19.2- | 1 | 55 | 5154017 | 60.0 | 267663 |
| | | distasonis | distasonis | 5.6 | | | | | |
| | | TSDC19.2-1.9 | | | | | | | |
| F61T1 | Dec. 1, 2008 | Parabacteroides | Parabacteroides | Parabacteroides | 16 | 56 | 6762098 | 19.1 | 59418 |
| | | goldsteinii | goldsteinii | goldsteinii TSDC19.2- | | | | | |
| | | TSDC19.2-1.1 | | 1.1 | | | | | |
| F61T1 | Dec. 1, 2008 | Parabacteroides | Parabacteroides | Parabacteroides | 16 | 56 | 6693425 | 178.7 | 115069 |
| | | goldsteinii | goldsteinii | goldsteinii TSDC19.2- | | | | | |
| | | TSDC19.2-1.2 | | 1.2 | | | | | |
| F61T1 | Dec. 1, 2008 | Roseburia | Roseburia | Roseburia intestinalis | 39 | 59 | 3304798 | 103.1 | 63159 |
| | | intestinalis | intestinalis | TSDC19.2-1.1 | | | | | |
| | | TSDC19.2-1.1 | | | | | | | |
| F61T1 | Dec. 1, 2008 | Ruminococcus sp | Ruminococcus sp | Ruminococcus sp DJF | 10 | 68 | 4075451 | 89.8 | 47006 |
| | | DJF VR70k1 | DJF VR70k1 | VR70k1 TSDC19.2-1.1 | | | | | |
| | | TSDC19.2-1.1 | | | | | | | |
| F61T1 | Dec. 1, 2008 | Ruminococcus sp | Ruminococcus sp | Ruminococcus sp DJF | 10 | 68 | 4081575 | 48.6 | 66935 |
| | | DJF VR70k1 | DJF VR70k1 | VR70k1 TSDC19.2-1.2 | | | | | |
| | | TSDC19.2-1.2 | | | | | | | |
| F61T1 | Dec. 1, 2008 | Ruminococcus | Ruminococcus | Ruminococcus torques | 32 | 69 | 3051029 | 50.6 | 102293 |
| | | torques | torques | TSDC19.2-2.2 | | | | | |
| | | TSDC19.2-1.1 | | | | | | | |
| F61T1 | Dec. 1, 2008 | Streptococcus | Streptococcus | Streptococcus gordonii | 29 | 71 | 2246344 | 34.0 | 114633 |
| | | gordonii | gordonii | TSDC19.2-1.1 | | | | | |
| | | TSDC19.2-1.1 | | | | | | | |
| F61T1 | Dec. 1, 2008 | Streptococcus | Streptococcus | Streptococcus | 28 | 72 | 2134596 | 403.3 | 166095 |
| | | parasanguinis | parasanguinis | parasanguinis | | | | | |
| | | TSDC19.2-1.1 | | TSDC19.2-1.1 | | | | | |
| F61T1 | Dec. 1, 2008 | Streptococcus | Streptococcus | Streptococcus | 6 | 73 | 2124637 | 87.6 | 134277 |
| | | thermophilus | thermophilus | thermophilus TSDC19.2- | | | | | |
| | | TSDC19.2-1.1 | | 1.1 | | | | | |
| F61T1 | Dec. 1, 2008 | Streptococcus | Streptococcus | Streptococcus | 6 | 73 | 2124600 | 170.3 | 143943 |
| | | thermophilus | thermophilus | thermophilus TSDC19.2- | | | | | |
| | | TSDC19.2-1.2 | | 1.2 | | | | | |
| F61T2 | Sep. 16, 2008 | Anaerococcus | Anaerococcus | Anaerococcus vaginalis | 12 | 2 | 1999434 | 96.6 | 166466 |
| | | vaginalis | vaginalis | TSDC20.1-1.1 | | | | | |
| | | TSDC20.1-1.1 | | | | | | | |
| F61T2 | Sep. 16, 2008 | Anaerococcus | Anaerococcus | Anaerococcus vaginalis | 12 | 2 | 1996380 | 34.3 | 72431 |
| | | vaginalis | vaginalis | TSDC20.1-1.2 | | | | | |
| | | TSDC20.1-1.2 | | | | | | | |
| F61T2 | Sep. 16, 2008 | Anaerofustis | Anaerofustis | Anaerofustis | 19 | 3 | 1915526 | 21.1 | 10292 |
| | | stercorihominis | stercorihominis | stercorihominis | | | | | |
| | | TSDC20.1-1.1 | | TSDC20.1-1.6 | | | | | |
| F61T2 | Sep. 16, 2008 | Bacteroides | Bacteroides fragilis | Bacteroides fragilis | 15 | 8 | 5301140 | 142.0 | 182192 |
| | | fragilis TSDC20.1- | | TSDC20.1-1.2 | | | | | |
| | | 1.1 | | | | | | | |
| F61T2 | Sep. 16, 2008 | Bacteroides | Bacteroides | Bacteroides | 5 | 9 | 7129730 | 65.5 | 273000 |
| | | intestinalis | intestinalis | cellulosilyticus | | | | | |
| | | TSDC20.1-1.1 | | TSDC20.1-1.6 | | | | | |
| F61T2 | Sep. 16, 2008 | Bacteroides | Bacteroides | Bacteroides | 5 | 9 | 7116847 | 122.8 | 273000 |
| | | intestinalis | intestinalis | cellulosilyticus | | | | | |
| | | TSDC20.1-1.2 | | TSDC20.1-1.7 | | | | | |
| F61T2 | Sep. 16, 2008 | Bacteroides | Bacteroides | Bacteroides | 5 | 9 | 7120846 | 31.6 | 128618 |
| | | intestinalis | intestinalis | cellulosilyticus | | | | | |
| | | TSDC20.1-1.3 | | TSDC20.1-1.8 | | | | | |
| F61T2 | Sep. 16, 2008 | Bacteroides | Bacteroides | Bacteroides uniformis | 3 | 14 | 5012603 | 66.3 | 188863 |
| | | uniformis | uniformis | TSDC20.1-1.6 | | | | | |
| | | TSDC20.1-1.1 | | | | | | | |
| F61T2 | Sep. 16, 2008 | Bacteroides | Bacteroides | Bacteroides vulgatus | 23 | 15 | 5141074 | 63.8 | 87019 |
| | | vulgatus | vulgatus | TSDC20.1-1.5 | | | | | |
| | | TSDC20.1-1.1 | | | | | | | |
| F61T2 | Sep. 16, 2008 | Bifidobacterium | Bifidobacterium | Bifidobacterium | 2 | 19 | 2377585 | 454.6 | 134284 |
| | | longum | longum | TSDC20.1-1.1 | | | | | |
| | | TSDC20.1-1.1 | | | | | | | |
| F61T2 | Sep. 16, 2008 | Bifidobacterium | Bifidobacterium | Bifidobacterium | 2 | 19 | 2376608 | 136.8 | 88265 |
| | | longum | longum | TSDC20.1-1.10 | | | | | |
| | | TSDC20.1-1.2 | | | | | | | |
| F61T2 | Sep. 16, 2008 | Bifidobacterium | Bifidobacterium | Bifidobacterium longum | 2 | 19 | 2376557 | 61.8 | 113894 |
| | | longum | longum | TSDC20.1-1.6 | | | | | |
| | | TSDC20.1-1.3 | | | | | | | |
| F61T2 | Sep. 16, 2008 | Bifidobacterium | Bifidobacterium | Bifidobacterium | 2 | 19 | 2377268 | 245.8 | 130111 |
| | | longum | longum | TSDC20.1-1.11 | | | | | |
| | | TSDC20.1-1.4 | | | | | | | |
| F61T2 | Sep. 16, 2008 | Bifidobacterium | Bifidobacterium | Bifidobacterium | 2 | 19 | 2376906 | 474.5 | 113101 |
| | | longum | longum | TSDC20.1-1.13 | | | | | |
| | | TSDC20.1-1.5 | | | | | | | |
| F61T2 | Sep. 16, 2008 | Clostridiales | Clostridiales | Clostridiales TSDC20.1- | 26 | 29 | 4257855 | 167.0 | 238142 |
| | | TSDC20.1-1.1 | | 1.3 | | | | | |
| F61T2 | Sep. 16, 2008 | Clostridium | Clostridium | Clostridium scindens | 1 | 36 | 3845147 | 138.1 | 295579 |
| | | scindens | scindens | TSDC20.1-1.2 | | | | | |
| | | TSDC20.1-1.1 | | | | | | | |
| F61T2 | Sep. 16, 2008 | Clostridium | Clostridium | Clostridium scindens | 1 | 36 | 3843613 | 122.6 | 246101 |
| | | scindens | scindens | TSDC20.1-1.3 | | | | | |
| | | TSDC20.1-1.2 | | | | | | | |
| F61T2 | Sep. 16, 2008 | Clostridium | Clostridium | Clostridium scindens | 1 | 36 | 3843965 | 275.3 | 236190 |
| | | scindens | scindens | TSDC20.1-1.5 | | | | | |
| | | TSDC20.1-1.3 | | | | | | | |
| F61T2 | Sep. 16, 2008 | Dorea longicatena | Dorea longicatena | Dorea longicatena | 10 | 42 | 3363213 | 219.5 | 68232 |
| | | TSDC20.1-1.1 | | TSDC20.1-1.3 | | | | | |
| F61T2 | Sep. 16, 2008 | Dorea longicatena | Dorea longicatena | Dorea longicatena | 10 | 42 | 3364555 | 108.0 | 82428 |
| | | TSDC20.1-1.2 | | TSDC20.1-1.4 | | | | | |
| F61T2 | Sep. 16, 2008 | Dorea longicatena | Dorea longicatena | Dorea longicatena | 10 | 42 | 3365684 | 134.8 | 77506 |
| | | TSDC20.1-1.3 | | TSDC20.1-1.6 | | | | | |
| F61T2 | Sep. 16, 2008 | Dorea longicatena | Dorea longicatena | Dorea longicatena | 10 | 42 | 3364256 | 87.3 | 70389 |
| | | TSDC20.1-1.4 | | TSDC20.1-1.7 | | | | | |
| F61T2 | Sep. 16, 2008 | Eggerthella lenta | Eggerthella lenta | Eggerthella lenta | 11 | 44 | 3296852 | 26.2 | 56034 |
| | | TSDC20.1-1.1 | | TSDC20.1-1.5 | | | | | |
| F61T2 | Sep. 16, 2008 | Eggerthella lenta | Eggerthella lenta | Eggerthella lenta | 11 | 44 | 3288980 | 42.8 | 91788 |
| | | TSDC20.1-1.2 | | TSDC20.1-1.6 | | | | | |
| F61T2 | Sep. 16, 2008 | Eggerthella lenta | Eggerthella lenta | Eggerthella lenta | 11 | 44 | 3281944 | 49.6 | 114198 |
| | | TSDC20.1-1.3 | | TSDC20.1-1.7 | | | | | |
| F61T2 | Sep. 16, 2008 | Eggerthella lenta | Eggerthella lenta | Eggerthella lenta | 11 | 44 | 3283114 | 33.1 | 117607 |
| | | TSDC20.1-1.4 | | TSDC20.1-1.8 | | | | | |
| F61T2 | Sep. 16, 2008 | Eggerthella lenta | Eggerthella lenta | Eggerthella lenta | 11 | 44 | 3289368 | 33.9 | 105492 |
| | | TSDC20.1-1.5 | | TSDC20.1-1.9 | | | | | |
| F61T2 | Sep. 16, 2008 | Eggerthella lenta | Eggerthella lenta | Eggerthella lenta | 11 | 44 | 3328830 | 10.6 | 22651 |
| | | TSDC20.1-1.6 | | TSDC20.1-2.2 | | | | | |
| F61T2 | Sep. 16, 2008 | Eggerthella lenta | Eggerthella lenta | Subdoligranulum | 11 | 44 | 3295928 | 27.0 | 83644 |
| | | TSDC20.1-1.7 | | variabile TSDC20.1-2.3 | | | | | |
| F61T2 | Sep. 16, 2008 | Eggerthella lenta | Eggerthella lenta | Subdoligranulum | 11 | 44 | 3296718 | 39.3 | 60137 |
| | | TSDC20.1-1.8 | | variabile TSDC20.1-2.5 | | | | | |
| F61T2 | Sep. 16, 2008 | Escherichia coli | Escherichia coli | Escherichia coli | 9 | 45 | 5145706 | 111.1 | 203790 |
| | | TSDC20.1-1.1 | | TSDC20.1-1.1 | | | | | |
| F61T2 | Sep. 16, 2008 | Escherichia coli | Escherichia coli | Escherichia coli | 9 | 45 | 5142290 | 102.2 | 109005 |
| | | TSDC20.1-1.2 | | TSDC20.1-1.3 | | | | | |
| F61T2 | Sep. 16, 2008 | Escherichia coli | Escherichia coli | Escherichia coli | 9 | 45 | 5153138 | 17.3 | 107857 |
| | | TSDC20.1-1.3 | | TSDC20.1-1.4 | | | | | |
| F61T2 | Sep. 16, 2008 | Escherichia coli | Escherichia coli | Escherichia coli | 9 | 45 | 5143520 | 58.1 | 140040 |
| | | TSDC20.1-1.4 | | TSDC20.1-1.6 | | | | | |
| F61T2 | Sep. 16, 2008 | Escherichia coli | Escherichia coli | Escherichia coli | 9 | 45 | 5144670 | 25.1 | 145310 |
| | | TSDC20.1-1.5 | | TSDC20.1-1.7 | | | | | |
| F61T2 | Sep. 16, 2008 | Escherichia coli | Escherichia coli | Escherichia coli | 9 | 45 | 5108167 | 147.1 | 202845 |
| | | TSDC20.1-1.6 | | TSDC20.1-1.8 | | | | | |
| F61T2 | Sep. 16, 2008 | Finegoldia magna | Finegoldia magna | Finegoldia magna | 13 | 50 | 1819524 | 116.8 | 153597 |
| | | TSDC20.1-1.1 | | TSDC20.1-1.1 | | | | | |
| F61T2 | Sep. 16, 2008 | Finegoldia magna | Finegoldia magna | Finegoldia magna | 13 | 50 | 1818662 | 322.2 | 133124 |
| | | TSDC20.1-1.2 | | TSDC20.1-1.2 | | | | | |
| F61T2 | Sep. 16, 2008 | Subdoligranulum | Subdoligranulum | Subdoligranulum | 24 | 74 | 3756225 | 21.2 | 45777 |
| | | variabile | variabile | variabile TSDC20.1-1.14 | | | | | |
| | | TSDC20.1-1.1 | | | | | | | |
| F61T2 | Sep. 16, 2008 | Subdoligranulum | Subdoligranulum | Subdoligranulum | 18 | 74 | 3636863 | 25.6 | 55086 |
| | | variabile | variabile | variabile TSDC20.1-2.13 | | | | | |
| | | TSDC20.1-2.1 | | | | | | | |
| F61T2 | Nov. 12, 2008 | Anaerofustis | Anaerofustis | Anaerofustis | 19 | 3 | 1890540 | 10.0 | 5523 |
| | | stercorihominis | stercorihominis | stercorihominis | | | | | |
| | | TSDC20.2-1.1 | | TSDC20.2-1.1 | | | | | |
| F61T2 | Nov. 12, 2008 | Anaerofustis | Anaerofustis | Anaerofustis | 19 | 3 | 1906015 | 8.0 | 5582 |
| | | stercorihominis | stercorihominis | stercorihominis | | | | | |
| | | TSDC20.2-1.2 | | TSDC20.2-1.3 | | | | | |
| F61T2 | Nov. 12, 2008 | Anaerofustis | Anaerofustis | Anaerofustis | 19 | 3 | 1875857 | 9.7 | 5199 |
| | | stercorihominis | stercorihominis | stercorihominis | | | | | |
| | | TSDC20.2-1.3 | | TSDC20.2-1.4 | | | | | |
| F61T2 | Nov. 12, 2008 | Anaerofustis | Anaerofustis | Anaerofustis | 19 | 3 | 1885035 | 7.7 | 4549 |
| | | stercorihominis | stercorihominis | stercorihominis | | | | | |
| | | TSDC20.2-1.4 | | TSDC20.2-1.5 | | | | | |
| F61T2 | Nov. 12, 2008 | Bacteroides | Bacteroides caccae | Bacteroides caccae | 16 | 6 | 5661179 | 57.3 | 104548 |
| | | caccae | | TSDC20.2-1.1 | | | | | |
| | | TSDC20.2-1.1 | | | | | | | |
| F61T2 | Nov. 12, 2008 | Bacteroides | Bacteroides caccae | Bacteroides caccae | 16 | 6 | 5659531 | 74.2 | 116103 |
| | | caccae | | TSDC20.2-1.2 | | | | | |
| | | TSDC20.2-1.2 | | | | | | | |
| F61T2 | Nov. 12, 2008 | Bacteroides | Bacteroides caccae | Bacteroides caccae | 16 | 6 | 5663388 | 90.1 | 113687 |
| | | caccae | | TSDC20.2-1.3 | | | | | |
| | | TSDC20.2-1.3 | | | | | | | |
| F61T2 | Nov. 12, 2008 | Bacteroides | Bacteroides caccae | Bacteroides caccae | 16 | 6 | 5664614 | 59.5 | 125425 |
| | | caccae | | TSDC20.2-1.4 | | | | | |
| | | TSDC20.2-1.4 | | | | | | | |
| F61T2 | Nov. 12, 2008 | Bacteroides | Bacteroides fragilis | Bacteroides fragilis | 15 | 8 | 5355029 | 10.6 | 14606 |
| | | fragilis TSDC20.2- | | TSDC20.2-1.1 | | | | | |
| | | 1.1 | | | | | | | |
| F61T2 | Nov. 12, 2008 | Bacteroides | Bacteroides fragilis | Bacteroides fragilis | 15 | 8 | 5327563 | 12.6 | 43246 |
| | | fragilis TSDC20.2- | | TSDC20.2-1.3 | | | | | |
| | | 1.2 | | | | | | | |
| F61T2 | Nov. 12, 2008 | Bacteroides | Bacteroides fragilis | Bacteroides fragilis | 15 | 8 | 5292626 | 27.7 | 83237 |
| | | fragilis TSDC20.2- | | TSDC20.2-1.4 | | | | | |
| | | 1.3 | | | | | | | |
| F61T2 | Nov. 12, 2008 | Bacteroides | Bacteroides fragilis | Bacteroides fragilis | 15 | 8 | 5307385 | 22.6 | 92632 |
| | | fragilis TSDC20.2- | | TSDC20.2-1.5 | | | | | |
| | | 1.4 | | | | | | | |
| F61T2 | Nov. 12, 2008 | Bacteroides | Bacteroides | Bacteroides | 5 | 9 | 7119644 | 27.7 | 273033 |
| | | intestinalis | intestinalis | cellulosilyticus | | | | | |
| | | TSDC20.2-1.1 | | TSDC20.2-1.2 | | | | | |
| F61T2 | Nov. 12, 2008 | Bacteroides | Bacteroides | Bacteroides | 5 | 9 | 7116994 | 29.5 | 210500 |
| | | intestinalis | intestinalis | cellulosilyticus | | | | | |
| | | TSDC20.2-1.2 | | TSDC20.2-1.4 | | | | | |
| F61T2 | Nov. 12, 2008 | Bacteroides | Bacteroides | Bacteroides | 20 | 9 | 7715252 | 11.5 | 29966 |
| | | intestinalis | intestinalis | cellulosilyticus | | | | | |
| | | TSDC20.2-2.1 | | TSDC20.2-1.5 | | | | | |
| F61T2 | Nov. 12, 2008 | Bacteroides | Bacteroides | Bacteroides | 7 | 13 | 6238981 | 62.2 | 115977 |
| | | thetaiotaomicron | thetaiotaomicron | thetaiotaomicron | | | | | |
| | | TSDC20.2-1.1 | | TSDC20.2-1.1 | | | | | |
| F61T2 | Nov. 12, 2008 | Bacteroides | Bacteroides | Bacteroides | 7 | 13 | 6240092 | 105.3 | 122359 |
| | | thetaiotaomicron | thetaiotaomicron | thetaiotaomicron | | | | | |
| | | TSDC20.2-1.2 | | TSDC20.2-1.2 | | | | | |
| F61T2 | Nov. 12, 2008 | Bacteroides | Bacteroides | Bacteroides | 7 | 13 | 6297958 | 12.3 | 24369 |
| | | thetaiotaomicron | thetaiotaomicron | thetaiotaomicron | | | | | |
| | | TSDC20.2-1.3 | | TSDC20.2-1.3 | | | | | |
| F61T2 | Nov. 12, 2008 | Bacteroides | Bacteroides | Bacteroides | 7 | 13 | 6238598 | 60.9 | 104652 |
| | | thetaiotaomicron | thetaiotaomicron | thetaiotaomicron | | | | | |
| | | TSDC20.2-1.4 | | TSDC20.2-1.4 | | | | | |
| F61T2 | Nov. 12, 2008 | Bacteroides | Bacteroides | Bacteroides | 7 | 13 | 6217411 | 59.4 | 115884 |
| | | thetaiotaomicron | thetaiotaomicron | thetaiotaomicron | | | | | |
| | | TSDC20.2-1.5 | | TSDC20.2-1.5 | | | | | |
| F61T2 | Nov. 12, 2008 | Bacteroides | Bacteroides | Bacteroides uniformis | 3 | 14 | 5004899 | 8.3 | 8698 |
| | | uniformis | uniformis | TSDC20.2-1.1 | | | | | |
| | | TSDC20.2-1.1 | | | | | | | |
| F61T2 | Nov. 12, 2008 | Bacteroides | Bacteroides | Bacteroides uniformis | 3 | 14 | 5082380 | 76.5 | 180959 |
| | | uniformis | uniformis | TSDC20.2-1.2 | | | | | |
| | | TSDC20.2-1.2 | | | | | | | |
| F61T2 | Nov. 12, 2008 | Bacteroides | Bacteroides | Bacteroides uniformis | 3 | 14 | 4992916 | 80.3 | 167532 |
| | | uniformis | uniformis | TSDC20.2-1.3 | | | | | |
| | | TSDC20.2-1.3 | | | | | | | |
| F61T2 | Nov. 12, 2008 | Bacteroides | Bacteroides | Bacteroides uniformis | 3 | 14 | 5014572 | 135.5 | 188849 |
| | | uniformis | uniformis | TSDC20.2-1.4 | | | | | |
| | | TSDC20.2-1.4 | | | | | | | |
| F61T2 | Nov. 12, 2008 | Bacteroides | Bacteroides | Bacteroides uniformis | 3 | 14 | 5084876 | 176.1 | 175616 |
| | | uniformis | uniformis | TSDC20.2-1.5 | | | | | |
| | | TSDC20.2-1.5 | | | | | | | |
| F61T2 | Nov. 12, 2008 | Bacteroides | Bacteroides | Bacteroides vulgatus | 22 | 15 | 4936884 | 5.3 | 1268 |
| | | vulgatus | vulgatus | TSDC20.2-1.2 | | | | | |
| | | TSDC20.2-1.1 | | | | | | | |
| F61T2 | Nov. 12, 2008 | Bifidobacterium | Bifidobacterium | Bifidobacterium longum | 2 | 19 | 2377568 | 93.3 | 130062 |
| | | longum | longum | TSDC20.2-1.4 | | | | | |
| | | TSDC20.2-1.1 | | | | | | | |
| F61T2 | Nov. 12, 2008 | Bifidobacterium | Bifidobacterium | Bifidobacterium longum | 2 | 19 | 2376259 | 112.6 | 113934 |
| | | longum | longum | TSDC20.2-1.5 | | | | | |
| | | TSDC20.2-1.2 | | | | | | | |
| F61T2 | Nov. 12, 2008 | Bifidobacterium | Bifidobacterium | Bifidobacterium | 2 | 19 | 2377991 | 97.5 | 110213 |
| | | longum | longum | TSDC20.2-1.9 | | | | | |
| | | TSDC20.2-1.3 | | | | | | | |
| F61T2 | Nov. 12, 2008 | Bifidobacterium | Bifidobacterium | Bifidobacterium | 2 | 19 | 2375105 | 54.8 | 87775 |
| | | longum | longum | TSDC20.2-2.3 | | | | | |
| | | TSDC20.2-1.4 | | | | | | | |
| F61T2 | Nov. 12, 2008 | Bifidobacterium | Bifidobacterium | Bifidobacterium | 2 | 19 | 2376537 | 156.8 | 114111 |
| | | longum | longum | TSDC20.2-2.6 | | | | | |
| | | TSDC20.2-1.5 | | | | | | | |
| F61T2 | Nov. 12, 2008 | Bifidobacterium | Bifidobacterium | Bifidobacterium | 2 | 19 | 2378044 | 311.4 | 78969 |
| | | longum | longum | TSDC20.2-2.8 | | | | | |
| | | TSDC20.2-1.6 | | | | | | | |
| F61T2 | Nov. 12, 2008 | Bifidobacterium | Bifidobacterium | Bifidobacterium | 2 | 19 | 2381733 | 299.2 | 81690 |
| | | longum | longum | TSDC20.2-3.2 | | | | | |
| | | TSDC20.2-1.7 | | | | | | | |
| F61T2 | Nov. 12, 2008 | Clostridium | Clostridium bolteae | Clostridium bolteae | 27 | 33 | 6534699 | 26.7 | 144947 |
| | | bolteae | | TSDC20.2-1.1 | | | | | |
| | | TSDC20.2-1.1 | | | | | | | |
| F61T2 | Nov. 12, 2008 | Clostridium | Clostridium | Clostridium hylemonae | 17 | 34 | 2596872 | 118.7 | 241944 |
| | | hylemonae | hylemonae | TSDC20.2-1.1 | | | | | |
| | | TSDC20.2-1.1 | | | | | | | |
| F61T2 | Nov. 12, 2008 | Clostridium | Clostridium | Clostridium hylemonae | 17 | 34 | 2561718 | 52.8 | 329981 |
| | | hylemonae | hylemonae | TSDC20.2-1.2 | | | | | |
| | | TSDC20.2-1.2 | | | | | | | |
| F61T2 | Nov. 12, 2008 | Clostridium | Clostridium | Clostridium scindens | 21 | 36 | 3722307 | 5.2 | 2045 |
| | | scindens | scindens | TSDC20.2-1.4 | | | | | |
| | | TSDC20.2-1.1 | | | | | | | |
| F61T2 | Nov. 12, 2008 | Escherichia coli | Escherichia coli | Escherichia coli | 9 | 45 | 5116861 | 8.2 | 5064 |
| | | TSDC20.2-1.1 | | TSDC20.2-1.5 | | | | | |
| F61T2 | Nov. 12, 2008 | Finegoldia magna | Finegoldia magna | Dialister invisus | 13 | 50 | 1819664 | 48.7 | 133373 |
| | | TSDC20.2-1.1 | | TSDC20.2-1.1 | | | | | |
| F61T2 | Nov. 12, 2008 | Ruminococcus | Ruminococcus | Ruminococcus gnavus | 25 | 65 | 3264682 | 79.8 | 92915 |
| | | gnavus | gnavus | TSDC20.2-1.1 | | | | | |
| | | TSDC20.2-1.1 | | | | | | | |
| F61T2 | Nov. 12, 2008 | Subdoligranulum | Subdoligranulum | Subdoligranulum | 18 | 74 | 3637766 | 21.0 | 48375 |
| | | variabile | variabile | variabile TSDC20.2-1.9 | | | | | |
| | | TSDC20.2-1.1 | | | | | | | |
| F61T2 | Nov. 12, 2008 | Veillonella parvula | Veillonella parvula | Veillonella parvula | 8 | 75 | 2049712 | 27.7 | 140948 |
| | | TSDC20.2-1.1 | | TSDC20.2-1.1 | | | | | |
| F61T2 | Nov. 12, 2008 | Veillonella parvula | Veillonella parvula | Veillonella parvula | 8 | 75 | 2048545 | 157.3 | 355470 |
| | | TSDC20.2-1.2 | | TSDC20.2-1.2 | | | | | |
| F61T2 | Nov. 12, 2008 | Veillonella parvula | Veillonella parvula | Veillonella parvula | 8 | 75 | 2070340 | 59.6 | 304021 |
| | | TSDC20.2-1.3 | | TSDC20.2-1.3 | | | | | |
| F61T2 | Nov. 12, 2008 | Veillonella parvula | Veillonella parvula | Veillonella parvula | 8 | 75 | 2062066 | 74.1 | 174140 |
| | | TSDC20.2-1.4 | | TSDC20.2-1.4 | | | | | |
| F61T2 | Nov. 12, 2008 | Veillonella parvula | Veillonella parvula | Veillonella parvula | 8 | 75 | 2049043 | 104.3 | 216536 |
| | | TSDC20.2-1.5 | | TSDC20.2-1.5 | | | | | |
| F61T2 | Nov. 12, 2008 | Veillonella | Veillonella parvula | Veillonella TSDC20.2- | 6 | 75 | 2133948 | 133.9 | 331005 |
| | | TSDC20.2-1.1 | | 1.2 | | | | | |
| F61T2 | Nov. 12, 2008 | Veillonella | Veillonella parvula | Veillonella TSDC20.2- | 6 | 75 | 2131306 | 33.4 | 64678 |
| | | TSDC20.2-1.2 | | 1.3 | | | | | |
| F61T2 | Nov. 12, 2008 | Veillonella | Veillonella parvula | Veillonella TSDC20.2- | 6 | 75 | 2132369 | 52.3 | 351963 |
| | | TSDC20.2-1.3 | | 1.4 | | | | | |

Isolates with the same Strain ID represent the same strain isolated and sequenced multiple times from a given sample or across different samples from the same individual.
Isolates with the same Species ID represent the same species (defined as a coverage score >0.50; see Table 1 for the species representation of each donor).
Species and strain names were assigned as the most abundant genus/species associated with a given cluster of genomes (with a cluster containing all strains with a coverage score >0.50)

TABLE 8
Fraction of bacterial strains (>96% coverage score) isolated across
multiple time points for a given individual, summarized at the
phylum level.

| | mean fraction of strains isolated |
|---|---|
| phylum | across multiple time points |
| Bacteroides | 0.52 |
| Proteobacteria | 0.50 |
| Actinobacteria | 0.36 |
| Firmicutes | 0.21 |

TABLE 9
The fractional abundance for every strain in the uneven mock communities.

| Phylum | Genus | Species | Accession Number | mock1.1 | | mock1.2 | | mock1.3 | | mock1.4 | | mock2.1 | | mock2.2 | | mock2.3 | | mock2.4 | |
|---|---|---|---|---|---|---|---|---|---|---|---|---|---|---|---|---|---|---|---|
| Actinobacteria | Bifidobacterium | pseudocatenulatum | NZ_ABXX00000000 | 0 | 0.0837 | 6 | 0.0013 | 7 | 0.0007 | 4 | 0.0052 | 4 | 0.0052 | 1 | 0.0418 | 0 | 0.0837 | 3 | 0.0105 |
| Actinobacteria | Bifidobacterium | bifidum | NC_Bbifidum_20456 | 7 | 0.0007 | 4 | 0.0052 | 5 | 0.0026 | 2 | 0.0209 | 7 | 0.0007 | 5 | 0.0026 | 2 | 0.0209 | 4 | 0.0052 |
| Actinobacteria | Collinsella | intestinalis | NZ_ABXH00000000 | 1 | 0.0418 | 2 | 0.0209 | 3 | 0.0105 | 0 | 0.0837 | 1 | 0.0418 | 2 | 0.0209 | 3 | 0.0105 | 4 | 0.0052 |
| Bacteroidetes | Alistipes | indistinctus | NZ_ADLD00000000 | 1 | 0.0418 | 4 | 0.0052 | 7 | 0.0007 | 6 | 0.0013 | 6 | 0.0013 | 4 | 0.0052 | 1 | 0.0418 | 7 | 0.0007 |
| Bacteroidetes | Bacteroides | cellulosilyticus | NZ_ACCH00000000 | 0 | 0.0837 | 1 | 0.0418 | 5 | 0.0026 | 6 | 0.0013 | 1 | 0.0418 | 4 | 0.0052 | 6 | 0.0013 | 5 | 0.0026 |
| Bacteroidetes | Bacteroides | ovatus | NZ_AAXF00000000 | 2 | 0.0209 | 3 | 0.0105 | 0 | 0.0837 | 5 | 0.0026 | 2 | 0.0209 | 3 | 0.0105 | 6 | 0.0013 | 0 | 0.0837 |
| Bacteroidetes | Bacteroides | uniformis | NZ_AAYH00000000 | 3 | 0.0105 | 4 | 0.0052 | 2 | 0.0209 | 7 | 0.0007 | 0 | 0.0837 | 5 | 0.0026 | 1 | 0.0418 | 6 | 0.0013 |
| Bacteroidetes | Bacteroides | dorei | NZ_ABWZ00000000 | 7 | 0.0007 | 1 | 0.0418 | 0 | 0.0837 | 3 | 0.0105 | 0 | 0.0837 | 2 | 0.0209 | 3 | 0.0105 | 1 | 0.0418 |
| Bacteroidetes | Bacteroides | eggerthii | NZ_ABVO00000000 | 6 | 0.0013 | 1 | 0.0418 | 3 | 0.0105 | 4 | 0.0052 | 1 | 0.0418 | 0 | 0.0837 | 5 | 0.0026 | 3 | 0.0105 |
| Bacteroidetes | Bacteroides | finegoldii | NZ_ABXI00000000 | 3 | 0.0105 | 7 | 0.0007 | 6 | 0.0013 | 0 | 0.0837 | 5 | 0.0026 | 7 | 0.0007 | 4 | 0.0052 | 2 | 0.0209 |
| Bacteroidetes | Bacteroides | intestinalis | NZ_ABJL00000000 | 5 | 0.0026 | 1 | 0.0418 | 3 | 0.0105 | 7 | 0.0007 | 0 | 0.0837 | 1 | 0.0418 | 3 | 0.0105 | 6 | 0.0013 |
| Bacteroidetes | Bacteroides | thetaiotaomicron | NC_Bthetaiotaomicron3731 | 5 | 0.0026 | 2 | 0.0209 | 4 | 0.0052 | 7 | 0.0007 | 7 | 0.0007 | 4 | 0.0052 | 3 | 0.0105 | 5 | 0.0026 |
| | | 3731 | | | | | | | | | | | | | | | | | |
| Bacteroidetes | Bacteroides | thetaiotaomicron | NC_Bthetaiotaomicron7330 | 2 | 0.0209 | 3 | 0.0105 | 4 | 0.0052 | 5 | 0.0026 | 6 | 0.0013 | 0 | 0.0837 | 1 | 0.0418 | 3 | 0.0105 |
| | | 7330 | | | | | | | | | | | | | | | | | |
| Bacteroidetes | Bacteroides | thetaiotaomicron | NC_004663 | 0 | 0.0837 | 3 | 0.0105 | 7 | 0.0007 | 1 | 0.0418 | 5 | 0.0026 | 0 | 0.0837 | 6 | 0.0013 | 1 | 0.0418 |
| | | VPI-5482 | | | | | | | | | | | | | | | | | |
| Bacteroidetes | Bacteroides | vulgatus | NC_009614 | 4 | 0.0052 | 2 | 0.0209 | 3 | 0.0105 | 1 | 0.0418 | 4 | 0.0052 | 7 | 0.0007 | 5 | 0.0026 | 1 | 0.0418 |
| Bacteroidetes | Bacteroides | xylanisolvens | FP929033 | 0 | 0.0837 | 5 | 0.0026 | 7 | 0.0007 | 3 | 0.0105 | 3 | 0.0105 | 2 | 0.0209 | 4 | 0.0052 | 6 | 0.0013 |
| Bacteroidetes | Parabacteroides | johnsonii | NZ_ABYH00000000 | 3 | 0.0105 | 6 | 0.0013 | 7 | 0.0007 | 2 | 0.0209 | 6 | 0.0013 | 7 | 0.0007 | 0 | 0.0837 | 5 | 0.0026 |
| Firmicute | Anaerocoecus | hydrogenalis | NZ_ABXA00000000 | 3 | 0.0105 | 0 | 0.0837 | 5 | 0.0026 | 4 | 0.0052 | 7 | 0.0007 | 1 | 0.0418 | 0 | 0.0837 | 5 | 0.0026 |
| Firmicute | Anaerotruncus | colihominis | NZ_ABGD00000000 | 2 | 0.0209 | 3 | 0.0105 | 0 | 0.0837 | 7 | 0.0007 | 1 | 0.0418 | 3 | 0.0105 | 7 | 0.0007 | 4 | 0.0052 |
| Firmicute | Blautia | hansenii | NZ_ABYU00000000 | 5 | 0.0026 | 3 | 0.0105 | 0 | 0.0837 | 2 | 0.0209 | 3 | 0.0105 | 5 | 0.0026 | 7 | 0.0007 | 6 | 0.0013 |
| Firmicute | Blautia | luti | NC_Bluti | 6 | 0.0013 | 7 | 0.0007 | 2 | 0.0209 | 4 | 0.0052 | 6 | 0.0013 | 3 | 0.0105 | 2 | 0.0209 | 1 | 0.0418 |
| Firmicute | Clostridium | leptum | NZ_ABCB00000000 | 7 | 0.0007 | 3 | 0.0105 | 4 | 0.0052 | 6 | 0.0013 | 5 | 0.0026 | 6 | 0.0013 | 2 | 0.0209 | 7 | 0.0007 |
| Firmicute | Clostridium | nexile-related | NC_Cnexile1787 | 5 | 0.0026 | 4 | 0.0052 | 1 | 0.0418 | 6 | 0.0013 | 4 | 0.0052 | 2 | 0.0209 | 5 | 0.0026 | 6 | 0.0013 |
| | | A2-232 | | | | | | | | | | | | | | | | | |
| Firmicute | Clostridium | saccharolyticum- | NZ_ACFX00000000 | 3 | 0.0105 | 1 | 0.0418 | 0 | 0.0837 | 5 | 0.0026 | 2 | 0.0209 | 6 | 0.0013 | 1 | 0.0418 | 4 | 0.0052 |
| | | related | | | | | | | | | | | | | | | | | |
| Firmicute | Clostridium | asparagiforme | NZ_ACCJ00000000 | 1 | 0.0418 | 2 | 0.0209 | 6 | 0.0013 | 0 | 0.0837 | 3 | 0.0105 | 4 | 0.0052 | 0 | 0.0837 | 1 | 0.0418 |
| Firmicute | Clostridium | hathewayi | NZ_ACIO00000000 | 3 | 0.0105 | 6 | 0.0013 | 5 | 0.0026 | 1 | 0.0418 | 1 | 0.0418 | 5 | 0.0026 | 6 | 0.0013 | 2 | 0.0209 |
| Firmicute | Clostridium | nexile | NZ_ABWO00000000 | 5 | 0.0026 | 7 | 0.0007 | 4 | 0.0052 | 3 | 0.0105 | 2 | 0.0209 | 6 | 0.0013 | 5 | 0.0026 | 4 | 0.0052 |
| Firmicute | Clostridium | sporogenes | NZ_ABKW00000000 | 7 | 0.0007 | 6 | 0.0013 | 2 | 0.0209 | 1 | 0.0418 | 7 | 0.0007 | 2 | 0.0209 | 4 | 0.0052 | 1 | 0.0418 |
| Firmicute | Coprococcus | comes | NZ_ABVR00000000 | 4 | 0.0052 | 5 | 0.0026 | 1 | 0.0418 | 3 | 0.0105 | 6 | 0.0013 | 3 | 0.0105 | 4 | 0.0052 | 7 | 0.0007 |
| Firmicute | Dorea | formicigenerans | NZ_AAXA00000000 | 4 | 0.0052 | 6 | 0.0013 | 1 | 0.0418 | 0 | 0.0837 | 2 | 0.0209 | 3 | 0.0105 | 7 | 0.0007 | 4 | 0.0052 |
| Firmicute | Dorea | longicatena | NZ_AAXB00000000 | 5 | 0.0026 | 0 | 0.0837 | 6 | 0.0013 | 7 | 0.0007 | 5 | 0.0026 | 4 | 0.0052 | 0 | 0.0837 | 3 | 0.0105 |
| Firmicute | Eubacterium | eligens | NC_012778 | 4 | 0.0052 | 7 | 0.0007 | 5 | 0.0026 | 6 | 0.0013 | 3 | 0.0105 | 1 | 0.0418 | 4 | 0.0052 | 0 | 0.0837 |
| Firmicute | Eubacterium | biforme | NZ_ABYT00000000 | 4 | 0.0052 | 5 | 0.0026 | 2 | 0.0209 | 1 | 0.0418 | 5 | 0.0026 | 0 | 0.0837 | 2 | 0.0209 | 3 | 0.0105 |
| Firmicute | Eubacterium | ventriosum | NZ_AAVL00000000 | 6 | 0.0013 | 4 | 0.0052 | 5 | 0.0026 | 3 | 0.0105 | 0 | 0.0837 | 6 | 0.0013 | 2 | 0.0209 | 7 | 0.0007 |
| Firmicute | Faecalibacterium | prausnitzii | NZ_ABED00000000 | 6 | 0.0013 | 4 | 0.0052 | 1 | 0.0418 | 2 | 0.0209 | 2 | 0.0209 | 6 | 0.0013 | 5 | 0.0026 | 3 | 0.0105 |
| | | M21/2 | | | | | | | | | | | | | | | | | |
| Firmicute | Roseburia | intestinalis | NZ_ABYJ00000000 | 2 | 0.0209 | 6 | 0.0013 | 0 | 0.0837 | 1 | 0.0418 | 7 | 0.0007 | 3 | 0.0105 | 5 | 0.0026 | 2 | 0.0209 |
| Firmicute | Ruminococcus | gnavus | NZ_AAYG00000000 | 2 | 0.0209 | 0 | 0.0837 | 4 | 0.0052 | 5 | 0.0026 | 5 | 0.0026 | 1 | 0.0418 | 3 | 0.0105 | 0 | 0.0837 |
| Firmicute | Ruminococcus | lactaris | NZ_ABOU00000000 | 0 | 0.0837 | 2 | 0.0209 | 4 | 0.0052 | 3 | 0.0105 | 0 | 0.0837 | 1 | 0.0418 | 7 | 0.0007 | 5 | 0.0026 |
| Firmicute | Ruminococcus | torques | NZ_AAVP00000000 | 1 | 0.0418 | 7 | 0.0007 | 2 | 0.0209 | 5 | 0.0026 | 7 | 0.0007 | 2 | 0.0209 | 3 | 0.0105 | 0 | 0.0837 |
| Firmicute | Streptococcus | infantarius | NZ_ABJK00000000 | 1 | 0.0418 | 5 | 0.0026 | 6 | 0.0013 | 2 | 0.0209 | 4 | 0.0052 | 5 | 0.0026 | 7 | 0.0007 | 6 | 0.0013 |
| Firmicute | Subdoligranulum | variabile | NZ_ACBY00000000 | 2 | 0.0209 | 5 | 0.0026 | 7 | 0.0007 | 6 | 0.0013 | 1 | 0.0418 | 0 | 0.0837 | 2 | 0.0209 | 7 | 0.0007 |
| Proteobacteria | Edwardsiella | tarda | NZ_ADGK00000000 | 1 | 0.0418 | 2 | 0.0209 | 6 | 0.0013 | 4 | 0.0052 | 4 | 0.0052 | 0 | 0.0837 | 6 | 0.0013 | 7 | 0.0007 |
| Proteobacteria | Enterobacter | cancerogenus | NC_Ecancerogenus | 4 | 0.0052 | 0 | 0.0837 | 3 | 0.0105 | 7 | 0.0007 | 3 | 0.0105 | 6 | 0.0013 | 4 | 0.0052 | 0 | 0.0837 |
| Proteobacteria | Escherichia | coli K12 | NC_000913 | 7 | 0.0007 | 0 | 0.0837 | 6 | 0.0013 | 2 | 0.0209 | 4 | 0.0052 | 7 | 0.0007 | 0 | 0.0837 | 2 | 0.0209 |
| Proteobacteria | Escherichia | fergusonii | NC_011740 | 7 | 0.0007 | 5 | 0.0026 | 3 | 0.0105 | 0 | 0.0837 | 6 | 0.0013 | 4 | 0.0052 | 7 | 0.0007 | 2 | 0.0209 |
| Proteobacteria | Proteus | penneri | NZ_ABVP00000000 | 6 | 0.0013 | 0 | 0.0837 | 1 | 0.0418 | 4 | 0.0052 | 3 | 0.0105 | 7 | 0.0007 | 6 | 0.0013 | 5 | 0.0026 |
| Proteobacteria | Providencia | alcalifaciens | NZ_ABXW00000000 | 6 | 0.0013 | 1 | 0.0418 | 2 | 0.0209 | 0 | 0.0837 | 2 | 0.0209 | 7 | 0.0007 | 1 | 0.0418 | 0 | 0.0837 |
| Verrucomicrobia | Akkermansia | muciniphila | NC_010655 | 0 | 0.0837 | 7 | 0.0007 | 1 | 0.0418 | 5 | 0.0026 | 0 | 0.0837 | 5 | 0.0026 | 1 | 0.0418 | 2 | 0.0209 |

TABLE 10
Primers to conserved regions flanking the V1V2 and V4V5 regions of
the bacterial 16S rRNA gene that were used for standard and LEA-Seq
amplicon generation.
V1V2 standard (MiSeq and 454) and LEA-Seq (HiSeq 2000)

| | |
|---|---|
| 16S 8F primer | 5′ AGAGTTTGATCCTGGCTCAG |
| 16S 338R primer | 5′ TGCTGCCTCCCGTAGGAGT |

these primers were used for standard amplicon sequencing and LEA-Seq
V4 standard (MiSeq)

| | |
|---|---|
| 16S 515F | 5′ GTGCCAGCAGCCGCGGTAA |
| 16S 806R consensus | 5′ GGACTACHVGGGTATCTAAT |
| 16S 806R majority | 5′ GGACTACCAGGGTATCTAAT |

these primers were used for standard amplicon sequencing
V4 LEA-Seq (HiSeq 2000)

| | |
|---|---|
| 16S 515F | 5′ GTGCCAGCAGCCGCGGTAA |
| 16S 806R consensus | 5′ GGACTACHVGGGTATCTAATCC |
| 16S 806R majority | 5′ GGACTACCAGGGTATCTAATCC |

these primers were used for LEA-Seq
V4 standard (MiSeq) phasing primers

| primer name | primer + phase nucleotides |
|---|---|
| 515F phase0 | 5′ GTGCCAGCAGCCGCGGTAA |
| 515F phase1 | 5′ CGTGCCAGCAGCCGCGGTAA |
| 515F phase2 | 5′ ACGTGCCAGCAGCCGCGGTAA |
| 515F phase3 | 5′ TATGTGCCAGCAGCCGCGGTAA |
| 806R phase0 | 5′ GGACTACCAGGGTATCTAAT |
| 806R phase1 | 5′ CGGACTACCAGGGTATCTAAT |
| 806R phase2 | 5′ AAGGACTACCAGGGTATCTAAT |
| 806R phase3 | 5′ TTCGGACTACCAGGGTATCTAAT |
| 806R phase4 | 5′ ATTCGGACTACCAGGGTATCTAAT |
| 806R phase5 | 5′ CACTAGGACTACCAGGGTATCTAAT |
| 806R phase6 | 5′ GCATATGGACTACCAGGGTATCTAAT |
| 806R phase7 | 5′ TCCATTTGGACTACCAGGGTATCTAAT |

TABLE 11
Human gut bacteria used to measure (in silico) the primer sensitivity and
seqeuence resolution of different variable regions of the bacterial 16S rRNA gene.

| Organism | Accession |
|---|---|
| Actinomyces odontolyticus ATCC 17982 | NZ_AAYI00000000 |
| Akkermansia muciniphila ATCC BAA-835 | NC_010655 |
| Alistipes putredinis DSM 17216 | NZ_ABFK00000000 |
| Anaerococcus hydrogenalis DSM 7454 | NZ_ABXA00000000 |
| Anaerofustis stercorihominis DSM 17244 | NZ_ABIL00000000 |
| Anaerostipes caccae DSM 14662 | NZ_ABAX00000000 |
| Anaerotruncus colihominis DSM 17241 | NZ_ABGD00000000 |
| Bacteroides caccae ATCC 43185 | NZ_AAVM00000000 |
| Bacteroides capillosus ATCC 29799 | NZ_AAXG00000000 |
| Bacteroides cellulosilyticus DSM 14838 | NZ_ACCH00000000 |
| Bacteroides coprocola DSM 17136 | NZ_ABIY00000000 |
| Bacteroides coprophilus DSM 18228 | NZ_ACBW00000000 |
| Bacteroides dorei DSM 17855 | NZ_ABWZ00000000 |
| Bacteroides eggerthii DSM 20697 | NZ_ABVO00000000 |
| Bacteroides finegoldii DSM 17565 | NZ_ABXI00000000 |
| Bacteroides fragilis 3_1_12 | NZ_ABZX00000000 |
| Bacteroides fragilis NCTC 9343 | NC_003228 |
| Bacteroides fragilis YCH46 | NC_006347 |
| Bacteroides intestinalis DSM 17393 | NZ_ABJL00000000 |
| Bacteroides ovatus ATCC 8483 | NZ_AAXF00000000 |
| Bacteroides plebeius DSM 17135 | NZ_ABQC00000000 |
| Bacteroides sp. 1_1_6 | NZ_ACIC00000000 |
| Bacteroides sp. D1 | NZ_ACAB00000000 |
| Bacteroides sp. D2 | NZ_ACGA00000000 |
| Bacteroides stercoris ATCC 43183 | NZ_ABFZ00000000 |
| Bacteroides thetaiotaomicron 3731 | NC_Bthetaiotaomicron3731 |
| Bacteroides thetaiotaomicron 7330 | NC_Bthetaiotaomicron7330 |
| Bacteroides thetaiotaomicronVPI-5482 | NC_004663 |
| Bacteroides uniformis ATCC 8492 | NZ_AAYH00000000 |
| Bacteroides vulgatus ATCC 8482 | NC_009614 |
| Bacteroides cellulosilyticus WH2 | NC_BWH2 |
| Bacteroides xylanisolvens XB1A | FP929033 |
| Bifidobacterium adolescentis ATCC 15703 | NC_008618 |
| Bifidobacterium adolescentis L2-32 | NZ_AAXD00000000 |
| Bifidobacterium angulatum DSM 20098 | NZ_ABYS00000000 |
| Bifidobacterium animalis subsp. lactis AD011 | NC_011835 |
| Bifidobacterium animalis subsp. lactis HN019 | NZ_ABOT00000000 |
| Bifidobacterium breve DSM 20213 | NZ_ACCG00000000 |
| Bifidobacterium catenulatum DSM 16992 | NZ_ABXY00000000 |
| Bifidobacterium dentium | NZ_ABIX00000000 |
| Bifidobacterium gallicum DSM 20093 | NZ_ABXB00000000 |
| Bifidobacterium longum DJO10A | NC_010816 |
| Bifidobacterium longum NCC2705 | NC_004307 |
| Bifidobacterium pseudocatenulatum DSM 20438 | NZ_ABXX00000000 |
| Blautia hansenii DSM 20583 | NZ_ABYU00000000 |
| Blautia hydrogenotrophica DSM 10507 | NZ_ACBZ00000000 |
| Bryantella formatexigens DSM 14469 | NZ_ACCL00000000 |
| Butyrivibrio crossotus DSM 2876 | NZ_ABWN00000000 |
| Catenibacterium mitsuokai DSM 15897 | NZ_ACCK00000000 |
| Citrobacter youngae ATCC 29220 | NZ_ABWL00000000 |
| Clostridium asparagiforme DSM 15981 | NZ_ACCJ00000000 |
| Clostridium bartlettii DSM 16795 | NZ_ABEZ00000000 |
| Clostridium bolteae ATCC BAA-613 | NZ_ABCC00000000 |
| Clostridium hiranonis DSM 13275 | NZ_ABWP00000000 |
| Clostridium hylemonae DSM 15053 | NZ_ABYI00000000 |
| Clostridium leptum DSM 753 | NZ_ABCB00000000 |
| Clostridium methylpentosum DSM 5476 | NZ_ACEC00000000 |
| Clostridium nexile DSM 1787 | NZ_ABWO00000000 |
| Clostridium ramosum DSM 1402 | NZ_ABFX00000000 |
| Clostridium scindens ATCC 35704 | NZ_ABFY00000000 |
| Clostridium sp. L2-50 | NZ_AAYW00000000 |
| Clostridium sp. M62/1 | NZ_ACFX00000000 |
| Clostridium sp. SS2/1 | NZ_ABGC00000000 |
| Clostridium spiroforme DSM 1552 | NZ_ABIK00000000 |
| Clostridium sporogenes ATCC 15579 | NZ_ABKW00000000 |
| Clostridium symbiosum | NC_Csymbiosum |
| Collinsella aerofaciens ATCC 25986 | NZ_AAVN00000000 |
| Collinsella intestinalis DSM 13280 | NZ_ABXH00000000 |
| Collinsella stercoris DSM 13279 | NZ_ABXJ00000000 |
| Coprococcus comes ATCC 27758 | NZ_ABVR00000000 |
| Coprococcus eutactus ATCC 27759 | NZ_ABEY00000000 |
| Desulfovibrio piger ATCC 29098 | NZ_ABXU00000000 |
| Desulfovibrio piger GOR1 | NC_DpigerGOR1 |
| Dorea formicigenerans ATCC 27755 | NZ_AAXA00000000 |
| Dorea longicatena DSM 13814 | NZ_AAXB00000000 |
| Enterobacter cancerogenus | NC_Ecancerogenus |
| Escherichia coli str. K-12 substr. MG1655 | NC_000913 |
| Escherichia fergusonii ATCC 35469 | NC_011740 |
| Eubacterium biforme DSM 3989 | NZ_ABYT00000000 |
| Eubacterium dolichum DSM 3991 | NZ_ABAW00000000 |
| Eubacterium eligens ATCC 27750 | NC_012778 |
| Eubacterium hallii DSM 3353 | NZ_ACEP00000000 |
| Eubacterium rectale ATCC 33656 | NC_012781 |
| Eubacterium rectale DSM17629 | FP929042 |
| Eubacterium ventriosum ATCC 27560 | NZ_AAVL00000000 |
| Faecalibacterium prausnitzii A2-165 | NZ_ACOP00000000 |
| Faecalibacterium prausnitzii M21/2 | NZ_ABED00000000 |
| Fusobacterium sp. 4_1_13 | NZ_ACDE00000000 |
| Fusobacterium varium ATCC 27725 | NZ_ACIE00000000 |
| Helicobacter pylori HPAG1 | NC_008086 |
| Holdemania filiformis DSM 12042 | NZ_ACCF00000000 |
| Lactobacillus casei ATCC 334 | NC_008526 |
| Lactobacillus delbrueckii subsp. bulgaricus ATCC 11842 | NC_008054 |
| Lactobacillus reuteri DSM 20016 | NC_009513 |
| Lactococcus lactis subsp. cremoris MG1363 | NC_009004 |
| Lactococcus lactis subsp. cremoris SK11 | NC_008527 |
| Lactococcus lactis subsp. lactis II1403 | NC_002662 |
| M23A | NC_M23A |
| Methanobrevibacter smithii ATCC 35061 | NC_009515 |
| Methanobrevibacter smithii DSM 2374 | NZ_ABYV00000000 |
| Methanobrevibacter smithii DSM 2375 | NZ_ABYW00000000 |
| Methanosphaera stadtmanae DSM 3091 | NC_007681 |
| Mitsuokella multacida DSM 20544 | NZ_ABWK00000000 |
| Parabacteroides distasonis ATCC 8503 | NC_009615 |
| Parabacteroides johnsonii DSM 18315 | NZ_ABYH00000000 |
| Parabacteroides merdae ATCC 43184 | NZ_AAXE00000000 |
| Parvimonas micra ATCC 33270 | NZ_ABEE00000000 |
| Prevotella copri DSM 18205 | NZ_ACBX00000000 |
| Proteus penneri ATCC 35198 | NZ_ABVP00000000 |
| Providencia alcalifaciens DSM 30120 | NZ_ABXW00000000 |
| Providencia rettgeri DSM 1131 | NZ_ACCI00000000 |
| Providencia rustigianii DSM 4541 | NZ_ABXV00000000 |
| Providencia stuartii ATCC 25827 | NZ_ABJD00000000 |
| Roseburia intestinalis L1-82 | NZ_ABYJ00000000 |
| Ruminococcus bromii L263 | FP929051 |
| Ruminococcus gnavus ATCC 29149 | NZ_AAYG00000000 |
| Ruminococcus lactaris ATCC 29176 | NZ_ABOU00000000 |
| Ruminococcus obeum ATCC 29174 | NZ_AAVO00000000 |
| Ruminococcus torques ATCC 27756 | NZ_AAVP00000000 |
| Shigella sp. D9 | NZ_ACDL00000000 |
| Streptococcus infantarius subsp. infantarius ATCC BAA-102 | NZ_ABJK00000000 |
| Streptococcus thermophilus CNRZ1066 | NC_006449 |
| Streptococcus thermophilus LMD-9 | NC_008532 |
| Streptococcus thermophilus LMG 18311 | NC_006448 |
| Subdoligranulum variabile DSM 15176 | NZ_ACBY00000000 |
| Vibrio cholerae O1 biovar eltor str. N16961 chromosome I | NC_002505 |
| Vibrio cholerae O1 biovar eltor str. N16961 chromosome II | NC_002506 |
| Victivallis vadensis ATCC BAA-548 | NZ_ABDE00000000 |

TABLE 12
Error rate of each read (as measured by the Illumina QC software
using a Phi X174 spike-in control) as a function of the phasing and
sequencing strategy used for amplicon sequencing on the Illumina
MiSeq instrument.

| | Sequencing | PhiX (% of total DNA in | Error Rate | Error Rate |
|---|---|---|---|---|
| Phasing | type | sample) | Read1 | Read2 |
| no | unidirectional | 14.1 | 0.9 | 6.6 |
| 4 nucleotides for 515F primer; 8 | bidirectional | 5.15 | 0.7 | 1.4 |
| nucleotides for 806R | | | | |
| 4 nucleotides for 515F primer; 8 | bidirectional | 9.5 | 0.5 | 0.9 |
| nucleotides for 806R | | | | |
| 4 nucleotides for 515F primer; 8 | bidirectional | 27.25 | 0.6 | 0.7 |
| nucleotides for 806R | | | | |

unidirectional = uses custom sequence primers and sequences each end of the amplicon in one direction only (see ref. 40 for details)
bidirectional = read1 and read2 start with both 515F and 806R primers

TABLE 13
Mean performance of 16S rRNA amplicon sequencing methods.

| | | | | | | Total | | | | | | |
|---|---|---|---|---|---|---|---|---|---|---|---|---|
| | | | | mock | Source of | Number of | | Precision at various abundance | | | | |
| Sequence | | | | community | Taq | Reads | Number of | thresholds | | | | |
| Run ID | Region | Type | Platform | type | polymerase | Generated | Amplicons | 1:500 | 1:1000 | 1:5000 | 1:10000 | 1:50000 |

A. Subsample 2000 reads

| | | | | | | | | | | | |
|---|---|---|---|---|---|---|---|---|---|---|---|
| 1 | V4 | phased | Illumina | even | 5Prime | 13336 | 13336 | 0.81 | 0.75 | 0.15 | 0.03 |
| | | | MiSeq | | | | | | | | |
| 1 | V4 | phased; | Illumina | even | 5Prime | 13336 | 2000 | 0.78 | 0.45 | | |
| | | subsampled | MiSeq | | | | | | | | |
| | | to 2000 | | | | | | | | | |
| | | reads | | | | | | | | | |

B. Subsample amplicon sequences to 2000, 10000, 20000, 50000, 100000 reads

| | | | | | | | | | | | | |
|---|---|---|---|---|---|---|---|---|---|---|---|---|
| 1 | V4 | phased | Illumina | even | 5Prime | 13336 | 13336 | 0.81 | 0.75 | 0.15 | 0.03 | |
| | | | MiSeq | | | | | | | | | |
| 1 | V4 | phased | Illumina | even | 5Prime | 13336 | 10000 | 0.80 | 0.75 | 0.11 | 0.01 | |
| | | | MiSeq | | | | | | | | | |
| 1 | V4 | phased | Illumina | even | 5Prime | 13336 | 2000 | 0.78 | 0.45 | | | |
| | | | MiSeq | | | | | | | | | |
| 2 | V4 | phased | Illumina | even | Phusion | 422960 | 422960 | 0.70 | 0.49 | 0.17 | 0.10 | 0.02 |
| | | | MiSeq | | | | | | | | | |
| 2 | V4 | phased | Illumina | even | Phusion | 422960 | 100000 | 0.73 | 0.51 | 0.15 | 0.09 | 0.01 |
| | | | MiSeq | | | | | | | | | |
| 2 | V4 | phased | Illumina | even | Phusion | 422960 | 50000 | 0.70 | 0.47 | 0.14 | 0.08 | 0.00 |
| | | | MiSeq | | | | | | | | | |
| 2 | V4 | phased | Illumina | even | Phusion | 422960 | 20000 | 0.63 | 0.40 | 0.13 | 0.06 | |
| | | | MiSeq | | | | | | | | | |
| 2 | V4 | phased | Illumina | even | Phusion | 422960 | 10000 | 0.61 | 0.41 | 0.10 | 0.02 | |
| | | | MiSeq | | | | | | | | | |
| 2 | V4 | phased | Illumina | even | Phusion | 422960 | 2000 | 0.49 | 0.28 | | | |
| | | | MiSeq | | | | | | | | | |

C. Comparison of different Taq DNA polymerases (all data subsampled to 10,000 reads)

| | | | | | | | | | | | |
|---|---|---|---|---|---|---|---|---|---|---|---|
| 3 | V4 | phased | Illumina | even | MTP | 10247 | 10000 | 0.79 | 0.81 | 0.17 | 0.02 |
| | | | MiSeq | | | | | | | | |
| 4 | V4 | phased | Illumina | even | MTP | 11994 | 10000 | 0.79 | 0.78 | 0.14 | 0.02 |
| | | | MiSeq | | | | | | | | |
| 5 | V4 | phased | Illumina | even | OKT | 128205 | 10000 | 0.79 | 0.80 | 0.14 | 0.02 |
| | | | MiSeq | | | | | | | | |
| 6 | V4 | phased | Illumina | even | OKT | 110474 | 10000 | 0.79 | 0.77 | 0.15 | 0.02 |
| | | | MiSeq | | | | | | | | |
| 7 | V4 | phased | Illumina | even | Takara | 6248 | 10000 | 0.80 | 0.79 | 0.08 | |
| | | | MiSeq | | | | | | | | |
| 8 | V4 | phased | Illumina | even | Takara | 10591 | 10000 | 0.80 | 0.81 | 0.13 | 0.02 |
| | | | MiSeq | | | | | | | | |
| 9 | V4 | phased | Illumina | even | ExTakara | 17346 | 10000 | 0.81 | 0.80 | 0.12 | 0.02 |
| | | | MiSeq | | | | | | | | |
| 10 | V4 | phased | Illumina | even | ExTakara | 25037 | 10000 | 0.80 | 0.81 | 0.12 | 0.02 |
| | | | MiSeq | | | | | | | | |
| 1 | V4 | phased | Illumina | even | 5Prime | 13336 | 10000 | 0.80 | 0.75 | 0.11 | 0.01 |
| | | | MiSeq | | | | | | | | |
| 11 | V4 | phased | Illumina | even | Phusion | 60107 | 10000 | 0.80 | 0.64 | 0.11 | 0.02 |
| | | | MiSeq | | | | | | | | |

TABLE 14
Quantitative performance of 16S rRNA amplicon sequencing methods
(correlation between known and measured fractional abundance of all
strains), empirical estimates of primer sensitivity (% not detected) and
masking (% non-unique)

| | | | % not | |
|---|---|---|---|---|
| | primer pair | correlation | detected | % non-unique |
| standard | V4 consensus primer | 0.77 | 7% | 18% |
| | V4 abundant primer | 0.82 | 7% | 18% |
| LEA-Seq | V1V2 | 0.76 | 13% | 13% |
| | V4 consensus primer | 0.80 | 4% | 22% |
| | V4 abundant primer | 0.80 | 4% | 22% |

Unless indicated, Phusion HF PCR master mix was used for the amplification
abundant = most abundant primer as defined based on the 128 genomes in Table S9
consensus = degenerate primer as defined based on the 128 genomes in Table S9

TABLE 15
Quantitative performance of 16S rRNA amplicon sequencing methods for each strain in the mock community.

| | | | | V1V2 LEA-Seq | | V4 LEA-Seq | | V4 MiSeq | |
|---|---|---|---|---|---|---|---|---|---|
| Phylum | Genus | Species | accession | corr (r) | slope | corr (r) | slope | corr (r) | slope |
| Actinobacteria | Bifidobacterium | pseudocatenulatum | NZ_ABXX00000000 | 0.980 | 0.985 | 0.994 | 0.952 | 0.997 | 1.041 |
| Actinobacteria | Bifidobacterium | bifidum | NC_Bbifidum_20456 | | | 0.992 | 0.968 | 0.994 | 0.891 |
| Actinobacteria | Collinsella | intestinalis | NZ_ABXH00000000 | 0.995 | 1.171 | 0.975 | 0.797 | 0.987 | 0.857 |
| Bacteroidetes | Alistipes | indistinctus | NC_Aindistictus | | | 0.991 | 0.927 | 0.995 | 0.988 |
| Bacteroidetes | Bacteroides | cellulosilyticus | NZ_ACCH00000000 | | | 0.991 | 0.952 | 0.996 | 0.942 |
| Bacteroidetes | Bacteroides | ovatus | NZ_AAXF00000000 | 0.776 | 0.557 | | | | |
| Bacteroidetes | Bacteroides | uniformis | NZ_AAYH00000000 | 0.996 | 1.113 | 0.990 | 0.964 | 0.993 | 0.891 |
| Bacteroidetes | Bacteroides | dorei | NZ_ABWZ00000000 | 0.995 | 0.959 | | | 0.985 | 1.259 |
| Bacteroidetes | Bacteroides | eggerthii | NZ_ABVO00000000 | 0.993 | 0.935 | 0.993 | 1.018 | 0.987 | 1.097 |
| Bacteroidetes | Bacteroides | finegoldii | NZ_ABXI00000000 | 0.996 | 1.081 | 0.995 | 0.928 | 0.996 | 0.920 |
| Bacteroidetes | Bacteroides | intestinalis | NZ_ABJL00000000 | | | 0.992 | 1.004 | 0.987 | 1.019 |
| Bacteroidetes | Bacteroides | thetaiotaomicron | NC_Bthetaiotaomicron3731 | | | | | | |
| | | 3731 | | | | | | | |
| Bacteroidetes | Bacteroides | thetaiotaomicron | NC_Bthetaiotaomicron7330 | | | | | | |
| | | 7330 | | | | | | | |
| Bacteroidetes | Bacteroides | thetaiotaomicron | NC_004663 | | | | | | |
| | | VPI-5482 | | | | | | | |
| Bacteroidetes | Bacteroides | vulgatus | NC_009614 | 0.992 | 1.011 | | | 0.987 | 0.962 |
| Bacteroidetes | Bacteroides | xylanisolvens | NC_BxylanisolvensXB1A | | | | | | |
| Bacteroidetes | Parabacteroides | johnsonii | NZ_ABYH00000000 | 0.998 | 1.090 | 0.996 | 0.943 | 0.995 | 0.945 |
| Firmicute | Anaerococcus | hydrogenalis | NZ_ABXA00000000 | 0.995 | 1.015 | 0.995 | 1.018 | 0.998 | 1.064 |
| Firmicute | Anaerotruncus | colihominis | NZ_ABGD00000000 | 0.994 | 0.893 | 0.994 | 1.022 | 0.997 | 1.012 |
| Firmicute | Blautia | hansenii | NZ_ABYU00000000 | | | 0.991 | 1.013 | 0.983 | 0.934 |
| Firmicute | Clostridium | leptum | NZ_ABCB00000000 | 0.981 | 1.002 | 0.988 | 1.003 | 0.970 | 0.965 |
| Firmicute | Clostridium | nexile-related A2- | NC_Cnexile1787 | | | | | | |
| | | 232 | | | | | | | |
| Firmicute | Clostridium | saccharolyticum- | NZ_ACFX00000000 | 0.993 | 1.040 | 0.991 | 0.994 | 0.985 | 0.963 |
| | | related | | | | | | | |
| Firmicute | Clostridium | asparagiforme | NZ_ACCJ00000000 | 0.996 | 1.128 | 0.990 | 0.859 | 0.959 | 0.874 |
| Firmicute | Clostridium | nexile | NZ_ABWO00000000 | 0.988 | 1.019 | | | | |
| Firmicute | Clostridium | sporogenes | NZ_ABKW00000000 | 0.995 | 0.927 | 0.996 | 1.020 | 0.994 | 1.066 |
| Firmicute | Coprococcus | comes | NZ_ABVR00000000 | 0.989 | 1.080 | | | | |
| Firmicute | Dorea | formicigenerans | NZ_AAXA00000000 | 0.993 | 0.978 | 0.993 | 0.952 | 0.988 | 0.953 |
| Firmicute | Dorea | longicatena | NZ_AAXB00000000 | 0.995 | 0.986 | 0.994 | 1.060 | 0.989 | 1.086 |
| Firmicute | Eubacterium | eligens | NC_012778 | 0.997 | 0.903 | 0.994 | 1.052 | 0.991 | 1.006 |
| Firmicute | Eubacterium | biforme | NZ_ABYT00000000 | 0.990 | 1.010 | 0.986 | 0.957 | 0.991 | 0.974 |
| Firmicute | Eubacterium | ventriosum | NZ_AAVL00000000 | 0.958 | | 0.991 | | | |
| Firmicute | Faecalibacterium | prausnitzii M21/2 | NZ_ABED00000000 | 0.976 | 1.124 | 0.990 | 0.980 | 0.971 | 0.896 |
| Firmicute | Roseburia | intestinalis | NZ_ABYJ00000000 | 0.994 | 1.003 | 0.991 | 0.999 | 0.987 | 1.016 |
| Firmicute | Ruminococcus | gnavus | NZ_AAYG00000000 | | | 0.995 | 1.058 | 0.993 | 1.093 |
| Firmicute | Ruminococcus | lactaris | NZ_ABOU00000000 | 0.993 | 1.025 | 0.993 | 0.935 | 0.989 | 0.911 |
| Firmicute | Ruminococcus | torques | NZ_AAVP00000000 | 0.997 | 0.959 | 0.996 | 1.047 | 0.998 | 1.040 |
| Firmicute | Streptococcus | infantarius | NZ_ABJK00000000 | 0.995 | 1.027 | 0.995 | 0.952 | 0.993 | 0.986 |
| Firmicute | Subdoligranulum | variabile | NZ_ACBY00000000 | 0.996 | 1.027 | 0.996 | 0.943 | 0.994 | 0.959 |
| Proteobacteria | Edwardsiella | tarda | NZ_ADGK00000000 | | | 0.992 | 0.941 | 0.988 | 0.937 |
| Proteobacteria | Enterobacter | cancerogenus | NC_Ecancerogenus | 0.998 | 0.962 | 0.992 | 1.072 | 0.993 | 1.080 |
| Proteobacteria | Escherichia | coli K12 | NC_000913 | 0.898 | 0.805 | | | | |
| Proteobacteria | Escherichia | fergusonii | NC_011740 | 0.998 | 1.075 | | | | |
| Verrucomicrobia | Akkermansia | muciniphila | NC_010655 | | | 0.995 | 0.971 | 0.970 | 1.045 |
| Bacteroidetes | Bacteroides | thetaiotaomicron | NC_Bthetaiotaomicron7330, | 0.938 | 0.806 | | | | |
| | | VPI-5482, 7330, | NC_004663, | | | | | | |
| | | 3731 | NC_Bthetaiotaomicron3731 | | | | | | |
| Bacteroidetes | Bacteroides | ovatus, | NZ_AAXF00000000, | 0.860 | 0.567 | 0.843 | 1.100 | 0.832 | 1.113 |
| | | xylanisolvens XB1A | NC_BxylanisolvensXB1A | | | | | | |
| Bacteroidetes | Bacteroides | intestinalis, | NZ_ABJL00000000, | 0.938 | 1.008 | | | | |
| | | cellulosilyticus | NZ_ACCH00000000 | | | | | | |
| Bacteroidetes | Bacteroides | thetaiotaomicron | NC_Bthetaiotaomicron7330, | | | 0.955 | 1.278 | 0.966 | 1.314 |
| | | VPI-5482, 3731 | NC_004663 | | | | | | |
| Proteobacteria | Escherichia | coli, fegusonii | NC_000913, NC_011740 | | | 0.909 | 0.944 | 0.896 | 0.964 |
| Bacteroidetes | Bacteroides | vulgatus, dorei | NC_009614, | | | 0.946 | 0.743 | | |
| | | | NZ_ABWZ00000000 | | | | | | |
| Firmicute | Clostridium, | nexile-related A2- | NC_Cnexile1787, | | | 0.984 | 0.964 | 0.986 | 0.973 |
| | Coprococcus | 232, comes | NZ_ABVR00000000 | | | | | | |
| Mean | | | | 0.975 | 0.978 | 0.983 | 0.981 | 0.981 | 1.001 |
| Min | | | | 0.776 | 0.557 | 0.843 | 0.743 | 0.832 | 0.857 |
| Max | | | | 0.998 | 1.171 | 0.996 | 1.278 | 0.998 | 1.314 |

KEY
Not Detected
Non-Unique
Not-Accurate (<0.7)
Strains not detected in any sample

| | | | |
|---|---|---|---|
| Firmicute | Blautia | luti | NC_Bluti |
| Firmicute | Clostridium | hathewayi | NZ_ACIO00000000 |
| Proteobacteria | Providencia | alcalifaciens | NZ_ABXW00000000 |
| Proteobacteria | Proteus | penneri | NZ_ABVP00000000 |

TABLE 16
Estimating the Jaccard index between samples with LEA-Seq.
A. Known proportion of shared strains (Jaccard Index) between
four bacterial DNA spike-in pools of differing strain composition.

| mock community | 3 member | 6 member | 32 member | 48 member |
|---|---|---|---|---|
| 3 member | 1.000 | 0.538 | 0.167 | 0.111 |
| 6 member | 0.538 | 1.000 | 0.310 | 0.158 |
| 32 member | 0.167 | 0.310 | 1.000 | 0.301 |
| 48 member | 0.111 | 0.158 | 0.301 | 1.000 |

B. Performance of LEA-Seq in measuring shared-strains
between two samples.

| | abs | | |
|---|---|---|---|
| | (known-measured) | | correlation |
| | mean | std | (known vs measured) |
| all samples | 0.1138 | 0.1253 | 0.9349 |
| samples on same run | 0.0269 | 0.0242 | 0.9963 |
| samples on different runs | 0.1821 | 0.1303 | 0.9894 |

abs = absolute value
known = known value of the Jaccard index
measured = value of Jaccard index determined with LEA-Seq

What is claimed is:

1. A method for sequencing, the method comprising:
a) contacting sample comprising nucleic acid with a finite amount of linear primer, wherein the linear primer comprises: (i) an adapter, (ii) a random component, and (iii) a target specific sequence;
b) performing linear PCR, wherein the performing linear PCR generates a finite number of products and wherein a product of linear PCR comprises the adapter, the random component and the target specific sequence;
c) contacting the product from (b) with 3 types of primers;
i. primer type 1 comprising an adapter complementary to the adapter from (a);
ii. primer type 2 comprising a target specific sequence that is 3′ of the target specific sequence in (a) and an adapter and wherein primer type 2 is diluted relative to primer type 1 and primer type 3; and
iii. primer type 3 comprising an adapter complementary to the adapter in (ii) and an index sequence;
d) performing exponential PCR, wherein the products from (b) are amplified and wherein the products of (d) comprise in the 5′ to 3′ direction: the adapter, the random component, the target specific sequences, the downstream adapter, and the index sequence and wherein steps (a)-(d) are performed in one reaction vial;
e) sequencing the product from (d), wherein redundant reads are generated and wherein the redundant reads are separated by the random component and a consensus sequence is identified such that the redundant reads improve the sequence quality.

2. The method of claim 1, wherein the adapter is an illumina adapter.

3. The method of claim 1, wherein the random component is about 16 to about 18 nucleotides.

4. The method of claim 1, wherein the target specific sequence is a sequence complementary to a 16S nucleic acid sequence.

5. The method of claim 4, wherein the 16S nucleic acid sequence is selected from the group consisting of the V1V2 region and the V4 region.

6. The method of claim 1, wherein the linear primer further comprises phasing nucleotides.

7. The method of claim 1, wherein primer type 2 further comprises phasing nucleotides

8. The method of claim 1, wherein primer type 2 is diluted about 1:20 to about 1:40 relative to primer type 2 and primer type 3.

9. The method of claim 1, wherein primer type 2 is diluted 1:30 relative to primer type 1 and primer type 3.

10. The method of claim 1, wherein the sample comprising nucleic acid is from a gut.

11. A method of sequencing microbial communities, the method comprising:
a) contacting sample comprising nucleic acid with a finite amount of linear primer, wherein the linear primer comprises: (i) an adapter, (ii) a random component, and (iii) a 16S sequence;
b) performing linear PCR, wherein the performing linear PCR generates a finite number of products and wherein a product of linear PCR comprises the adapter, the random component and the 16S sequence;
c) contacting the product from (b) with 3 types of primers;
i. primer type 1 comprising an adapter complementary to the adapter from (a);
ii. primer type 2 comprising a 16S sequence that is 3′ of the 16S sequence in (a) and an adapter and wherein primer type 2 is diluted relative to primer type 1 and primer type 3; and
iii. primer type 3 comprising an adapter complementary to the adapter in (ii) and an index sequence;
d) performing exponential PCR, wherein the products from (b) are amplified and wherein the products of (d) comprise in the 5′ to 3′ direction: the adapter, the random component, the 16S sequence, the downstream adapter, and the index sequence and wherein steps (a)-(d) are performed in one reaction vial;
e) sequencing the product from (d), wherein redundant reads are generated and wherein the redundant reads are separated by the random component and a consensus sequence is identified such that the redundant reads improve the sequence quality.

12. The method of claim 11, wherein the 16S sequence is selected from the group consisting of the V1V2 region and the V4 region.

13. The method of claim 11, wherein the sample is selected from the group consisting of a gut sample and an environmental sample.

14. A method to improve sequencing quality and depth, the method comprising:
a) performing linear PCR, wherein the linear PCR reaction comprises sample comprising nucleic acid and a finite amount of linear primer comprising a random component and a target specific sequence and wherein the linear PCR generates less product than the sequencing depth;
b) performing exponential PCR, wherein the exponential PCR reaction amplifies the linear PCR product from (a)
c) sequencing the exponential PCR product from (b), wherein the sequence quality and depth is improved.

15. The method of claim 14, wherein the linear primer further comprises an adapter.

16. The method of claim 14, wherein steps (a) and (b) are performed in the same reaction vial.

17. The method of claim 14, wherein the exponential PCR reaction comprises three types of primers that amplify the target specific sequence.

18. The method of claim 14, wherein the sequencing generates redundant reads which are error-corrected to generate a consensus sequence.


SEQUENCE LISTING (36 sequences)

SEQ ID NO: 1 - DNA, 37 bp, Artificial Sequence
  SYNTHESIZED
ccgtgtctcagtcccaatgtggccggtcaccctctca

SEQ ID NO: 2 - DNA, 37 bp, Artificial Sequence
  SYNTHESIZED
ccgtgactcagtcccaatgtggccggtcaccctctca

SEQ ID NO: 3 - DNA, 37 bp, Artificial Sequence
  SYNTEHSIZED
ccgtgtctcagtcccaatgtggccggtcaccctctaa

SEQ ID NO: 4 - DNA, 65 bp, Artificial Sequence
  SYNTHESIZED
gtcagtcagcagtaaattgaagagtttgatcatggctcagattgaacggcaggcctaacacatgc

SEQ ID NO: 5 - DNA, 31 bp, Artificial Sequence
  SYNTHESIZED
actcctacgggaggcagcatcagctagcagt

SEQ ID NO: 6 - DNA, 31 bp, Artificial Sequence
  SYNTHESIZED
actgctagctgatgctgcctcccgtaggagt

SEQ ID NO: 7 - DNA, 65 bp, Artificial Sequence
  SYNTHESIZED
gcatgtgttaggcctgccgttcaatctgagccatgatcaaactcttcaatttactgctgactgac

SEQ ID NO: 8 - DNA, 53 bp, Artificial Sequence
  SYNTHESIZED
cgacgctcttccgatctnnnhnnnwnnnhnnnhagagtttgatcctggctcag

SEQ ID NO: 9 - DNA, 54 bp, Artificial Sequence
  SYNTHESIZED
cgacgctcttccgatctnnnhnnnwnnnhnnnhnagagtttgatcctggctcag

SEQ ID NO: 10 - DNA, 55 bp, Artificial Sequence
  SYNTHESIZED
cgacgctcttccgatctnnnhnnnwnnnhnnnhnnagagtttgatcctggctcag

SEQ ID NO: 11 - DNA, 58 bp, Artificial Sequence
  SYNTHESIZED
aatgatacggcgaccaccgagatctacactctttccctacacgacgctcttccgatct

SEQ ID NO: 12 - DNA, 80 bp, Artificial Sequence
  SYNTHESIZED
cgacgctcttccgatctnnnhnnnwnnnhnnnhnnagagtttgatcctggctcagattgaacggcaggcctaacacatgc

SEQ ID NO: 13 - DNA, 31 bp, Artificial Sequence; identical to SEQ ID NO: 5
  SYNTHESIZED

SEQ ID NO: 14 - DNA, 54 bp, Artificial Sequence
  SYNTHESIZED
gtgactggagttcagacgtgtgctcttccgatctttgctgcctcccgtaggagt

SEQ ID NO: 15 - DNA, 55 bp, Artificial Sequence
  SYNTHESIZED
gtgactggagttcagacgtgtgctcttccgatcttgtgctgcctcccgtaggagt

SEQ ID NO: 16 - DNA, 56 bp, Artificial Sequence
  SYNTHESIZED
gtgactggagttcagacgtgtgctcttccgatctcactgctgcctcccgtaggagt

SEQ ID NO: 17 - DNA, 51 bp, Artificial Sequence
  SYNTHESIZED
caagcagaagacggcatacgagatnnnnnnngtgactggagttcagacgtg

SEQ ID NO: 18 - DNA, 121 bp, Artificial Sequence
  SYNTHESIZED
aatgatacggcgaccaccgagatctacactctttccctacacgacgctcttccgatctnnnhnnnwnnnhnnnhnnagagtttgatcctggctcagattgaacggcaggcctaacacatgc

SEQ ID NO: 19 - DNA, 87 bp, Artificial Sequence
  SYNTHESIZED
actcctacgggaggcagcanngagatcggaagagcacacgtctgaactccagtcacnnnnnnnatctcgtatgccgtcttctgcttg

SEQ ID NO: 20 - DNA, 87 bp, Artificial Sequence
  SYNTHESIZED
caagcagaagacggcatacgagatnnnnnnngtgactggagttcagacgtgtgctcttccgatctcnntgctgcctcccgtaggagt

SEQ ID NO: 21 - DNA, 121 bp, Artificial Sequence
  SYNTHESIZED
gcatgtgttaggcctgccgttcaatctgagccaggatcaaactctnnhnnnhnnnwnnhnnnnagatcggaagagcgtcgtgtagggaaagagtgtagatctcggtggtcgccgtatcatt

SEQ ID NO: 22 - DNA, 77 bp, Artificial Sequence
  SYNTHESIZED
aatgatacggcgaccaccgagatctacactctttccctacacgacgctcttccgatctgtgccagcagccgcggtaa

SEQ ID NO: 23 - DNA, 78 bp, Artificial Sequence
  SYNTHESIZED
aatgatacggcgaccaccgagatctacactctttccctacacgacgctcttccgatctcgtgccagcagccgcggtaa

SEQ ID NO: 24 - DNA, 79 bp, Artificial Sequence
  SYNTHESIZED
aatgatacggcgaccaccgagatctacactctttccctacacgacgctcttccgatctacgtgccagcagccgcggtaa

SEQ ID NO: 25 - DNA, 20 bp, Artificial Sequence
  SYNTHESIZED
ggactaccagggtatctaat

SEQ ID NO: 26 - DNA, 25 bp, Artificial Sequence
  SYNTHESIZED
ttaccgcggctgctggcacggagtt

SEQ ID NO: 27 - DNA, 19 bp, Artificial Sequence
  SYNTHESIZED
gtgccagcagccgcggtaa

SEQ ID NO: 28 - DNA, 34 bp, Artificial Sequence
  SYNTHESIZED
attagataccctggtagtccacgccgtaaacgat

SEQ ID NO: 29 - DNA, 56 bp, Artificial Sequence
  SYNTHESIZED
gtgactggagttcagacgtgtgctcttccgatctcaggactaccagggtatctaat

SEQ ID NO: 30 - DNA, 55 bp, Artificial Sequence
  SYNTHESIZED
gtgactggagttcagacgtgtgctcttccgatctaggactaccagggtatctaat

SEQ ID NO: 31 - DNA, 54 bp, Artificial Sequence
  SYNTHESIZED
gtgactggagttcagacgtgtgctcttccgatctggactaccagggtatctaat

SEQ ID NO: 32 - DNA, 51 bp, Artificial Sequence
  SYNTHESIZED
caagcagaagacggcatacgagatacgcctcgtgactggagttcagacgtg

SEQ ID NO: 33 - DNA, 77 bp, Artificial Sequence; identical to SEQ ID NO: 22
  SYNTHESIZED

SEQ ID NO: 34 - DNA, 85 bp, Artificial Sequence
  SYNTHESIZED
attagataccctggtagtccagatcggaagagcacacgtctgaactccagtcacgaggcgtatctcgtatgccgtcttctgcttg

SEQ ID NO: 35 - DNA, 85 bp, Artificial Sequence
  SYNTHESIZED
caagcagaagacggcatacgagatacgcctcgtgactggagttcagacgtgtgctcttccgatctggactaccagggtatctaat

SEQ ID NO: 36 - DNA, 77 bp, Artificial Sequence
  SYNTHESIZED
ttaccgcggctgctggcacagatcggaagagcgtcgtgtagggaaagagtgtagatctcggtggtcgccgtatcatt